(19) United States
(12) Patent Application Publication
(10) Pub. No.: US 2024/0217969 A1
(43) Pub. Date: Jul. 4, 2024

(54) SUBSTITUTED PYRROLE CARBOXAMIDES, PROCESS FOR THEIR PREPARATION AND THEIR USE AS KINASE INHIBITORS

(71) Applicant: NERVIANO MEDICAL SCIENCES S.R.L., Nerviano (MI) (JP)

(72) Inventors: Helena POSTERI, Travedona Monate (VA) (IT); Laura BUFFA, Rossiglione (GE) (IT); Danilo MIRIZZI, Nerviano (MI) (IT); Ilaria MOTTO, Nerviano (IT); Maria MENICHINCHERI, Milan (IT) (IT)

(73) Assignee: NERVIANO MEDICAL SCIENCES S.R.L., Nerviano (MI) (IT)

(21) Appl. No.: 18/285,323

(22) PCT Filed: Mar. 22, 2022
(86) PCT No.: PCT/EP2022/057452

§ 371 Date: Oct. 2, 2023

(30) Foreign Application Priority Data
Apr. 2, 2021 (EP) 21166838.9

(51) Int. Cl. C07D 471/04; A61K 31/4155; A61K 31/427; A61K 31/437; A61K 31/506; A61K 31/5377; C07D 403/04; C07D 417/04

(52) U.S. Cl. CPC C07D 471/04; A61K 31/4155; A61K 31/427; A61K 31/437; A61K 31/506; A61K 31/5377; C07D 403/04; C07D 417/04

(57) ABSTRACT

The application relates to substituted pyrrole carboxamide derivatives of formula (I) which modulate the activity of cycle 7-related protein kinase (Cdc7). The compounds of this invention are therefore useful in treating diseases related to dysregulated kinases activity, for example cancer, cell proliferative disorders, viral infections, immune disorders, neurodegenerative disorders, cardiovascular diseases and bone related diseases. The application also provides methods for preparing these compounds, pharmaceutical compositions comprising these compounds, and their medical uses.

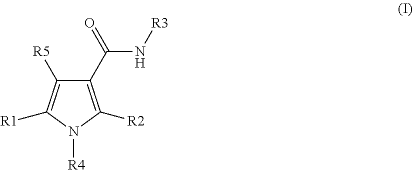

The present invention relates to certain substituted pyrrole compounds which modulate the activity of protein kinases. The compounds of this invention are therefore useful in treating diseases related to dysregulated kinases activity, for example cancer, cell proliferative disorders, viral infections, immune disorders, neurodegenerative disorders, cardiovascular diseases and bone related diseases.

The present invention also provides methods for preparing these compounds, pharmaceutical compositions comprising these compounds, and methods of treating diseases utilizing pharmaceutical compositions comprising these compounds.

### BACKGROUND OF THE INVENTION

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases. For a general reference to PKs malfunctioning or dysregulation see, for instance, Current Opinion in Chemical Biology 1999, 3, 459-465.

Among the several protein kinases known in the art as being implicated in the growth of cancer cells is cell division cycle 7-related protein kinase (Cdc7), a key cell cycle protein required for DNA replication by catalyzing MCM helicase activation at DNA replication origins. Cdc7 kinase and its regulatory subunit Dbf4 are overexpressed in many tumors and overexpression correlates with poor prognosis and advanced tumor grade. Cdc7 is also implicated in mediating the processing of stalled replication forks once the replication stress or damage has been resolved and in translesion DNA repair (see Montagnoli A. et al., EMBO Journal, 2002, Vol. 21, No. 12, 3171; Montagnoli A. et al., Cancer Research 2004, Vol. 64, October 1, 7110; Hou Y. et al., Mol Oncol 2012, Vol. 29, 3498; Day T A et al, 2010, J Cell Biol Vol. 191, 953).

Pyyrrole carboxamide derivatives are known in the art as protein kinase inhibitors. Among them, for instance, WO2009/040399 reports pyrimidinyl-pyrrole derivatives useful in the therapy of diseases associated with dysregulated protein kinase activity, particularly the Polo Like Kinase (PLK) family; WO2013/014039 and WO2014/019908 disclose pyrimidinyl-pyrrole derivatives endowed with Janus Kinase (JAK) and Src inhibitory activity, while WO2007/110344 claimes pyrimidinyl-pyrrole compounds endowed with inhibitory activity toward Cdc7 protein kinase activity.

Drug Metabolism and Pharmacokinetics (DMPK) is primarily associated with safety evaluation in drug discovery and in the drug development process. In addition to adequate potency against the target protein, an acceptable safety profile is required to increase the chance of a candidate drug becoming a successful therapy.

Taking the above into consideration, there is a strong need for the development of Cdc7 inhibitors for the treatment of cancer and other diseases. The present inventors have now identified novel pyrrole carboxamide compounds endowed with inhibitory activity toward Cdc7 protein kinase. The compounds of the invention potent inhibitors of the Cdc7 kinase and they surprisingly show enhanced metabolic stability properties compared to compounds of the same chemical class disclosed in the prior art. These improved properties will be discussed in more details in the chapter “Experimental part” below.

Due to the key role of PKs, in particular of Cdc7, in the regulation of cellular proliferation, these pyrrole carboxamide derivatives are particularly useful in the treatment of cancer as well as in the treatment of a variety of cell proliferative disorders and immune-related disorders.

### SUMMARY OF THE INVENTION

Accordingly, a first object of the present invention is to provide a substituted pyrrole carboxamide compounds represented by formula (I)

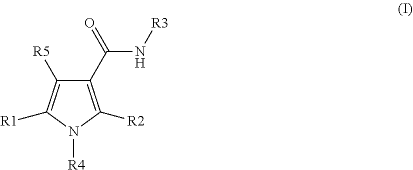

wherein:
R1 is a heteroaryl group selected from the group consisting of:

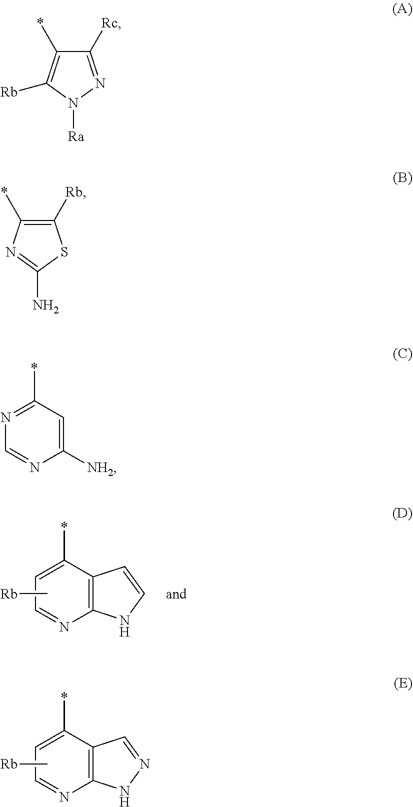

wherein:
   Ra, Rb and Rc are independently hydrogen, an optionally substituted straight or branched (C₁-C₆) alkyl or an optionally substituted straight or branched (C₂-C₆) alkenyl;
R2 is a substituted aryl or a substituted heteroaryl ring bearing from one up to three substituents selected from halogen, nitro, amino, (C₁-C₆) alkyl amino, aminocarbonyl, an optionally substituted straight or branched (C₁-C₆) alkyl, an optionally substituted straight or branched (C₁-C₆) alkoxy, an optionally substituted straight or branched polyfluorinated (C₁-C₆) alkyl and optionally substituted straight or branched polyfluorinated (C₁-C₆) alkoxy;
   provided that, 2,5-disubstituted phenyl group is excluded;
R3 is hydrogen, an optionally substituted straight or branched (C₁-C₄) alkyl, an optionally substituted (C₃-C₆) cycloalkyl group, or an optionally substituted (C₅-C₆) heterocyclyl group;
R4 is hydrogen, an optionally substituted straight or branched (C₁-C₆) alkyl or an optionally substituted straight or branched (C₂-C₆) alkenyl; and
R5 is hydrogen, halogen or an optionally substituted straight or branched (C₁-C₃) alkyl;

or a pharmaceutically acceptable salt thereof.

Preferred compounds of formula (I) are the compounds wherein:
R1 is an optionally substituted heteroaryl group selected from the group (A), (B), (C), (D) and (E);
   wherein:
   Ra, Rb and Rc are independently hydrogen or an optionally substituted straight or branched (C1-C6) alkyl;
R2 is a 2,4-disubstituted phenyl, 4,6-disubstituted pyridin-3-yl, 2,6-disubstituted pyridin-3-yl or 3,5-disubstituted pyridin-2-yl; and
R3, R4 and R5 are as defined above.

More preferred compounds of formula (I) are the compounds wherein:
R2 is a 2,4-disubstituted phenyl;
R3 is hydrogen or an optionally substituted straight or branched (C₁-C₄) alkyl chain;
R5 is hydrogen; and
R1 and R4 are as defined above.

Even more preferred compounds of formula (I) are the compounds wherein:
R4 is hydrogen; and
R1, R2, R3 and R5 are as defined above.

Preferred specific compounds of formula (I), or a pharmaceutically acceptable salt thereof, are the compounds listed below:
2-(3-chloro-2-fluorophenyl)-5-(1H-pyrrolo[2,3-b]pyridin-4-yl)-1H-pyrrole-3-carboxamide (comp 1);
2-(4-chloro-2-fluorophenyl)-5-(1H-pyrrolo[2,3-b]pyridin-4-yl)-1H-pyrrole-3-carboxamide (comp 2);
2-(2-chloro-4-fluorophenyl)-5-(1H-pyrrolo[2,3-b]pyridin-4-yl)-1H-pyrrole-3-carboxamide (comp 3);
2-(2,4-difluorophenyl)-5-(1H-pyrrolo[2,3-b]pyridin-4-yl)-1H-pyrrole-3-carboxamide (comp 4);
2-[2-chloro-4-(trifluoromethyl)phenyl]-5-(1H-pyrrolo[2,3-b]pyridin-4-yl)-1H-pyrrole-3-carboxamide (comp 5);
2-(2,3-difluorophenyl)-5-(1H-pyrrolo[2,3-b]pyridin-4-yl)-1H-pyrrole-3-carboxamide (comp 6);
2-(2,3-dichlorophenyl)-5-(1H-pyrrolo[2,3-b]pyridin-4-yl)-1H-pyrrole-3-carboxamide (comp 7);
2-[4-methyl-2-(trifluoromethyl)phenyl]-5-(1H-pyrrolo[2,3-b]pyridin-4-yl)-1H-pyrrole-3-carboxamide (comp 8);
2-(2-chloro-4-methylphenyl)-5-(1H-pyrrolo[2,3-b]pyridin-4-yl)-1H-pyrrole-3-carboxamide (comp 9);
2-(2,3-difluoro-4-methylphenyl)-5-(1H-pyrrolo[2,3-b]pyridin-4-yl)-1H-pyrrole-3-carboxamide (comp 10);
2-[2-methyl-4-(trifluoromethyl)phenyl]-5-(1H-pyrrolo[2,3-b]pyridin-4-yl)-1H-pyrrole-3-carboxamide (comp 11);
2-(2-fluoro-3-methoxyphenyl)-5-(1H-pyrrolo[2,3-b]pyridin-4-yl)-1H-pyrrole-3-carboxamide (comp 12);
2-(2-chloro-3-fluorophenyl)-5-(1H-pyrrolo[2,3-b]pyridin-4-yl)-1H-pyrrole-3-carboxamide (comp 13);
2-(2-fluoro-3-methylphenyl)-5-(1H-pyrrolo[2,3-b]pyridin-4-yl)-1H-pyrrole-3-carboxamide (comp 14);
2-[2-methyl-3-(trifluoromethyl)phenyl]-5-(1H-pyrrolo[2,3-b]pyridin-4-yl)-1H-pyrrole-3-carboxamide (comp 15);
2-[4-methoxy-2-(trifluoromethyl)phenyl]-5-(1H-pyrrolo[2,3-b]pyridin-4-yl)-1H-pyrrole-3-carboxamide (comp 16);
2-[2-chloro-4-(difluoromethoxy)phenyl]-5-(1H-pyrrolo[2,3-b]pyridin-4-yl)-1H-pyrrole-3-carboxamide (comp 17);
2-(3,4-dichlorophenyl)-5-(1H-pyrrolo[2,3-b]pyridin-4-yl)-1H-pyrrole-3-carboxamide (comp 18);
2-(3,4-difluorophenyl)-5-(1H-pyrrolo[2,3-b]pyridin-4-yl)-1H-pyrrole-3-carboxamide (comp 19);
2-(3-ethoxy-2-fluorophenyl)-5-(1H-pyrrolo[2,3-b]pyridin-4-yl)-1H-pyrrole-3-carboxamide (comp 20);
2-(4-methyl-3-nitrophenyl)-5-(1H-pyrrolo[2,3-b]pyridin-4-yl)-1H-pyrrole-3-carboxamide (comp 21);
2-(3-carbamoyl-4-fluorophenyl)-5-(1H-pyrrolo[2,3-b]pyridin-4-yl)-1H-pyrrole-3-carboxamide (comp 22);
2-(2-fluoro-4-methylphenyl)-N-[2-(pyrrolidin-1-yl)ethyl]-5-(1H-pyrrolo[2,3-b]pyridin-4-yl)-1H-pyrrole-3-carboxamide (comp 23);
N-[2-(dimethylamino)ethyl]-2-(2-fluoro-4-methylphenyl)-5-(1H-pyrrolo[2,3-b]pyridin-4-yl)-1H-pyrrole-3-carboxamide (comp 24);
2-(2-fluoro-4-methylphenyl)-N-[2-(morpholin-4-yl)ethyl]-5-(1H-pyrrolo[2,3-b]pyridin-4-yl)-1H-pyrrole-3-carboxamide (comp 25);
N-[(1S,2R)-2-aminocyclohexyl]-2-(2-fluoro-4-methylphenyl)-5-(1H-pyrrolo[2,3-b]pyridin-4-yl)-1H-pyrrole-3-carboxamide (comp 26);
2-(2-fluoro-4-methylphenyl)-N-(furan-2-ylmethyl)-5-(1H-pyrrolo[2,3-b]pyridin-4-yl)-1H-pyrrole-3-carboxamide (comp 27);
N-(fluoroethyl)-2-(2-fluoro-4-methylphenyl)-5-(1H-pyrrolo[2,3-b]pyridin-4-yl)-1H-pyrrole-3-carboxamide (comp 28);
2-(2-fluoro-4-methylphenyl)-N-[2-(methylamino)ethyl]-5-(1H-pyrrolo[2,3-b]pyridin-4-yl)-1H-pyrrole-3-carboxamide (comp 29);
2-(2-fluoro-4-methylphenyl)-N-(1-methylpiperidin-4-yl)-5-(1H-pyrrolo[2,3-b]pyridin-4-yl)-1H-pyrrole-3-carboxamide (comp 30);
2-(dibenzo[b,d]thiophen-4-yl)-5-(1H-pyrrolo[2,3-b]pyridin-4-yl)-1H-pyrrole-3-carboxamide (comp 31);
2-(4-methylnaphthalen-1-yl)-5-(1H-pyrrolo[2,3-b]pyridin-4-yl)-1H-pyrrole-3-carboxamide (comp 32);
2-(3-fluorophenyl)-5-(1H-pyrrolo[2,3-b]pyridin-4-yl)-1H-pyrrole-3-carboxamide (comp 33);
5-(1H-pyrrolo[2,3-b]pyridin-4-yl)-2-[4-(trifluoromethoxy)phenyl]-1H-pyrrole-3-carboxamide (comp 34);
2-(1-benzothiophen-3-yl)-5-(1H-pyrrolo[2,3-b]pyridin-4-yl)-1H-pyrrole-3-carboxamide (comp 35);
2-(2,3-dihydro-1,4-benzodioxin-6-yl)-5-(1H-pyrrolo[2,3-b]pyridin-4-yl)-1H-pyrrole-3-carboxamide (comp 36);
2-(4-fluoro-2-methylphenyl)-5-(1H-pyrrolo[2,3-b]pyridin-4-yl)-1H-pyrrole-3-carboxamide (comp 37);
2-(2-fluoro-4-methylphenyl)-4-iodo-5-(1H-pyrrolo[2,3-b]pyridin-4-yl)-1H-pyrrole-3-carboxamide (comp 38);
4-bromo-2-(2-fluoro-4-methylphenyl)-5-(1H-pyrrolo[2,3-b]pyridin-4-yl)-1H-pyrrole-3-carboxamide (comp 39);
4-ethyl-2-(2-fluoro-4-methylphenyl)-5-(1H-pyrrolo[2,3-b]pyridin-4-yl)-1H-pyrrole-3-carboxamide (comp 40);
2-(2-fluoro-4-methylphenyl)-4-(propan-2-yl)-5-(1H-pyrrolo[2,3-b]pyridin-4-yl)-1H-pyrrole-3-carboxamide (comp 41);
5-(6-aminopyrimidin-4-yl)-2-(2,4-dichlorophenyl)-1H-pyrrole-3-carboxamide (comp 42)
2-(2,4-dichlorophenyl)-5-(1H-pyrazol-4-yl)-1H-pyrrole-3-carboxamide (comp 43);
2-(2,4-dichlorophenyl)-5-(3-methyl-1H-pyrazol-4-yl)-1H-pyrrole-3-carboxamide (comp 44);
5-(2-amino-1,3-thiazol-4-yl)-2-(2,4-dichlorophenyl)-1H-pyrrole-3-carboxamide (comp 45);
2-(2,4-dichlorophenyl)-5-(1H-pyrrolo[2,3-b]pyridin-4-yl)-1H-pyrrole-3-carboxamide (comp 46);
2-(2,4-dichlorophenyl)-5-(1H-pyrazolo[3,4-b]pyridin-4-yl)-1H-pyrrole-3-carboxamide (comp 47);
2-(2,4-dichlorophenyl)-5-[3-(trifluoromethyl)-1H-pyrazol-4-yl]-1H-pyrrole-3-carboxamide (comp 48);
2-(2-fluoro-4-methylphenyl)-5-(1H-pyrazol-4-yl)-1H-pyrrole-3-carboxamide (comp 49);
5-(3,5-dimethyl-1H-pyrazol-4-yl)-2-(2-fluoro-4-methylphenyl)-1H-pyrrole-3-carboxamide (comp 50);
2-(2-fluoro-4-methylphenyl)-5-(1-methyl-1H-pyrazol-4-yl)-1H-pyrrole-3-carboxamide (comp 51);
2-(2-fluoro-4-methylphenyl)-5-(3-methyl-1H-pyrazol-4-yl)-1H-pyrrole-3-carboxamide (comp 52);
2-(2-fluoro-4-methylphenyl)-5-(1H-pyrrolo[2,3-b]pyridin-4-yl)-1H-pyrrole-3-carboxamide (comp 53);
2-(2,4-dichlorophenyl)-1-(2-hydroxyethyl)-5-(1H-pyrrolo[2,3-b]pyridin-4-yl)-1H-pyrrole-3-carboxamide (comp 54);
2-(2,4-dichlorophenyl)-5-(1H-pyrrolo[2,3-b]pyridin-4-yl)-1-(3,3,3-trifluoropropyl)-1H-pyrrole-3-carboxamide (comp 55);
2-(2,4-dichlorophenyl)-1-methyl-5-(1H-pyrrolo[2,3-b]pyridin-4-yl)-1H-pyrrole-3-carboxamide (comp 56); and
2-(2,4-dichlorophenyl)-1-ethyl-5-(1H-pyrrolo[2,3-b]pyridin-4-yl)-1H-pyrrole-3-carboxamide (comp 57).

If a stereogenic center or another form of an asymmetric center is present in a compound of the present invention, all forms of such optical isomer or isomers, including enantiomers and diastereomers, are intended to be covered herein. Compounds containing a stereogenic center may be used as a racemic mixture, an enantiomerically enriched mixture, or the racemic mixture may be separated using well-known techniques and an individual enantiomer may be used. Such procedures comprise standard chromatographic techniques, including chromatography using a chiral stationary phase, or crystallization. General methods for separation of compounds containing one or more asymmetric centers are reported, for instance, in Jacques, Jean; Collet, Andre; Wilen, Samuel H., Enantiomers, Racemates, and Resolutions, John Wiley & Sons Inc., New York (NY), 1981.

In cases in which compounds have unsaturated carbon-carbon double bonds, both the cis (Z) and trans (E) isomers are within the scope of this invention.

In cases wherein compounds may exist in tautomeric forms, such as keto-enol tautomers, each tautomeric form is contemplated as being included within this invention whether existing in equilibrium or predominantly in one form.

Pharmaceutically acceptable salts of the compounds of formula (I) include the salts with inorganic or organic acids, e.g. nitric, hydrochloric, hydrobromic, sulfuric, perchloric, phosphoric, acetic, trifluoroacetic, propionic, glycolic, lactic, oxalic, fumaric, malonic, malic, maleic, tartaric, citric, benzoic, cinnamic, mandelic, methanesulphonic, isethionic and salicylic acid.

Pharmaceutically acceptable salts of the compounds of formula (I) also include the salts with inorganic or organic bases, e.g. alkali or alkaline-earth metals, especially sodium, potassium, calcium, ammonium or magnesium hydroxides, carbonates or bicarbonates, acyclic or cyclic amines.

Further object of the present invention are compounds of formula (I) wherein one or more hydrogen/s is/are replaced by one or more deutherium atom/s.

With the term “(C₁-C₆) alkyl”, we intend an aliphatic (C₁-C₆) hydrocarbon chain, containing carbon-carbon single bonds only, which can be straight or branched. Representative examples include, but are not limited to, methyl, ethyl, n-propyl, isopropyl, n-butyl, isobutyl, tert-butyl, sec-butyl, n-pentyl, n-hexyl, and the like.

With the term “(C₃-C₆) cycloalkyl”, we intend, unless otherwise provided, 3- to 6-membered all-carbon monocyclic ring, which may contain one or more double bonds, but does not have a completely conjugated π-electron system.

Examples of (C₃-C₆) cycloalkyl groups, without limitation, are cyclopropyl, cyclobutyl, cyclopentyl, cyclopentenyl, cyclohexanyl, cyclohexenyl and cyclohexadienyl.

With the term “(C₅-C₆) heterocyclyl”, we intend a 5 to 6-membered, saturated or partially unsaturated carbocyclic ring where one or more carbon atoms are replaced by heteroatoms such as nitrogen, oxygen and sulfur. Non limiting examples of heterocyclyl groups are, for instance, pyranyl, tetrahydropyranyl, pyrrolidinyl, pyrrolinyl, imidazolinyl, imidazolidinyl, pyrazolidinyl, pyrazolinyl, thiazolinyl, thiazolidinyl, dihydrofuranyl, tetrahydrofuranyl, tetrahydropyridinyl, 1,3-dioxolanyl, piperidinyl, piperazinyl, morpholinyl and the like. The heterocyclyl ring can be optionally further fused or linked to aromatic and non-aromatic carbocyclic or heterocyclic rings.

With the term “(C₂-C₆) alkenyl”, we intend an aliphatic straight or branched (C₂-C₆) hydrocarbon chain containing at least one carbon-carbon double bond. Representative examples include, but are not limited to, ethenyl, 1-propenyl, 2-propenyl, 1- or 2-butenyl, and the like.

The term “aryl” refers to a mono-, bi- or poly-carbocyclic hydrocarbon with from 1 to 4 ring systems, optionally further fused or linked to each other by single bonds, wherein at least one of the carbocyclic rings is “aromatic”, wherein the term “aromatic” refers to completely conjugated π-electron bond system. Non limiting examples of such aryl groups are phenyl, α- or β-naphthyl, α- or β-tetrahydronaphthalenyl, biphenyl, and indanyl groups.

The term “heteroaryl” refers to aromatic heterocyclic rings, typically 5- to 6-membered heterocycles with from 1 to 3 heteroatoms selected among N, O or S; the heteroaryl ring can be optionally further fused or linked to aromatic and non-aromatic carbocyclic and heterocyclic rings. Not limiting examples of such heteroaryl groups are, for instance, pyridyl, pyrazinyl, pyrimidinyl, pyridazinyl, indolyl, imidazolyl, thiazolyl, isothiazolyl, pyrrolyl, furanyl, oxazolyl, isoxazolyl, pyrazolyl, thiophenyl, thiadiazolyl, isoxazolyl, isothiazolyl, oxadiazolyl, indazolyl, cinnolinyl, benzo[1,3]dioxolyl, benzo[1,4]dioxinyl, benzothiazolyl, benzothiophenyl, benzofuranyl, isoindolinyl, benzoimidazolyl, benzoxazolyl, quinolinyl, isoquinolinyl, 1,2,3-triazolyl, 1-phenyl-1,2,3-triazolyl, 2,3-dihydroindolyl, 2,3-dihydrobenzofuranyl, 2,3-dihydrobenzothiophenyl, benzopyranyl, 2,3-dihydrobenzoxazinyl, 2,3-dihydroquinoxalinyl and the like.

With the term “halogen”, we intend fluoro, chloro, bromo or iodo.

With the term “polyfluorinated (C₁-C₆)alkyl” or “polyfluorinated (C₁-C₆)alkoxy”, we intend any of the above defined (C₁-C₆) alkyl or (C₁-C₆) alkoxy groups which are substituted by more than one fluoro atom such as, for instance, trifluoromethyl, trifluoroethyl, 1,1,1,3,3,3-hexafluoropropyl, trifluoromethoxy and the like.

With the term “hydroxy(C₁-C₆)alkyl” we intend any of the above defined (C₁-C₆)alkyl groups, bearing a hydroxyl group such as, for instance, hydroxymethyl, 2-hydroxyethyl, 3-hydroxypropyl and the like.

According to the present invention and unless otherwise provided, R1, R2, R3, R4, R5, Ra, Rb and Rc may be optionally substituted, in any of their free positions, by one or more groups, for instance 1 to 6 groups, independently selected from: hydroxyl, (C₁-C₆)alkoxy hydroxy(C₁-C₆)alkyl, halogen, nitro, oxo group (═O), cyano, (C₁-C₆)alkyl, polyfluorinated (C₁-C₆)alkyl, polyfluorinated (C₁-C₆)alkoxy, (C₂-C₆)alkenyl, (C₂-C₆)alkynyl, aryl, aryl(C₁-C₆)alkyl, (C₁-C₆)alkylaryl, aryl(C₁-C₆)alkoxy, heteroaryl, heteroaryl(C₁-C₆)alkyl, (C₁-C₆)alkylheteroaryl, heterocyclyl, heterocyclyl(C₁-C₆)alkyl, (C₁-C₆)alkylheterocyclyl, (C₁-C₆)alkylheterocyclyl(C₁-C₆)alkyl, tri(C₁-C₆)alkylsilyl, (C₃-C₇)cycloalkyl, aryloxy, heterocyclyloxy, methylenedioxy, (C₁-C₆)alkylcarbonyloxy, arylcarbonyloxy, di(C₁-C₆)alkylaminoheterocyclyl(C₁-C₆)alkyl, (C₃-C₇)cycloalkenyloxy, heterocyclylcarbonyloxy, (C₁-C₆)alkylideneaminooxy, carboxy, (C₁-C₆)alkoxycarbonyl, aryloxycarbonyl, (C₃-C₇)cycloalkyloxycarbonyl, nitro, amino, heterocyclyl(C₁-C₆)alkoxycarbonylamino, ureido, (C₁-C₆)alkylamino, amino(C₁-C₆)alkyl, di(C₁-C₆)alkylamino, arylamino, diarylamino, heterocyclylamino, formylamino, (C₁-C₆)alkylcarbonylamino, arylcarbonylamino, heterocyclylcarbonylamino, aminocarbonyl, (C₁-C₆)alkylaminocarbonyl, di(C₁-C₆)alkylaminocarbonyl, arylaminocarbonyl, heteroarylaminocarbonyl, arylaminocarbonyl(C₁-C₆)alkyl, (C₃-C₇)cycloalkylaminocarbonyl, heterocyclylaminocarbonyl, (C₁-C₆)alkoxycarbonylamino, hydroxyaminocarbonyl, (C₁-C₆)alkoxyimino, (C₁-C₆)alkylsulfonylamino, arylsulfonylamino, heterocyclylsulfonylamino, formyl, (C₁-C₆)alkylcarbonyl, arylcarbonyl, (C₃-C₇)cycloalkylcarbonyl, heterocyclylcarbonyl, heterocyclylcarbonyl(C₁-C₆)alkyl, (C₁-C₆)alkylsulfonyl, polyfluorinated (C₁-C₆)alkylsulfonyl, arylsulfonyl, aminosulfonyl, (C₁-C₆)alkylaminosulfonyl, di(C₁-C₆)alkylaminosulfonyl, arylaminosulfonyl, heterocyclylaminosulfonyl, arylthio, (C₁-C₆)alkylthio; in their turn, whenever appropriate, each of the above substituents may be further substituted by one or more of the aforementioned groups.

From all of the above, it is clear to the skilled person that any group which name is a composite name such as, for instance, “arylamino” has to be intended as conventionally construed by the parts from which it derives, e.g. by an amino group which is substituted by aryl, wherein aryl is as above defined.

Likewise, any of the terms such as, for instance, (C₁-C₆)alkylthio, (C₁-C₆)alkylamino, di(C₁-C₆)alkylamino, (C₁-C₆)alkoxycarbonyl, (C₁-C₆)alkoxycarbonylamino, heterocyclylcarbonyl, heterocyclylcarbonylamino, (C₃-C₇)cycloalkyloxycarbonyl and the like, include groups wherein the (C₁-C₆)alkyl, (C₁-C₆)alkoxy, aryl, (C₃-C₇)cycloalkyl and heterocyclyl moieties are as above defined.

The present invention also provides processes for the preparation of the compound of general formula (I) as defined above, by using the reaction routes and synthetic schemes described below, employing the techniques available in the art and starting materials readily available. The preparation of certain embodiments of the present invention is described in the examples that follow, but those of ordinary skill in the art will recognize that the preparations described may be readily adapted to prepare other embodiments of the present invention. For example, the synthesis of non-exemplified compounds according to the invention may be performed by apparent modifications to those skilled in the art, for instance by appropriately protecting interfering groups, by suitably replacing reagents with others known in the art, or by making routine modifications of reaction conditions. Alternatively, other reactions referred to herein or known in the art will be recognized as having adaptability for preparing other compounds of the invention.

The compounds of this invention can be prepared from readily available starting materials using the following general methods and procedures. Unless otherwise indicated, the starting materials are known compounds or may be prepared from known compounds according to well known procedures. It will be appreciated that, where typical or preferred process conditions (i.e., reaction temperatures, times, mole ratios of reactants, solvents, pressures) are described, different process conditions can also be used unless otherwise stated. Optimum reaction conditions may vary with the reactants or solvents used, but such conditions can be determined by one skilled in the art by routine optimization procedures.

The compound of general formula (I), as defined above, can be prepared according to the general synthetic processes described hereafter in Schemes A, B and C.

Preparation of a compound of formula (I) wherein R1 is a heteroaryl selected from a group of formula (A), (B), (C), (D) and (E); R2 is a substituted aryl or a substituted heteroaryl ring bearing from one up to three substituents selected from halogen, nitro, amino, (C₁-C₆) alkyl amino, aminocarbonyl, an optionally substituted straight or branched (C₁-C₆) alkyl, an optionally substituted straight or branched (C₁-C₆) alkoxy, an optionally substituted straight or branched polyfluorinated (C₁-C₆)alkyl and optionally substituted straight or branched polyfluorinated (C₁-C₆)alkoxy; R3 is H, an optionally substituted straight or branched (C₁-C₄) alkyl chain, an optionally substituted (C₃-C₆) cycloalkyl group, or an optionally substituted (C₅-C₆) heterocyclyl group; R4 is hydrogen and R5 is hydrogen, halogen or an optionally substituted straight or branched (C₁-C₃) alkyl can be obtained following Scheme A below:

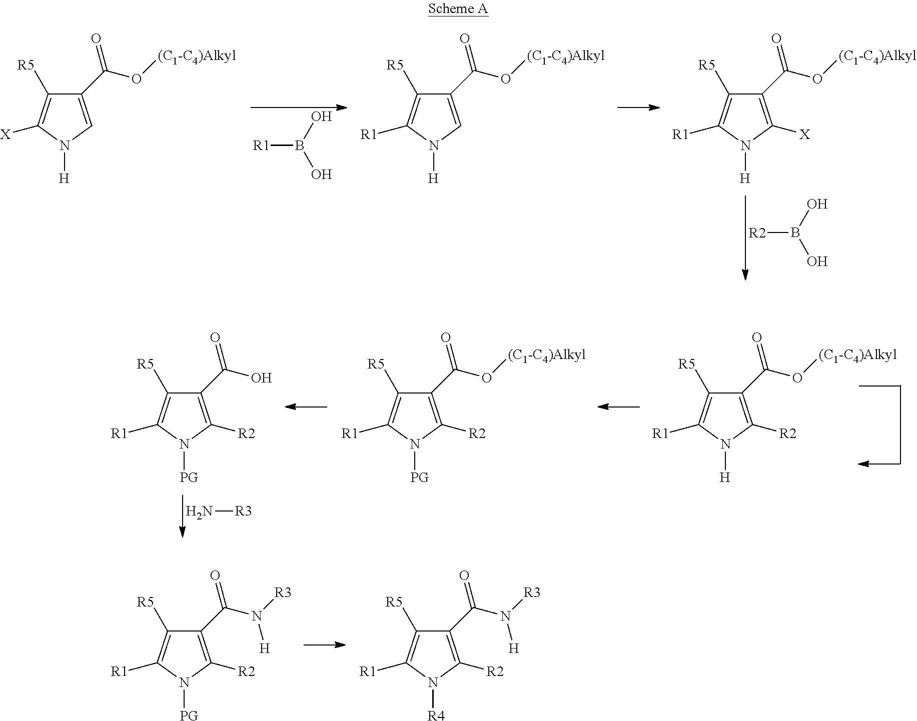

Accordingly, the process foresees the following steps:

Step 1) metal-catalyzed coupling reaction of a compound of formula (II):

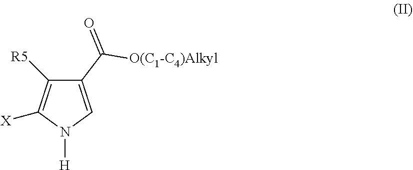

wherein R5 is hydrogen or an optionally substituted straight or branched (C₁-C₃) alkyl and X is halogen, with a suitable organoboronic acid derivative of formula (III):

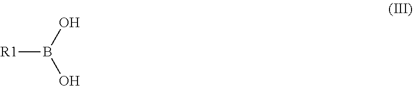

wherein R1 is a heteroaryl group of formula (A), (B), (C) (D) or (E);

Step 2) halogenation of the so obtained compound of formula (IV):

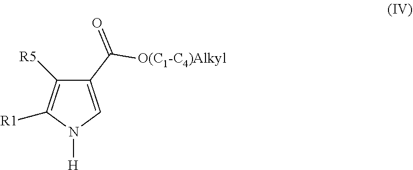

wherein R1 and R5 are as defined above in Step 1, thus to obtain a compound of formula (V):

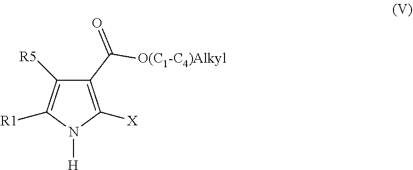

wherein R1 and R5 are as defined above in Step 1 and X is halogen;

Step 3) metal-catalyzed coupling reaction of a compound of formula (V) with a suitable organoboronic acid derivative of formula (VI):

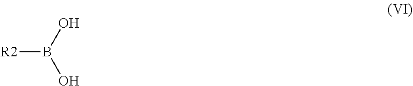

wherein R2 is a substituted aryl or a substituted heteroaryl ring, bearing from one up to three substituents selected from halogen, nitro, amino, (C₁-C₆) alkyl amino, aminocarbonyl, an optionally substituted straight or branched (C₁-C₆) alkyl, an optionally substituted straight or branched (C₁-C₆) alkoxy, an optionally substituted straight or branched polyfluorinated (C₁-C₆)alkyl and optionally substituted straight or branched polyfluorinated (C₁-C₆)alkoxy, so to obtain a compound of formula (VII):

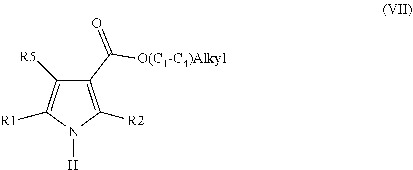

wherein R1, and R5 are as defined above in Step 1 and R2 is as defined in Step 3; conv. 1) a compound of formula (VII) obtained from Step 3 wherein R5 is hydrogen can be converted in a compound of formula (VII) wherein R5 is halogen (X), following the conditions already reported in Step 2 above;

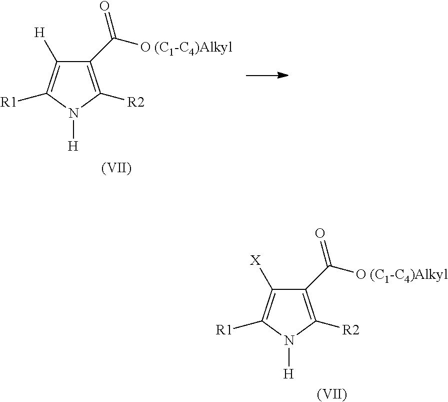

Step 4) protection of the compound of formula (VII) obtained from Step 3 or conv.1:

wherein R1 and R2 are as defined above in Step 1 and in Step 3, respectively and R5 is hydrogen, halogen or an optionally substituted straight or branched (C₁-C₃) alkyl, by reaction with the suitable protecting group, so to obtain the carboxylic ester of formula (VIII):

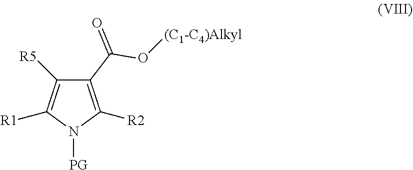

wherein R1, R2 and R5 are as defined above and PG is a protecting group such as trimethylsilylethoxymethyl (SEM), tert-Butyloxycarbonyl (BOC) or benzenesulfonyl;

Step 5) hydrolysis under basic condition of the carboxylic ester of formula (VIII), so to yield the carboxylic acid of formula (IX):

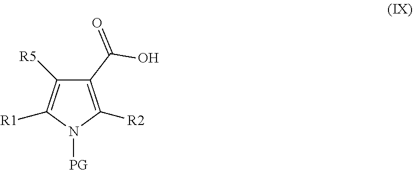

wherein R1, R2, R5 and PG are as defined above in Step 4;

Step 6) amidation of the intermediate of formula (IX) by reaction with an amine derivative of formula (X):

H₂N—R3 (X)

wherein R3 is hydrogen, an optionally substituted straight or branched (C₁-C₄) alkyl chain, an optionally substituted (C₃-C₆) cycloalkyl group, or an optionally substituted (C₅-C₆) heterocyclyl group;

Step 7) deprotection of the resultant compound of formula (XI):

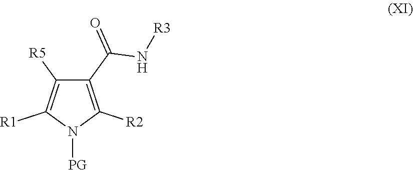

wherein R1, R2, R5 and PG are as defined above under Step 5 and R3 is as defined under Step 6, to give a compound of formula (I):

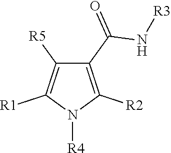

wherein R1, R2, R3 and R5 are as defined above and R4 is hydrogen; or

an intermediate compound of formula (VIII), wherein R5 is halogen, can be converted into an intermediate of formula (XI), wherein R5 is a straight or branched C₁-C₃ alkyl chain, following the Scheme A1 below:

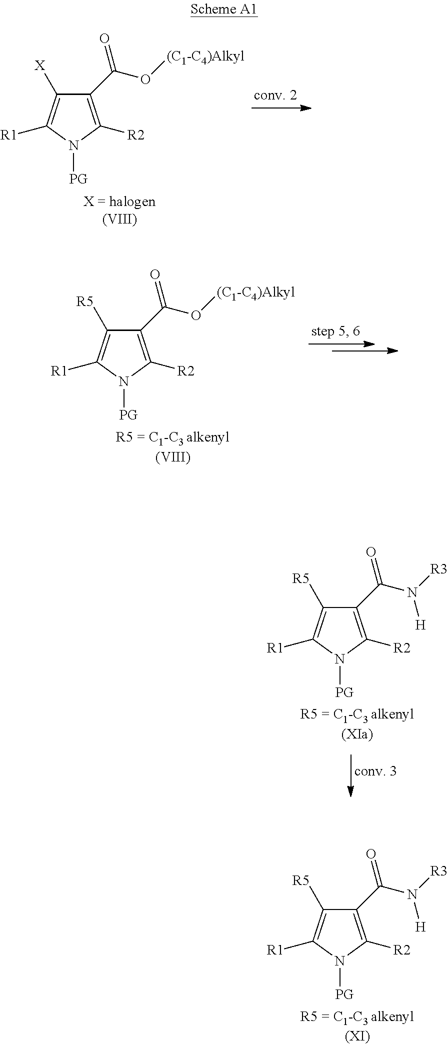

Accordingly, the process foresees the following steps:

conv. 2) converting a compound of formula (VIII):

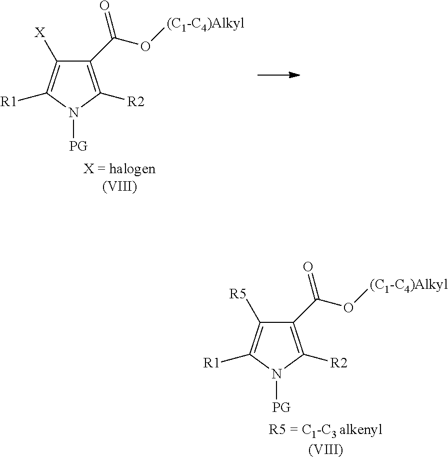

wherein R1 and R2 are as defined above under Step 1, into a compound of formula (VIII) wherein R5 is an optionally substituted straight or branched (C₁-C₃) alkenyl chain, following the condition known in the art for palladium-catalyzed reaction, already reported in Step 3 of Scheme A; then reacting the compound (VIII) under conditions reported in Step 5 and 6 of the Scheme A, thus to obtain a compound (XIa) wherein R1, R2 and R5 are as defined above;

conv. 3) converting the so obtained compound of formula (XIa):

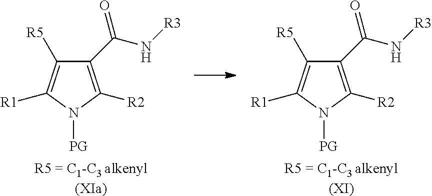

wherein R5 is as defined above into a compound of formula (XI) wherein R5 is an optionally substituted straight or branched (C₁-C₃) alkyl, following the condition known in the art for reduction of double bond/hydrogenation.

Alternatively, the compound of formula (I) wherein R1 is an optionally substituted heteroaryl group of formula (A), (B), (C) (D) or (E); R2 is a substituted aryl or a substituted heteroaryl ring bearing from one up to three substituents; R3 and R4 are hydrogen and R5 is hydrogen or an optionally substituted straight or branched (C₁-C₃) alkyl, can be prepared following the Scheme B below:

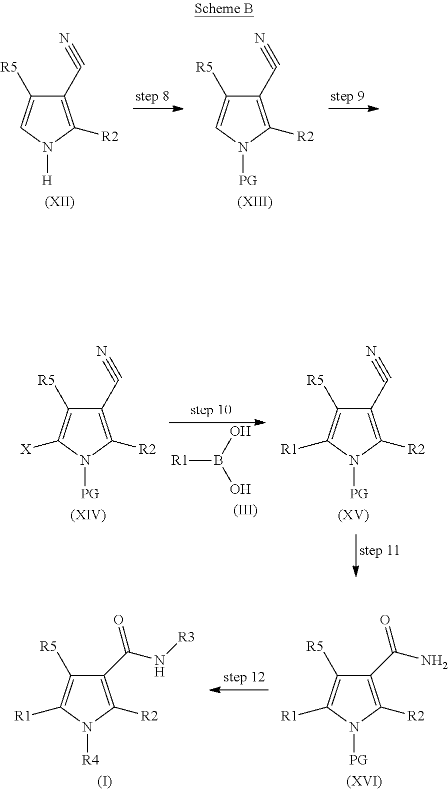

Accordingly, the process foresees the following steps:

Step 8) protection of a compound of formula (XII):

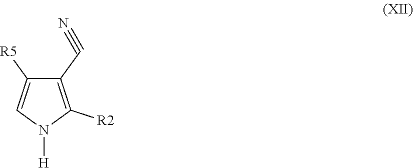

wherein R2 is a substituted aryl or a substituted heteroaryl ring bearing from one up to three substituents and R5 is hydrogen or an optionally substituted straight or branched (C₁-C₃) alkyl;

Step 9) halogenation of the so obtained compound of formula (XIII):

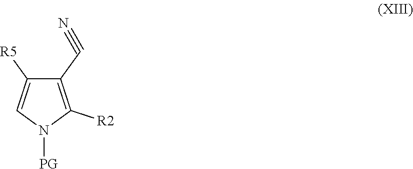

wherein R2 and R5 are as defined above under Step 8 and PG is a protecting group such as SEM, BOC or benzenesulfonyl;

Step 10) metal-catalyzed coupling reaction of the resultant compound of formula (XIV):

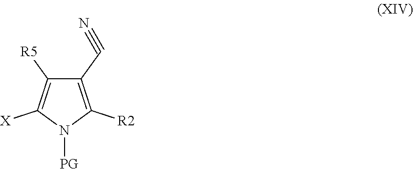

wherein R2, R5 and PG are as defined above in Step 9 and X is halogen, with a suitable organoboronic acid derivative of formula (III):

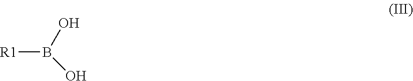

wherein R1 si a heteroaryl group of formula (A), (B), (C) (D) or (E);

Step 11) hydrolysis of the so obtained compound of formula (XV):

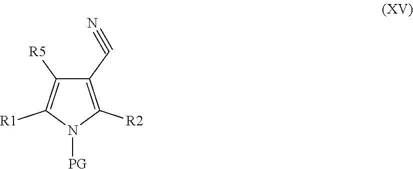

wherein R1, R2, R5 and PG are as defined above in Step 10, thus to yield the corresponding amide intermediate of formula (XVI);

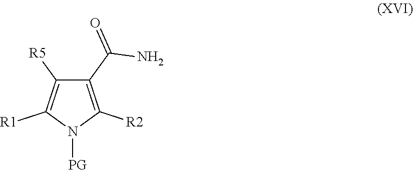

Step 12) deprotection of the compound of formula (XVI) to give a compound of formula (I):

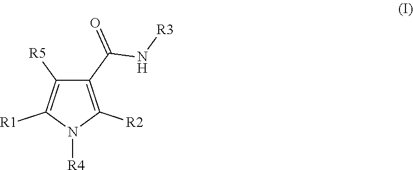

wherein R1, R2 and R5 are as defined above and R3 and R4 are hydrogen.

Alternatively, the compound of formula (I) wherein R1 is a heteroaryl group of formula (A), (B), (C) (D) or (E); R2 is a substituted aryl or a substituted heteroaryl ring bearing from one up to three substituents; R3 is hydrogen, R4 is hydrogen, an optionally substituted straight or branched (C₁-C₆) alkyl or an optionally substituted straight or branched (C₂-C₆) alkenyl, and R5 is hydrogen or an optionally substituted straight or branched (C₁-C₄) alkyl chain, can be prepared following Scheme C below:

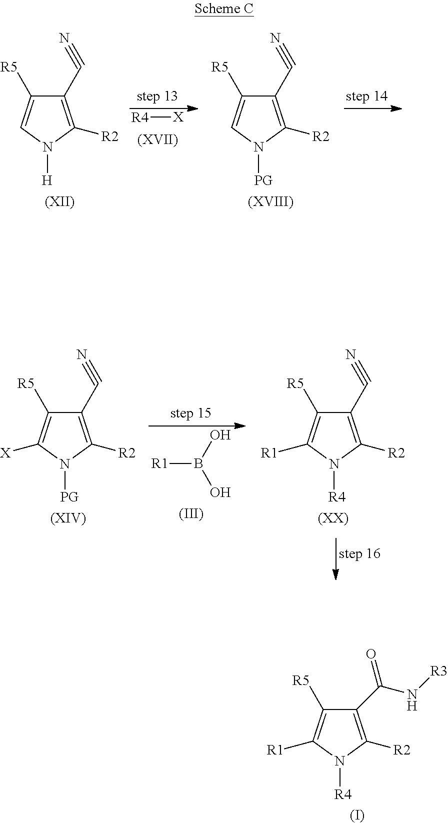

Accordingly, the process foresees the following steps:

Step 13) reaction of a derivative of formula (XII):

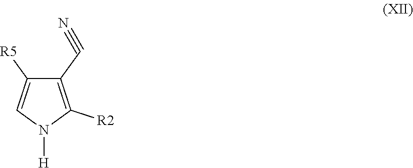

wherein R2 is a substituted aryl or a substituted heteroaryl ring bearing from one up to three substituents and R5 is hydrogen or an optionally substituted straight or branched (C₁-C₃) alkyl, with a halo derivative of formula (XVII):

R4-X (XVII)

wherein R4 is an optionally substituted straight or branched C₁-C₆ alkyl, or an optionally substituted straight or branched C₂-C₆ alkenyl and X is halogen, in the presence of a base or by addition of a metal catalyst;

Step 14) halogenation of the so obtained compound of formula (XVIII):

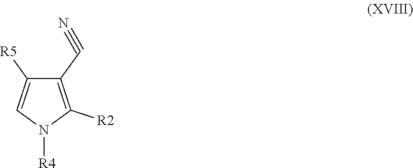

wherein R2, R4 and R5 are as defined above in Step 13;

Step 15) metal-catalyzed coupling reaction of the resultant compound of formula (XIX):

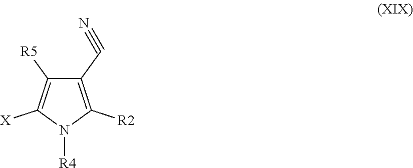

wherein X is halogen and R2, R3, and R4 are as defined above, with a suitable organoboronic acid derivative of formula (III):

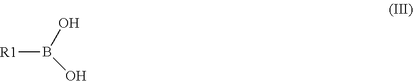

wherein R1 is a heteroaryl group (A), (B), (C), (D) or (E);

Step 16) hydrolysis of the so obtained intermediate of formula (XX):

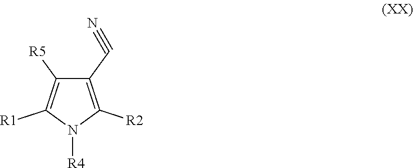

to give a compound of formula (I):

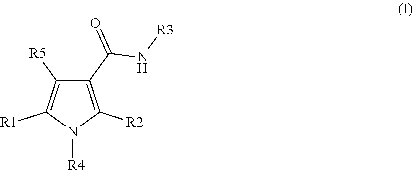

wherein R1, R2, R4 and R5 are as defined above and R3 is hydrogen.

According to the Step 1 of Scheme A, metal-catalyzed coupling reaction of a compound of formula (II) with a organoboronic derivative of general formula (III) to give a compound of formula (IV) can be accomplished in a variety of ways. Preferably, a compound of formula (IV) can be prepared from an intermediate of formula (II) by Pd-catalyzed Suzuki-Miyaura coupling. Transition metal-catalyzed couplings of (hetero)aryl halides with (hetero)aryl boronic acids or boronic-esters are well known to the person skilled in the art, see references: a) Miyaura, Norio; Suzuki, Akira (1979). “Palladium-Catalyzed Cross-Coupling Reactions of Organoboron Compounds”. Chemical Reviews 95 (7): 2457-2483; b) Suzuki, A. In Metal-Catalyzed Cross-Coupling Reactions, Diederich, F., and Stang, P. J., Eds.; Wiley-VCH: New York, 1998, pp. 49-97. In the so called Suzuki-Miyaura reaction, coupling reaction of (hetero)aryl boronic acids or boronic-esters with (hetero)aryl halides is typically triggered by palladium complex. Phosphine-palladium complexes such as [1,1′-bis(diphenylphosphino) ferrocene] dichloro palladium(II) are used for this reaction but also bis(triphenylphosphine)palladium(II) chloride, tetrakis(triphenylphosphine)palladium(0) may be employed. A base such as potassium phosphate, sodium carbonate, cesium carbonate, potassium carbonate, potassium t-butoxide, tetraethyl ammonium hydroxide, triethylamine is added and tetrahydrofurane, dioxane, N,N-dimethylformamide, ethanol, toluene, water or a mixture thereof may be used as reaction media. Typically, temperatures range from room temperature to 150° C. Conventional heating along with microwave irradiation may be employed. Reaction duration ranges from about 30 min to about 96 hours. Various Pd-catalyst/base/solvent combinations have been described in the literature, which allow the fine-tuning of the reaction conditions in order to allow for a broad set of additional functional groups on both coupling partners.

According to Step 2 of Scheme A, a compound of formula (V) can be obtained by halogenating a compound of formula (IV) in a variety of ways and experimental conditions known in the art. Preferably this reaction is conducted in the presence of N-bromosuccinimide, N-iodosuccinimmide, N-chlorosuccinimide, bromine, iodine, hydrobromic acid/hydrogen peroxide, in a suitable solvent, such as acetonitrile, methanol, tetrahydrofuran, N,N-dimethylformamide, dioxane, dimethylsulfoxide, acetic acid, water or a mixture thereof at a temperature ranging from about 0° C. to reflux and for a period of time varying from about 1 hour to about 96 hours.

According to the Step 3 of Scheme A, metal-catalyzed coupling reaction of a compound of formula (V) with a organoboronic derivative of general formula (VI) to give a compound of formula (VII) can be accomplished in a variety of ways already described in Step 1 of Scheme A.

According to Step 4 of Scheme A, a compound of formula (VII) may be transformed into a compound of formula (VIII) in a variety of ways and experimental conditions which are widely known in the art for protection of secondary amino group. Preferably the reaction is carried out by treatment with an excess of (trimethylsilyl)ethoxymethyl chloride in a suitable solvent, such as tetrahydrofuran, dichloromethane in the presence of a base such as, for instance, sodium hydride. Typically, the reaction is carried out at a temperature ranging from 0° C. to reflux and for a time varying from about 30 minutes to about 96 hours. Benzenesulfonyl group may be introduced by reaction with benzensulfonul chloride, in a solvent such as dichloromethane, acetonitrile in the presence of a proton scavenger such as, for example, triethylamine, N,N-diisopropylethylamine at temperatures ranging from room temperature to reflux. Tert-butoxycarbonyl (Boc) group may be introduced by treatment with an excess of di-tert-butyldicarbonate in a presence of a base, such as sodium bicarbonate, triethylamine, N,N-diisopropylethylamine, in a solvent such as tetrahydrofuran, dioxane, dichloromethane at temperatures ranging from room temperature to reflux.

According to the Step 5 of Scheme A, hydrolysis of the carboxylic ester of formula (VIII) into the carboxylic acid of formula (IX) can be accomplished in a variety of ways. Preferably this reaction is carried out in a suitable solvent such as, for instance, methanol, ethanol, 1,4-dioxane, tetrahydrofuran in the presence of a suitable base such as, for instance, sodium hydroxide, potassium hydroxide or lithium hydroxide.H₂O in tetrahydrofuran. Typically, the reaction is carried out at a temperature ranging from room temperature to 150° C. and for a time varying from about 1 hour to about 96 hours. Conventional heating along with microwave irradiation may be employed

According to the Step 6 of Scheme A, the conversion of a carboxylic acid of formula (IX) into a carboxamide of formula (XI) can be accomplished in a variety of ways and experimental conditions, which are widely known in the art for the preparation of carboxamides. As an example, a compound of formula (IX) can be reacted with the ammonium salt of 1-hydroxybenzotriazole or with an amine NH₂R₃(X) in the presence of 0-(Benzotriazol-1-yl)-N,N,N′,N′-tetramethyluronium tetrafluoroborateor, hydroxybenzotriazole, dicyclohexyl carbodiimide, diisopropyl carbodiimide, 1-ethyl-3-(3′-dimethylamino)carbodiimide hydrochloric acid salt. Preferably, this reaction is carried out in a suitable solvent such as, for instance, tetrahydrofuran, dichloromethane, toluene, dioxane, N,N-dimethylformamide, N,N-dimethylacetamide and in the presence of a proton scavenger such as, for example, triethylamine, N,N-diisopropylethylamine, at a temperature ranging from 0° C. to reflux, for a time ranging from about 30 min to about 96 hours. Alternatively, a compound of formula (IX) can be converted into its corresponding acyl chloride in the presence of thionyl chloride or oxalyl chloride, in a suitable solvent, such as toluene, dichloromethane, chloroform, diethyl ether, tetrahydrofuran, dioxane, at a temperature ranging from about −10° C. to reflux and for a period of time varying from about 1 hour to about 96 hours. The acyl chloride can be isolated by evaporation of the solvent and further reacted with 33% ammonium hydroxide solution or with an amine NH₂R₃(X) in a suitable solvent, such as toluene, dichloromethane, chloroform, diethyl ether, tetrahydrofuran, dioxane, at a temperature ranging from about −10° C. to reflux and for a period of time varying from about 1 hour to about 96 hours.

According to the Step 7 of Scheme A, the removal of the protecting group PG on the pyrrole ring of a compound of formula (XI) may be carried out following procedures which are well known in the art. Depending on the protecting group of choice, the following conditions may be employed: 2-(trimethylsilyl)ethoxymethyl (SEM) may be removed with tetra-n-butylammonium floride, hydrogen fluoride pyridine or trifluoroacetic acid in solvents such as tetrahydrofuran, dichloromethane at room temperature or below; benzenesulfonyl (Bs) groups may be removed with potassium hydroxide, sodium hydroxide, potassium carbonate, lithium hydroxide, in solvents such as methanol, tetrahydrofurane, dioxane at temperatures ranging from room temperature to reflux; tert-butoxycarbonyl (Boc) may be removed in the presence of trifluoroacetic acid in dichloromethane or by sodium carbonate in dimethoxyethane, N,N-dimethylformamide at a temperature ranging from room temperature to 130° C.

According to the Step 8 of Scheme B, the conversion of a compound of general formula (XII) into a compound of formula (XIII) can be accomplished by reaction already described in Step 4 of Scheme A.

According to Step 9 of Scheme B, the halogenation of compound of formula (XIII) to obtain a compound of formula (XIV) can be carried out as described above in Step 2 of Scheme A.

According to the Step 10 of Scheme B, metal-catalyzed coupling reaction of a compound of formula (XIV) with a organoboronic derivative of general formula (III) to give a compound of formula (XV) can be accomplished in a variety of ways already described in Step 1 of Scheme A.

According to Step 11 of Scheme B, the hydrolysis of a compound of formula (XV) to a compound of formula (XVI) can be carried out in a variety of ways, according to conventional methods for transforming a cyano group to amide. Preferably, this reaction is carried out in a suitable solvent such as, for instance, methanol, ethanol, n-butanol, 1,4-dioxane, toluene, water, or a mixture thereof, in the presence of a suitable acid or base, such as, for instance, sulfuric acid, hydrochloric acid, methanesulfonic acid, indium chloride, sodium or potassium hydroxide, sodium or potassium carbonate or a suitable reagent such as hydrogen peroxide, sodium perborate or acetaldoxime. Typically, the reaction is carried out at a temperature ranging from room temperature to reflux and for a time ranging from about 1 hour to about 96 hours.

According to Step 12 of Scheme B, the removal of the protecting group PG on the pyrrole ring of a compound of formula (XVI) to obtain a compound of formula (I) can be performed as described above in Step 7 of Scheme A.

According to the Step 13 of Scheme C, the reaction of a compound of formula (XII) with a halo derivative of general formula (XVII) to give a compound of formula (XVIII) may be carried out in the presence of a base such as sodium hydride and tetrahydrofurane or dioxane may be used as reaction media. Typically, temperatures range from 5° C. to reflux. Reaction duration ranges from about 30 min to about 24 hours. Alternately, metal-catalyzed coupling reaction of a compound of formula (XIV) with a halo derivative of general formula (V) to give a compound of formula (Ic) can be accomplished in the presence of tris(dibenzylideneacetone)dipalladium and tri-tert-butylphosphine. A base such as sodium carbonate, cesium carbonate, potassium carbonate is added and tetrahydrofurane, dioxane, N,N-dimethylformamide and toluene may be used as reaction media. Typically, temperatures range from room temperature to 150° C. Conventional heating along with microwave irradiation may be employed. Reaction duration ranges from about 30 min to about 24 hours.

According to Step 14 of Scheme C, the halogenation of compound of formula (XVIII) to obtain a compound of formula (XIX) can be carried out as described above in Step 2 of Scheme A.

According to the Step 15 of Scheme C, metal-catalyzed coupling reaction of a compound of formula (XIX) with a organoboronic derivative of general formula (III) to give a compound of formula (XX) can be accomplished in a variety of ways already described in Step 1 of Scheme A.

According to Step 16 of Scheme C, the hydrolysis of a compound of formula (XX) to a compound of formula (I) can be carried out as described above in Step 11 of Scheme B.

According to the conversion described under conv.1) the transformation of a compound of formula (VII) into a compound of formula (VII) can be carried out in different ways and experimental conditions. Preferably it is carried out in a way analogous to that reported for Step 2 of Scheme A.

According to the conversion described under conv. 2) the derivatization of a compound of formula (VIII) into a compound of formula (VIII) can be accomplished in a variety of ways already described in Step 3 of Scheme A.

According to the conversion described under conv. 3) the transformation of a compound of formula (XIa) into a compound of formula (XI) can be accomplished in a variety of ways and experimental conditions, which are widely known in the art for reduction of double bond. Preferably this reaction is carried out in a suitable solvent such as, for instance, methanol, ethanol, toluene, tetrahydrofuran in the presence of a suitable catalyst such as, for instance, palladium on carbon (10%), palladium acetate, rhodium catalyst. Typically, the reaction is carried out at a temperature ranging from room temperature to 150° C. and for a time varying from about 1 hour to about 96 hours.

From all of the above it is clear to the skilled person that any compound of formula (I) bearing a functional group which can be further derivatized to another functional group, by working according to methods well known in the art thus leading to other compounds of the formula (I), is intended to be comprised within the scope of the present invention.

When preparing the compounds of general formula (I) according to any of the above variants of the process, optional functional groups within the starting materials, the reagents or the intermediates thereof, and which could give rise to unwanted side reactions, need to be properly protected according to conventional techniques (see e.g., Green, Theodora W. and Wuts, Peter G. M.—Protective Groups in Organic Synthesis, Third Edition, John Wiley & Sons Inc., New York (NY), 1999). Likewise, the conversion of these latter into the free deprotected compounds may be carried out according to known procedures.

The compounds of every general formula can be further transformed in other compounds of the same general formula according to methods well known in the literature, as reported in the experimental section.

According to any variant of the process for preparing the compounds of the formula (I), the starting materials and any other reactants are known or easily prepared according to known methods.

The compounds of the formula (XII) can be prepared as described in WO2009133170A1.

The compounds of the formula (II), (III), (VI), (X) and (XVII) are commercially available.

The final compounds may be isolated and purified using conventional procedures, for example chromatography and/or crystallization and salt formation.

The compounds of general formula (I) as defined above can be converted into pharmaceutically acceptable salts. The compounds of general formula (I) as defined above, or the pharmaceutically acceptable salts thereof, can be subsequently formulated with a pharmaceutically acceptable carrier or diluent to provide a pharmaceutical composition.

The synthesis of a compound of general formula (I), according to the synthetic processes described above, can be conducted in a stepwise manner, whereby each intermediate is isolated and purified if needed by standard purification techniques, like, for example, column chromatography, before carrying out the subsequent reaction. Alternatively, two or more steps of the synthetic sequence can be carried out in a so-called “one-pot” procedure, as known in the art, whereby only the compound resultant from the two or more steps is isolated and purified.

The present invention also provides a method of treating a disease caused by and/or associated with dysregulated Cdc7 kinase activity, which comprises administering to a mammal, preferably a human, in need thereof, an effective amount of a compound of formula (I) as defined above.

Furthermore the present invention provides a compound of formula (I) or a pharmaceutically acceptable salt thereof, as defined above, for use in a method of treating a disease caused by and/or associated with dysregulated Cdk7 kinase activity, which comprises administering to a mammal, preferably a human, in need thereof, an effective amount of a compound of formula (I) as defined above.

Additionally, the invention provides the use of a compound of formula (I) or a pharmaceutically acceptable salt thereof, as defined above, in the manufacture of a medicament for treating a disease caused by and/or associated with dysregulated Cdc7 kinase activity.

Preferably the disease is selected from the group consisting of cancer, cell proliferative disorders, immune-related disorders. More preferably, the disease is cancer.

According to a most preferred embodiment of the present invention the cancer is selected from the group consisting of: carcinomas, such as bladder, breast, kidney, liver, colon, lung, including small cell lung cancer, esophagus, gall-bladder, ovary, pancreas, stomach, cervix, prostate, head and neck and skin, including squamous cell carcinoma; hematopoietic tumors of lymphoid lineage including leukemia, acute lymphocitic leukemia, acute lymphoblastic leukemia, B-cell lymphoma, angioimmunoblastic T-cell lymphoma, Hodgkin's lymphoma, non-Hodgkin's lymphoma, hairy cell lymphoma mantle cell lymphoma and Burkitt's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including glioma, glioblastoma, glioblastoma multiforme, astrocytoma, oligodendroglioma, paraglioma, neuroblastoma, and schwannomas; and other tumors, including melanoma, seminoma, teratocarcinoma, osteosarcoma, xeroderma pigmentosum, keratoxanthoma, thyroid cancers, such as papillary thyroid carcinoma and medullary thyroid carcinoma, Kaposi's sarcoma, chondrosarcoma, cholangiocarcinoma, head and neck tumors.

Other preferred diseases caused by and/or associated with dysregulated Cdc7 kinase activity, are cellular proliferation disorders such as, for example, benign prostate hyperplasia, familial adenomatosis, polyposis, neurofibromatosis, psoriasis, vascular smooth cell proliferation associated with atherosclerosis, pulmonary fibrosis, arthritis, glomerulonephritis and post-surgical stenosis and restenosis.

Further preferred diseases caused by and/or associated with dysregulated Cdc7 kinase activity, are immune-related disorders including but not limited to: transplant rejection, skin disorders like psoriasis, allergies, asthma and autoimmune-mediated diseases such as rheumatoid arthritis (RA), systemic lupus erythematosus (SLE), Crohn's disease and amyotrophic lateral sclerosis

Moreover, the present invention provides a compound of formula (I), or a pharmaceutically acceptable salt thereof, as defined above, for use in a method of treating a mammal in need thereof in combination with radiation therapy or in combination with a chemotherapy, target therapy or immunotherapy regimen.

In one embodiment the chemotherapy regimen and/or the target therapy regimen comprises at least one cytostatic or cytotoxic agent.

Cytostatic or cytotoxic agents include, but are not limited to, antibiotic-type agents, alkylating agents, antimetabolite agents, hormonal agents, immunological agents, interferon-type agents, cyclooxygenase inhibitors (e.g. COX-2 inhibitors), matrixmetalloprotease inhibitors, telomerase inhibitors, tyrosine kinase inhibitors, anti-growth factor receptor agents, anti-HER2 agents, anti-EGFR agents, anti-angiogenesis agents (e.g. angiogenesis inhibitors), farnesyl transferase inhibitors, ras-raf signal transduction pathway inhibitors, cell cycle inhibitors, cdks inhibitors, tubulin binding agents, topoisomerase I inhibitors, topoisomerase II inhibitors, aromatase inhibitors, inhibitors of kinesins, therapeutic monoclonal antibodies, inhibitors of mTOR, histone deacetylase inhibitors, platinum, inhibitors of hypoxic response. Immunotherapy agents includePD-1 antagonists, antibodies which specifically binds to PD-1 or PD-Li.

If formulated as a fixed dose, such combination products employ the compounds of this invention within the dosage range described below and the other pharmaceutically active agent within the approved dosage range.

Compounds of formula (I) may be used sequentially with known anticancer agents when a combination formulation is inappropriate.

The compounds of formula (I) of the present invention, suitable for administration to a mammal, e.g. to humans, can be administered by the usual routes and the dosage level depends upon the age, weight, and conditions of the patient and administration route.

For example, a suitable dosage adopted for oral administration of a compound of formula (I) may range from about 10 to about 1000 mg per dose, from 1 to 5 times daily. The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or through intravenous and/or intrathecal and/or intraspinal injection or infusion.

The pharmaceutical compositions containing the compounds of the invention are usually prepared following conventional methods and are administered in a suitable pharmaceutical form.

For example, the solid oral forms may contain, together with the active compound, diluents, e.g. lactose, dextrose, saccharose, sucrose, cellulose, corn starch or potato starch; lubricants, e.g. silica, talc, stearic acid, magnesium or calcium stearate, and/or polyethylene glycols; binding agents, e.g. starches, arabic gum, gelatine methylcellulose, carboxymethylcellulose or polyvinyl pyrrolidone; disintegrating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. These pharmaceutical preparations may be manufactured in known manner, for example, by means of mixing, granulating, tabletting, sugar-coating, or film-coating processes.

The liquid dispersions for oral administration may be, e.g. syrups, emulsions and suspensions.

As example the syrups may contain, as a carrier, saccharose or saccharose with glycerine and/or mannitol and sorbitol.

The suspensions and the emulsions may contain, as examples of carriers, natural gum, agar, sodium alginate, pectin, methylcellulose, carboxymethylcellulose or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol and, if desired, a suitable amount of lidocaine hydrochloride.

The solutions for intravenous injections or infusions may contain, as a carrier, sterile water or preferably they may be in the form of sterile, aqueous, isotonic, saline solutions or they may contain propylene glycol as a carrier.

The suppositories may contain, together with the active compound, a pharmaceutically acceptable carrier, e.g. cocoa butter, polyethylene glycol, a polyoxyethylene sorbitan fatty acid ester surfactant or lecithin.

The present invention also provides a pharmaceutical composition comprising a therapeutically effective amount of a compound of formula (I) or a pharmaceutically acceptable salt thereof, as defined above, and at least one pharmaceutically acceptable excipient, carrier or diluent.

The present invention further provides a pharmaceutical composition of a compound of formula (I) further comprising one or more chemotherapeutic agents.

Moreover, the invention provides an in vitro method for inhibiting Cdc7 protein activity which comprises contacting the said protein with an effective amount of a compound of formula (I) as defined above.

Additionally, the invention provides a product comprising a compound of formula (I) or a pharmaceutically acceptable salt thereof, as defined above, and one or more chemotherapeutic agents, as a combined preparation for simultaneous, separate or sequential use in anticancer therapy.

Finally, the invention provides a compound of formula (I) or a pharmaceutically acceptable salt thereof, as defined above, for use as a medicament.

### EXPERIMENTAL PART

The short forms and abbreviations used herein have the following meaning:
g gram mg milligram
mL milliliter L microliter
mM millimolar mmol millimole
μM (micromolar) MHz (Mega-Hertz)
h hour(s) Hz (Hertz)
mm (millimetres) min (minutes)
μm (micron) M (molar)
BSA bovine serum albumine DTT dithiothreitol
NADPH Nicotinamide adenine dinucleotide phosphate Rt retention time
2-HG 2-Hydroxy glutaric acid KOtBu (potassium tert-butoxide)
rt (room temperature) TEA (triethylamine)
DMAP (4-dimethylaminopyridine) DME (1,2-dimethoxyethane)
TFA (trifluoroacetic acid) Na₂SO₄ (sodium sulphate)
AcOH (acetic acid) ESI (electrospray ionization)
Na₂CO₃ (sodium carbonate) K₂CO₃ (potassium carbonate)
Cs₂CO₃ (caesium carbonate) K₃PO₄ (potassium phosphate)
LiOH (lithium hydroxide) NaOH (sodium hydroxide)
KOH (potassium hydroxide) p-TsOH (p-toluensulfonic acid)
EtOAc (ethyl acetate) LiHMDS (lithium bis(trimethylsilyl)amide)
NMP (N-methyl-2-pyrrolidone) NaH (sodium hydride)
DMA (N,N-dimethylacetamide) KH (potassium hydride)
DMF (N,N-dimethylformamide) DCM (dichloromethane)
DIPEA (N,N-diisopropyl-N-ethylamine) hex (hexane)
THE (tetrahydrofuran) DMSO (dimethylsulfoxide)
MeOH (methanol) ACN (acetonitrile)
EtOH (ethanol) Bn (benzyl)
—OMs (mesylate) —OTs (tosylate)
HOBT (N-hydroxy-benzotriazole) DCC (1,3-dicyclohexylcarbodiimide)
NMR Nuclear magnetic resonance MS Mass spectroscopy
m/z mass to charge ratio LC Liquid chromatography
MgCl₂ Magnesium chloride
EDCl (1-ethyl-3-(3-dimethylaminopropyl)carbodiimide hydrochloride)
TBTU (N,N,N′,N′-tetramethyl-O-(benzotriazol-1-yl)uronium-tetrafluoroborate)
RP-HPLC (reverse phase high performance liquid chromatography)

### Biochemical Assay

The inhibiting activity of putative Cdc7 inhibitors and the potency of selected compounds is determined through the method reported below.

### ADPGlo Assay Format.

The biochemical activity of compounds was determined by incubation with specific enzymes and substrate, followed by quantification of the ADP product. Compounds were 4-fold serially diluted from 10 to 0.0006 uM; in the case of the pre-incubation step, the enzyme is added to the inhibitor solution and the mixture is left to incubate for 30 minutes at room temperature (r.t.). The reaction started with the addition of the substrate and the ATP, when the enzyme was already present in the preincubation step, or substrate, ATP and enzyme when the preincubation step was not present. The concentrations of ATP, substrate and reaction buffer are described above. The assays were run in a robotized format on 384-well plates. Each 384-well plate contained some reference wells (total enzyme activity vs enzyme fully inhibited by specific Inhibitor) that were used for the Z′ and signal to background evaluation. At the end of the incubation time, an equal volume of ADPGlo Reagent 1 (Promega) was added to stop the reaction and to remove all unreacted ATP. After 60 minutes, an equal volume of ADPGlo Reagent 2 (Promega) was added to convert ADP in ATP and then ATP in light by luciferase reaction. After 15 minutes at r.t. the luminescence signal was read with Plate reader The data, obtained with both assay formats, were analyzed by an internally customized version of the SW package “Assay Explorer” which provides sigmoidal fittings of the eight-dilution curves for IC₅₀ determination using a 4-parameter logistic equation:

y=bottom+(top−bottom)/(1+10\hat{ }((log IC₅₀−x)*slope))

where x is the logarithm of the inhibitor concentration, y is the response; y starts at bottom and goes to top with a sigmoid shape.

All information about plate dilution, distribution, type of assay, targets and raw data of inhibition are tracked via barcode reading and stored in an Oracle DB.

Biochemical activity of the compounds of the present invention, obtained with the methods above-reported, are summarized in the table 1 below.

TABLE 1

| Compd | CDC7 IC₅₀ |
|---|---|
| Number | μM ADPGlo |
| 1 | 0.004 |
| 2 | 0.003 |
| 3 | 0.001 |
| 4* | 0.004 |
| 5 | 0.001 |
| 6* | 0.005 |
| 7 | 0.001 |
| 8 | 0.003 |
| 9 | 0.001 |
| 10 | 0.003 |
| 11 | 0.007 |
| 12 | 0.004 |
| 13 | 0.001 |
| 14 | 0.010 |
| 15* | 0.006 |
| 16 | 0.006 |
| 17 | 0.001 |
| 18 | 0.031 |
| 19 | 0.015 |
| 20 | 0.005 |
| 21 | 0.034 |
| 22 | 0.027 |
| 24** | 0.741 |
| 26** | 0.012 |
| 27 | 0.061 |
| 28 | 0.067 |
| 30 | 3.134 |
| 31 | 0.759 |
| 32 | 0.017 |
| 33 | 0.014 |
| 34 | 0.042 |
| 35 | 0.007 |
| 36 | 0.010 |
| 37 | 0.002 |
| 38 | 0.101 |
| 39 | 0.159 |
| 40 | 0.717 |
| 42 | 0.090 |
| 43 | 0.013 |
| 44 | 0.048 |
| 46 | 0.001 |
| 47 | 0.004 |
| 48 | 0.188 |
| 49 | 0.018 |
| 54 | 0.418 |
| 55 | 0.996 |
| 56 | 0.014 |
| 57 | 0.017 |

*compound tested as chloridrate salt
**compound tested as TFA salt
ND: not determined

As summarized in the table 1 the compounds of the present invention show a remarkable activity on cell division cycle 7-related protein kinase (Cdc7).

### Metabolic Stability Assay

The aldehyde oxidases involvement in the metabolism of the test item was determined by comparison of the intrinsic clearance values in the presence and absence of aldehyde oxidases inhibitor hydralazine hydrochloride. The purpose of the study was to evaluate the in-vitro intrinsic clearance, the metabolic stability towards aldehyde oxidases (AO) activity and the metabolism of the test item in human liver cytosol. The intrinsic clearance was determined using the half-life approach, by measuring the substrate disappearance along 60 minutes incubation with human liver cytosol. Incubations were performed at the concentration of 1 μM. The starting concentration of 1 μM was assumed to be << of Km. HPLC-MS/MS was used for the detection of the compound left over during the incubation. For intrinsic clearance determination samples were analyzed by high performance liquid chromatography (HPLC) on-line with mass spectrometry (MS) equipment. For the determination of aldehyde oxidases activity (i.e. positive control) of cytosol, phthalazine was incubated with human liver cytosol in presence and absence of the aldehyde oxidases inhibitor. On the day of the incubation, the compound was dissolved in DMSO at the concentration of 10 mM. Aliquots of this solution were added to the test system to a final concentration of 1. The percentage of DMSO in the final incubates was 0.10%. Aliquots of the test item were added to human liver cytosol (1 mg/mL protein content) in Dulbecco's buffer, pH 7.4 at 37° C., to reach final concentration of 1 μM. Incubations were performed in a 48-well plate, under shaking. At 0, 5, 10, 20, 30 and 60 minutes incubation, 50 μL aliquots of the incubate were sampled, poured in 80 μL of ice-cold acetonitrile and 20 μL of 1 μM warfarin in acetonitrile (injection control) and centrifuged at 2500 rpm for 20 minutes. The supernatant was immediately analysed by LC-MS/MS.

The test item was incubated in parallel, in duplicate, at the concentration of 1 μM in Dulbecco's buffer, pH 7.4 for 60 min at 37° C. in the presence of 10 μM hydralazine hydrochloride (aldehyde oxidases inhibitor).

The chemical stability of the test item was checked in parallel by incubating the test item in single at the concentration of 1 μM in Dulbecco's buffer, pH 7.4 for 60 min at 37° C. (negative control).

The intrinsic clearance (CLint) was calculated using the half-life approach. Half-life and CLint were determined from the concentration (area counts) remaining at the different sampling points using the LC-MS/MS method. By plotting the natural logarithmic area of the compound remaining against the time, the slope was calculated by linear regression analysis and converted into the half-life (t½) and CLint expressed as μL/min/mg protein according to the following formulae:

$$t 1 / 2 ( min ) = \frac{0.693}{- Slope}$$

$${CL}_{in} t = \frac{- Slope \times 1000 ( μ L / mL )}{proteinconc ( mg protein / mL )} ( μ L / min / mg protein$$

The formation of the Phthalazine metabolite Phthalazone was determined from the concentration at the different sampling points using the HPLC-MS/MS method. By plotting the concentration of the metabolite against the time, the slope was calculated, and its maximum value converted into metabolite formation rate expressed in pmol/min/mg.

Table 2 below reports the methabolic stability results of the compounds of the present invention in comparison with reference compound A (Ref. compd. A) and reference compound B (Ref. compd. B), that have been identified as the closest prior art.

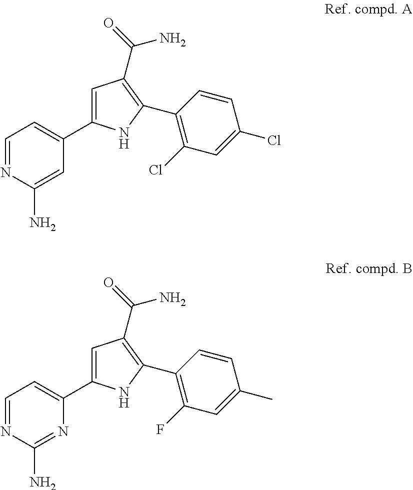 [SMILES: NC(=O)c1cc(-c2ccnc(N)c2)[nH]c1-c1ccc(Cl)cc1Cl ; Cc1ccc(-c2[nH]c(-c3ccnc(N)n3)cc2C(N)=O)c(F)c1]

Reference compound A and reference compound B correspond to compound (53) and (55), respectively, of the International PCT application WO2007/110344

TABLE 2

| | Aldehyde Oxidase Test | | |
|---|---|---|---|
| | HLC % remaining | HLC | |
| Compd Number | (1 h incub.) | T½ min | IntCl |
| Ref. compd A | 42 | 14.8 | 46.7 |
| Ref. compd. B | 70 | 154 | 4.5 |
| 1* | 100 | 239 | 2.9 |
| 2 | 98 | 239 | 2.9 |
| 3 | 97 | 239 | 2.9 |
| 4* | 100 | 239 | 2.9 |
| 5* | 100 | 239 | 2.9 |
| 6* | 100 | 239 | 2.9 |
| 7* | 95 | 239 | 2.9 |
| 8* | 85 | 239 | 2.9 |
| 9* | 100 | 239 | 2.9 |
| 10* | 95 | 239 | 2.9 |
| 11* | 100 | 239 | 2.9 |
| 12 | 97 | 239 | 2.9 |
| 13 | 100 | 239 | 2.9 |
| 14 | 100 | 239 | 2.9 |
| 15 | 100 | 239 | 2.9 |
| 42* | 100 | 239 | 2.9 |
| 43 | 100 | 239 | 2.9 |
| 44 | 100 | 239 | 2.9 |
| 45** | 98 | 239 | 2.9 |
| 46 | 100 | 239 | 2.9 |
| 48 | 100 | 239 | 2.9 |
| 49 | 100 | 239 | 2.9 |
| 52 | 100 | 239 | 2.9 |
| 53 | 100 | 239 | 2.9 |

HLC: Human Liver Cytosol
IntCl: Intrinsic Clearance
*compound tested as chloridrate salt
**compound tested as TFA salt

As shown in Table 2, either reference compound A than reference compound B show poor metabolic stability when incubated in human liver cytosol demonstrating a low half-life (T1/1=14.8 min and 154 min, respectively) and a low percentage of compound remaining after 60 min of incubation (40% and 70%, respectively). It was surprisingly found that the compounds of the present invention, under the same experimental conditions, show a significantly better metabolic stability than prior art compounds.

In particular, the new compounds display an increased half-live (225 min), a higher remaining compound percentage (85% in the worst case) and a decreased Intrinsic Clearance value, thus resulting superior in terms of DMPK properties respect to the reference prior art compounds, therefore increasing the chance of becoming drug candidates.

### Preparation of Compounds of Formula (I)

For a reference to any specific compound of formula (I) of the invention, optionally in the form of a pharmaceutically acceptable salt, see the experimental section and claims. Referring to the examples that follow, compounds of the present invention were synthesized using the methods described herein, or other methods, which are well known in the art.

With the aim at better illustrating the present invention, without posing any limitation to it, the following examples are given.

As used herein the symbols and conventions used in the processes, schemes and examples are consistent with those used in the contemporary scientific literature, for example, the Journal of the American Chemical Society or the Journal of Biological Chemistry.

Compound names are IUPAC names, generated by using ACD Name (by Advanced Chemistry Development, Inc.). Unless otherwise noted, all materials, including anhydrous solvent such as DMF, THF, DCM, were obtained from commercial suppliers, of the best grade and used without further purification. All reactions involving air- or moisture-sensitive compounds were performed under nitrogen or argon atmosphere.

### General Purification and Analytical Methods

Flash Chromatography was performed on silica gel (Merck grade 9395, 60A).

The HPLC equipment consisted of a Waters Alliance™ HT 2795 system equipped with a Waters 996 PDA detector and Waters mod. ZQ 2000 single quadrupole mass spectrometer, equipped with an electrospray (ESI) ion source. Instrument control, data acquisition and data processing were provided by Empower 2 and MassLynx 4.1 softwares. HPLC was carried out at 25° C. at a flow rate of 1.2 mL/min using a YMC-Triart C18 (4.6×50 mm, 3 μm) column. Mobile phase B was ammonium acetate 5 mM pH=5.2 buffer with acetonitrile (95:5), and mobile phase C was H₂O/acetonitrile (5:95); the gradient was from 10 to 90% C in 5 minutes then ramp to 100% C in 0.1 minutes. The injection volume was 10 μL. The mass spectrometer operated in positive and in negative ion mode, the capillary voltage was set up at 3.5 kV (ES⁺) and 2.8 kV (ES⁻); cone voltage was 14 V (ES⁺) and 28 V (ES⁻); the source temperature was 120° C.; full scan, mass range from 100 to 800 amu was set up.

The preparative HPLC equipment consisted of a Shimadzu HPLC system equipped with SCL-8A System Controller, two LC-8A Pumps, SPD-6A UV Spectrophotometric Detector and manual Rheodyne injection system. Data acquisition (analogic signal) and data processing were provided by Empower 2 software. Purification was carried out at 25° C. at a flow rate of 15 mL/min using a Waters X-Terra MS RP18 (150×30 mm, 10 μm) column. Mobile phase A was 0.1% TFA in water/acetonitrile (95:5) or, alternatively, Mobile phase A was 0.05% NH₃ in water/acetonitrile (95:5) and mobile phase B was H₂O/acetonitrile (5:95); the gradient was from 10 to 90% B in 15 minutes then ramp to 100% B in 0.1 minutes. Injection volume max 500 μL.

¹H-NMR spectra were recorded at a constant temperature of 28° C. on a Varian INOVA 400 spectrometer operating at 400.5 MHz and equipped with a 5 mm ¹H{¹⁵N-³¹P} z-axis PFG Indirect Detection probe and on a Varian INOVA 500 spectrometer operating at 499.7 MHz and equipped with a 5 mm ¹H{¹³C-¹⁵N} triple resonance Indirect Detection probe. Chemical shifts were referenced with respect to the residual solvent signals (DMSO-d₆: 2.50 ppm for ¹H). Data are reported as follows: chemical shift (δ), multiplicity (s=singlet, d=doublet, t=triplet, q=quartet, br. s=broad singlet, dd=doublet of doublets, ddd=doublet of doublets of doublets, m=multiplet), coupling constants (J, Hz) and number of protons.

As formerly reported (M. Colombo, F. R. Sirtori, V. Rizzo, Rapid Commun Mass Spectrom 2004, 18(4), 511-517), ESI(+) high-resolution mass spectra (HRMS) were obtained on a Q-Tof Ultima (Waters, Manchester, UK) mass spectrometer directly connected with an Agilent 1100 micro-HPLC system (Palo Alto, US).

### Example A

### Step 1

### methyl 5-[1-(phenylsulfonyl)-1H-pyrrolo[2,3-b]pyridin-4-yl]-1H-pyrrole-3-carboxylate (IV)

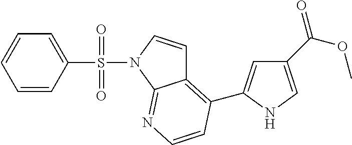 [SMILES: COC(=O)c1c[nH]c(-c2ccnc3c2ccn3S(=O)(=O)c2ccccc2)c1]

In a reactor, under argon atmosphere, methyl 5-bromo-1H-pyrrole-3-carboxylate (1 eq., 5 g, 24.51 mmol), 1-(phenylsulfonyl)-4-(4,4,5,5-tetramethyl-1,3,2-dioxaborolan-2-yl)-1H-pyrrolo[2,3-b]pyridine (1 eq., 9.41 g, 24.51 mmol), Na₂CO₃ (3 eq., 7.79 g, 73.53 mmol), 1,4-dioxane degassed (25 ml) and distilled water degassed (6.25 ml) were added. After three cycles of vacuum/argon, the catalyst [1,1′-Bis(diphenylphosphino)ferrocene]dichloropalladium(II), complex with dichloromethane (0.1 eq., 2 g, 2.45 mmol) was added. After three cycles of vacuum/argon the reaction mixture was heated at T=100° C. for 30 minutes. Distilled water was added and the product was extracted with AcOEt (3 times). The organic layer was washed with distilled water and brine, dried over anhydrous Na₂SO₄ and evaporated to dryness. The crude was purified by flash-chromatography (DCM/Acetone 95/5-9/1) affording the title compound (solid, 4.58 g, Y=49%).

¹H NMR (500 MHz, DMSO-d₆) δ ppm 3.75 (s, 3H) 7.14 (s, 1H) 7.16 (d, J=4.12 Hz, 1H) 7.52 (d, J=5.19 Hz, 1H) 7.60-7.65 (m, 2H) 7.70 (s, 1H) 7.71-7.75 (m, 1H) 7.97 (d, J=4.27 Hz, 1H) 8.09-8.16 (m, 2H) 8.35 (d, J=5.19 Hz, 1H) 12.30 (br. s., 1H). LCMS: m/z 382 [M+H]⁺. HRMS (ESI) calcd for C₁₉H₁₅N₃O₄S [M+H]⁺ 382.0856 found 382.0855;

### Step 2

### methyl 2-bromo-5-[1-(phenylsulfonyl)-1H-pyrrolo[2,3-b]pyridin-4-yl]-1H-pyrrole-3-carboxylate (V)

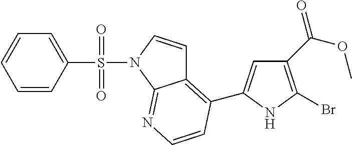 [SMILES: COC(=O)c1cc(-c2ccnc3c2ccn3S(=O)(=O)c2ccccc2)[nH]c1Br]

To a solution of methyl 5-[1-(phenylsulfonyl)-1H-pyrrolo[2,3-b]pyridin-4-yl]-1H-pyrrole-3-carboxylate (1 eq., 4 g, 10.499 mmol) in MeOH (410 ml) and THE (205 ml) at T=0° C., 0.33 eq. of N-bromosuccinimide (566.3 mg, 3.18 mmol) was added. The reaction mixture was stirred at T=0° C. for 30 minutes and then 0.33 eq. of N-bromosuccinimide (566.3 mg, 3.18 mmol) was added. The reaction mixture was stirred at T=0° C. for 30 minutes and then the last portion of N-bromosuccinimide (0.33 eq, 566.3 mg, 3.18 mmol) was added. The reaction was stirred for 1 hour and 30 minutes at T=0° C. Distilled water was added and the product was extracted 3 times with AcOEt. The organic layer was washed with brine, dried over anhydrous Na₂SO₄ and evaporated to dryness. The crude was purified by flash-chromatography (DCM/Acetone 99/1-9/1) affording the title compound (white solid, 3.62 g, Y=75%).

¹H NMR (500 MHz, DMSO-d6) δ ppm 3.76 (s, 3H) 7.10 (d, J=4.12 Hz, 1H) 7.15 (s, 1H) 7.53 (d, J=5.19 Hz, 1H) 7.60-7.66 (m, 2H) 7.70-7.75 (m, 1H) 7.97 (d, J=4.12 Hz, 1H) 8.11-8.15 (m, 2H) 8.36 (d, J=5.19 Hz, 1H) 12.93 (s, 1H). LCMS: m/z 459 [M+H]⁺. HRMS (ESI) calcd for C₁₉H₁₄BrN₃O₄S [M+H]⁺ 459.9961 found 459.996;

### Step 3

### Methyl-2-(3-chloro-2-fluorophenyl)-5-[1-(phenylsulfonyl)-1H-pyrrolo[2,3-b]pyridin-4-yl]-1H-pyrrole-3-carboxylate [(VII)]

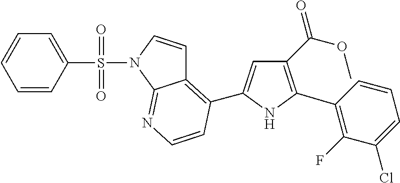 [SMILES: COC(=O)c1cc(-c2ccnc3c2ccn3S(=O)(=O)c2ccccc2)[nH]c1-c1cccc(Cl)c1F]

In a reactor, under argon atmosphere, methyl 2-bromo-5-[1-(phenylsulfonyl)-1H-pyrrolo[2,3-b]pyridin-4-yl]-1H-pyrrole-3-carboxylate (1 eq., 150 mg, 0.33 mmol), (3-chloro-2-fluorophenyl)boronic acid (1.2 eq., 68 mg, 0.39 mmol), Na₂CO₃ (3 eq., 103.7 mg, 0.978 mmol), 1,4-dioxane degassed (4 ml) and distilled water degassed (1 ml) were added. After three cycles of vacuum/argon, the catalyst [1,1′-Bis(diphenylphosphino)ferrocene]dichloropalladium(II), complex with dichloromethane (0.1 eq., 26.6 mg, 0.033 mmol) was added. After three cycles of vacuum/argon the reaction mixture was heated at T=100° C. for 2 hours and 30 minutes. Distilled water was added and the product was extracted with AcOEt (3 times). The organic layer was washed with distilled water and brine, dried over anhydrous Na₂SO₄ and evaporated to dryness. The crude was purified by flash-chromatography (DCM/Acetone 98/2) affording the title compound (solid, 115 mg, Y=69%).

¹H NMR (500 MHz, DMSO-d₆) δ ppm 3.66 (s, 3H) 7.17 (d, J=4.27 Hz, 1H) 7.23 (s, 1H) 7.34 (t, J=7.85 Hz, 1H) 7.53-7.60 (m, 2H) 7.61-7.66 (m, 2H) 7.67-7.71 (m, 1H) 7.71-7.76 (m, 1H) 8.00 (d, J=4.12 Hz, 1H) 8.12-8.15 (m, 2H) 8.37 (d, J=5.19 Hz, 1H) 12.49 (s, 1H). LCMS: m/z 510 [M+H]⁺. HRMS (ESI) calcd for C₂₅H₁₇ClFN₃O₄S [M+H]⁺ 510.0685 found 510.0681;

Operating in an analogous way, but employing suitable substituted starting material the following compounds were obtained:

### Methyl-2-(2-fluoro-4-methylphenyl)-5-[1-(phenylsulfonyl)-1H-pyrrolo[2,3-b]pyridin-4-yl]-1H-pyrrole-3-carboxylate (VII)

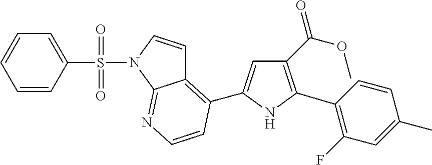 [SMILES: COC(=O)c1cc(-c2ccnc3c2ccn3S(=O)(=O)c2ccccc2)[nH]c1-c1ccc(C)cc1F]

¹H NMR (500 MHz, DMSO-d₆) δ ppm 2.39 (s, 3H) 3.63 (s, 3H) 7.08-7.17 (m, 3H) 7.20 (d, J=1.37 Hz, 1H) 7.44 (t, J=7.78 Hz, 1H) 7.58 (d, J=5.19 Hz, 1H) 7.63 (t, J=1.00 Hz, 2H) 7.73 (t, J=1.00 Hz, 1H) 7.98 (d, J=4.12 Hz, 1H) 8.11-8.17 (m, 2H) 8.35 (d, J=5.19 Hz, 1H) 12.32 (br. s., 1H). LCMS: m/z 490 [M+H]⁺. HRMS (ESI) calcd for C₂₆H₂₀FN₃O₄S [M+H]⁺ 490.1232 found 490.1209;

### Methyl-2-(4-chloro-2-fluorophenyl)-5-[1-(phenylsulfonyl)-1H-pyrrolo[2,3-b]pyridin-4-yl]-1H-pyrrole-3-carboxylate (VII)

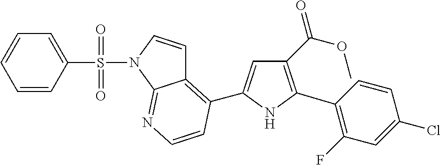 [SMILES: COC(=O)c1cc(-c2ccnc3c2ccn3S(=O)(=O)c2ccccc2)[nH]c1-c1ccc(Cl)cc1F]

¹H NMR (500 MHz, DMSO-d₆) δ ppm 3.65 (s, 3H) 7.16 (d, J=4.12 Hz, 1H) 7.22 (d, J=2.59 Hz, 1H) 7.42 (dd, J=8.31, 2.06 Hz, 1H) 7.55-7.59 (m, 2H) 7.61-7.67 (m, 3H) 7.72 (d, J=7.47 Hz, 1H) 7.99 (d, J=4.12 Hz, 1H) 8.14 (dd, J=8.46, 1.14 Hz, 2H) 8.37 (d, J=5.19 Hz, 1H) 12.43 (d, J=1.52 Hz, 1H). LCMS: m/z 510 [M+H]⁺. HRMS (ESI) calcd for C₂₅H₁₇ClFN₃O₄S [M+H]⁺ 510.0685 found 510.067;

### Methyl-2-(2-chloro-4-fluorophenyl)-5-[1-(phenylsulfonyl)-1H-pyrrolo[2,3-b]pyridin-4-yl]-1H-pyrrole-3-carboxylate (VII)

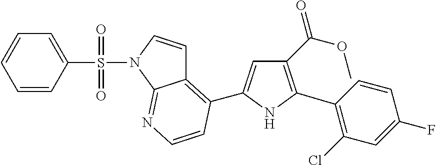 [SMILES: COC(=O)c1cc(-c2ccnc3c2ccn3S(=O)(=O)c2ccccc2)[nH]c1-c1ccc(F)cc1Cl]

¹H NMR (500 MHz, DMSO-d₆) δ ppm 3.61 (s, 3H) 7.16-7.19 (m, 1H) 7.22 (d, J=2.75 Hz, 1H) 7.33 (td, J=8.54, 2.59 Hz, 1H) 7.56 (d, J=5.19 Hz, 1H) 7.58-7.67 (m, 4H) 7.71-7.75 (m, 1H) 7.99 (d, J=4.12 Hz, 1H) 8.10-8.16 (m, 2H) 8.35 (d, J=5.18 Hz, 1H) 12.42 (d, J=1.98 Hz, 1H). LCMS: m/z 510 [M+H]⁺. HRMS (ESI) calcd for C₂₅H₇ClFN₃O₄S [M+H]⁺ 510.0685 found 510.0689;

### Methyl-2-(2,4-difluorophenyl)-5-[1-(phenylsulfonyl)-1H-pyrrolo[2,3-b]pyridin-4-yl]-1H-pyrrole-3-carboxylate (VII)

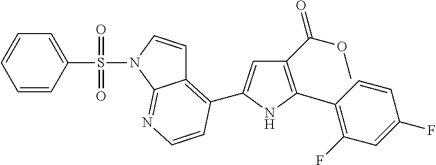 [SMILES: COC(=O)c1cc(-c2ccnc3c2ccn3S(=O)(=O)c2ccccc2)[nH]c1-c1ccc(F)cc1F]

¹H NMR (500 MHz, DMSO-d₆) δ ppm 3.65 (s, 3H) 7.17 (d, J=4.12 Hz, 1H) 7.19-7.25 (m, 2H) 7.39 (td, J=9.84, 2.29 Hz, 1H) 7.58 (d, J=5.19 Hz, 1H) 7.61-7.67 (m, 3H) 7.71-7.75 (m, 1H) 7.99 (d, J=4.12 Hz, 1H) 8.14 (d, J=7.47 Hz, 2H) 8.37 (d, J=5.19 Hz, 1H) 12.41 (br. s., 1H). LCMS: m/z 494 [M+H]⁺. HRMS (ESI) calcd for C₂₅H₁₇F₂N₃O₄S [M+H]⁺ 494.0981 found 494.0977;

### Methyl-2-[2-chloro-4-(trifluoromethyl)phenyl]-5-[1-(phenylsulfonyl)-1H-pyrrolo[2,3-b]pyridin-4-yl]-1H-pyrrole-3-carboxylate (VII)

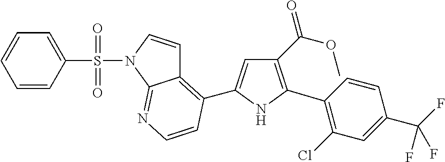 [SMILES: COC(=O)c1cc(-c2ccnc3c2ccn3S(=O)(=O)c2ccccc2)[nH]c1-c1ccc(C(F)(F)F)cc1Cl]

¹H NMR (500 MHz, DMSO-d₆) δ ppm 3.62 (s, 3H) 7.19 (d, J=3.78 Hz, 1H) 7.24 (s, 1H) 7.55 (d, J=5.25 Hz, 1H) 7.60-7.67 (m, 2H) 7.71-7.75 (m, 1H) 7.77-7.85 (m, 2H) 7.99 (d, J=3.78 Hz, 1H) 8.01 (s, 1H) 8.13 (d, J=7.69 Hz, 2H) 8.36 (d, J=5.13 Hz, 1H) 12.48 (br. s., 1H). LCMS: m/z 560 [M+H]⁺. HRMS (ESI) calcd for C₂₅H₁₇ClF₃N₃O₄S [M+H]⁺ 560.0653 found 560.0656;

### Methyl-2-(2,3-difluorophenyl)-5-[1-(phenylsulfonyl)-1H-pyrrolo[2,3-b]pyridin-4-yl]-1H-pyrrole-3-carboxylate (VII)

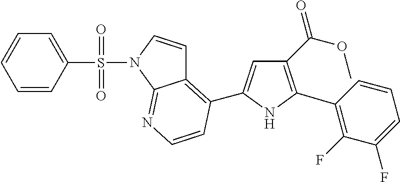 [SMILES: COC(=O)c1cc(-c2ccnc3c2ccn3S(=O)(=O)c2ccccc2)[nH]c1-c1cccc(F)c1F]

¹H NMR (500 MHz, DMSO-d₆) δ ppm 3.66 (s, 3H) 7.17 (d, J=4.12 Hz, 1H) 7.23 (d, J=1.22 Hz, 1H) 7.33 (dd, J=7.78, 5.03 Hz, 1H) 7.39-7.44 (m, 1H) 7.51-7.57 (m, 1H) 7.59 (d, J=5.34 Hz, 1H) 7.61-7.67 (m, 2H) 7.71-7.76 (m, 1H) 8.00 (d, J=4.12 Hz, 1H) 8.14 (dd, J=8.46, 1.14 Hz, 2H) 8.38 (d, J=5.19 Hz, 1H) 12.49 (s, 1H). LCMS: m/z 494 [M+H]⁺. HRMS (ESI) calcd for C₂₅H₁₇F₂N₃O₄S [M+H]⁺ 494.0981 found 494.0981;

### Methyl-2-(2,3-dichlorophenyl)-5-[1-(phenylsulfonyl)-1H-pyrrolo[2,3-b]pyridin-4-yl]-1H-pyrrole-3-carboxylate (VII)

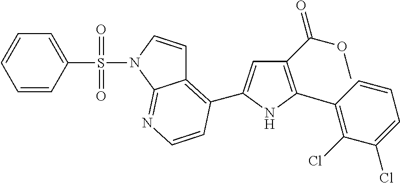 [SMILES: COC(=O)c1cc(-c2ccnc3c2ccn3S(=O)(=O)c2ccccc2)[nH]c1-c1cccc(Cl)c1Cl]

¹H NMR (500 MHz, DMSO-d₆) δ ppm 3.62 (s, 3H) 7.18 (d, J=4.27 Hz, 1H) 7.22 (s, 1H) 7.43-7.48 (m, 1H) 7.50-7.53 (m, 1H) 7.55 (d, J=5.19 Hz, 1H) 7.60-7.67 (m, 2H) 7.70-7.78 (m, 2H) 7.99 (d, J=4.12 Hz, 1H) 8.11-8.16 (m, 2H) 8.36 (d, J=5.19 Hz, 1H) 12.47 (s, 1H). LCMS: m/z 526 [M+H]⁺. HRMS (ESI) calcd for C₂₅H₁₇Cl₂N₃O₄S [M+H]⁺ 526.039 found 526.0383;

### Methyl-2-[4-methyl-2-(trifluoromethyl)phenyl]-5-[1-(phenylsulfonyl)-1H-pyrrolo[2,3-b]pyridin-4-yl]-1H-pyrrole-3-carboxylate (VII)

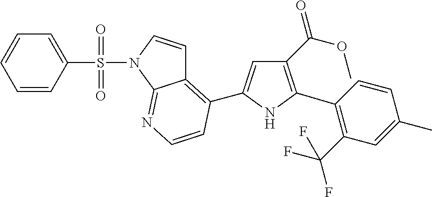 [SMILES: COC(=O)c1cc(-c2ccnc3c2ccn3S(=O)(=O)c2ccccc2)[nH]c1-c1ccc(C)cc1C(F)(F)F]

¹H NMR (500 MHz, DMSO-d₆) δ ppm 2.47 (s, 3H) 3.54 (s, 3H) 7.16 (d, J=4.12 Hz, 1H) 7.19 (d, J=2.75 Hz, 1H) 7.44 (d, J=7.78 Hz, 1H) 7.52 (d, J=5.34 Hz, 1H) 7.55 (d, J=7.63 Hz, 1H) 7.60-7.65 (m, 2H) 7.68 (s, 1H) 7.70-7.75 (m, 1H) 7.98 (d, J=4.12 Hz, 1H) 8.10-8.14 (m, 2H) 8.33 (d, J=5.19 Hz, 1H) 12.39 (d, J=2.14 Hz, 1H). LCMS: m/z 540 [M+H]⁺. HRMS (ESI) calcd for C₂₇H₂₀F₃N₃O₄S [M+H]⁺ 540.1199 found 540.1193;

### Methyl-2-(2-chloro-4-methylphenyl)-5-[1-(phenylsulfonyl)-1H-pyrrolo[2,3-b]pyridin-4-yl]-1H-pyrrole-3-carboxylate (VII)

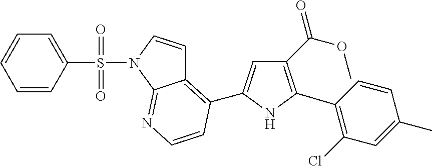 [SMILES: COC(=O)c1cc(-c2ccnc3c2ccn3S(=O)(=O)c2ccccc2)[nH]c1-c1ccc(C)cc1Cl]

¹H NMR (500 MHz, DMSO-d₆) δ ppm 2.38 (s, 3H) 3.60 (s, 3H) 7.16 (d, J=4.12 Hz, 1H) 7.20 (d, J=2.90 Hz, 1H) 7.24 (dd, J=7.78, 0.76 Hz, 1H) 7.39 (s, 2H) 7.56 (d, J=5.19 Hz, 1H) 7.61-7.66 (m, 2H) 7.71-7.76 (m, 1H) 7.98 (d, J=4.12 Hz, 1H) 8.10-8.16 (m, 2H) 8.34 (d, J=5.34 Hz, 1H) 12.34 (d, J=2.29 Hz, 1H). LCMS: m/z 506 [M+H]⁺.

HRMS (ESI) calcd for C₂₅H₂₀ClN₃O₄S [M+H]⁺ 506.0936 found 506.0938;

### Methyl-2-(2,3-difluoro-4-methylphenyl)-5-[1-(phenylsulfonyl)-1H-pyrrolo[2,3-b]pyridin-4-yl]-1H-pyrrole-3-carboxylate (VII)

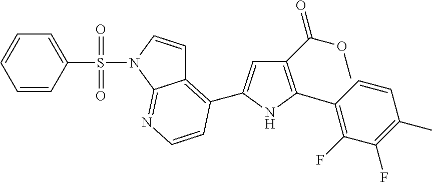 [SMILES: COC(=O)c1cc(-c2ccnc3c2ccn3S(=O)(=O)c2ccccc2)[nH]c1-c1ccc(C)c(F)c1F]

¹H NMR (500 MHz, DMSO-d₆) δ ppm 2.36 (d, J=1.53 Hz, 3H) 3.66 (s, 3H) 7.16 (d, J=4.12 Hz, 1H) 7.18-7.21 (m, 1H) 7.22 (d, J=2.75 Hz, 1H) 7.27-7.32 (m, 1H) 7.59 (d, J=5.34 Hz, 1H) 7.62-7.67 (m, 2H) 7.70-7.76 (m, 1H) 7.99 (d, J=4.12 Hz, 1H) 8.14 (dd, J=8.46, 1.14 Hz, 2H) 8.37 (d, J=5.18 Hz, 1H) 12.43 (d, J=2.14 Hz, 1H). LCMS: m/z 508 [M+H]⁺. HRMS (ESI) calcd for C₂₅H₁₉F₂N₃O₄S [M+H]⁺ 508.1137 found 508.1132;

### Methyl-2-[2-methyl-4-(trifluoromethyl)phenyl]-5-[1-(phenylsulfonyl)-1H-pyrrolo[2,3-b]pyridin-4-yl]-1H-pyrrole-3-carboxylate (VII)

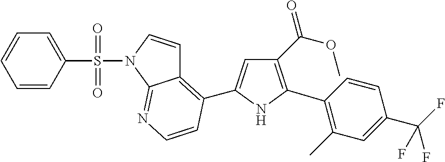 [SMILES: COC(=O)c1cc(-c2ccnc3c2ccn3S(=O)(=O)c2ccccc2)[nH]c1-c1ccc(C(F)(F)F)cc1C]

¹H NMR (500 MHz, DMSO-d₆) δ ppm 2.25 (s, 3H) 3.62 (s, 3H) 7.18 (d, J=4.27 Hz, 1H) 7.25 (s, 1H) 7.52-7.58 (m, 2H) 7.63 (t, J=7.93 Hz, 3H) 7.69-7.76 (m, 2H) 7.99 (d, J=4.12 Hz, 1H) 8.13 (dd, J=8.54, 1.07 Hz, 2H) 8.34 (d, J=5.19 Hz, 1H) 12.36 (s, 1H). LCMS: m/z 540 [M+H]⁺. HRMS (ESI) calcd for C₂₇H₂₀F₃N₃O₄S [M+H]⁺ 540.12 found 540.1207;

### Methyl-2-(2-fluoro-3-methoxyphenyl)-5-[1-(phenylsulfonyl)-1H-pyrrolo[2,3-b]pyridin-4-yl]-1H-pyrrole-3-carboxylate (VII)

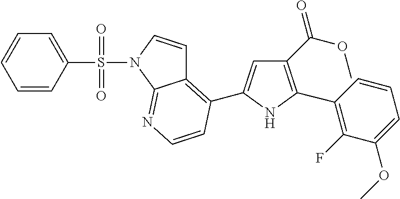 [SMILES: COC(=O)c1cc(-c2ccnc3c2ccn3S(=O)(=O)c2ccccc2)[nH]c1-c1cccc(OC)c1F]

¹H NMR (500 MHz, DMSO-d₆) δ ppm 3.64 (s, 3H) 3.88 (s, 3H) 7.10 (td, J=6.83, 1.75 Hz, 1H) 7.16 (d, J=4.12 Hz, 1H) 7.18-7.30 (m, 3H) 7.59 (d, J=5.19 Hz, 1H) 7.60-7.67 (m, 2H) 7.71-7.77 (m, 1H) 7.98 (d, J=4.12 Hz, 1H) 8.13 (dd, J=8.46, 0.99 Hz, 2H) 8.36 (d, J=5.19 Hz, 1H) 12.39 (br. s., 1H). LCMS: m/z 506 [M+H]⁺. HRMS (ESI) calcd for C₂₅H₂₀FN₃O₅S [M+H]⁺ 506.1181 found 506.1177;

### Methyl-2-(2-chloro-3-fluorophenyl)-5-[1-(phenylsulfonyl)-1H-pyrrolo[2,3-b]pyridin-4-yl]-1H-pyrrole-3-carboxylate (VII)

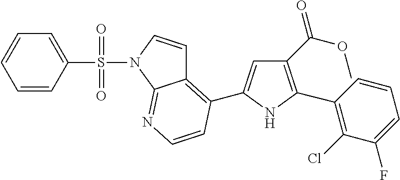 [SMILES: COC(=O)c1cc(-c2ccnc3c2ccn3S(=O)(=O)c2ccccc2)[nH]c1-c1cccc(F)c1Cl]

¹H NMR (500 MHz, DMSO-d₆) δ ppm 3.61 (s, 3H) 7.17 (d, J=4.12 Hz, 1H) 7.22 (s, 1H) 7.39 (d, J=7.02 Hz, 1H) 7.44-7.50 (m, 1H) 7.50-7.54 (m, 1H) 7.55 (d, J=5.19 Hz, 1H) 7.60-7.66 (m, 2H) 7.70-7.75 (m, 1H) 7.99 (d, J=4.12 Hz, 1H) 8.12 (dd, J=8.46, 1.14 Hz, 2H) 8.35 (d, J=5.34 Hz, 1H) 12.48 (br. s., 1H). LCMS: m/z 510 [M+H]⁺. HRMS (ESI) calcd for C₂₅H₁₇ClFN₃O₄S [M+H]⁺ 510.0685 found 510.0686;

### Methyl-2-(2-fluoro-3-methylphenyl)-5-[1-(phenylsulfonyl)-1H-pyrrolo[2,3-b]pyridin-4-yl]-1H-pyrrole-3-carboxylate (VII)

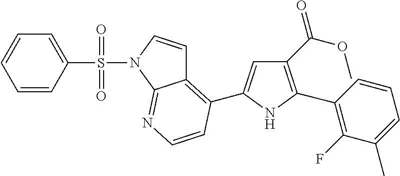 [SMILES: COC(=O)c1cc(-c2ccnc3c2ccn3S(=O)(=O)c2ccccc2)[nH]c1-c1cccc(C)c1F]

¹H NMR (500 MHz, DMSO-d₆) δ ppm 2.30 (d, J=1.59 Hz, 3H) 3.64 (s, 3H) 7.09-7.24 (m, 3H) 7.37 (t, J=7.26 Hz, 2H) 7.59 (d, J=5.25 Hz, 1H) 7.61-7.66 (m, 2H) 7.70-7.76 (m, 1H) 7.98 (d, J=4.15 Hz, 1H) 8.11-8.16 (m, 2H) 8.35 (d, J=5.13 Hz, 1H) 12.34 (br. s., 1H). LCMS: m/z 490 [M+H]⁺. HRMS (ESI) calcd for C₂₆H₂₀FN₃O₄S [M+H]⁺ 490.1232 found 490.1225;

### Methyl-2-[2-methyl-3-(trifluoromethyl)phenyl]-5-[1-(phenylsulfonyl)-1H-pyrrolo[2,3-b]pyridin-4-yl]-1H-pyrrole-3-carboxylate (VII)

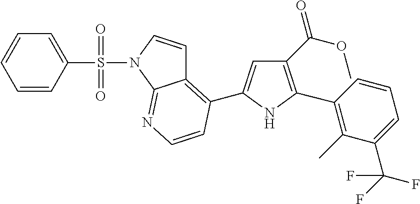 [SMILES: COC(=O)c1cc(-c2ccnc3c2ccn3S(=O)(=O)c2ccccc2)[nH]c1-c1cccc(C(F)(F)F)c1C]

¹H NMR (500 MHz, DMSO-d₆) δ ppm 2.25 (s, 3H) 3.61 (s, 3H) 7.17-7.20 (m, 1H) 7.24-7.26 (m, 1H) 7.44-7.51 (m, 1H) 7.55-7.58 (m, 1H) 7.60-7.67 (m, 3H) 7.70-7.75 (m, 1H) 7.78-7.82 (m, 1H) 7.98-8.00 (m, 1H) 8.10-8.15 (m, 2H) 8.31-8.36 (m, 1H) 12.38 (d, J=2.29 Hz, 1H). LCMS: m/z 540 [M+H]⁺. HRMS (ESI) calcd for C₂₇H₂₀F₃N₃O₄S [M+H]⁺ 540.11994 found 540.1203;

### Methyl-2-[4-methoxy-2-(trifluoromethyl)phenyl]-5-[1-(phenylsulfonyl)-1H-pyrrolo[2,3-b]pyridin-4-yl]-1H-pyrrole-3-carboxylate (VII)

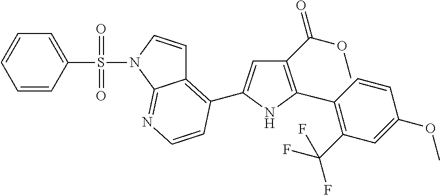 [SMILES: COC(=O)c1cc(-c2ccnc3c2ccn3S(=O)(=O)c2ccccc2)[nH]c1-c1ccc(OC)cc1C(F)(F)F]

¹H NMR (500 MHz, DMSO-d₆) δ ppm 3.55 (s, 3H) 3.90 (s, 3H) 7.15 (d, J=4.12 Hz, 1H) 7.19 (d, J=2.75 Hz, 1H) 7.30 (dd, J=8.46, 2.52 Hz, 1H) 7.33 (d, J=2.59 Hz, 1H) 7.47-7.50 (m, 1H) 7.52 (d, J=5.19 Hz, 1H) 7.61-7.66 (m, 2H) 7.71-7.75 (m, 1H) 7.98 (d, J=4.12 Hz, 1H) 8.09-8.14 (m, 2H) 8.33 (d, J=5.34 Hz, 1H) 12.37 (d, J=2.29 Hz, 1H). LCMS: m/z 556 [M+H]⁺. HRMS (ESI) calcd for C₂₇H₂₀F₃N₃O₅S [M+H]⁺ 556.1149 found 556.1144;

### Methyl-2-[2-chloro-4-(difluoromethoxy)phenyl]-5-[1-(phenylsulfonyl)-1H-pyrrolo[2,3-b]pyridin-4-yl]-1H-pyrrole-3-carboxylate (VII)

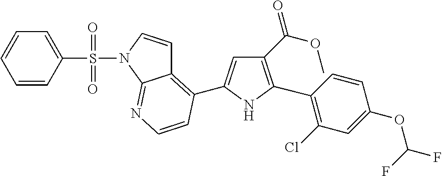 [SMILES: COC(=O)c1cc(-c2ccnc3c2ccn3S(=O)(=O)c2ccccc2)[nH]c1-c1ccc(OC(F)F)cc1Cl]

¹H NMR (500 MHz, DMSO-d₆) δ ppm 3.94 (s, 3H) 7.18 (d, J=4.12 Hz, 1H) 7.22 (d, J=2.75 Hz, 1H) 7.27 (dd, J=8.35, 2.52 Hz, 1H) 7.48 (d, J=2.59 Hz, 1H) 7.55-7.56 (m, 1H) 7.63-7.65 (m, 2H) 7.71-7.74 (m, 1H) 7.98 (d, J=4.10 Hz, 1H) 8.12-8.14 (m, 2H) 8.35 (d, J=5.31 Hz, 1H) 12.42 (d, J=2.24 Hz, 1H). LCMS: m/z 558 [M+H]⁺. HRMS (ESI) calcd for C₂₅H₁₈ClF₂N₃O₅S [M+H]⁺ 558.0697 found 558.0714;

### Methyl-2-(3,4-dichlorophenyl)-5-[1-(phenylsulfonyl)-1H-pyrrolo[2,3-b]pyridin-4-yl]-1H-pyrrole-3-carboxylate (VII)

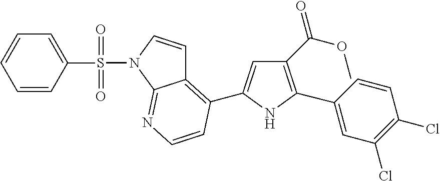 [SMILES: COC(=O)c1cc(-c2ccnc3c2ccn3S(=O)(=O)c2ccccc2)[nH]c1-c1ccc(Cl)c(Cl)c1]

¹H NMR (500 MHz, DMSO-d₆) δ ppm 3.71 (s, 3H) 7.15 (d, J=4.12 Hz, 1H) 7.21 (d, J=2.59 Hz, 1H) 7.61-7.66 (m, 3H) 7.66-7.70 (m, 1H) 7.71-7.76 (m, 2H) 7.94-8.02 (m, 2H) 8.14 (dd, J=8.39, 1.07 Hz, 2H) 8.39 (d, J=5.19 Hz, 1 H) 12.32 (d, J=1.98 Hz, 1H). LCMS: m/z 526 [M+H]⁺. HRMS (ESI) calcd for C₂₅H₁₇Cl₂N₃O₄S [M+H]⁺ 526.039 found 526.0387;

### Methyl-2-(3,4-difluorophenyl)-5-[1-(phenylsulfonyl)-1H-pyrrolo[2,3-b]pyridin-4-yl]-1H-pyrrole-3-carboxylate (VII)

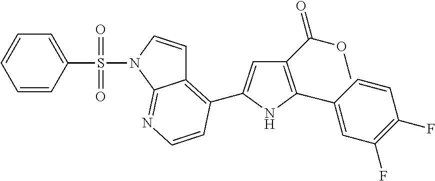 [SMILES: COC(=O)c1cc(-c2ccnc3c2ccn3S(=O)(=O)c2ccccc2)[nH]c1-c1ccc(F)c(F)c1]

¹H NMR (500 MHz, DMSO-d₆) δ ppm 3.70 (s, 3H) 7.15 (d, J=4.12 Hz, 1H) 7.20 (s, 1H) 7.53-7.57 (m, 1H) 7.62-7.67 (m, 3H) 7.70-7.76 (m, 1H) 7.77-7.84 (m, 1H) 7.99 (d, J=4.12 Hz, 1H) 8.12-8.16 (m, 2H) 8.38 (d, J=5.19 Hz, 1H) 12.27 (s, 1H). LCMS: m/z 494 [M+H]⁺. HRMS (ESI) calcd for C₂₅H₁₇F₂N₃O₄S [M+H]⁺ 494.0981 found 494.0982;

### Methyl-2-(3-ethoxy-2-fluorophenyl)-5-[1-(phenylsulfonyl)-1H-pyrrolo[2,3-b]pyridin-4-yl]-1H-pyrrole-3-carboxylate (VII)

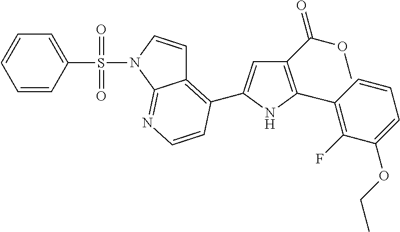 [SMILES: CCOc1cccc(-c2[nH]c(-c3ccnc4c3ccn4S(=O)(=O)c3ccccc3)cc2C(=O)OC)c1F]

¹H NMR (500 MHz, DMSO-d₆) δ ppm 1.37 (t, J=6.94 Hz, 1H) 3.65 (s, 3H) 4.15 (q, J=6.91 Hz, 1H) 7.08 (td, J=6.90, 1.60 Hz, 1H) 7.16 (d, J=4.27 Hz, 1H) 7.17-7.22 (m, 2H) 7.22-7.27 (m, 1H) 7.59 (d, J=5.19 Hz, 1H) 7.61-7.67 (m, 2H) 7.70-7.76 (m, 1H) 7.99 (d, J=4.12 Hz, 1H) 8.13 (dd, J=8.39, 1.07 Hz, 2H) 8.36 (d, J=5.19 Hz, 1H) 12.38 (d, J=2.14 Hz, 1H). LCMS: m/z 520 [M+H]⁺. HRMS (ESI) calcd for C₂₇H₂₂FN₃O₅S [M+H]⁺ 520.1337 found 520.1335;

### Methyl-2-(4-methyl-3-nitrophenyl)-5-[1-(phenylsulfonyl)-1H-pyrrolo[2,3-b]pyridin-4-yl]-1H-pyrrole-3-carboxylate (VII)

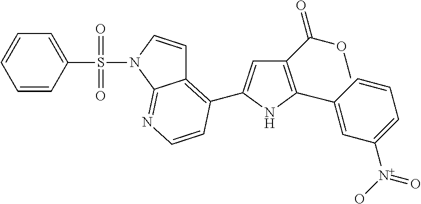

¹H NMR (500 MHz, DMSO-d₆) d ppm 2.58 (s, 3H) 3.69-3.72 (m, 3H) 7.16 (d, J=4.12 Hz, 1H) 7.23 (s, 1H) 7.57-7.68 (m, 4H) 7.70-7.77 (m, 1H) 7.94 (dd, J=7.93, 1.83 Hz, 1H) 8.00 (d, J=4.12 Hz, 1H) 8.12-8.18 (m, 2H) 8.32 (d, J=1.68 Hz, 1H) 8.39 (d, J=5.18 Hz, 1H) 12.36 (s, 1H). LCMS: m/z 517 [M+H]⁺. HRMS (ESI) calcd for C₂₆H₂₀N₄O₆S [M+H]⁺ 517.1177 found 517.118;

### Methyl-2-(4-cyano-3-fluorophenyl)-5-[1-(phenylsulfonyl)-1H-pyrrolo[2,3-b]pyridin-4-yl]-1H-pyrrole-3-carboxylate (VII)

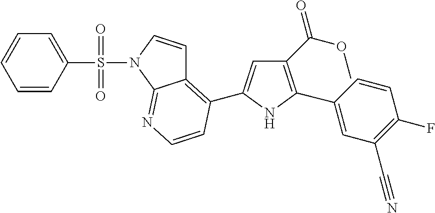 [SMILES: COC(=O)c1cc(-c2ccnc3c2ccn3S(=O)(=O)c2ccccc2)[nH]c1-c1ccc(F)c(C#N)c1]

¹H NMR (500 MHz, DMSO-d₆) δ ppm 3.70 (s, 3H) 7.17 (d, J=4.12 Hz, 1H) 7.21 (d, J=2.75 Hz, 1H) 7.61-7.69 (m, 4H) 7.72-7.77 (m, 1H) 8.00 (d, J=4.12 Hz, 1H) 8.06-8.18 (m, 3H) 8.27 (dd, J=6.18, 2.36 Hz, 1H) 8.39 (d, J=5.19 Hz, 1H) 12.35 (d, J=2.14 Hz, 1H). LCMS: m/z 501 [M+H]⁺. HRMS (ESI) calcd for C₂₆H₁₇FN₄O₄S [M+H]⁺ 501.1028 found 501.102;

### Methyl-2-(dibenzo[b,d]thiophen-4-yl)-5-[1-(phenylsulfonyl)-1H-pyrrolo[2,3-b]pyridin-4-yl]-1H-pyrrole-3-carboxylate (VII)

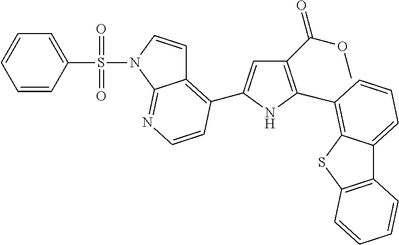 [SMILES: COC(=O)c1cc(-c2ccnc3c2ccn3S(=O)(=O)c2ccccc2)[nH]c1-c1cccc2c1sc1ccccc12]

¹H NMR (500 MHz, DMSO-d₆) δ ppm 3.51-3.60 (m, 3H) 7.23 (d, J=4.27 Hz, 1H) 7.31 (d, J=2.59 Hz, 1H) 7.51-7.56 (m, 2H) 7.59-7.68 (m, 5H) 7.69-7.77 (m, 1H) 7.94-8.05 (m, 2H) 8.09-8.17 (m, 2H) 8.37 (d, J=5.19 Hz, 1H) 8.40-8.51 (m, 2H) 12.60 (d, J=2.14 Hz, 1H). LCMS: m/z 564 [M+H]⁺. HRMS (ESI) calcd for C₃₁H₂₁N₃O₄S₂[M+H]⁺ 564.1046 found 564.1054;

### Methyl-2-(4-methylnaphthalen-1-yl)-5-[1-(phenylsulfonyl)-1H-pyrrolo[2,3-b]pyridin-4-yl]-1H-pyrrole-3-carboxylate (VII)

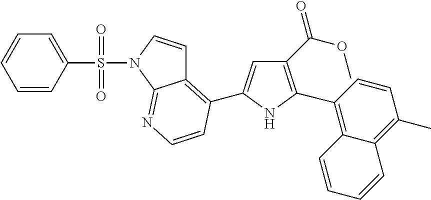 [SMILES: COC(=O)c1cc(-c2ccnc3c2ccn3S(=O)(=O)c2ccccc2)[nH]c1-c1ccc(C)c2ccccc12]

¹H NMR (500 MHz, DMSO-d₆) δ ppm 2.74 (s, 3H) 3.49 (s, 3H) 7.21 (d, J=4.27 Hz, 1H) 7.32 (d, J=2.90 Hz, 1H) 7.42-7.52 (m, 3H) 7.55-7.68 (m, 5H) 7.71-7.78 (m, 1H) 7.99 (d, J=4.12 Hz, 1H) 8.07-8.17 (m, 3H) 8.33 (d, J=5.19 Hz, 1H) 12.44 (d, J=2.14 Hz, 1H). LCMS: m/z 522 [M+H]⁺. HRMS (ESI) calcd for C₃₀H₂₃N₃O₄S [M+H]⁺ 522.1482 found 522.1477;

### Methyl 2-(3-fluorophenyl)-5-[1-(phenylsulfonyl)-1H-pyrrolo[2,3-b]pyridin-4-yl]-1H-pyrrole-3-carboxylate (VII)

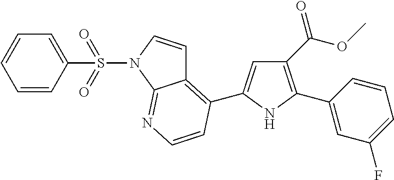 [SMILES: COC(=O)c1cc(-c2ccnc3c2ccn3S(=O)(=O)c2ccccc2)[nH]c1-c1cccc(F)c1]

1H NMR (500 MHz, DMSO-d6) δ ppm 3.70 (s, 3H) 7.15 (d, J=4.27 Hz, 1H) 7.21 (d, J=2.75 Hz, 1H) 7.23-7.30 (m, 1H) 7.44-7.56 (m, 3H) 7.60-7.68 (m, 3H) 7.73 (d, J=7.47 Hz, 1H) 7.99 (d, J=4.27 Hz, 1H) 8.04-8.17 (m, 2H) 8.37 (d, J=5.34 Hz, 1H) 12.26 (d, J=2.14 Hz, 1H). LCMS: m/z 476 [M+H]⁺. HRMS (ESI) calcd for C₂₅H₁₈FN₃O₄S [M+H]⁺ 476.1075 found 476.1072;

### Methyl 5-[1-(phenylsulfonyl)-1H-pyrrolo[2,3-b]pyridin-4-yl]-2-[4-(trifluoromethoxy)phenyl]-1H-pyrrole-3-carboxylate (VII)

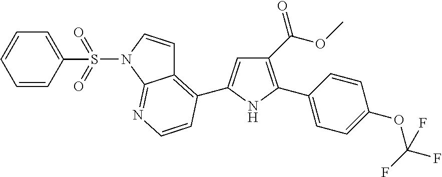 [SMILES: COC(=O)c1cc(-c2ccnc3c2ccn3S(=O)(=O)c2ccccc2)[nH]c1-c1ccc(OC(F)(F)F)cc1]

¹H NMR (500 MHz, DMSO-d6) δ ppm 3.69 (s, 3H) 7.16 (d, J=4.12 Hz, 1H) 7.21 (d, J=2.75 Hz, 1H) 7.47 (d, J=8.08 Hz, 2H) 7.57-7.67 (m, 3H) 7.70-7.76 (m, 1H) 7.77-7.84 (m, 2H) 7.99 (s, 1H) 8.07-8.18 (m, 2H) 8.37 (d, J=5.19 Hz, 1H) 12.28 (d, J=2.29 Hz, 1H). LCMS: m/z 542 [M+H]⁺. HRMS (ESI) calcd for C₂₆H₁₈F₃N₃O₅S [M+H]⁺ 542.0992 found 542.0999;

### Methyl 2-(1-benzothiophen-3-yl)-5-[1-(phenylsulfonyl)-1H-pyrrolo[2,3-b]pyridin-4-yl]-1H-pyrrole-3-carboxylate (VII)

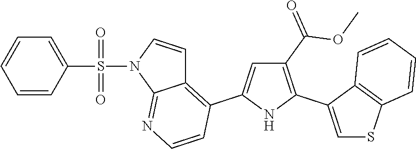 [SMILES: COC(=O)c1cc(-c2ccnc3c2ccn3S(=O)(=O)c2ccccc2)[nH]c1-c1csc2ccccc12]

¹H NMR (500 MHz, DMSO-d₆) δ ppm 3.57 (s, 3H) 7.20 (d, J=4.12 Hz, 1H) 7.29 (d, J=2.59 Hz, 1H) 7.37-7.45 (m, 2H) 7.57 (m, J=1.98 Hz, 1H) 7.64 (m, J=5.19 Hz, 3H) 7.73 (tt, J=7.50, 1.40 Hz, 1H) 8.00 (m, J=4.12 Hz, 2H) 8.07 (m, J=1.83 Hz, 1H) 8.14 (m, J=8.46, 1.14 Hz, 2H) 8.36 (d, J=5.34 Hz, 1H) 12.45 (d, J=1.83 Hz, 1H). LCMS: m/z 514 [M+H]⁺. HRMS (ESI) calcd for C₂₇H₁₉N₃O₄S₂[M+H]⁺ 514.089 found 514.0878;

### Methyl 2-(2,3-dihydro-1,4-benzodioxin-6-yl)-5-[1-(phenylsulfonyl)-1H-pyrrolo[2,3-b]pyridin-4-yl]-1H-pyrrole-3-carboxylate (VII)

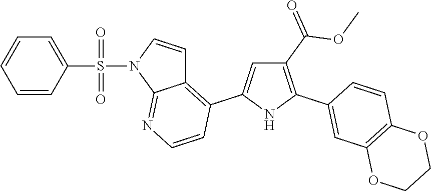 [SMILES: COC(=O)c1cc(-c2ccnc3c2ccn3S(=O)(=O)c2ccccc2)[nH]c1-c1ccc2c(c1)OCCO2]

¹H NMR (500 MHz, DMSO-d6) δ ppm 3.68 (s, 3H) 4.22-4.35 (m, 4H) 6.93 (d, J=8.39 Hz, 1H) 7.12 (d, J=4.12 Hz, 1H) 7.13-7.18 (m, 2H) 7.20 (d, J=2.14 Hz, 1H) 7.58-7.68 (m, 3H) 7.69-7.76 (m, 1H) 7.97 (d, J=4.12 Hz, 1H) 8.13 (dd, J=8.46, 0.99 Hz, 2H) 8.34 (d, J=5.19 Hz, 1H) 12.06 (s, 1H). LCMS: m/z 516 [M+H]⁺. HRMS (ESI) calcd for C₂₇H₂₁N₃O₆S [M+H]⁺ 516.1224 found 516.1206;

### Methyl 2-(4-fluoro-2-methylphenyl)-5-[1-(phenylsulfonyl)-1H-pyrrolo[2,3-b]pyridin-4-yl]-1H-pyrrole-3-carboxylate (VII)

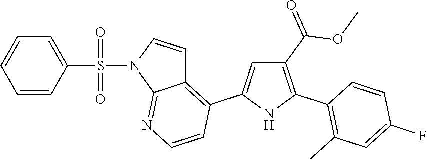 [SMILES: COC(=O)c1cc(-c2ccnc3c2ccn3S(=O)(=O)c2ccccc2)[nH]c1-c1ccc(F)cc1C]

¹H NMR (500 MHz, DMSO-d₆) δ ppm 2.16 (s, 1H) 3.61 (s, 3H) 7.10 (td, J=8.50, 2.67 Hz, 1H) 7.16 (d, J=4.12 Hz, 1H) 7.18-7.22 (m, 2H) 7.36 (dd, J=8.39, 6.10 Hz, 1H) 7.57 (d, J=5.19 Hz, 1H) 7.61-7.65 (m, 2H) 7.70-7.75 (m, 1H) 7.97 (d, J=4.12 Hz, 1H) 8.11-8.13 (m, 2H) 8.33 (d, J=5.19 Hz, 1H) 12.26 (br. s., 1H). LCMS: m/z 490 [M+H]⁺. HRMS (ESI) calcd for C₂₆H₂₀FN₃O₄S [M+H]⁺ 490.1232 found 490.1224;

### Conversion 1

### Methyl 2-(2-fluoro-4-methylphenyl)-4-iodo-5-[1-(phenylsulfonyl)-1H-pyrrolo[2,3-b]pyridin-4-yl]-1H-pyrrole-3-carboxylate (VII)

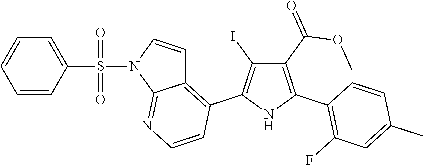 [SMILES: COC(=O)c1c(-c2ccc(C)cc2F)[nH]c(-c2ccnc3c2ccn3S(=O)(=O)c2ccccc2)c1I]

To a solution of methyl-2-(2-fluoro-4-methylphenyl)-5-[1-(phenylsulfonyl)-1H-pyrrolo[2,3-b]pyridin-4-yl]-1H-pyrrole-3-carboxylate (1 eq., 200 mg, 0.40 mmol) in DMF (2 ml), N-iodosuccinimide (1.15 eq., 106 mg, 0.47 mmol) was added. The reaction mixture was stirred at room temperature for 15 h. Ice and distilled water were added and the formation of a precipitate was observed. The solid was filtered and was washed three times with distilled water (and three times with Et₂O to achieve the title compound (beige solid, 960 mg, Y=Quant. %).

¹H NMR (500 MHz, DMSO-d₆) δ ppm 2.36 (s, 3H) 3.58 (s, 3H) 6.84 (d, J=3.81 Hz, 1H) 7.05-7.16 (m, 2H) 7.45 (t, J=8.01 Hz, 1H) 7.48-7.53 (m, 1H) 7.60-7.69 (m, 2H) 7.70-7.77 (m, 1H) 8.00 (d, J=4.12 Hz, 1H) 8.10-8.21 (m, 2H) 8.46 (d, J=4.88 Hz, 1H) 12.49 (br. s., 1H). LCMS: m/z 616 [M+H]⁺. HRMS (ESI) calcd for C₂₆H₁₉FlN₃O₄S [M+H]⁺ 616.0198 found 616.0182;

### Methyl 2-(2-fluoro-4-methylphenyl)-4-bromo-5-[1-(phenylsulfonyl)-1H-pyrrolo[2,3-b]pyridin-4-yl]-1H-pyrrole-3-carboxylate (VII)

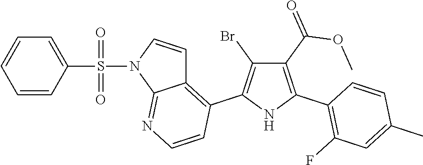 [SMILES: COC(=O)c1c(-c2ccc(C)cc2F)[nH]c(-c2ccnc3c2ccn3S(=O)(=O)c2ccccc2)c1Br]

To a solution of methyl-2-(2-fluoro-4-methylphenyl)-5-[1-(phenylsulfonyl)-1H-pyrrolo[2,3-b]pyridin-4-yl]-1H-pyrrole-3-carboxylate (1 eq., 300 mg, 0.61 mmol) in MeOH (24.4 ml) and THE (6.1 ml) at T=0° C., 0.33 eq. of N-bromosuccinimide (36 mg, 0.3 mmol) was added. The reaction mixture was stirred at T=0° C. for 30 minutes and then 0.33 eq. of N-bromosuccinimide (36 mg, 0.3 mmol) was added. The reaction mixture was stirred at T=0° C. for 30 minutes and then the last portion of N-bromosuccinimide (0.33 eq, 36 mg, 0.3 mmol) was added. The reaction was stirred for 1 hour and 30 minutes at T=0° C. Distilled water was added and the product was extracted 3 times with AcOEt. The organic layer was washed with brine, dried over anhydrous Na₂SO₄ and evaporated to dryness. The crude was purified by flash-chromatography (Hexane/AcOEt 6/4) affording the title compound (white solid, 188 mg, Y=54%). LCMS: m/z 568 [M+H]⁺. HRMS (ESI) calcd for C₂₆H₁₉BrFN₃O₄S [M+H]⁺ 568.0336 found 568.0331;

### Step 4

### Methyl-2-(3-chloro-2-fluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

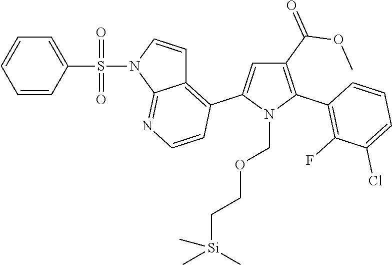 [SMILES: COC(=O)c1cc(-c2ccnc3c2ccn3S(=O)(=O)c2ccccc2)n(COCC[Si](C)(C)C)c1-c1cccc(Cl)c1F]

To a solution of methyl 2-(3-chloro-2-fluorophenyl)-5-[1-(phenylsulfonyl)-1H-pyrrolo[2,3-b]pyridin-4-yl]-1H-pyrrole-3-carboxylate (1 eq., 100 mg, 0.196 mmol) in THE dry (1 ml) at T=0° C. NaH 60% in mineral oil (1.7 eq., 13.4 mg, 0.33 mmol) was added. The reaction mixture was stirred at T=0° C. for 20 minutes and SEMCl (1.8 eq., 63 μl, 0.35 mmol) was added. After 10 minutes at T=0° C. the reaction was warmed at room temperature and was stirred for 3 hours. Distilled water was added and the product was extracted with AcOEt. The organic layer was washed with distilled water and brine, dried over anhydrous Na₂SO₄ and evaporated to dryness. The crude was purified by flash-chromatography (Hex/AcOEt 85/15-7/3) affording the title compound (solid, 118 mg, Y=94%).

¹H NMR (500 MHz, DMSO-d₆) d ppm −0.34-−0.21 (m, 9H) 0.40-0.59 (m, 2H) 2.89-2.98 (m, 2H) 3.62 (s, 3H) 4.96-5.21 (m, 2H) 6.90 (d, J=4.12 Hz, 1H) 6.92 (s, 1H) 7.36 (t, J=7.93 Hz, 1H) 7.50 (d, J=5.03 Hz, 1H) 7.52-7.56 (m, 1H) 7.62-7.68 (m, 2H) 7.71-7.77 (m, 2H) 8.01 (d, J=3.97 Hz, 1H) 8.17 (dd, J=8.46, 1.14 Hz, 2H) 8.45 (d, J=5.03 Hz, 1H). LCMS: m/z 640 [M+H]⁺. HRMS (ESI) calcd for C₃₁H₃₁ClFN₃O₅SSi [M+H]⁺ 640.1499 found 640.149; Operating in an analogous way, but employing suitable substituted starting material the following compounds were obtained:

### Methyl-2-(2-fluoro-4-methylphenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

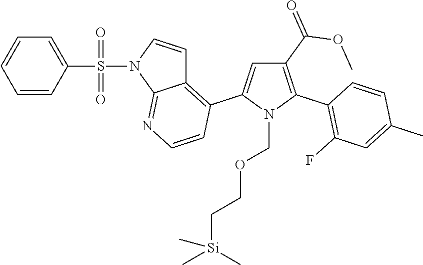 [SMILES: COC(=O)c1cc(-c2ccnc3c2ccn3S(=O)(=O)c2ccccc2)n(COCC[Si](C)(C)C)c1-c1ccc(C)cc1F]

¹H NMR (500 MHz, DMSO-d₆) d ppm −0.33-−0.26 (m, 9H) 0.47 (t, J=8.31 Hz, 1H) 2.40 (s, 3H) 2.93 (q, J=8.29 Hz, 1H) 3.60 (s, 3H) 4.98-5.18 (m, 2H) 6.87-6.94 (m, 2H) 7.11-7.19 (m, 2H) 7.36-7.41 (m, 1H) 7.51 (d, J=5.19 Hz, 1H) 7.62-7.68 (m, 2H) 7.71-7.79 (m, 1H) 8.00 (d, J=4.12 Hz, 1H) 8.13-8.22 (m, 2H) 8.44 (d, J=5.03 Hz, 1H). LCMS: m/z 620 [M+H]⁺. HRMS (ESI) calcd for C₃₁H₃₁ClFN₃O₅SSi [M+H]⁺ 620.2045 found 620.2047;

### Methyl-2-(4-chloro-2-fluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

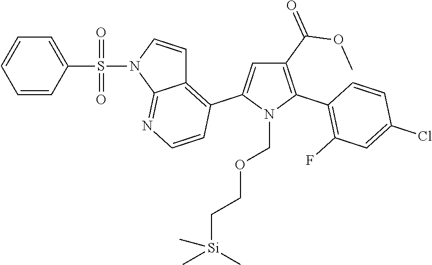 [SMILES: COC(=O)c1cc(-c2ccnc3c2ccn3S(=O)(=O)c2ccccc2)n(COCC[Si](C)(C)C)c1-c1ccc(Cl)cc1F]

¹H NMR (500 MHz, DMSO-d₆) d ppm −0.30-−0.28 (m, 9H) 0.44-0.50 (m, 2H) 2.91-2.97 (m, 2H) 3.59-3.64 (m, 3H) 5.00-5.18 (m, 2H) 6.89 (d, J=4.12 Hz, 1H) 6.91 (s, 1H) 7.44 (dd, J=8.24, 1.98 Hz, 1H) 7.47-7.51 (m, 1H) 7.57-7.62 (m, 2H) 7.63-7.67 (m, 2H) 7.73-7.77 (m, 1H) 8.01 (d, J=3.97 Hz, 1H) 8.17 (dd, J=8.46, 1.14 Hz, 2H) 8.45 (d, J=5.19 Hz, 1H). LCMS: m/z 640 [M+H]⁺. HRMS (ESI) calcd for C₃₁H₃₁ClFN₃O₅SSi [M+H]⁺ 640.1499 found 640.1;

### Methyl-2-(2-chloro-4-fluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

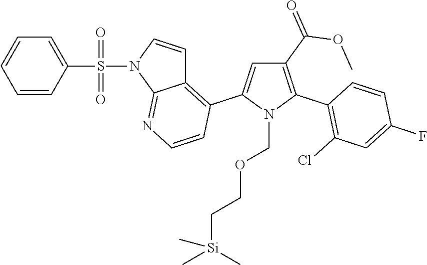 [SMILES: COC(=O)c1cc(-c2ccnc3c2ccn3S(=O)(=O)c2ccccc2)n(COCC[Si](C)(C)C)c1-c1ccc(F)cc1Cl]

¹H NMR (500 MHz, DMSO-d₆) d ppm −0.31-−0.27 (m, 9H) 0.47 (dd, J=9.38, 6.94 Hz, 2H) 2.93 (dd, J=9.23, 7.55 Hz, 2H) 3.59 (s, 3H) 4.93 (d, J=11.13 Hz, 1H) 5.10 (d, J=11.13 Hz, 1H) 6.86 (d, J=4.12 Hz, 1H) 6.89 (s, 1H) 7.36 (td, J=8.46, 2.59 Hz, 1H) 7.48 (d, J=5.19 Hz, 1H) 7.60 (dd, J=8.62, 6.18 Hz, 1H) 7.62-7.68 (m, 3H) 7.71-7.77 (m, 1H) 8.01 (d, J=4.12 Hz, 1H) 8.16 (dd, J=8.46, 1.14 Hz, 2H) 8.44 (d, J=5.03 Hz, 1H). LCMS: m/z 640 [M+H]⁺. HRMS (ESI) calcd for C₃₁H₃₁ClFN₃O₅SSi [M+H]⁺ 640.1499 found 640.1491;

### Methyl-2-(2,4-difluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

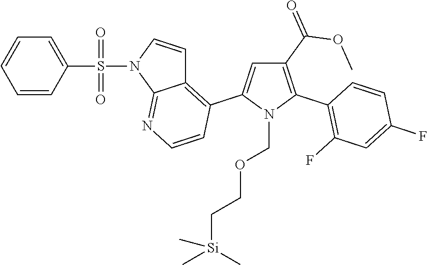 [SMILES: COC(=O)c1cc(-c2ccnc3c2ccn3S(=O)(=O)c2ccccc2)n(COCC[Si](C)(C)C)c1-c1ccc(F)cc1F]

LCMS: m/z 624 [M+H]⁺. HRMS (ESI) calcd for C₃₁H₃₁F₂N₃O₅SSi [M+H]⁺ 624.1795 found 624.1788;

### Methyl-2-[2-chloro-4-(trifluoromethyl)phenyl]-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

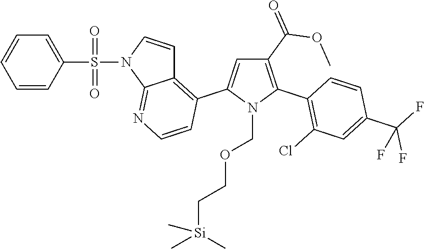 [SMILES: COC(=O)c1cc(-c2ccnc3c2ccn3S(=O)(=O)c2ccccc2)n(COCC[Si](C)(C)C)c1-c1ccc(C(F)(F)F)cc1Cl]

¹H NMR (500 MHz, DMSO-d₆) d ppm −0.32-−0.30 (m, 8H) 0.40-0.48 (m, 2H) 2.87-2.95 (m, 2H) 3.57-3.62 (m, 3H) 4.93-5.14 (m, 2H) 6.87 (d, J=4.12 Hz, 1H) 6.92 (s, 1H) 7.45-7.51 (m, 1H) 7.62-7.68 (m, 2H) 7.72-7.77 (m, 1H) 7.79-7.82 (m, 1H) 7.84-7.88 (m, 1H) 8.02 (d, J=4.12 Hz, 1H) 8.05 (d, J=0.76 Hz, 1H) 8.17 (dd, J=8.54, 1.07 Hz, 2H) 8.46 (d, J=5.19 Hz, 1H). LCMS: m/z 690 [M+H]⁺. HRMS (ESI) calcd for C₃₂H₃₁ClF₃N₃O₅SSi [M+H]⁺ 690.1467 found 690.1469;

### Methyl-2-(2,3-difluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

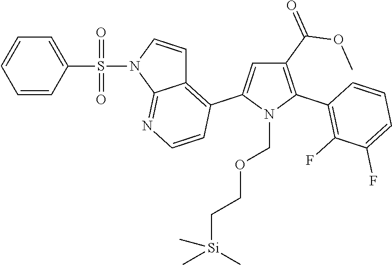 [SMILES: COC(=O)c1cc(-c2ccnc3c2ccn3S(=O)(=O)c2ccccc2)n(COCC[Si](C)(C)C)c1-c1cccc(F)c1F]

LCMS: m/z 624 [M+H]⁺. HRMS (ESI) calcd for C₃₁H₃₁F₂N₃O₅SSi [M+H]⁺ 624.1795 found 624.1791;

### Methyl-2-(2,3-dichlorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

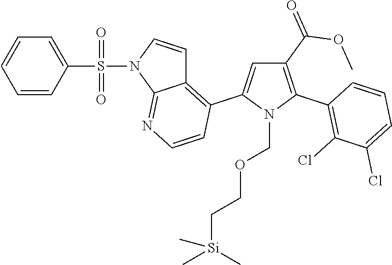 [SMILES: COC(=O)c1cc(-c2ccnc3c2ccn3S(=O)(=O)c2ccccc2)n(COCC[Si](C)(C)C)c1-c1cccc(Cl)c1Cl]

LCMS: m/z 656 [M+H]⁺. HRMS (ESI) calcd for C₃₁H₃₁Cl₂N₃O₅SSi [M+H]⁺ 656.1204 found 656.1207;

### Methyl-2-[4-methyl-2-(trifluoromethyl)phenyl]-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

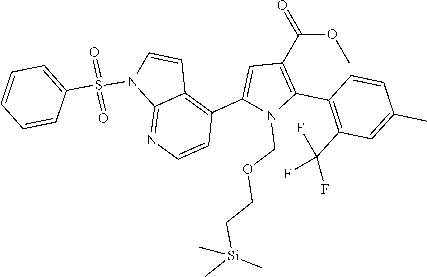 [SMILES: COC(=O)c1cc(-c2ccnc3c2ccn3S(=O)(=O)c2ccccc2)n(COCC[Si](C)(C)C)c1-c1ccc(C)cc1C(F)(F)F]

¹H NMR (500 MHz, DMSO-d₆) d ppm −0.33-−0.29 (m, 9H) 0.45 (dd, J=9.23, 7.55 Hz, 2H) 2.48 (s, 3H) 2.90 (dd, J=9.30, 7.47 Hz, 2H) 3.51-3.53 (m, 3H) 4.75 (d, J=10.98 Hz, 1H) 5.07 (d, J=10.98 Hz, 1H) 6.77 (d, J=4.12 Hz, 1H) 6.85 (s, 1H) 7.39-7.45 (m, 2H) 7.58 (d, J=7.93 Hz, 1H) 7.62-7.67 (m, 2H) 7.70 (s, 1H) 7.72-7.76 (m, 1H) 8.01 (d, J=3.97 Hz, 1H) 8.16 (dd, J=8.62, 1.14 Hz, 2H) 8.44 (d, J=5.03 Hz, 1H). LCMS: m/z 670 [M+H]⁺. HRMS (ESI) calcd for C₃₃H₃₄F₃N₃O₅SSi [M+H]⁺ 670.2014 found 670.2007;

### Methyl-2-(2-chloro-4-methylphenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

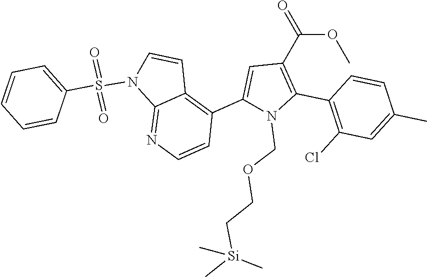 [SMILES: COC(=O)c1cc(-c2ccnc3c2ccn3S(=O)(=O)c2ccccc2)n(COCC[Si](C)(C)C)c1-c1ccc(C)cc1Cl]

LCMS: m/z 636 [M+H]⁺. HRMS (ESI) calcd for C₃₂H₃₄ClN₃O₅SSi [M+H]⁺ 636.1750 found 636.1753;

### Methyl-2-(2,3-difluoro-4-methylphenyl)-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

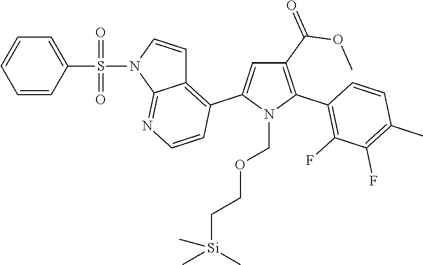 [SMILES: COC(=O)c1cc(-c2ccnc3c2ccn3S(=O)(=O)c2ccccc2)n(COCC[Si](C)(C)C)c1-c1ccc(C)c(F)c1F]

LCMS: m/z 638 [M+H]⁺. HRMS (ESI) calcd for C₃₂H₃₃F₂N₃O₅SSi [M+H]⁺ 638.1951 found 638.1956;

### Methyl-2-[2-methyl-4-(trifluoromethyl)phenyl]-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

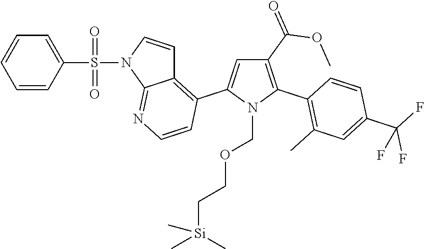 [SMILES: COC(=O)c1cc(-c2ccnc3c2ccn3S(=O)(=O)c2ccccc2)n(COCC[Si](C)(C)C)c1-c1ccc(C(F)(F)F)cc1C]

LCMS: m/z 670 [M+H]⁺. HRMS (ESI) calcd for C₃₃H₃₄F₃N₃O₅SSi [M+H]⁺ 670.2013 found 670.2017;

### Methyl-2-(2-fluoro-3-methoxyphenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

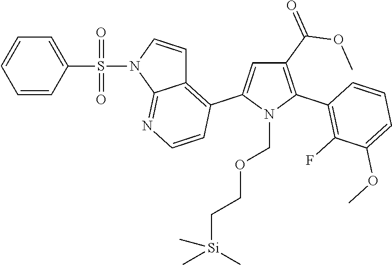 [SMILES: COC(=O)c1cc(-c2ccnc3c2ccn3S(=O)(=O)c2ccccc2)n(COCC[Si](C)(C)C)c1-c1cccc(OC)c1F]

LCMS: m/z 636 [M+H]⁺. HRMS (ESI) calcd for C₃₂H₃₄FN₃O₆SSi [M+H]⁺ 636.1994 found 636.1993;

### Methyl-2-(2-chloro-3-fluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

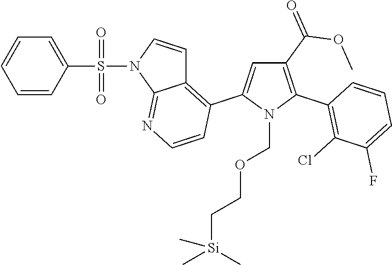 [SMILES: COC(=O)c1cc(-c2ccnc3c2ccn3S(=O)(=O)c2ccccc2)n(COCC[Si](C)(C)C)c1-c1cccc(F)c1Cl]

LCMS: m/z 640 [M+H]⁺. HRMS (ESI) calcd for C₃₁H₃₁ClFN₃O₅SSi [M+H]⁺ 640.1499 found 640.1495;

### Methyl-2-(2-fluoro-3-methylphenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

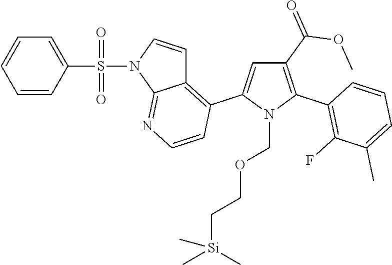 [SMILES: COC(=O)c1cc(-c2ccnc3c2ccn3S(=O)(=O)c2ccccc2)n(COCC[Si](C)(C)C)c1-c1cccc(C)c1F]

LCMS: m/z 620 [M+H]⁺. HRMS (ESI) calcd for C₃₂H₃₄FN₃O₅SSi [M+H]⁺ 620.2045 found 620.2039;

### Methyl-2-[2-methyl-3-(trifluoromethyl)phenyl]-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

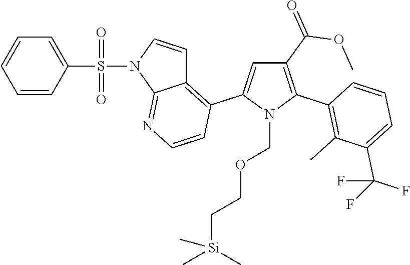 [SMILES: COC(=O)c1cc(-c2ccnc3c2ccn3S(=O)(=O)c2ccccc2)n(COCC[Si](C)(C)C)c1-c1cccc(C(F)(F)F)c1C]

LCMS: m/z 670 [M+H]⁺. HRMS (ESI) calcd for C₃₃H₃₄F₃N₃O₅SSi [M+H]⁺670.2013 found 670.2008;

### Methyl-2-[4-methoxy-2-(trifluoromethyl)phenyl]-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

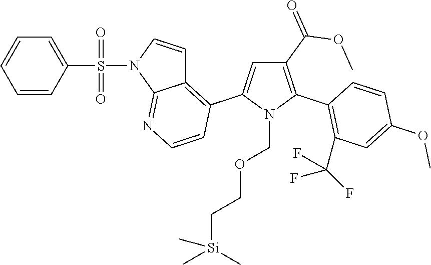 [SMILES: COC(=O)c1cc(-c2ccnc3c2ccn3S(=O)(=O)c2ccccc2)n(COCC[Si](C)(C)C)c1-c1ccc(OC)cc1C(F)(F)F]

LCMS: m/z 686 [M+H]⁺. HRMS (ESI) calcd for C₃₃H₃₄F₃N₃O₆SSi [M+H]⁺ 686.1963 found 686.1961;

### Methyl-2-[2-chloro-4-(difluoromethoxy)phenyl]-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

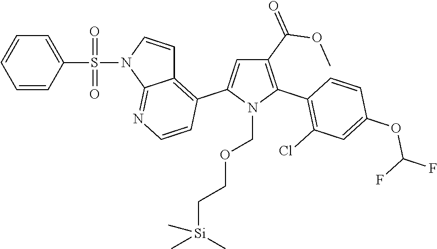 [SMILES: COC(=O)c1cc(-c2ccnc3c2ccn3S(=O)(=O)c2ccccc2)n(COCC[Si](C)(C)C)c1-c1ccc(OC(F)F)cc1Cl]

¹H NMR (500 MHz, DMSO-d₆) d ppm −0.31-−0.28 (m, 9H) 0.44-0.49 (m, 2H) 2.91-2.95 (m, 2H) 3.59 (s, 3H) 4.42-5.12 (m, 2H) 6.85 (d, J=3.85 Hz, 1H) 6.89 (s, 1H) 7.59 (d, J=3.88 Hz, 1H) 7.63-7.66 (m, 2H) 7.73-7.76 (m, 1H) 8.01 (d, J=4.22 Hz, 1H) 8.15-8.17 (m, 2H) 8.45 (d, J=5.22 Hz, 1H). LCMS: m/z 688 [M+H]⁺. HRMS (ESI) calcd for C₃₂H₃₂ClF₂N₃OSSi [M+H]⁺ 688.1511 found 688.1508;

### Methyl-2-(3,4-dichlorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

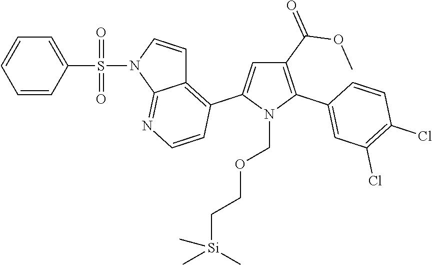 [SMILES: COC(=O)c1cc(-c2ccnc3c2ccn3S(=O)(=O)c2ccccc2)n(COCC[Si](C)(C)C)c1-c1ccc(Cl)c(Cl)c1]

¹H NMR (500 MHz, DMSO-d₆) d ppm −0.27-−0.24 (m, 8H) 0.47-0.57 (m, 2H) 2.97-3.04 (m, 2H) 3.62 (s, 3H) 5.05 (s, 2H) 6.89 (s, 1H) 6.93 (d, J=3.97 Hz, 1H) 7.45-7.54 (m, 2H) 7.62-7.69 (m, 2H) 7.71-7.78 (m, 2H) 7.81 (d, J=1.98 Hz, 1H) 8.01 (d, J=4.12 Hz, 1H) 8.13-8.21 (m, 2H) 8.45 (d, J=5.19 Hz, 1H). LCMS: m/z 656 [M+H]⁺. HRMS (ESI) calcd for C₃₁H₃₁Cl₂N₃O₅SSi [M+H]⁺ 656.1204 found 656.1215;

### Methyl-2-(3,4-difluorophenyl)-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

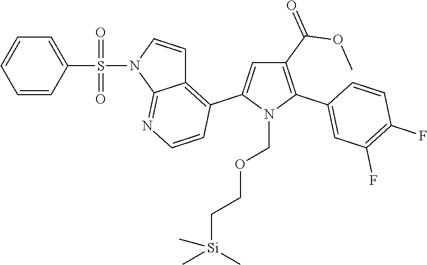 [SMILES: COC(=O)c1cc(-c2ccnc3c2ccn3S(=O)(=O)c2ccccc2)n(COCC[Si](C)(C)C)c1-c1ccc(F)c(F)c1]

¹H NMR (500 MHz, DMSO-d₆) d ppm −0.28-−0.26 (m, 8H) 0.48-0.56 (m, 2H) 2.96-3.03 (m, 2H) 3.61 (s, 3H) 5.06 (s, 2H) 6.88 (s, 1H) 6.92 (d, J=4.12 Hz, 1H) 7.34-7.39 (m, 1H) 7.47-7.51 (m, 1H) 7.53-7.67 (m, 4H) 7.72-7.78 (m, 1H) 8.01 (d, J=3.97 Hz, 1H) 8.14-8.21 (m, 2H) 8.45 (d, J=5.03 Hz, 1H). LCMS: m/z 624 [M+H]⁺. HRMS (ESI) calcd for C₃₁H₃₁F₂N₃O₅SSi [M+H]⁺ 624.1795 found 624.1816;

### Methyl-2-(3-ethoxy-2-fluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

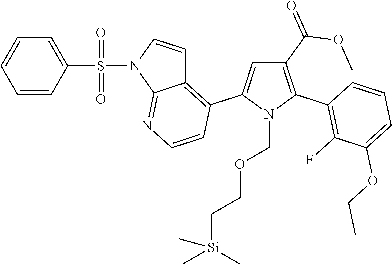 [SMILES: CCOc1cccc(-c2c(C(=O)OC)cc(-c3ccnc4c3ccn4S(=O)(=O)c3ccccc3)n2COCC[Si](C)(C)C)c1F]

¹H NMR (500 MHz, DMSO-d₆) d ppm −0.31-−0.26 (m, 9H) 0.47 (t, J=8.31 Hz, 2H) 1.37 (t, J=7.02 Hz, 1H) 2.87-3.02 (m, 2H) 3.61 (s, 3H) 4.04-4.22 (m, 1H) 4.98-5.18 (m, 2H) 6.84-6.94 (m, 1H) 6.97-7.09 (m, 2H) 7.19-7.25 (m, 1H) 7.26-7.32 (m, 1H) 7.52 (d, J=5.03 Hz, 1H) 7.62-7.68 (m, 2H) 7.72-7.77 (m, 1H) 8.00 (d, J=4.12 Hz, 1H) 8.17 (dd, J=8.46, 1.14 Hz, 2H) 8.44 (d, J=5.19 Hz, 1H). LCMS: m/z 650 [M+H]⁺. HRMS (ESI) calcd for C₃₃H₃₆FN₃O₆SSi [M+H]⁺ 650.2151 found 650.2164;

### Methyl-2-(4-methyl-3-nitrophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

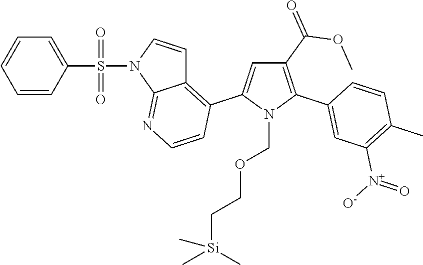 [SMILES: COC(=O)c1cc(-c2ccnc3c2ccn3S(=O)(=O)c2ccccc2)n(COCC[Si](C)(C)C)c1-c1ccc(C)c([N+](=O)[O-])c1]

¹H NMR (500 MHz, DMSO-d₆) d ppm −0.28-−0.25 (m, 8H) 0.52-0.58 (m, 2H) 2.60 (s, 3H) 2.98-3.05 (m, 2H) 3.62 (s, 3H) 5.04 (s, 2H) 6.91 (s, 1H) 6.93 (d, J=4.12 Hz, 1H) 7.51 (d, J=5.03 Hz, 1H) 7.59-7.69 (m, 3H) 7.71-7.78 (m, 2H) 8.01 (d, J=4.12 Hz, 1H) 8.14-8.24 (m, 3H) 8.45 (d, J=5.03 Hz, 1H). LCMS: m/z 647 [M+H]⁺. HRMS (ESI) calcd for C₃₂H₃₄N₄O₇SSi [M+H]⁺ 647.1990 found 647.2003;

### Methyl-2-(4-cyano-3-fluorophenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

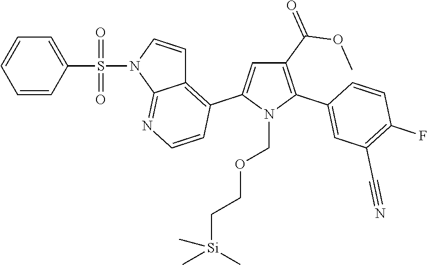 [SMILES: COC(=O)c1cc(-c2ccnc3c2ccn3S(=O)(=O)c2ccccc2)n(COCC[Si](C)(C)C)c1-c1ccc(F)c(C#N)c1]

¹H NMR (500 MHz, DMSO-d₆) d ppm −0.28-−0.24 (m, 9H) 0.46-0.57 (m, 2H) 2.92-3.04 (m, 2H) 3.62 (s, 3H) 5.05 (s, 2H) 6.90 (s, 1H) 6.92 (d, J=4.12 Hz, 1H) 7.47 (d, J=5.03 Hz, 1H) 7.62-7.71 (m, 3H) 7.72-7.77 (m, 1H) 7.87-7.97 (m, 1H) 8.02 (d, J=4.12 Hz, 1H) 8.12 (dd, J=6.25, 2.14 Hz, 1H) 8.15-8.20 (m, 2H) 8.46 (d, J=5.03 Hz, 1H). LCMS: m/z 631 [M+H]⁺. HRMS (ESI) calcd for C₃₂H₃₁FN₄O₅SSi [M+H]⁺ 631.1841 found 631.1846;

### Methyl-2-(dibenzo[b,d]thiophen-4-yl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

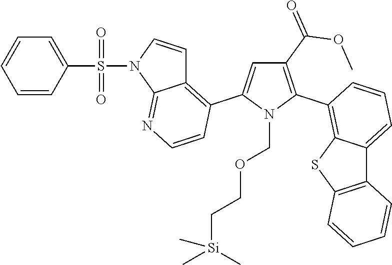 [SMILES: COC(=O)c1cc(-c2ccnc3c2ccn3S(=O)(=O)c2ccccc2)n(COCC[Si](C)(C)C)c1-c1cccc2c1sc1ccccc12]

LCMS: m/z 694 [M+H]⁺. HRMS (ESI) calcd for C₃₇H₃₅N₃O₅S₂Si [M+H]⁺ 694.1860 found 694.1863;

### Methyl-2-(4-methylnaphthalen-1-yl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

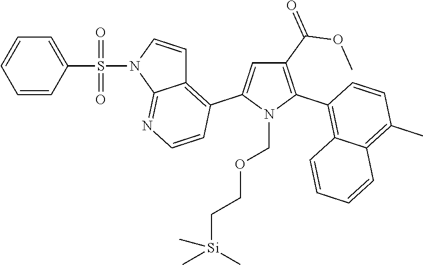 [SMILES: COC(=O)c1cc(-c2ccnc3c2ccn3S(=O)(=O)c2ccccc2)n(COCC[Si](C)(C)C)c1-c1ccc(C)c2ccccc12]

¹H NMR (500 MHz, DMSO-d₆) d ppm −0.40-−0.35 (m, 9H) 0.19-0.32 (m, 2H) 2.67-2.81 (m, 5H) 3.44 (s, 3H) 4.74-5.08 (m, 2H) 6.91-7.01 (m, 2H) 7.43-7.51 (m, 4H) 7.53-7.61 (m, 2H) 7.62-7.71 (m, 2H) 7.73-7.78 (m, 1H) 8.02 (d, J=4.12 Hz, 1H) 8.10 (d, J=8.39 Hz, 1H) 8.17 (dd, J=8.46, 0.99 Hz, 1H) 8.44 (d, J=5.03 Hz, 1H). LCMS: m/z 652 [M+H]⁺. HRMS (ESI) calcd for C₃H₃₇N₃O₅SSi [M+H]⁺ 652.2296 found 652.2303;

### Methyl 2-(3-fluorophenyl)-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

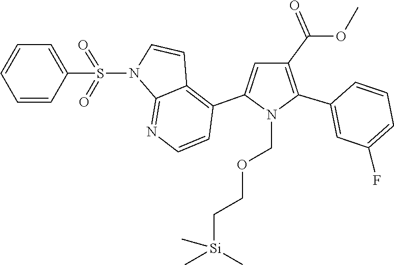 [SMILES: COC(=O)c1cc(-c2ccnc3c2ccn3S(=O)(=O)c2ccccc2)n(COCC[Si](C)(C)C)c1-c1cccc(F)c1]

¹H NMR (500 MHz, DMSO-d₆) d ppm −0.29-−0.24 (m, 9H) 0.49-0.56 (m, 2H) 2.94-3.02 (m, 2H) 3.60 (s, 3H) 5.05 (s, 2H) 6.89 (s, 1H) 6.92 (d, J=3.97 Hz, 1H) 7.29-7.40 (m, 3H) 7.47-7.56 (m, 2H) 7.60-7.68 (m, 2H) 7.70-7.77 (m, 1H) 8.01 (d, J=4.12 Hz, 1H) 8.17 (dd, J=8.46, 1.14 Hz, 2H) 8.45 (d, J=5.03 Hz, 1H). LCMS: m/z 606 [M+H]⁺. HRMS (ESI) calcd for C₃₁H₃₂FN₃O₅SSi [M+H]⁺ 606.1889 found 606.1885;

### Methyl 5-[1-(phenylsulfonyl)-1H-pyrrolo[2,3-b]pyridin-4-yl]-2-[4-(trifluoromethoxy)phenyl]-1-{[2-(trimethylsilyl) ethoxy]methyl}-1H-pyrrole-3-carboxylate (VIII)

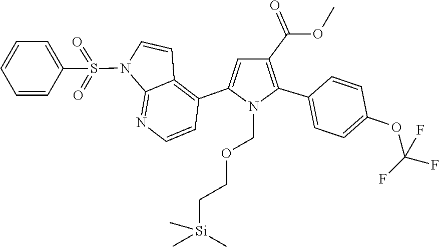 [SMILES: COC(=O)c1cc(-c2ccnc3c2ccn3S(=O)(=O)c2ccccc2)n(COCC[Si](C)(C)C)c1-c1ccc(OC(F)(F)F)cc1]

¹H NMR (500 MHz, DMSO-d6) d ppm −0.30-−0.26 (m, 9H) 0.46-0.55 (m, 2H) 2.94-3.01 (m, 2H) 3.60 (s, 3H) 5.04 (s, 2H) 6.90 (s, 1H) 6.92 (d, J=4.12 Hz, 1H) 7.44-7.53 (m, 3H) 7.61-7.68 (m, 4H) 7.71-7.78 (m, 1H) 8.01 (d, J=4.12 Hz, 1H) 8.17 (dd, J=8.46, 1.14 Hz, 2H) 8.45 (d, J=5.03 Hz, 1H). LCMS: m/z 672 [M+H]⁺. HRMS (ESI) calcd for C₃₂H₃₂F₃N₃O₆SSi [M+H]⁺ 672.1806 found 672.1808;

### Methyl 2-(1-benzothiophen-3-yl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

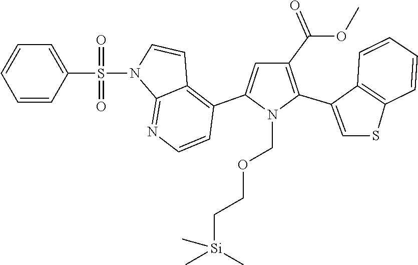 [SMILES: COC(=O)c1cc(-c2ccnc3c2ccn3S(=O)(=O)c2ccccc2)n(COCC[Si](C)(C)C)c1-c1csc2ccccc12]

¹H NMR (500 MHz, DMSO-d6) d ppm −0.39-−0.33 (m, 9H) 0.31-0.43 (m, 2H) 2.78-2.89 (m, 2H) 3.49 (s, 3H) 4.97 (d, J=10.98 Hz, 1H) 5.21 (d, J=10.98 Hz, 1H) 6.93 (d, J=4.12 Hz, 1H) 6.99 (s, 1H) 7.33-7.45 (m, 3H) 7.56 (d, J=5.03 Hz, 1H) 7.62-7.69 (m, 2H) 7.71-7.78 (m, 1H) 7.99 (s, 1H) 8.02 (d, J=4.12 Hz, 1H) 8.04-8.12 (m, 1H) 8.14-8.22 (m, 2H) 8.45 (d, J=5.19 Hz, 1H). LCMS: m/z 644 [M+H]⁺. HRMS (ESI) calcd for C₃₃H₃₃N₃O₅S₂Si [M+H]⁺ 644.1704 found 644.17;

### Methyl 2-(2,3-dihydro-1,4-benzodioxin-6-yl)-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

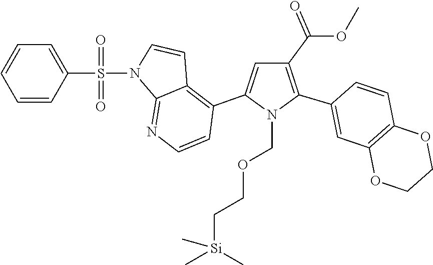 [SMILES: COC(=O)c1cc(-c2ccnc3c2ccn3S(=O)(=O)c2ccccc2)n(COCC[Si](C)(C)C)c1-c1ccc2c(c1)OCCO2]

¹H NMR (500 MHz, DMSO-d6) d ppm −0.25 (s, 9H) 0.50-0.59 (m, 2H) 2.98-3.07 (m, 2H) 3.61 (s, 3H) 4.29 (q, J=4.98 Hz, 4H) 5.04 (s, 2H) 6.86 (s, 1H) 6.91 (d, J=4.12 Hz, 1H) 6.92-6.96 (m, 2H) 7.00 (m, J=1.22 Hz, 1H) 7.52 (d, J=5.19 Hz, 1H) 7.61-7.67 (m, 2H) 7.74 (m, J=7.47 Hz, 1H) 7.99 (d, J=4.12 Hz, 1H) 8.17 (dd, J=8.39, 1.07 Hz, 2H) 8.43 (d, J=5.19 Hz, 1H). LCMS: m/z 646 [M+H]⁺. HRMS (ESI) calcd for C₃₃H₃₅N₃O₇SSi [M+H]⁺ 646.2038 found 646.2045;

### Methyl 2-(4-fluoro-2-methylphenyl)-5-[1-(phenylsulfonyl)-1H-pyrrolo[2,3-b]pyridin-4-yl]-1-{[2-(trimethylsilyl) ethoxy]methyl}-1H-pyrrole-3-carboxylate (VIII)

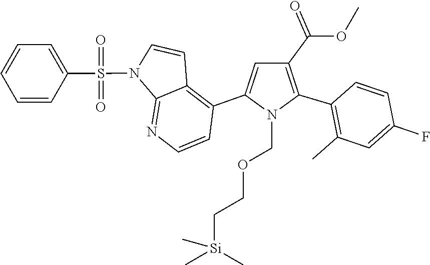 [SMILES: COC(=O)c1cc(-c2ccnc3c2ccn3S(=O)(=O)c2ccccc2)n(COCC[Si](C)(C)C)c1-c1ccc(F)cc1C]

¹H NMR (500 MHz, DMSO-d₆) d ppm −0.32-−0.28 (m, 9H) 0.40-0.50 (m, 2H) 2.08 (s, 3H) 2.82-3.04 (m, 2H) 3.57 (s, 3H) 4.79-5.05 (m, 2H) 6.84-6.92 (m, 2H) 7.12 (d, J=2.59 Hz, 1H) 7.21 (dd, J=10.07, 2.59 Hz, 1H) 7.33 (dd, J=8.46, 6.02 Hz, 1H) 7.47-7.52 (m, 1H) 7.58-7.68 (m, 2H) 7.70-7.77 (m, 1H) 8.00 (d, J=3.97 Hz, 1H) 8.16 (dd, J=8.46, 1.14 Hz, 2H) 8.43 (d, J=5.19 Hz, 1H). LCMS: m/z 620 [M+H]⁺. HRMS (ESI) calcd for C₃₂H₃₄FN₃O₅SSi [M+H]⁺ 620.2045 found 620.2034;

### Methyl-2-(2-fluoro-3-methylphenyl)-4-iodo-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

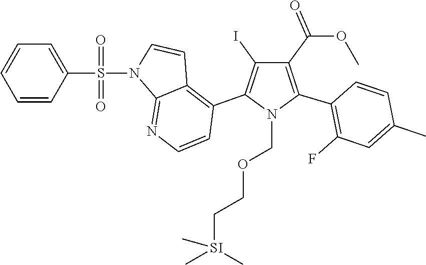

¹H NMR (500 MHz, DMSO-d₆) d ppm −0.30 (s, 9H) 0.32 (m, J=9.65, 9.65, 6.63 Hz, 2H) 2.38 (s, 3H) 2.67-2.86 (m, 2H) 3.53 (s, 3H) 4.58-5.07 (m, 2H) 6.37-6.81 (m, 1H) 7.09-7.19 (m, 2H) 7.39 (d, J=5.03 Hz, 2H) 7.61-7.71 (m, 2H) 7.73-7.79 (m, 1H) 8.02 (d, J=3.97 Hz, 1H) 8.19 (d, J=7.47 Hz, 2H) 8.51 (d, J=5.03 Hz, 1H). LCMS: m/z 746 [M+H]⁺. HRMS (ESI) calcd for C₃₃H₃₃FlN₃O₅S [M+H]⁺ 746.1012 found 746.1024;

### Methyl-2-(2-fluoro-3-methylphenyl)-4-bromo-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

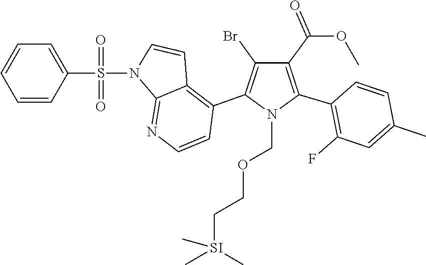

¹H NMR (500 MHz, DMSO-d₆) d ppm −0.34-−0.27 (m, 9H) 0.26-0.41 (m, 2H) 2.39 (s, 3H) 2.80 (m, J=11.13, 11.13, 6.56 Hz, 2H) 3.55 (s, 3H) 4.68-5.18 (m, 2H) 6.47-6.85 (m, 1H) 7.08-7.22 (m, 2H) 7.30-7.48 (m, 2H) 7.59-7.70 (m, 2H) 7.70-7.82 (m, 1H) 8.03 (d, J=3.97 Hz, 1H) 8.18-8.22 (m, 2H) 8.51 (d, J=5.03 Hz, 1H). LCMS: m/z 698 [M+H]⁺. HRMS (ESI) calcd for C₃₃H₃₃BrFN₃O₅S [M+H]⁺ 698.1151 found 698.1159;

### Conversion 2

### Methyl 4-ethenyl-2-(2-fluoro-4-methylphenyl)-5-[1-(phenylsulfonyl)-1H-pyrrolo[2,3-b]pyridin-4-yl]-1-{[2-(trimethylsilyl)ethoxy]methyl}-1H-pyrrole-3-carboxylate (VIII)

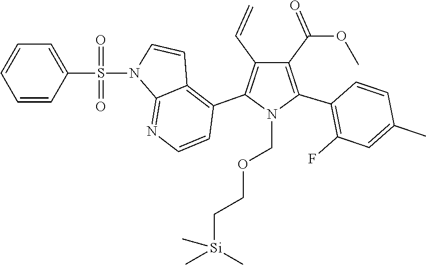 [SMILES: C=Cc1c(C(=O)OC)c(-c2ccc(C)cc2F)n(COCC[Si](C)(C)C)c1-c1ccnc2c1ccn2S(=O)(=O)c1ccccc1]

In a reactor, under argon atmosphere, methyl 2-(2-fluoro-4-methylphenyl)-4-iodo-5-[1-(phenylsulfonyl)-1H-pyrrolo[2,3-b]pyridin-4-yl]-1-{[2-(trimethylsilyl)ethoxy]methyl}-1H-pyrrole-3-carboxylate (1 eq., 50 mg, 0.06 mmol), ethenylboronic acid (2 eq., 23 μl, 0.13 mmol), Na₂CO₃ (3 eq., 21.3 mg, 0.2 mmol), 1,4-dioxane degassed (1 ml) and distilled water degassed (0.25 ml) were added. After three cycles of vacuum/argon, the catalyst Tetrakis(triphenylphosphine)-palladium(0) (0.1 eq., 7.7 mg, 0.006 mmol) was added. After three cycles of vacuum/argon the reaction mixture was heated at T=100° C. for 2 hours. Distilled water was added and the product was extracted with AcOEt (3 times). The organic layer was washed with distilled water and brine, dried over anhydrous Na₂SO₄ and evaporated to dryness. The crude was purified by flash-chromatography (Hexane/Ethyl Acetate 8/2) affording the title compound (white solid, 28 mg, Y=72%).

¹H NMR (500 MHz, DMSO-d₆) d ppm −0.32-−0.28 (m, 9H) 0.27-0.37 (m, 2H) 2.36-2.40 (m, 3H) 2.77 (td, J=10.29, 6.41 Hz, 2H) 3.50-3.54 (m, 3H) 4.64 (d, J=17.54 Hz, 1H) 4.84 (dd, J=11.44, 1.68 Hz, 1H) 4.86-5.02 (m, 2H) 6.79 (br. s., 2H) 7.08-7.18 (m, 2H) 7.28-7.47 (m, 2H) 7.60-7.69 (m, 2H) 7.74 (t, J=7.40 Hz, 1H) 7.93-8.04 (m, 1H) 8.11-8.22 (m, 2H) 8.45-8.53 (m, 1H). LCMS: m/z 645 [M+H]⁺. HRMS (ESI) calcd for C₃₄H₃₆FN₃O₅SSi [M+H]⁺ 646.2202 found 646.2194;

Operating in an analogous way, but employing suitable substituted starting material the following compound was obtained:

### Methyl 2-(2-fluoro-4-methylphenyl)-5-[1-(phenylsulfonyl)-1H-pyrrolo[2,3-b]pyridin-4-yl]-4-(prop-1-en-2-yl)-1-{[2-(trimethylsilyl)ethoxy]methyl}-1H-pyrrole-3-carboxylate12 ((VIII)

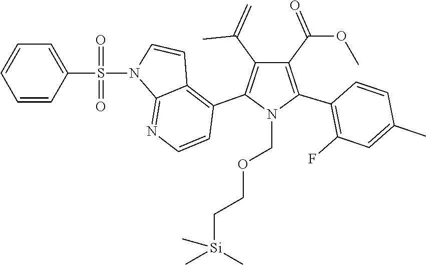 [SMILES: C=C(C)c1c(C(=O)OC)c(-c2ccc(C)cc2F)n(COCC[Si](C)(C)C)c1-c1ccnc2c1ccn2S(=O)(=O)c1ccccc1]

¹H NMR (500 MHz, DMSO-d₆) d ppm −0.34-−0.23 (m, 9H) 0.27-0.39 (m, 2H) 1.88 (s, 3H) 2.38 (s, 3H) 2.74-2.81 (m, 2H) 3.49 (s, 3H) 4.44 (d, J=1.37 Hz, 1H) 4.76 (s, 3H) 6.39-6.76 (m, 1H) 7.07-7.17 (m, 3H) 7.32 (d, J=4.88 Hz, 1H) 7.62-7.68 (m, 2H) 7.70-7.80 (m, 1H) 7.95 (d, J=4.12 Hz, 1H) 8.17 (d, J=7.47 Hz, 2H) 8.42 (d, J=5.03 Hz, 1H). LCMS: m/z 659 [M+H]⁺. HRMS (ESI) calcd for C₃₅H₃₅FN₃O₅SSi [M+H]⁺ 660.2358 found 660.2358;

### Step 5

### 2-(3-chloro-2-fluorophenyl)-5-(1H-pyrrolo[2,3-b]pyridin-4-yl)-1-{[2-(trimethylsilyl)ethoxy]methyl}-1H-pyrrole-3-carboxylic acid (IX)

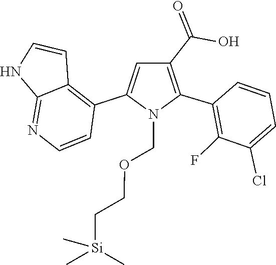 [SMILES: C[Si](C)(C)CCOCn1c(-c2ccnc3[nH]ccc23)cc(C(=O)O)c1-c1cccc(Cl)c1F]

To a solution of methyl 2-(3-chloro-2-fluorophenyl)-5-[1-(phenylsulfonyl)-1H-pyrrolo[2,3-b]pyridin-4-yl]-1-{[2-(trimethylsilyl)ethoxy]methyl}-1H-pyrrole-3-carboxylate (1 eq., 100 mg, 0.156 mmol) in Dioxane (0.5 ml) a solution of NaOH 4M (0.5 ml) was added. The reaction mixture was stirred at T=100° C. for 4 hours. After cooling at room temperature, acetic acid was added to reach pH=6. Distilled water was added and the product was extracted 3 times with AcOEt. The organic layer was washed with brine, dried over anhydrous Na₂SO₄ and evaporated to dryness. The crude was used in next step without further purification (white solid, 70 mg, Y=92%).

¹H NMR (500 MHz, DMSO-d₆) d ppm −0.24-−0.18 (m, 9H) 0.46-0.58 (m, 2H) 2.90-3.02 (m, 2H) 5.04-5.21 (m, 2H) 6.53 (dd, J=3.43, 1.91 Hz, 1H) 6.86 (s, 1H) 7.23 (d, J=5.03 Hz, 1H) 7.35 (t, J=7.85 Hz, 1H) 7.53-7.61 (m, 2H) 7.67-7.73 (m, 1H) 8.29 (d, J=4.88 Hz, 1H) 11.86 (br. s., 1H) 12.04 (br. s., 1H). LCMS: m/z 486 [M+H]⁺. HRMS (ESI) calcd for C₂₄H₂₅ClFN₃O₃Si [M+H]⁺ 486.1411 found 486.1397;

Operating in an analogous way, but employing suitable substituted starting material the following compounds were obtained:

### 2-(4-chloro-2-fluorophenyl)-5-(1H-pyrrolo[2,3-b]pyridin-4-yl)-1-{[2-(trimethylsilyl)ethoxy]methyl}-1H-pyrrole-3-carboxylic acid (IX)

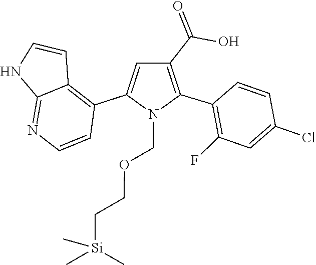 [SMILES: C[Si](C)(C)CCOCn1c(-c2ccnc3[nH]ccc23)cc(C(=O)O)c1-c1ccc(Cl)cc1F]

¹H NMR (500 MHz, DMSO-d₆) d ppm −0.23-−0.18 (m, 9H) 0.48-0.56 (m, 2H) 2.94-3.00 (m, 2H) 5.04-5.22 (m, 2H) 6.52 (dd, J=3.43, 1.91 Hz, 1H) 6.85 (s, 1H) 7.22 (d, J=4.88 Hz, 1H) 7.42 (dd, J=8.31, 2.06 Hz, 1H) 7.53-7.58 (m, 2H) 7.61 (t, J=8.08 Hz, 1H) 8.28 (d, J=5.03 Hz, 1H) 11.85 (br. s., 1H) 12.08 (br. s., 1H). LCMS: m/z 486 [M+H]⁺.

HRMS (ESI) calcd for C₂₄H₂₅ClFN₃O₃Si [M+H]⁺ 486.1411 found 486.1417;

### 2-(2-chloro-4-fluorophenyl)-5-(1H-pyrrolo[2,3-b]pyridin-4-yl)-1-{[2-(trimethylsilyl)ethoxy]methyl}-1H-pyrrole-3-carboxylic acid (IX)

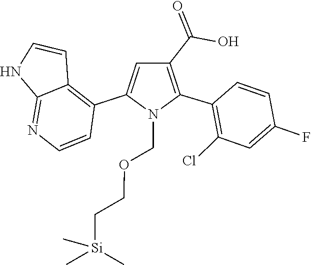 [SMILES: C[Si](C)(C)CCOCn1c(-c2ccnc3[nH]ccc23)cc(C(=O)O)c1-c1ccc(F)cc1Cl]

LCMS: m/z 486 [M+H]⁺. HRMS (ESI) calcd for C₂₄H₂₅ClFN₃O₃Si [M+H]⁺ 486.1411 found 486.1408;

### 2-(2,4-difluorophenyl)-5-[1-(phenylsulfonyl)-1H-pyrrolo[2,3-b]pyridin-4-yl]-1-{[2-(trimethylsilyl)ethoxy]methyl}-1H-pyrrole-3-carboxylic acid (IX)

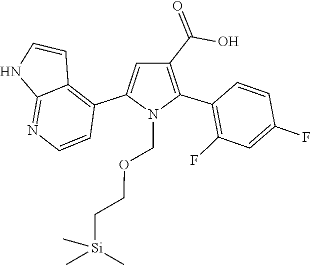 [SMILES: C[Si](C)(C)CCOCn1c(-c2ccnc3[nH]ccc23)cc(C(=O)O)c1-c1ccc(F)cc1F]

LCMS: m/z 470 [M+H]⁺. HRMS (ESI) calcd for C₂₄H₂₅F₂N₃O₃Si [M+H]⁺ 470.1706 found 470.1711;

### 2-[2-chloro-4-(trifluoromethyl)phenyl]-5-(1H-pyrrolo[2,3-b]pyridin-4-yl)-1-{[2-(trimethylsilyl)ethoxy]methyl}-1H-pyrrole-3-carboxylic acid (IX)

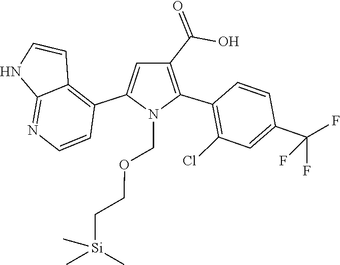 [SMILES: C[Si](C)(C)CCOCn1c(-c2ccnc3[nH]ccc23)cc(C(=O)O)c1-c1ccc(C(F)(F)F)cc1Cl]

¹H NMR (500 MHz, DMSO-d₆) d ppm −0.24-−0.22 (m, 7H) 0.42-0.52 (m, 2H) 2.89-2.98 (m, 2H) 4.96-5.18 (m, 2H) 6.49 (dd, J=3.36, 1.83 Hz, 1H) 6.85 (s, 1H) 7.20 (d, J=4.88 Hz, 1H) 7.56-7.59 (m, 1H) 7.80-7.87 (m, 2H) 8.03 (s, 1H) 8.29 (d, J=4.88 Hz, 1H) 11.87 (br. s., 1H). LCMS: m/z 536 [M+H]⁺. HRMS (ESI) calcd for C₂₅H₂₅ClF₃N₃O₃Si [M+H]⁺ 536.1379 found 536.1384;

### 2-(2,3-difluorophenyl)-5-(1H-pyrrolo[2,3-b]pyridin-4-yl)-1-{[2-(trimethylsilyl)ethoxy]methyl}-1H-pyrrole-3-carboxylic acid (IX)

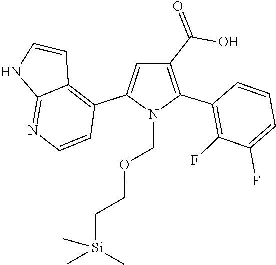 [SMILES: C[Si](C)(C)CCOCn1c(-c2ccnc3[nH]ccc23)cc(C(=O)O)c1-c1cccc(F)c1F]

LCMS: m/z 470 [M+H]⁺. HRMS (ESI) calcd for C₂₄H₂₅F₂N₃O₃Si [M+H]⁺ 470.1706 found 470.1701;

### 2-(2,3-dichlorophenyl)-5-(1H-pyrrolo[2,3-b]pyridin-4-yl)-1-{[2-(trimethylsilyl)ethoxy]methyl}-1H-pyrrole-3-carboxylic acid (IX)

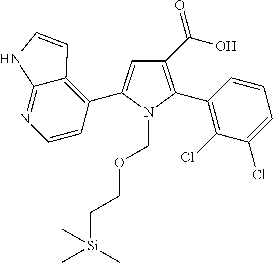 [SMILES: C[Si](C)(C)CCOCn1c(-c2ccnc3[nH]ccc23)cc(C(=O)O)c1-c1cccc(Cl)c1Cl]

LCMS: m/z 502 [M+H]⁺. HRMS (ESI) calcd for C₂₄H₂₅Cl₂N₃O₃Si [M+H]⁺ 502.1112 found 502.1119;

### 2-[4-methyl-2-(trifluoromethyl)phenyl]-5-(1H-pyrrolo[2,3-b]pyridin-4-yl)-1-{[2-(trimethylsilyl)ethoxy]methyl}-1H-pyrrole-3-carboxylic acid (IX)

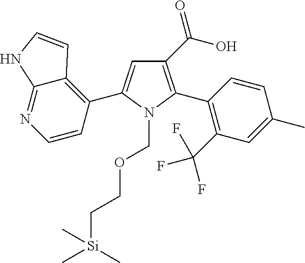 [SMILES: Cc1ccc(-c2c(C(=O)O)cc(-c3ccnc4[nH]ccc34)n2COCC[Si](C)(C)C)c(C(F)(F)F)c1]

¹H NMR (500 MHz, DMSO-d₆) d ppm −0.25-−0.19 (m, 9H) 0.49 (ddd, J=9.76, 7.02, 2.29 Hz, 2H) 2.47 (s, 3H) 2.86-2.99 (m, 2H) 4.76 (d, J=10.98 Hz, 1H) 5.11 (d, J=10.98 Hz, 1H) 6.39 (dd, J=3.36, 1.83 Hz, 1H) 6.79 (s, 1H) 7.16 (d, J=5.03 Hz, 1H) 7.43 (d, J=7.78 Hz, 1H) 7.52-7.60 (m, 2H) 7.68 (s, 1H) 8.28 (d, J=4.88 Hz, 1H) 11.85 (br. s., 1H). LCMS: m/z 516 [M+H]⁺. HRMS (ESI) calcd for C₂₆H₂₅F₃N₃O₃Si [M+H]⁺ 516.1925 found 516.1931;

### 2-(2-chloro-4-methylphenyl)-5-(1H-pyrrolo[2,3-b]pyridin-4-yl)-1-{[2-(trimethylsilyl)ethoxy]methyl}-1H-pyrrole-3-carboxylic acid (IX)

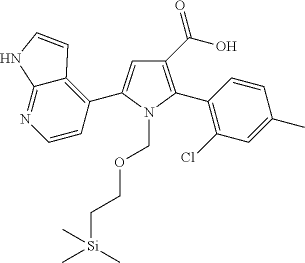 [SMILES: Cc1ccc(-c2c(C(=O)O)cc(-c3ccnc4[nH]ccc34)n2COCC[Si](C)(C)C)c(Cl)c1]

LCMS: m/z 482 [M+H]⁺. HRMS (ESI) calcd for C₂₅H₂₅ClN₃O₃Si [M+H]⁺ 482.1661 found 482.1664;

### 2-(2,3-difluoro-4-methylphenyl)-5-(1H-pyrrolo[2,3-b]pyridin-4-yl)-1-{[2-(trimethylsilyl)ethoxy]methyl}-1H-pyrrole-3-carboxylic acid (IX)

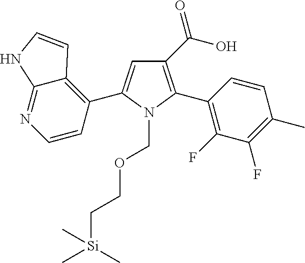 [SMILES: Cc1ccc(-c2c(C(=O)O)cc(-c3ccnc4[nH]ccc34)n2COCC[Si](C)(C)C)c(F)c1F]

LCMS: m/z 484 [M+H]⁺. HRMS (ESI) calcd for C₂₅H₂₇F₂N₃O₃Si [M+H]⁺ 484.1863 found 484.1866;

### 2-[2-methyl-4-(trifluoromethyl)phenyl]-5-(1H-pyrrolo[2,3-b]pyridin-4-yl)-1-{[2-(trimethylsilyl)ethoxy]methyl}-1H-pyrrole-3-carboxylic acid (IX)

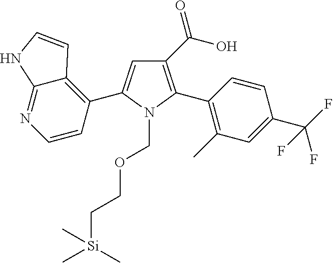 [SMILES: Cc1cc(C(F)(F)F)ccc1-c1c(C(=O)O)cc(-c2ccnc3[nH]ccc23)n1COCC[Si](C)(C)C]

LCMS: m/z 516 [M+H]⁺. HRMS (ESI) calcd for C₂₆H₂₅F₃N₃O₃Si [M+H]⁺ 516.1925 found 516.1927;

### 2-(2-fluoro-3-methoxyphenyl)-5-(1H-pyrrolo[2,3-b]pyridin-4-yl)-1-{[2-(trimethylsilyl)ethoxy]methyl}-1H-pyrrole-3-carboxylic acid (IX)

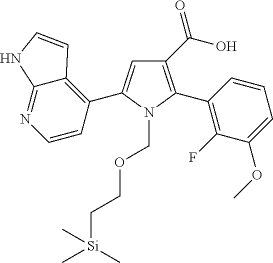 [SMILES: COc1cccc(-c2c(C(=O)O)cc(-c3ccnc4[nH]ccc34)n2COCC[Si](C)(C)C)c1F]

LCMS: m/z 482 [M+H]⁺. HRMS (ESI) calcd for C₂₅H₂₅FN₃O₄Si [M+H]⁺ 482.1906 found 482.1904;

### 2-(2-chloro-3-fluorophenyl)-5-(1H-pyrrolo[2,3-b]pyridin-4-yl)-1-{[2-(trimethylsilyl)ethoxy]methyl}-1H-pyrrole-3-carboxylic acid (IX)

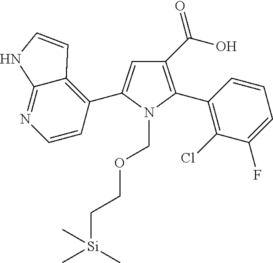 [SMILES: C[Si](C)(C)CCOCn1c(-c2ccnc3[nH]ccc23)cc(C(=O)O)c1-c1cccc(F)c1Cl]

LCMS: m/z 486 [M+H]⁺. HRMS (ESI) calcd for C₂₄H₂₅ClFN₃O₃Si [M+H]⁺ 486.1411 found 486.1409;

### 2-(2-fluoro-3-methylphenyl)-5-(1H-pyrrolo[2,3-b]pyridin-4-yl)-1-{[2-(trimethylsilyl)ethoxy]methyl}-1H-pyrrole-3-carboxylic acid (IX)

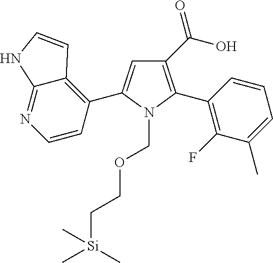 [SMILES: Cc1cccc(-c2c(C(=O)O)cc(-c3ccnc4[nH]ccc34)n2COCC[Si](C)(C)C)c1F]

LCMS: m/z 466 [M+H]⁺. HRMS (ESI) calcd for C₂₅H₂₅FN₃O₃Si [M+H]⁺ 466.1957 found 466.1951;

### 2-[2-methyl-3-(trifluoromethyl)phenyl]-5-(1H-pyrrolo[2,3-b]pyridin-4-yl)-1-{[2-(trimethylsilyl)ethoxy]methyl}-1H-pyrrole-3-carboxylic acid (IX)

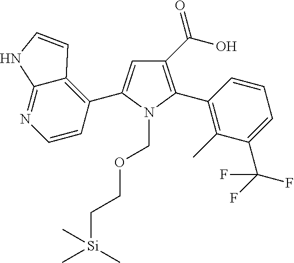 [SMILES: Cc1c(-c2c(C(=O)O)cc(-c3ccnc4[nH]ccc34)n2COCC[Si](C)(C)C)cccc1C(F)(F)F]

LCMS: m/z 516 [M+H]⁺. HRMS (ESI) calcd for C₂₆H₂₅F₃N₃O₃Si [M+H]⁺ 516.1925 found 516.1919;

### 2-[4-methoxy-2-(trifluoromethyl)phenyl]-5-(1H-pyrrolo[2,3-b]pyridin-4-yl)-1-{[2-(trimethylsilyl)ethoxy]methyl}-1H-pyrrole-3-carboxylic acid (IX)

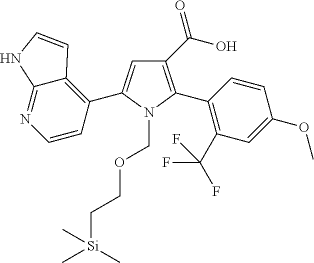 [SMILES: COc1ccc(-c2c(C(=O)O)cc(-c3ccnc4[nH]ccc34)n2COCC[Si](C)(C)C)c(C(F)(F)F)c1]

LCMS: m/z 532 [M+H]⁺. HRMS (ESI) calcd for C₂₆H₂₅F₃N₃O₄Si [M+H]⁺ 532.1874 found 532.1871;

### 2-[2-chloro-4-(difluoromethoxy)phenyl]-5-(1H-pyrrolo[2,3-b]pyridin-4-yl)-1-{[2-(trimethylsilyl)ethoxy]methyl}-1H-pyrrole-3-carboxylic acid (IX)

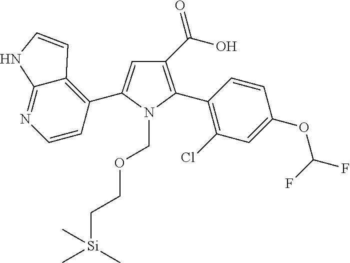 [SMILES: C[Si](C)(C)CCOCn1c(-c2ccnc3[nH]ccc23)cc(C(=O)O)c1-c1ccc(OC(F)F)cc1Cl]

LCMS: m/z 534 [M+H]⁺. HRMS (ESI) calcd for C₂₅H₂₆ClF₂N₃O₄Si [M+H]⁺ 534.1422 found 534.1423;

### 2-(3,4-dichlorophenyl)-5-(1H-pyrrolo[2,3-b]pyridin-4-yl)-1-{[2-(trimethylsilyl)ethoxy]methyl}-1H-pyrrole-3-carboxylic acid (IX)

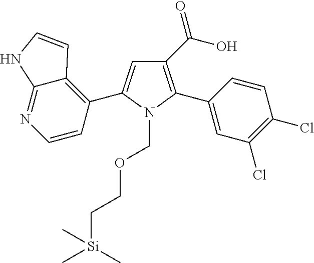 [SMILES: C[Si](C)(C)CCOCn1c(-c2ccnc3[nH]ccc23)cc(C(=O)O)c1-c1ccc(Cl)c(Cl)c1]

LCMS: m/z 502 [M+H]⁺. HRMS (ESI) calcd for C₂₄H₂₅Cl₂N₃O₃Si [M+H]⁺ 502.1115 found 502.1121;

### 2-(3,4-difluorophenyl)-5-(1H-pyrrolo[2,3-b]pyridin-4-yl)-1-{[2-(trimethylsilyl)ethoxy]methyl}-1H-pyrrole-3-carboxylic acid (IX)

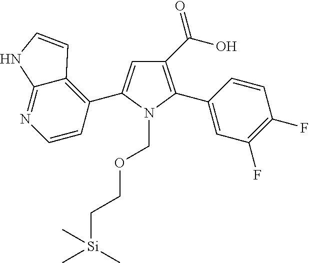 [SMILES: C[Si](C)(C)CCOCn1c(-c2ccnc3[nH]ccc23)cc(C(=O)O)c1-c1ccc(F)c(F)c1]

LCMS: m/z 470 [M+H]⁺. HRMS (ESI) calcd for C₂₄H₂₅F₂N₃O₃Si [M+H]⁺ 470.1706 found 470.1715;

### 2-(3-ethoxy-2-fluorophenyl)-5-(1H-pyrrolo[2,3-b]pyridin-4-yl)-1-{[2-(trimethylsilyl)ethoxy]methyl}-1H-pyrrole-3-carboxylic acid (IX)

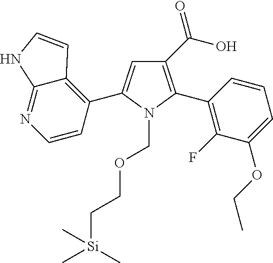 [SMILES: CCOc1cccc(-c2c(C(=O)O)cc(-c3ccnc4[nH]ccc34)n2COCC[Si](C)(C)C)c1F]

¹H NMR (500 MHz, DMSO-d₆) d ppm −0.24-−0.16 (m, 9H) 0.48-0.58 (m, 2H) 1.38 (t, J=6.94 Hz, 1H) 2.87-3.08 (m, 2H) 4.04-4.25 (m, 1H) 4.95-5.23 (m, 2H) 6.52 (dd, J=3.43, 1.91 Hz, 1H) 6.81-6.91 (m, 1H) 7.02-7.11 (m, 1H) 7.18-7.32 (m, 3H) 7.55-7.61 (m, 1H) 8.28 (d, J=4.88 Hz, 1H) 11.85 (br. s., 1H) 11.95 (br. s., 1H). LCMS: m/z 496 [M+H]⁺. HRMS (ESI) calcd for C₂₆H₃₀FN₃O₄Si [M+H]⁺ 496.2063 found 496.2069;

### 2-(4-methyl-3-nitrophenyl)-5-(1H-pyrrolo[2,3-b]pyridin-4-yl)-1-{[2-(trimethylsilyl)ethoxy]methyl}-1H-pyrrole-3-carboxylic acid (IX)

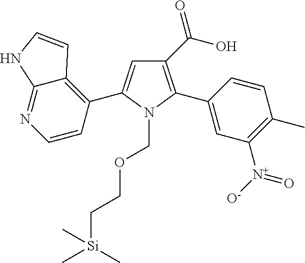 [SMILES: Cc1ccc(-c2c(C(=O)O)cc(-c3ccnc4[nH]ccc34)n2COCC[Si](C)(C)C)cc1[N+](=O)[O-]]

¹H NMR (500 MHz, DMSO-d₆) d ppm −0.20-−0.14 (m, 2H) 0.57-0.67 (m, 1H) 2.61 (s, 1H) 2.99-3.11 (m, 1H) 5.09 (s, 1H) 6.57 (dd, J=3.36, 1.98 Hz, 1H) 6.88 (s, 1H) 7.26 (d, J=5.03 Hz, 1H) 7.55-7.65 (m, 1H) 7.80 (dd, J=7.85, 1.75 Hz, 1H) 8.18 (d, J=1.68 Hz, 1H) 8.30 (d, J=4.88 Hz, 1H) 11.60-12.24 (m, 1H). LCMS: m/z 493 [M+H]⁺. HRMS (ESI) calcd for C₂₅H₂₅N₄O₅Si [M+H]⁺ 493.1902 found 493.1903;

### 2-(3-carboxy-4-fluorophenyl)-5-(1H-pyrrolo[2,3-b]pyridin-4-yl)-1-{[2-(trimethylsilyl)ethoxy]methyl}-1H-pyrrole-3-carboxylic acid (IX)

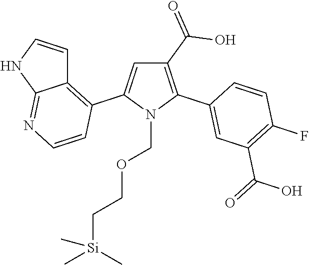 [SMILES: C[Si](C)(C)CCOCn1c(-c2ccnc3[nH]ccc23)cc(C(=O)O)c1-c1ccc(F)c(C(=O)O)c1]

LCMS: m/z 496 [M+H]⁺. HRMS (ESI) calcd for C₂₅H₂₆FN₃O₅Si [M+H]⁺ 496.1699 found 496.1701;

### 2-(2-fluoro-4-methylphenyl)-5-(1H-pyrrolo[2,3-b]pyridin-4-yl)-1-{[2-(trimethylsilyl)ethoxy]methyl}-1H-pyrrole-3-carboxylic acid (IX)

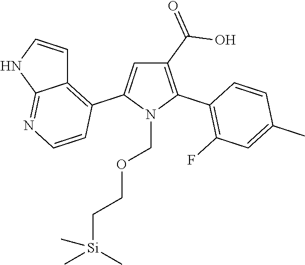 [SMILES: Cc1ccc(-c2c(C(=O)O)cc(-c3ccnc4[nH]ccc34)n2COCC[Si](C)(C)C)c(F)c1]

LCMS: m/z 466 [M+H]⁺. HRMS (ESI) calcd for C₂₅H₂₅FN₃O₃Si [M+H]⁺ 466.1957 found 466.1959;

### 2-(dibenzo[b,d]thiophen-4-yl)-5-(1H-pyrrolo[2,3-b]pyridin-4-yl)-1-{[2-(trimethylsilyl)ethoxy]methyl}-1H-pyrrole-3-carboxylic acid (IX)

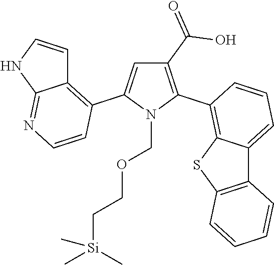 [SMILES: C[Si](C)(C)CCOCn1c(-c2ccnc3[nH]ccc23)cc(C(=O)O)c1-c1cccc2c1sc1ccccc12]

LCMS: m/z 540 [M+H]⁺. HRMS (ESI) calcd for C₃₀H₂₈N₃O₃SSi [M+H]⁺ 540.1772 found 540.1775;

### 2-(4-methylnaphthalen-1-yl)-5-(1H-pyrrolo[2,3-b]pyridin-4-yl)-1-{[2-(trimethylsilyl)ethoxy]methyl}-1H-pyrrole-3-carboxylic acid (IX)

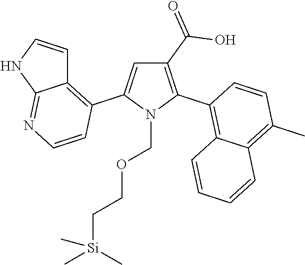 [SMILES: Cc1ccc(-c2c(C(=O)O)cc(-c3ccnc4[nH]ccc34)n2COCC[Si](C)(C)C)c2ccccc12]

LCMS: m/z 498 [M+H]⁺. HRMS (ESI) calcd for C₂₉H₃₁N₃O₃Si [M+H]⁺ 498.2207 found 498.2209;

### 2-(3-fluorophenyl)-5-(1H-pyrrolo[2,3-b]pyridin-4-yl)-1-{[2-(trimethylsilyl)ethoxy]methyl}-1H-pyrrole-3-carboxylic acid (IX)

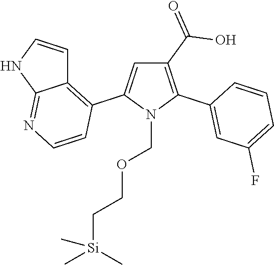 [SMILES: C[Si](C)(C)CCOCn1c(-c2ccnc3[nH]ccc23)cc(C(=O)O)c1-c1cccc(F)c1]

LCMS: m/z 452 [M+H]⁺. HRMS (ESI) calcd for C₂₄H₂₆FN₃O₃Si [M+H]⁺ 452.1800 found 452.1811;

### 5-(1H-pyrrolo[2,3-b]pyridin-4-yl)-2-[4-(trifluoromethoxy)phenyl]-1-{[2-(trimethylsilyl)ethoxy]methyl}-1H-pyrrole-3-carboxylic acid (IX)

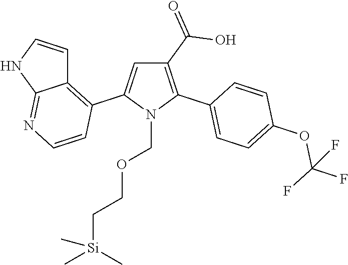 [SMILES: C[Si](C)(C)CCOCn1c(-c2ccnc3[nH]ccc23)cc(C(=O)O)c1-c1ccc(OC(F)(F)F)cc1]

LCMS: m/z 518 [M+H]⁺. HRMS (ESI) calcd for C₂₅H₂₆F₃N₃O₄Si [M+H]⁺ 518.1718 found 518.1725;

### 2-(1-benzothiophen-3-yl)-5-(1H-pyrrolo[2,3-b]pyridin-4-yl)-1-{[2-(trimethylsilyl)ethoxy]methyl}-1H-pyrrole-3-carboxylic acid (IX)

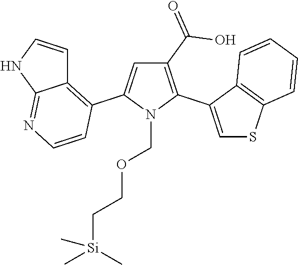 [SMILES: C[Si](C)(C)CCOCn1c(-c2ccnc3[nH]ccc23)cc(C(=O)O)c1-c1csc2ccccc12]

LCMS: m/z 490 [M+H]⁺. HRMS (ESI) calcd for C₂₆H₂₇N₃O₃SSi [M+H]⁺ 490.1615 found 490.1624;

### 2-(2,3-dihydro-1,4-benzodioxin-6-yl)-5-(1H-pyrrolo[2,3-b]pyridin-4-yl)-1-{[2-(trimethylsilyl)ethoxy]methyl}-1H-pyrrole-3-carboxylic acid (IX)

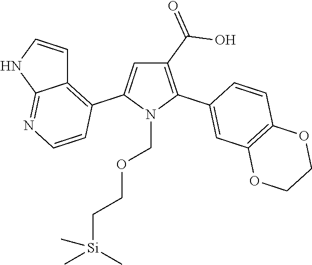 [SMILES: C[Si](C)(C)CCOCn1c(-c2ccnc3[nH]ccc23)cc(C(=O)O)c1-c1ccc2c(c1)OCCO2]

LCMS: m/z 492 [M+H]⁺. HRMS (ESI) calcd for C₂₆H₂₉N₃O₅Si [M+H]⁺ 492.1949 found 492.1952;

### 2-(4-fluoro-2-methylphenyl)-5-(1H-pyrrolo[2,3-b]pyridin-4-yl)-1-{[2-(trimethylsilyl)ethoxy]methyl}-1H-pyrrole-3-carboxylic acid (IX)

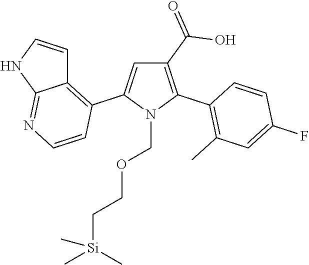 [SMILES: Cc1cc(F)ccc1-c1c(C(=O)O)cc(-c2ccnc3[nH]ccc23)n1COCC[Si](C)(C)C]

LCMS: m/z 466 [M+H]⁺. HRMS (ESI) calcd for C₂₅H₂₅N₃O₃Si [M+H]⁺ 466.1957 found 466.1959;

### 2-(2-fluoro-4-methylphenyl)-4-iodo-5-(1H-pyrrolo[2,3-b]pyridin-4-yl)-1-{[2-(trimethylsilyl)ethoxy]methyl}-1H-pyrrole-3-carboxylic acid (IX)

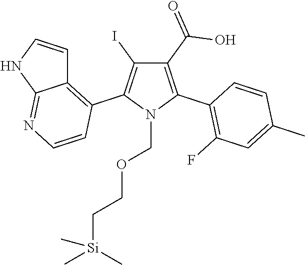 [SMILES: Cc1ccc(-c2c(C(=O)O)c(I)c(-c3ccnc4[nH]ccc34)n2COCC[Si](C)(C)C)c(F)c1]

LCMS: m/z 592 [M+H]⁺. HRMS (ESI) calcd for C₂₅H₂₇FlN₃O₃Si [M+H]⁺592.0923 found 592.0929.

### 2-(2-fluoro-4-methylphenyl)-4-bromo-5-(1H-pyrrolo[2,3-b]pyridin-4-yl)-1-{[2-(trimethylsilyl)ethoxy]methyl}-1H-pyrrole-3-carboxylic acid (IX)

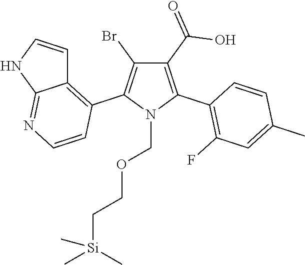 [SMILES: Cc1ccc(-c2c(C(=O)O)c(Br)c(-c3ccnc4[nH]ccc34)n2COCC[Si](C)(C)C)c(F)c1]

LCMS: m/z 544 [M+H]⁺. HRMS (ESI) calcd for C₂₅H₂₇BrFN₃O₃Si [M+H]⁺ 544.1062 found 544.1067;

### 4-ethenyl-2-(2-fluoro-4-methylphenyl)-5-(1H-pyrrolo[2,3-b]pyridin-4-yl)-1-{[2-(trimethylsilyl)ethoxy]methyl}-1H-pyrrole-3-carboxylic acid (IX)

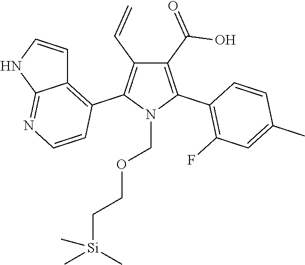 [SMILES: C=Cc1c(C(=O)O)c(-c2ccc(C)cc2F)n(COCC[Si](C)(C)C)c1-c1ccnc2[nH]ccc12]

LCMS: m/z 492 [M+H]⁺. HRMS (ESI) calcd for C₂₇H₃₀FN₃O₃Si [M+H]⁺ 492.2113 found 492.2114;

### 2-(2-fluoro-4-methylphenyl)-4-(prop-1-en-2-yl)-5-(1H-pyrrolo[2,3-b]pyridin-4-yl)-1-{[2-(trimethylsilyl) ethoxy]methyl}-1H-pyrrole-3-carboxylic acid (IX)

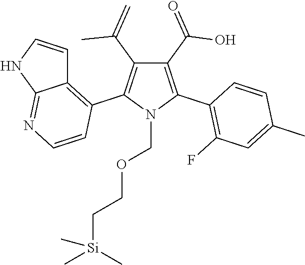 [SMILES: C=C(C)c1c(C(=O)O)c(-c2ccc(C)cc2F)n(COCC[Si](C)(C)C)c1-c1ccnc2[nH]ccc12]

LCMS: m/z 506 [M+H]⁺. HRMS (ESI) calcd for C₂₈H₃₂FN₃O₃Si [M+H]⁺ 506.2270 found 506.2271;

### Step 6

### 2-(3-chloro-2-fluorophenyl)-5-(1H-pyrrolo[2,3-b]pyridin-4-yl)-1-{[2-(trimethylsilyl)ethoxy]methyl}-1H-pyrrole-3-carboxamide (XI)

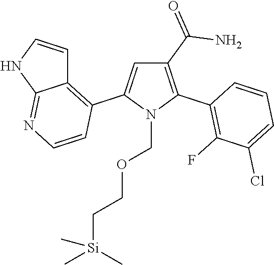 [SMILES: C[Si](C)(C)CCOCn1c(-c2ccnc3[nH]ccc23)cc(C(N)=O)c1-c1cccc(Cl)c1F]

To a solution of methyl 2-(3-chloro-2-fluorophenyl)-5-(1H-pyrrolo[2,3-b]pyridin-4-yl)-1-{[2-(trimethylsilyl)ethoxy]methyl}-1H-pyrrole-3-carboxylic acid (1 eq., 70 mg, 0.144 mmol) in DMA (1 ml), DIPEA (6 eq., 143 g, 0.87 mmol), TBTU (2 eq., 93 mg, 0.289 mmol), HOBt NH₃ (2 eq., 44 mg, 0.289 mmol) were added. The reaction mixture was stirred at room temperature overnight. Distilled water was added, and the product was extracted 3 times with AcOEt. The organic layer was washed with NaOH 0.5 M and brine, dried over anhydrous Na₂SO₄ and evaporated to dryness. The crude was purified by flash-chromatography (DCM/Acetone 7/3) affording the title compound (with solid, 67 mg, Y=96%). ¹H NMR (500 MHz, DMSO-d₆) d ppm −0.21-−0.18 (m, 9H) 0.52-0.58 (m, 2H) 2.95-3.02 (m, 2H) 5.01-5.19 (m, 2H) 6.67 (dd, J=3.43, 1.91 Hz, 1H) 6.87 (br. s., 1H) 7.08 (s, 1H) 7.23 (d, J=4.88 Hz, 1H) 7.30 (t, J=7.93 Hz, 1H) 7.46-7.51 (m, 1H) 7.54 (br. s., 1H) 7.56-7.58 (m, 1H) 7.62-7.68 (m, 1H) 8.28 (d, J=5.03 Hz, 1H) 11.82 (br. s., 1H). LCMS: m/z 485 [M+H]⁺. HRMS (ESI) calcd for C₂₄H₂₆ClFN₄O₂Si [M+H]⁺ 485.1571 found 485.1555;

Operating in an analogous way, but employing suitable substituted starting material the following compounds were obtained:

### 2-(4-chloro-2-fluorophenyl)-5-(1H-pyrrolo[2,3-b]pyridin-4-yl)-1-{[2-(trimethylsilyl)ethoxy]methyl}-1H-pyrrole-3-carboxamide (XI)

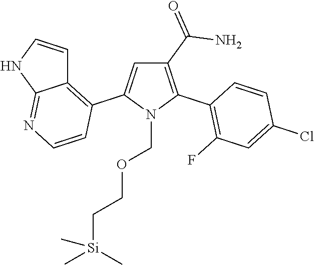 [SMILES: C[Si](C)(C)CCOCn1c(-c2ccnc3[nH]ccc23)cc(C(N)=O)c1-c1ccc(Cl)cc1F]

¹H NMR (500 MHz, DMSO-d₆) d ppm −0.23-−0.16 (m, 9H) 0.50-0.59 (m, 2H) 2.94-3.04 (m, 2H) 4.96-5.17 (m, 2H) 6.65 (dd, J=3.43, 1.91 Hz, 1H) 6.86 (br. s., 1H) 7.06 (s, 1H) 7.22 (d, J=4.88 Hz, 1H) 7.38 (dd, J=8.24, 1.83 Hz, 1H) 7.46-7.60 (m, 4H) 8.27 (d, J=5.03 Hz, 1H) 11.82 (br. s., 1H). LCMS: m/z 485 [M+H]⁺. HRMS (ESI) calcd for C₂₄H₂₆ClFN₄O₂Si [M+H]⁺ 485.1571 found 485.1567;

### 2-(2-chloro-4-fluorophenyl)-5-(1H-pyrrolo[2,3-b]pyridin-4-yl)-1-{[2-(trimethylsilyl)ethoxy]methyl}-1H-pyrrole-3-carboxamide (XI)

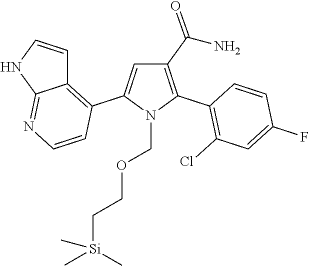 [SMILES: C[Si](C)(C)CCOCn1c(-c2ccnc3[nH]ccc23)cc(C(N)=O)c1-c1ccc(F)cc1Cl]

LCMS: m/z 485 [M+H]⁺. HRMS (ESI) calcd for C₂₄H₂₆ClFN₄O₂Si [M+H]⁺ 485.1571 found 485.1566;

### 2-(2,4-difluorophenyl)-5-[1-(phenylsulfonyl)-1H-pyrrolo[2,3-b]pyridin-4-yl]-1-{[2-(trimethylsilyl)ethoxy]methyl}-1H-pyrrole-3-carboxamide (XI)

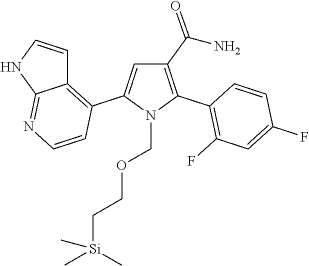 [SMILES: C[Si](C)(C)CCOCn1c(-c2ccnc3[nH]ccc23)cc(C(N)=O)c1-c1ccc(F)cc1F]

LCMS: m/z 469 [M+H]⁺. HRMS (ESI) calcd for C₂₄H₂₆F₂N₄O₂Si [M+H]⁺ 469.1866 found 469.1861;

### 2-[2-chloro-4-(trifluoromethyl)phenyl]-5-(1H-pyrrolo[2,3-b]pyridin-4-yl)-1-{[2-(trimethylsilyl)ethoxy]methyl}-1H-pyrrole-3-carboxamide (XI)

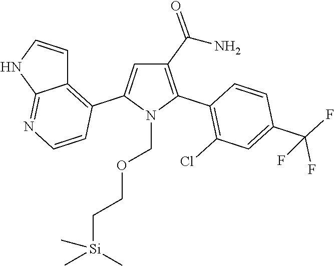 [SMILES: C[Si](C)(C)CCOCn1c(-c2ccnc3[nH]ccc23)cc(C(N)=O)c1-c1ccc(C(F)(F)F)cc1Cl]

¹H NMR (500 MHz, DMSO-d₆) d ppm −0.24-−0.20 (m, 9H) 0.45-0.55 (m, 0H) 2.90-2.96 (m, 2H) 4.87-5.20 (m, 2H) 6.62 (dd, J=3.43, 1.91 Hz, 1H) 6.87 (br. s., 1H) 7.08 (s, 1H) 7.20 (d, J=4.88 Hz, 1H) 7.54 (br. s., 1H) 7.57-7.60 (m, 1H) 7.71-7.76 (m, 1H) 7.77-7.82 (m, 1H) 7.97 (d, J=0.92 Hz, 1H) 8.28 (d, J=4.88 Hz, 1H) 11.84 (s, 1H). LCMS: m/z 535 [M+H]⁺. HRMS (ESI) calcd for C₂₅H₂₆ClF₃N₄O₂Si [M+H]⁺ 535.1539 found 535.1536;

### 2-(2,3-difluorophenyl)-5-(1H-pyrrolo[2,3-b]pyridin-4-yl)-1-{[2-(trimethylsilyl)ethoxy]methyl}-1H-pyrrole-3-carboxamide (XI)

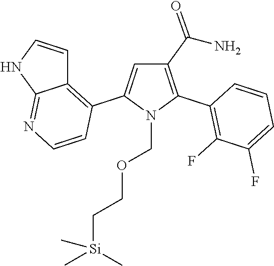 [SMILES: C[Si](C)(C)CCOCn1c(-c2ccnc3[nH]ccc23)cc(C(N)=O)c1-c1cccc(F)c1F]

LCMS: m/z 469 [M+H]⁺. HRMS (ESI) calcd for C₂₄H₂₆F₂N₄O₂Si [M+H]⁺ 469.1866 found 469.1862;

### 2-(2,3-dichlorophenyl)-5-(1H-pyrrolo[2,3-b]pyridin-4-yl)-1-{[2-(trimethylsilyl)ethoxy]methyl}-1H-pyrrole-3-carboxamide (XI)

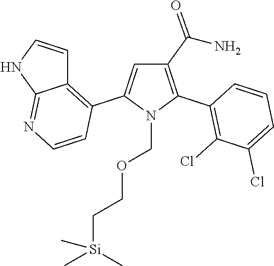 [SMILES: C[Si](C)(C)CCOCn1c(-c2ccnc3[nH]ccc23)cc(C(N)=O)c1-c1cccc(Cl)c1Cl]

LCMS: m/z 501 [M+H]⁺. HRMS (ESI) calcd for C₂₄H₂₅Cl₂N₄O₂Si [M+H]⁺ 501.1275 found 501.1278;

### 2-[4-methyl-2-(trifluoromethyl)phenyl]-5-(1H-pyrrolo[2,3-b]pyridin-4-yl)-1-{[2-(trimethylsilyl)ethoxy]methyl}-1H-pyrrole-3-carboxamide (XI)

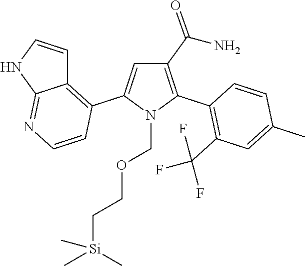 [SMILES: Cc1ccc(-c2c(C(N)=O)cc(-c3ccnc4[nH]ccc34)n2COCC[Si](C)(C)C)c(C(F)(F)F)c1]

¹H NMR (500 MHz, DMSO-d₆) d ppm −0.23-−0.19 (m, 9H) 0.46-0.57 (m, 2H) 2.46 (s, 3H) 2.83-3.02 (m, 2H) 4.71 (d, J=10.83 Hz, 1H) 5.09 (d, J=10.98 Hz, 1H) 6.52 (dd, J=3.43, 1.91 Hz, 1H) 6.73 (br. s., 1H) 7.00 (s, 1H) 7.16 (d, J=4.88 Hz, 1H) 7.24 (br. s., 1H) 7.38 (d, J=7.93 Hz, 1H) 7.53 (d, J=7.93 Hz, 1H) 7.55-7.57 (m, 1H) 7.64 (s, 1H) 8.26 (d, J=4.88 Hz, 1H) 11.81 (br. s., 1H). LCMS: m/z 515 [M+H]⁺. HRMS (ESI) calcd for C₂₆H₂₉F₃N₄O₂Si [M+H]⁺ 515.2085 found 515.2093;

### 2-(2-chloro-4-methylphenyl)-5-(1H-pyrrolo[2,3-b]pyridin-4-yl)-1-{[2-(trimethylsilyl)ethoxy]methyl}-1H-pyrrole-3-carboxamide (XI)

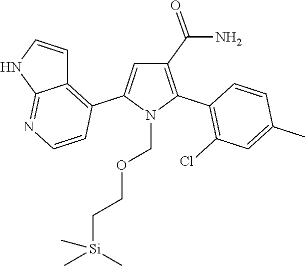 [SMILES: Cc1ccc(-c2c(C(N)=O)cc(-c3ccnc4[nH]ccc34)n2COCC[Si](C)(C)C)c(Cl)c1]

LCMS: m/z 481 [M+H]⁺. HRMS (ESI) calcd for C₂₅H₂₉ClN₄O₂Si [M+H]⁺ 481.1821 found 481.1825;

### 2-(2,3-difluoro-4-methylphenyl)-5-(1H-pyrrolo[2,3-b]pyridin-4-yl)-1-{[2-(trimethylsilyl)ethoxy]methyl}-1H-pyrrole-3-carboxamide (XI)

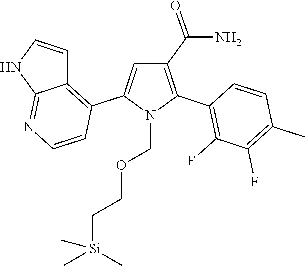 [SMILES: Cc1ccc(-c2c(C(N)=O)cc(-c3ccnc4[nH]ccc34)n2COCC[Si](C)(C)C)c(F)c1F]

LCMS: m/z 483 [M+H]⁺. HRMS (ESI) calcd for C₂₅H₂₅F₂N₄O₂Si [M+H]⁺ 483.2022 found 483.2025;

### 2-[2-methyl-4-(trifluoromethyl)phenyl]-5-(1H-pyrrolo[2,3-b]pyridin-4-yl)-1-{[2-(trimethylsilyl)ethoxy]methyl}-1H-pyrrole-3-carboxamide (XI)

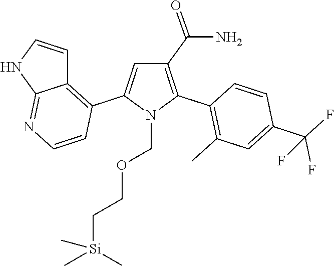 [SMILES: Cc1cc(C(F)(F)F)ccc1-c1c(C(N)=O)cc(-c2ccnc3[nH]ccc23)n1COCC[Si](C)(C)C]

¹H NMR (500 MHz, DMSO-d₆) d ppm −0.25-−0.19 (m, 9H) 0.46-0.54 (m, 2H) 2.20 (s, 3H) 2.82-2.98 (m, 2H) 4.89 (d, J=10.83 Hz, 1H) 5.01 (d, J=10.83 Hz, 1H) 6.65 (dd, J=3.43, 1.91 Hz, 1H) 6.83 (br. s., 1H) 7.08 (s, 1H) 7.21 (d, J=5.03 Hz, 1H) 7.41 (br. s., 1H) 7.51 (d, J=7.78 Hz, 1H) 7.55-7.57 (m, 1H) 7.60 (d, J=7.78 Hz, 1H) 7.67 (s, 1H) 8.27 (d, J=4.88 Hz, 1H) 11.81 (br. s., 1H). LCMS: m/z 515 [M+H]⁺. HRMS (ESI) calcd for C₂₆H₂₉F₃N₄O₂Si [M+H]⁺ 515.2085 found 515.2084;

### 2-(2-fluoro-3-methoxyphenyl)-5-(1H-pyrrolo[2,3-b]pyridin-4-yl)-1-{[2-(trimethylsilyl)ethoxy]methyl}-1H-pyrrole-3-carboxamide (XI)

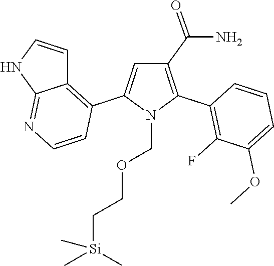 [SMILES: COc1cccc(-c2c(C(N)=O)cc(-c3ccnc4[nH]ccc34)n2COCC[Si](C)(C)C)c1F]

¹H NMR (500 MHz, DMSO-d₆) d ppm −0.22-−0.17 (m, 9H) 0.54 (t, J=8.39 Hz, 2H) 2.92-3.04 (m, 2H) 3.87 (s, 3H) 4.99-5.05 (m, 1H) 5.12-5.17 (m, 1H) 6.66 (dd, J=3.43, 1.91 Hz, 1H) 6.81 (br. s., 1H) 6.98-7.03 (m, 1H) 7.04 (s, 1H) 7.17-7.21 (m, 1H) 7.21-7.27 (m, 1H) 7.38 (br. s., 1H) 7.53-7.57 (m, 1H) 8.26 (d, J=4.88 Hz, 1H) 11.80 (s, 1H). LCMS: m/z 481 [M+H]⁺. HRMS (ESI) calcd for C₂₅H₂₉FN₄O₃Si [M+H]⁺ 481.2066 found 481.2061;

### 2-(2-chloro-3-fluorophenyl)-5-(1H-pyrrolo[2,3-b]pyridin-4-yl)-1-{[2-(trimethylsilyl)ethoxy]methyl}-1H-pyrrole-3-carboxamide (XI)

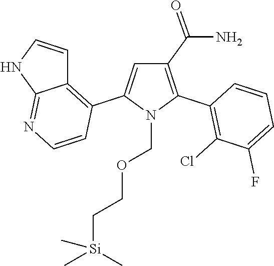 [SMILES: C[Si](C)(C)CCOCn1c(-c2ccnc3[nH]ccc23)cc(C(N)=O)c1-c1cccc(F)c1Cl]

¹H NMR (500 MHz, DMSO-d₆) d ppm −0.24-−0.17 (m, 9H) 0.48-0.56 (m, 2H) 2.90-3.00 (m, 2H) 4.96 (d, J=11.13 Hz, 1H) 5.13 (d, J=11.13 Hz, 1H) 6.62 (dd, J=3.43, 1.91 Hz, 1H) 6.83 (br. s., 1H) 7.07 (s, 1H) 7.21 (d, J=4.88 Hz, 1H) 7.35 (dd, J=7.47, 1.22 Hz, 1H) 7.42-7.53 (m, 3H) 7.55-7.59 (m, 1H) 8.27 (d, J=5.03 Hz, 1H) 11.82 (br. s., 1H). LCMS: m/z 485 [M+H]⁺. HRMS (ESI) calcd for C₂₄H₂₆ClFN₄O₂Si [M+H]⁺ 485.1571 found 485.1562;

### 2-(2-fluoro-3-methylphenyl)-5-(1H-pyrrolo[2,3-b]pyridin-4-yl)-1-{[2-(trimethylsilyl)ethoxy]methyl}-1H-pyrrole-3-carboxamide (XI)

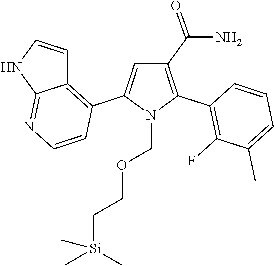 [SMILES: Cc1cccc(-c2c(C(N)=O)cc(-c3ccnc4[nH]ccc34)n2COCC[Si](C)(C)C)c1F]

¹H NMR (500 MHz, DMSO-d₆) d ppm −0.21-−0.17 (m, 9H) 0.55 (t, J=8.39 Hz, 2H) 2.27 (s, 3H) 2.95-3.04 (m, 2H) 4.97-5.04 (m, 1H) 5.12-5.17 (m, 1H) 6.66 (dd, J=3.43, 1.91 Hz, 1H) 6.79 (br. s., 1H) 7.04 (s, 1H) 7.14-7.18 (m, 1H) 7.25 (d, J=5.03 Hz, 1H) 7.28-7.32 (m, 1H) 7.33-7.41 (m, 2H) 7.55-7.57 (m, 1H) 8.26 (d, J=5.03 Hz, 1H) 11.80 (br. s., 1H). LCMS: m/z 465 [M+H]⁺. HRMS (ESI) calcd for C₂₅H₂₉FN₄O₂Si [M+H]⁺ 465.2117 found 465.2108;

### 2-[2-methyl-3-(trifluoromethyl)phenyl]-5-(1H-pyrrolo[2,3-b]pyridin-4-yl)-1-{[2-(trimethylsilyl)ethoxy]methyl}-1H-pyrrole-3-carboxamide (XI)

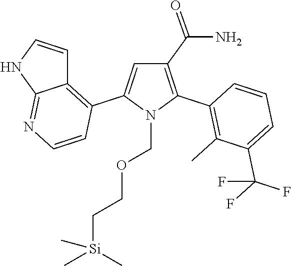 [SMILES: Cc1c(-c2c(C(N)=O)cc(-c3ccnc4[nH]ccc34)n2COCC[Si](C)(C)C)cccc1C(F)(F)F]

LCMS: m/z 515 [M+H]⁺. HRMS (ESI) calcd for C₂₆H₂₉F₃N₄O₂Si [M+H]⁺ 515.2085 found 515.2081;

### 2-[4-methoxy-2-(trifluoromethyl)phenyl]-5-(1H-pyrrolo[2,3-b]pyridin-4-yl)-1-{[2-(trimethylsilyl)ethoxy]methyl}-1H-pyrrole-3-carboxamide (XI)

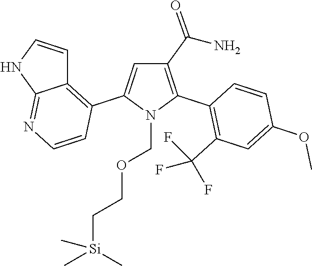 [SMILES: COc1ccc(-c2c(C(N)=O)cc(-c3ccnc4[nH]ccc34)n2COCC[Si](C)(C)C)c(C(F)(F)F)c1]

LCMS: m/z 531 [M+H]⁺. HRMS (ESI) calcd for C₂₆H₂₉F₃N₄O₃Si [M+H]⁺ 531.2034 found 531.2029;

### 2-[2-chloro-4-(difluoromethoxy)phenyl]-5-(1H-pyrrolo[2,3-b]pyridin-4-yl)-1-{[2-(trimethylsilyl)ethoxy]methyl}-1H-pyrrole-3-carboxamide (XI)

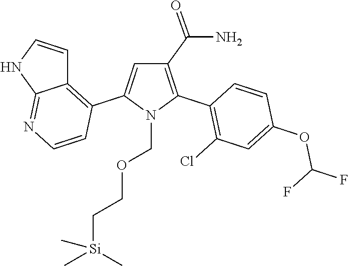 [SMILES: C[Si](C)(C)CCOCn1c(-c2ccnc3[nH]ccc23)cc(C(N)=O)c1-c1ccc(OC(F)F)cc1Cl]

¹H NMR (500 MHz, DMSO-d₆) d ppm −0.22-−0.19 (m, 9H) 0.48-0.55 (m, 2H) 2.91-3.00 (m, 2H) 4.91 (d, J=10.98 Hz, 1H) 5.14 (d, J=11.13 Hz, 1H) 6.61 (dd, J=3.36, 1.98 Hz, 1H) 6.81 (br. s., 1H) 7.05 (s, 1H) 7.16-7.28 (m, 3H) 7.37-7.45 (m, 3H) 7.52-7.59 (m, 3H) 8.25-8.29 (m, 1H) 11.82 (br. s., 1H). LCMS: m/z 533 [M+H]⁺. HRMS (ESI) calcd for C₂₅H₂₇ClF₂N₄O₃Si [M+H]⁺ 533.1582 found 533.1585;

### 2-(3,4-dichlorophenyl)-5-(1H-pyrrolo[2,3-b]pyridin-4-yl)-1-{[2-(trimethylsilyl)ethoxy]methyl}-1H-pyrrole-3-carboxamide (XI)

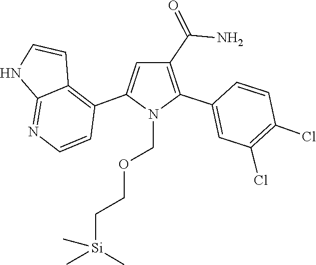 [SMILES: C[Si](C)(C)CCOCn1c(-c2ccnc3[nH]ccc23)cc(C(N)=O)c1-c1ccc(Cl)c(Cl)c1]

¹H NMR (500 MHz, DMSO-d₆) d ppm −0.17-0.13 (m, 8H) 0.61-0.68 (m, 1H) 1.95 (s, 1H) 2.78 (s, 1H) 2.94 (s, 1H) 3.05-3.12 (m, 1H) 5.06 (s, 1H) 6.71 (dd, J=3.36, 1.98 Hz, 1H) 6.93 (br. s., 1H) 7.02 (s, 1H) 7.25 (d, J=5.03 Hz, 1H) 7.46-7.50 (m, 1H) 7.51-7.54 (m, 1H) 7.56-7.58 (m, 1H) 7.70 (d, J=8.24 Hz, 1H) 7.76 (d, J=1.98 Hz, 1H) 8.27 (d, J=5.03 Hz, 1H) 11.82 (br. s., 1H). LCMS: m/z 501 [M+H]⁺. HRMS (ESI) calcd for C₂₄H₂₆Cl₂N₄O₂Si [M+H]⁺ 501.1275 found 501.1282;

### 2-(3,4-difluorophenyl)-5-(1H-pyrrolo[2,3-b]pyridin-4-yl)-1-{[2-(trimethylsilyl)ethoxy]methyl}-1H-pyrrole-3-carboxamide (XI)

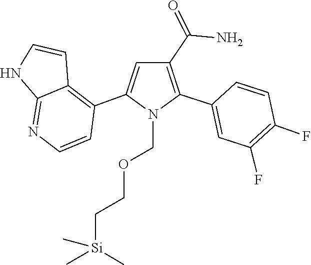 [SMILES: C[Si](C)(C)CCOCn1c(-c2ccnc3[nH]ccc23)cc(C(N)=O)c1-c1ccc(F)c(F)c1]

¹H NMR (500 MHz, DMSO-d₆) d ppm −0.18-−0.13 (m, 8H) 0.58-0.66 (m, 2H) 3.03-3.11 (m, 2H) 5.07 (s, 2H) 6.69 (dd, J=3.43, 1.91 Hz, 1H) 6.89 (br. s., 1H) 7.00 (s, 1H) 7.25 (d, J=4.88 Hz, 1H) 7.31-7.38 (m, 1H) 7.44 (br. s., 1H) 7.48-7.60 (m, 3H) 8.27 (d, J=4.88 Hz, 1H) 11.81 (br. s., 1H). LCMS: m/z 469 [M+H]⁺. HRMS (ESI) calcd for C₂₄H₂₆F₂N₄O₂Si [M+H]⁺ 469.1866 found 469.1864;

### 2-(3-ethoxy-2-fluorophenyl)-5-(1H-pyrrolo[2,3-b]pyridin-4-yl)-1-{[2-(trimethylsilyl)ethoxy]methyl}-1H-pyrrole-3-carboxamide (XI)

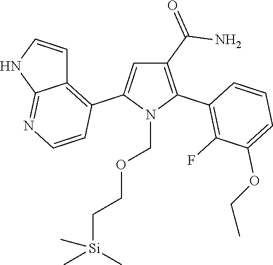 [SMILES: CCOc1cccc(-c2c(C(N)=O)cc(-c3ccnc4[nH]ccc34)n2COCC[Si](C)(C)C)c1F]

¹H NMR (500 MHz, DMSO-d₆) d ppm −0.22-−0.18 (m, 8H) 0.55 (t, J=8.39 Hz, 2H) 1.34-1.40 (m, 3H) 4.12 (quin, J=7.40 Hz, 2H) 4.98-5.19 (m, 2H) 6.66 (dd, J=3.36, 1.98 Hz, 1H) 6.81 (br. s., 1H) 6.96-7.02 (m, 1H) 7.04 (s, 1H) 7.13-7.29 (m, 3H) 7.37 (br. s., 1H) 7.53-7.59 (m, 1H) 8.26 (d, J=5.03 Hz, 1H) 11.80 (br. s., 1H). LCMS: m/z 495 [M+H]⁺. HRMS (ESI) calcd for C₂₆H₃₁FN₄O₃Si [M+H]⁺ 495.2222 found 495.2216;

### 2-(4-methyl-3-nitrophenyl)-5-(1H-pyrrolo[2,3-b]pyridin-4-yl)-1-{[2-(trimethylsilyl)ethoxy]methyl}-1H-pyrrole-3-carboxamide (XI)

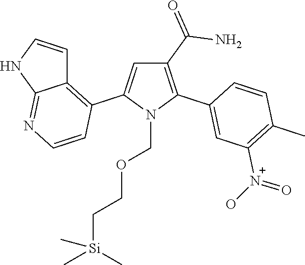

¹H NMR (500 MHz, DMSO-d₆) d ppm −0.22-−0.09 (m, 1H) 0.57-0.73 (m, 1H) 2.59 (s, 1H) 3.06-3.14 (m, 1H) 5.05 (s, 1H) 6.72 (dd, J=3.36, 1.98 Hz, 1H) 6.93 (br. s., 1H) 7.05 (s, 1H) 7.27 (d, J=5.03 Hz, 1H) 7.52-7.60 (m, 1H) 7.74 (dd, J=7.85, 1.75 Hz, 1H) 8.15 (d, J=1.68 Hz, 1H) 8.28 (d, J=5.03 Hz, 1H) 11.82 (br. s., 1H). LCMS: m/z 492 [M+H]⁺. HRMS (ESI) calcd for C₂₅H₂₉N₅O₄Si [M+H]⁺ 492.2062 found 492.2056;

### 2-(3-carbamoyl-4-fluorophenyl)-5-(1H-pyrrolo[2,3-b]pyridin-4-yl)-1-{[2-(trimethylsilyl)ethoxy]methyl}-1H-pyrrole-3-carboxamide (XI)

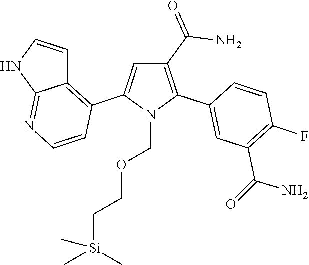 [SMILES: C[Si](C)(C)CCOCn1c(-c2ccnc3[nH]ccc23)cc(C(N)=O)c1-c1ccc(F)c(C(N)=O)c1]

¹H NMR (500 MHz, DMSO-d₆) d ppm −0.13 (s, 9H) 0.63-0.75 (m, 2H) 3.03-3.16 (m, 2H) 5.00-5.07 (m, 2H) 6.67-6.77 (m, 1H) 6.85-6.93 (m, 1H) 6.99-7.07 (m, 1H) 7.27-7.33 (m, 1H) 7.33-7.40 (m, 1H) 7.41-7.48 (m, 1H) 7.56-7.60 (m, 1H) 7.61-7.66 (m, 1H) 7.68-7.73 (m, 1H) 7.74-7.79 (m, 1H) 7.82-7.87 (m, 1H) 8.26-8.31 (m, 1H) 11.66-11.98 (m, 1H). LCMS: m/z 494 [M+H]⁺. HRMS (ESI) calcd for C₂₅H₂₈FN₅O₃Si [M+H]⁺ 494.2018 found 494.2011;

### 2-(2-fluoro-4-methylphenyl)-N-[2-(morpholin-4-yl)ethyl]-5-(1H-pyrrolo[2,3-b]pyridin-4-yl)-1-{[2-(trimethylsilyl) ethoxy]methyl}-1H-pyrrole-3-carboxamide (XI)

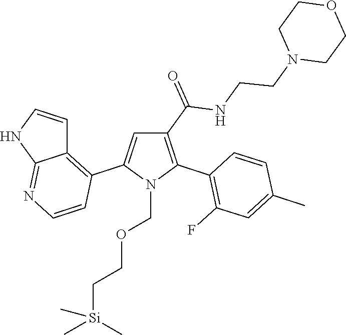 [SMILES: Cc1ccc(-c2c(C(=O)NCCN3CCOCC3)cc(-c3ccnc4[nH]ccc34)n2COCC[Si](C)(C)C)c(F)c1]

To a solution of 2-(2-fluoro-4-methylphenyl)-5-(1H-pyrrolo[2,3-b]pyridin-4-yl)-1-{[2-(trimethylsilyl)ethoxy]methyl}-1H-pyrrole-3-carboxylic acid (1 eq., 90 mg, 0.19 mmol) in DMA (1.5 ml), DIPEA (3 eq., 131 μl, 07.6 mmol), TBTU (2 eq., 118.5 mg, 0.38 mmol), 2-(morpholin-1-yl)ethanamine (1.5 eq., 38 μl, 0.28 mmol) were added. The reaction mixture was stirred at room temperature overnight. Distilled water was added, and the product was extracted 3 times with AcOEt. The organic layer was washed with NaOH 0.5 M and brine, dried over anhydrous Na₂SO₄ and evaporated to dryness. The crude was purified by flash-chromatography (DCM/MeOH 9/1) affording the title compound (beige solid, 92 mg, Y=84%).

¹H NMR (500 MHz, DMSO-d₆) d ppm −0.24-−0.17 (m, 2H) 0.54 (t, J=8.39 Hz, 1H) 2.27-2.41 (m, 2H) 2.90-3.04 (m, 1H) 3.19-3.28 (m, 1H) 3.52 (t, J=4.50 Hz, 1H) 4.92-5.27 (m, 1H) 6.64 (dd, J=3.43, 1.91 Hz, 1H) 6.99 (s, 1H) 7.07-7.15 (m, 1H) 7.24 (d, J=4.88 Hz, 1H) 7.38 (t, J=7.85 Hz, 1H) 7.55-7.60 (m, 1H) 7.65 (t, J=5.57 Hz, 1H) 8.26 (d, J=5.03 Hz, 1H) 11.81 (s, 1H). LCMS: m/z 578 [M+H]⁺. HRMS (ESI) calcd for C₃₁H₄₀FN₅O₃Si [M+H]⁺ 578.2957 found 578.2952;

Operating in an analogous way, but employing suitable substituted starting material the following compounds were obtained:

### N-[2-(dimethylamino)ethyl]-2-(2-fluoro-4-methylphenyl)-5-(1H-pyrrolo[2,3-b]pyridin-4-yl)-1-{[2-(trimethylsilyl) ethoxy]methyl}-1H-pyrrole-3-carboxamide (XI)

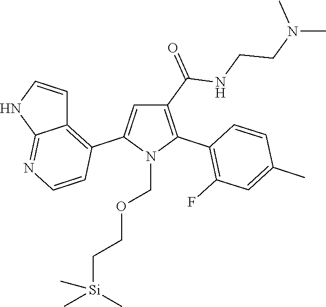 [SMILES: Cc1ccc(-c2c(C(=O)NCCN(C)C)cc(-c3ccnc4[nH]ccc34)n2COCC[Si](C)(C)C)c(F)c1]

¹H NMR (500 MHz, DMSO-d₆) d ppm −0.23-−0.16 (m, 9H) 0.54 (t, J=8.39 Hz, 1H) 2.10 (s, 6H) 2.27 (t, J=6.79 Hz, 2H) 2.39 (s, 3H) 2.90-3.04 (m, 2H) 3.13-3.24 (m, 2H) 4.94-5.23 (m, 2H) 6.64 (dd, J=3.35, 1.98 Hz, 1H) 7.00 (s, 1H) 7.04-7.15 (m, 2H) 7.24 (d, J=5.03 Hz, 1H) 7.36 (t, J=7.85 Hz, 1H) 7.57 (t, J=2.97 Hz, 1H) 7.65 (t, J=5.49 Hz, 1H) 8.26 (d, J=4.88 Hz, 1H) 11.81 (br. s., 1H). LCMS: m/z 536 [M+H]⁺. HRMS (ESI) calcd for C₂₉H₃₈FN₅O₂Si [M+H]⁺ 536.2852 found 536.2849;

### 2-(2-fluoro-4-methylphenyl)-N-[2-(pyrrolidin-1-yl)ethyl]-5-(1H-pyrrolo[2,3-b]pyridin-4-yl)-1-{[2-(trimethylsilyl) ethoxy]methyl}-1H-pyrrole-3-carboxamide (XI)

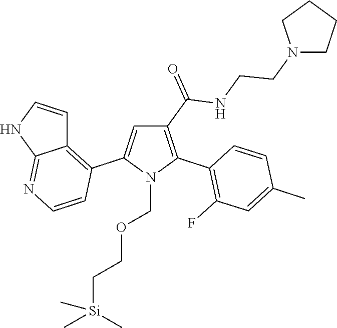 [SMILES: Cc1ccc(-c2c(C(=O)NCCN3CCCC3)cc(-c3ccnc4[nH]ccc34)n2COCC[Si](C)(C)C)c(F)c1]

LCMS: m/z 562 [M+H]⁺. HRMS (ESI) calcd for C₃₁H₄₀FN₅O₂Si [M+H]⁺ 562.3008 found 562.3012;

### tert-butyl-[(1R,2S)-2-({[2-(2-fluoro-4-methylphenyl)-5-(1H-pyrrolo[2,3-b]pyridin-4-yl)-1-{[2-(trimethylsilyl) ethoxy]methyl}-1H-pyrrol-3-yl]carbonyl}amino)cyclohexyl]carbamate (XI)

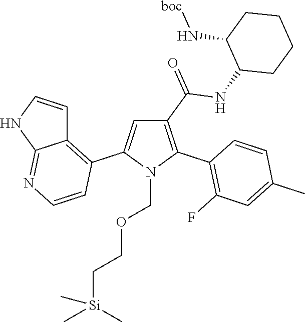

¹H NMR (500 MHz, DMSO-d₆) d ppm −0.21 (s, 9H) 0.52 (t, J=8.39 Hz, 2H) 1.16-1.67 (m, 15H) 2.38 (s, 3H) 2.92-3.01 (m, 2H) 3.52-3.66 (m, 1H) 3.89 (br. s., 1H) 4.92-5.26 (m, 2H) 6.47-6.64 (m, 2H) 6.91 (br. s., 1H) 7.11 (d, J=10.22 Hz, 3H) 7.23 (d, J=4.88 Hz, 1H) 7.35-7.48 (m, 1H) 7.56 (t, J=2.97 Hz, 1H) 8.27 (d, J=4.88 Hz, 1H) 11.82 (br. s., 1H). LCMS: m/z 662 [M+H]⁺. HRMS (ESI) calcd for C₃₆H₄₈FN₅O₄Si [M+H]⁺ 662.3533 found 662.3553;

### 2-(2-fluoro-4-methylphenyl)-N-(furan-2-ylmethyl)-5-(1H-pyrrolo[2,3-b]pyridin-4-yl)-1-{[2-(trimethylsilyl) ethoxy]methyl}-1H-pyrrole-3-carboxamide (XI)

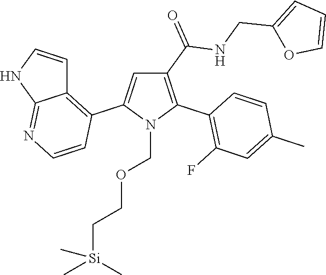 [SMILES: Cc1ccc(-c2c(C(=O)NCc3ccco3)cc(-c3ccnc4[nH]ccc34)n2COCC[Si](C)(C)C)c(F)c1]

¹H NMR (500 MHz, DMSO-d₆) d ppm −0.25-−0.16 (m, 9H) 0.55 (t, J=8.31 Hz, 2H) 2.37-2.41 (m, 3H) 2.95-3.05 (m, 2H) 4.27-4.39 (m, 2H) 4.95-5.26 (m, 2H) 6.18 (dd, J=3.13, 0.69 Hz, 1H) 6.37 (dd, J=3.20, 1.83 Hz, 1H) 6.68 (dd, J=3.36, 1.98 Hz, 1H) 7.04-7.14 (m, 3H) 7.25 (d, J=5.03 Hz, 1H) 7.36 (t, J=7.70 Hz, 1H) 7.52-7.59 (m, 2H) 8.26 (d, J=4.88 Hz, 1H) 8.42 (t, J=5.87 Hz, 1H) 11.81 (br. s., 1H). LCMS: m/z 545 [M+H]⁺. HRMS (ESI) calcd for C₃₀H₃₃FN₄O₃Si [M+H]⁺ 545.2379 found 545.2383;

### tert-butyl-[2-({[2-(2-fluoro-4-methylphenyl)-5-(1H-pyrrolo[2,3-b]pyridin-4-yl)-1-{[2-(trimethylsilyl)ethoxy]methyl}-1H-pyrrol-3-yl]carbonylamino)ethyl]methylcarbamate (XI)

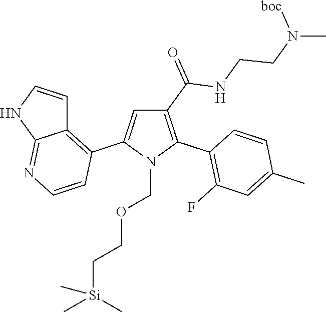

¹H NMR (500 MHz, DMSO-d₆) d ppm −0.24-−0.18 (m, 9H) 0.54 (t, J=8.39 Hz, 2H) 1.27-1.41 (m, 9H) 2.36-2.42 (m, 3H) 2.77 (d, J=10.37 Hz, 2H) 2.89-3.05 (m, 2H) 3.14-3.29 (m, 2H) 4.87-5.31 (m, 2H) 6.64 (d, J=8.69 Hz, 1H) 6.97-7.12 (m, 3H) 7.24 (d, J=4.88 Hz, 1H) 7.34 (br. s., 1H) 7.57 (t, J=2.82 Hz, 1H) 7.99 (br. s., 1H) 8.26 (d, J=5.03 Hz, 1H) 11.81 (br. s., 1H). LCMS: m/z 622 [M+H]⁺. HRMS (ESI) calcd for C₃₃H₄₄FN₅O₄Si [M+H]⁺ 622.322 found 622.3234;

### N-(2-fluoroethyl)-2-(2-fluoro-4-methylphenyl)-5-(1H-pyrrolo[2,3-b]pyridin-4-yl)-1-{[2-(trimethylsilyl)ethoxy]methyl}-1H-pyrrole-3-carboxamide (XI)

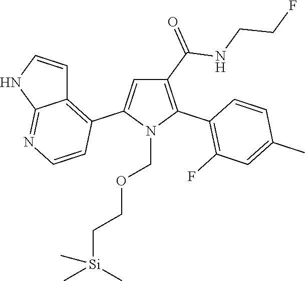 [SMILES: Cc1ccc(-c2c(C(=O)NCCF)cc(-c3ccnc4[nH]ccc34)n2COCC[Si](C)(C)C)c(F)c1]

¹H NMR (500 MHz, DMSO-d₆) d ppm −0.21-−0.17 (m, 9H) 0.55 (t, J=8.39 Hz, 2H) 2.36-2.41 (m, 3H) 2.95-3.05 (m, 2H) 3.38-3.47 (m, 1H) 4.39 (t, J=5.19 Hz, 1H) 4.48 (t, J=5.19 Hz, 1H) 4.89-5.28 (m, 2H) 6.68 (dd, J=3.43, 1.91 Hz, 1H) 7.05-7.13 (m, 3H) 7.26 (d, J=4.88 Hz, 1H) 7.36 (t, J=7.63 Hz, 1H) 7.52-7.62 (m, 1H) 8.16 (t, J=5.57 Hz, 1H) 8.26 (d, J=5.03 Hz, 1H) 11.81 (br. s., 1H). LCMS: m/z 511 [M+H]⁺. HRMS (ESI) calcd for C₂₇H₃₂F₂N₄O₂Si [M+H]⁺511.2336 found 511.2336;

### 2-(2-fluoro-4-methylphenyl)-N-(1-methylpiperidin-4-yl)-5-(1H-pyrrolo[2,3-b]pyridin-4-yl)-1-{[2-(trimethylsilyl) ethoxy]methyl}-1H-pyrrole-3-carboxamide (XI)

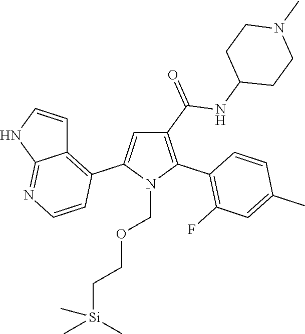 [SMILES: Cc1ccc(-c2c(C(=O)NC3CCN(C)CC3)cc(-c3ccnc4[nH]ccc34)n2COCC[Si](C)(C)C)c(F)c1]

¹H NMR (500 MHz, DMSO-d₆) d ppm −0.28-−0.14 (m, 9H) 0.54 (t, J=8.39 Hz, 2H) 1.53 (d, J=9.15 Hz, 2H) 1.74 (br. s., 2H) 2.15-2.35 (m, 2H) 2.38 (s, 3H) 2.74-3.05 (m, 4H) 3.65 (br. s., 1H) 4.91-5.29 (m, 2H) 6.65 (dd, J=3.36, 1.98 Hz, 1H) 7.05 (s, 1H) 7.07-7.12 (m, 2H) 7.24 (d, J=4.88 Hz, 1H) 7.37 (t, J=7.78 Hz, 1H) 7.53-7.59 (m, 1H) 7.63 (br. s., 1H) 8.26 (d, J=4.88 Hz, 1H) 11.81 (br. s., 1H). LCMS: m/z 562 [M+H]⁺. HRMS (ESI) calcd for C₃₁H₄₀FN₅O₂Si [M+H]⁺ 562.3008 found 562.2991;

### 2-(dibenzo[b,d]thiophen-4-yl)-5-(1H-pyrrolo[2,3-b]pyridin-4-yl)-1-{[2-(trimethylsilyl)ethoxy]methyl}-1H-pyrrole-3-carboxamide (XI)

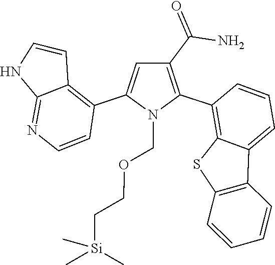 [SMILES: C[Si](C)(C)CCOCn1c(-c2ccnc3[nH]ccc23)cc(C(N)=O)c1-c1cccc2c1sc1ccccc12]

LCMS: m/z 539 [M+H]⁺. HRMS (ESI) calcd for C₃₀H₃₀N₄O₂SSi [M+H]⁺ 539.1932 found 539.1938;

### 2-(4-methylnaphthalen-1-yl)-5-(1H-pyrrolo[2,3-b]pyridin-4-yl)-1-{[2-(trimethylsilyl)ethoxy]methyl}-1H-pyrrole-3-carboxamide (XI)

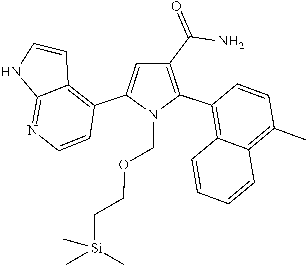 [SMILES: Cc1ccc(-c2c(C(N)=O)cc(-c3ccnc4[nH]ccc34)n2COCC[Si](C)(C)C)c2ccccc12]

¹H NMR (500 MHz, DMSO-d₆) d ppm −0.32-−0.26 (m, 9H) 0.26-0.36 (m, 2H) 2.68-2.83 (m, 5H) 4.73-5.08 (m, 2H) 6.65-6.77 (m, 2H) 6.86-6.99 (m, 1H) 7.12 (s, 1H) 7.28 (d, J=5.03 Hz, 1H) 7.43-7.54 (m, 4H) 7.55-7.61 (m, 2H) 8.09 (d, J=8.39 Hz, 1H) 8.26 (d, J=5.03 Hz, 1H) 11.81 (br. s., 1H). LCMS: m/z 497 [M+H]⁺. HRMS (ESI) calcd for C₂₉H₃₂N₄O₂Si [M+H]⁺ 497.2368 found 497.2369;

### 2-(3-fluorophenyl)-5-(1H-pyrrolo[2,3-b]pyridin-4-yl)-1-{[2-(trimethylsilyl)ethoxy]methyl}-1H-pyrrole-3-carboxamide (XI)

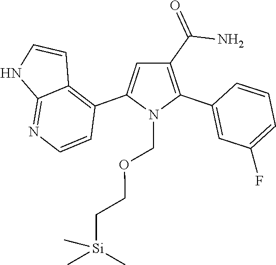 [SMILES: C[Si](C)(C)CCOCn1c(-c2ccnc3[nH]ccc23)cc(C(N)=O)c1-c1cccc(F)c1]

¹H NMR (500 MHz, DMSO-d6) d ppm −0.20-−0.12 (m, 9H) 0.57-0.67 (m, 2H) 3.01-3.10 (m, 2H) 5.06 (s, 2H) 6.70 (dd, J=3.43, 1.91 Hz, 1H) 6.88 (br. s., 1H) 6.99 (s, 1H) 7.22-7.30 (m, 2H) 7.30-7.36 (m, 2H) 7.39 (br. s., 1H) 7.44-7.52 (m, 1H) 7.52-7.60 (m, 1H) 8.27 (d, J=4.88 Hz, 1H) 11.80 (br. s., 1H). LCMS: m/z 451 [M+H]⁺. HRMS (ESI) calcd for C₂₄H₂₇FN₄O₂Si [M+H]⁺ 451.196 found 451.1948;

### 5-(1H-pyrrolo[2,3-b]pyridin-4-yl)-2-[4-(trifluoromethoxy)phenyl]-1-{[2-(trimethylsilyl)ethoxy]methyl}-1H-pyrrole-3-carboxamide (XI)

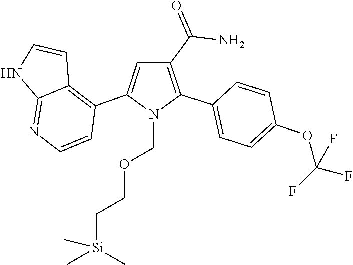 [SMILES: C[Si](C)(C)CCOCn1c(-c2ccnc3[nH]ccc23)cc(C(N)=O)c1-c1ccc(OC(F)(F)F)cc1]

¹H NMR (500 MHz, DMSO-d6) d ppm −0.22-−0.14 (m, 9H) 0.52-0.67 (m, 2H) 2.92-3.11 (m, 2H) 5.05 (s, 2H) 6.70 (dd, J=3.36, 1.98 Hz, 1H) 6.82-6.91 (m, 1H) 7.01 (s, 1H) 7.26 (d, J=5.03 Hz, 1H) 7.43 (m, 3H) 7.53-7.58 (m, 1H) 7.59-7.66 (m, 2H) 8.27 (d, J=4.88 Hz, 1H) 11.80 (br. s., 1H). LCMS: m/z 517 [M+H]⁺. HRMS (ESI) calcd for C₂₅H₂₇F₃N₄O₃Si [M+H]⁺ 517.1878 found 517.1857;

### 2-(1-benzothiophen-3-yl)-5-(1H-pyrrolo[2,3-b]pyridin-4-yl)-1-{[2-(trimethylsilyl)ethoxy]methyl}-1H-pyrrole-3-carboxamide (XI)

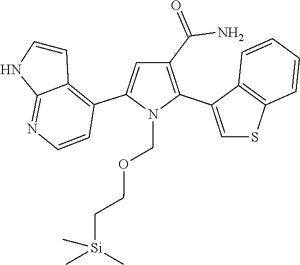 [SMILES: C[Si](C)(C)CCOCn1c(-c2ccnc3[nH]ccc23)cc(C(N)=O)c1-c1csc2ccccc12]

¹H NMR (500 MHz, DMSO-d₆) d ppm −0.28-−0.25 (m, 9H) 0.39-0.49 (m, 2H) 2.88 (dd, J=9.00, 7.78 Hz, 2H) 4.97 (d, J=10.83 Hz, 1H) 5.22 (d, J=10.83 Hz, 1H) 6.70 (dd, J=3.43, 1.91 Hz, 1H) 6.78-6.86 (m, 1H) 7.10 (s, 1H) 7.24 (br. s., 1H) 7.29 (d, J=5.03 Hz, 1H) 7.34-7.42 (m, 2H) 7.43-7.47 (m, 1H) 7.57 (t, J=1.00 Hz, 1H) 7.93 (s, 1H) 8.04 (m, J=7.09, 1.14 Hz, 1H) 8.27 (d, J=5.03 Hz, 1H) 11.81 (br. s., 1H). LCMS: m/z 489 [M+H]⁺. HRMS (ESI) calcd for C₂₆H₂₅N₄O₂SSi [M+H]⁺ 489.1775 found 489.1761;

### 2-(2,3-dihydro-1,4-benzodioxin-6-yl)-5-(1H-pyrrolo[2,3-b]pyridin-4-yl)-1-{[2-(trimethylsilyl)ethoxy]methyl}-1H-pyrrole-3-carboxamide (XI)

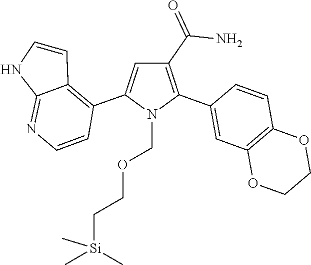 [SMILES: C[Si](C)(C)CCOCn1c(-c2ccnc3[nH]ccc23)cc(C(N)=O)c1-c1ccc2c(c1)OCCO2]

¹H NMR (500 MHz, DMSO-d₆) d ppm −0.17-−0.12 (m, 9H) 0.61-0.70 (m, 2H) 3.03-3.13 (m, 2H) 4.21-4.34 (m, 4H) 5.04 (s, 2H) 6.68 (dd, J=3.43, 1.91 Hz, 1H) 6.84 (br. s., 1H) 6.89-6.98 (m, 3H) 7.01 (d, J=1.98 Hz, 1H) 7.06 (br. s., 1H) 7.27 (d, J=5.03 Hz, 1H) 7.54 (t, J=1.00 Hz, 1H) 8.25 (d, J=5.03 Hz, 1H) 11.77 (br. s., 1H). LCMS: m/z 491 [M+H]⁺. HRMS (ESI) calcd for C₂₆H₃₀N₄O₄Si [M+H]⁺ 491.2109 found 491.2094;

### 2-(4-fluoro-2-methylphenyl)-5-(1H-pyrrolo[2,3-b]pyridin-4-yl)-1-{[2-(trimethylsilyl)ethoxy]methyl}-1H-pyrrole-3-carboxamide (XI)

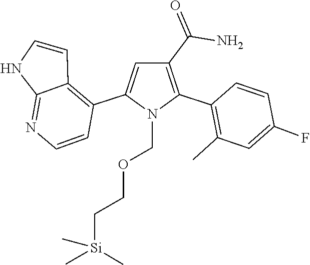 [SMILES: Cc1cc(F)ccc1-c1c(C(N)=O)cc(-c2ccnc3[nH]ccc23)n1COCC[Si](C)(C)C]

¹H NMR (500 MHz, DMSO-d₆) d ppm −0.22-−0.19 (m, 9H) 0.54 (t, J=8.39 Hz, 2H) 2.12 (s, 3H) 2.86-3.01 (m, 2H) 4.88 (d, J=10.83 Hz, 1H) 5.01 (d, J=10.68 Hz, 1H) 6.63 (dd, J=3.36, 1.98 Hz, 1H) 6.79 (br. s., 1H) 7.03 (s, 1H) 7.09 (td, J=8.58, 2.82 Hz, 1H) 7.12-7.19 (m, 2H) 7.22 (d, J=5.03 Hz, 1H) 7.32 (dd, J=8.39, 6.10 Hz, 1H) 7.53-7.57 (m, 1H) 8.25 (d, J=5.03 Hz, 1H) 11.79 (br. s., 1H). LCMS: m/z 465 [M+H]⁺. HRMS (ESI) calcd for C₂₅H₂₉FN₄O₂Si [M+H]⁺ 465.2117 found 465.2126;

### 2-(2-fluoro-4-methylphenyl)-4-iodo-5-(1H-pyrrolo[2,3-b]pyridin-4-yl)-1-{[2-(trimethylsilyl)ethoxy]methyl}-1H-pyrrole-3-carboxamide (XI)

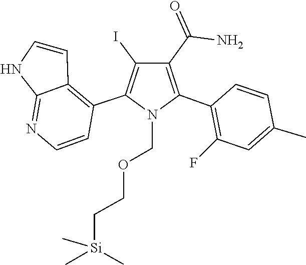 [SMILES: Cc1ccc(-c2c(C(N)=O)c(I)c(-c3ccnc4[nH]ccc34)n2COCC[Si](C)(C)C)c(F)c1]

¹H NMR (500 MHz, DMSO-d₆) d ppm −0.27-−0.23 (m, 9H) 0.36 (t, J=7.70 Hz, 2H) 2.36-2.40 (m, 3H) 2.74-2.89 (m, 2H) 4.93 (br. s., 1H) 6.04-6.40 (m, 1H) 6.99 (d, J=4.88 Hz, 2H) 7.07-7.21 (m, 3H) 7.43 (br. s., 1H) 7.54-7.61 (m, 1H) 8.28-8.38 (m, 1H) 11.87 (br. s., 1H). LCMS: m/z 591 [M+H]⁺. HRMS (ESI) calcd for C₂₅H₂₅FlN₄O₂Si [M+H]⁺ 591.1083 found 591.1073;

### 2-(2-fluoro-4-methylphenyl)-4-bromo-5-(1H-pyrrolo[2,3-b]pyridin-4-yl)-1-{[2-(trimethylsilyl)ethoxy]methyl}-1H-pyrrole-3-carboxamide (XI)

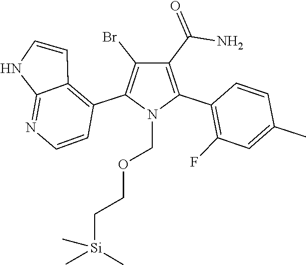 [SMILES: Cc1ccc(-c2c(C(N)=O)c(Br)c(-c3ccnc4[nH]ccc34)n2COCC[Si](C)(C)C)c(F)c1]

¹H NMR (500 MHz, DMSO-d₆) d ppm −0.35-−0.19 (m, 9H) 0.37 (t, J=7.78 Hz, 2H) 2.38 (s, 3H) 2.76-2.92 (m, 2H) 4.90-5.23 (m, 2H) 6.32 (br. s., 1H) 6.84-7.25 (m, 4H) 7.43 (br. s., 1H) 7.59 (t, J=2.97 Hz, 1H) 8.33 (d, J=4.88 Hz, 1H) 11.88 (br. s., 1H). LCMS: m/z 543 [M+H]⁺. HRMS (ESI) calcd for C₂₅H₂₅BrFN₄O₂Si [M+H]⁺ 543.1222 found 543.1221;

### 4-ethenyl-2-(2-fluoro-4-methylphenyl)-5-(1H-pyrrolo[2,3-b]pyridin-4-yl)-1-{[2-(trimethylsilyl)ethoxy]methyl}-1H-pyrrole-3-carboxamide (XIa)

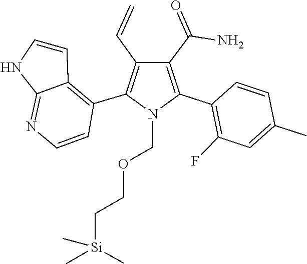 [SMILES: C=Cc1c(C(N)=O)c(-c2ccc(C)cc2F)n(COCC[Si](C)(C)C)c1-c1ccnc2[nH]ccc12]

¹H NMR (500 MHz, DMSO-d₆) d ppm −0.32-−0.18 (m, 9H) 0.38 (br. s., 2H) 2.33-2.42 (m, 3H) 2.72-2.91 (m, 2H) 4.90 (d, J=12.81 Hz, 3H) 5.28 (d, J=17.69 Hz, 1H) 6.22 (br. s., 1H) 6.44 (dd, J=17.77, 11.67 Hz, 1H) 7.01-7.29 (m, 5H) 7.44 (t, J=7.85 Hz, 1H) 7.56 (br. s., 1H) 8.26-8.39 (m, 1H) 11.84 (br. s., 1H). LCMS: m/z 491 [M+H]⁺. HRMS (ESI) calcd for C₂₇H₃₁FN₄O₂Si [M+H]⁺ 491.2273 found 491.2267;

### 2-(2-fluoro-4-methylphenyl)-4-(prop-1-en-2-yl)-5-(1H-pyrrolo[2,3-b]pyridin-4-yl)-1-{[2-(trimethylsilyl)ethoxy]methyl}-1H-pyrrole-3-carboxamide (XIa)

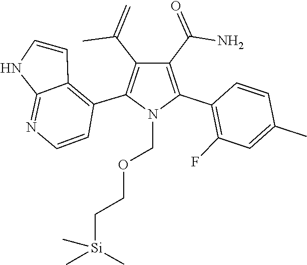 [SMILES: C=C(C)c1c(C(N)=O)c(-c2ccc(C)cc2F)n(COCC[Si](C)(C)C)c1-c1ccnc2[nH]ccc12]

¹H NMR (500 MHz, DMSO-d₆) d ppm −0.23 (s, 9H) 0.38 (t, J=8.31 Hz, 2H) 1.76 (s, 3H) 2.36-2.39 (m, 3H) 2.71-2.89 (m, 2H) 4.75 (d, J=1.68 Hz, 1H) 4.86 (br. s., 3H) 6.22 (br. s., 1H) 6.75 (br. s., 1H) 6.93-7.06 (m, 2H) 7.07-7.17 (m, 2H) 7.37-7.66 (m, 2H) 8.25 (d, J=4.88 Hz, 1H) 11.77 (br. s., 1H). LCMS: m/z 505 [M+H]⁺. HRMS (ESI) calcd for C₂₈H₃₃FN₄O₂Si [M+H]⁺ 505.243 found 505.2415;

### Conversion 3

### 2-(2-fluoro-4-methylphenyl)-4-(propan-2-yl)-5-(1H-pyrrolo[2,3-b]pyridin-4-yl)-1-{[2-(trimethylsilyl)ethoxy]methyl}-1H-pyrrole-3-carboxamide (XI)

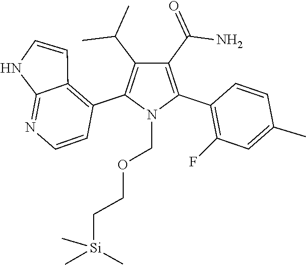 [SMILES: Cc1ccc(-c2c(C(N)=O)c(C(C)C)c(-c3ccnc4[nH]ccc34)n2COCC[Si](C)(C)C)c(F)c1]

A solution of 2-(2-fluoro-4-methylphenyl)-4-(prop-1-en-2-yl)-5-(1H-pyrrolo[2,3-b]pyridin-4-yl)-1-{[2-(trimethylsilyl)ethoxy]methyl}-1H-pyrrole-3-carboxamide (1 eq., 50 mg, 0.1 mmol) in DCM/MeOH 1/1 (2 ml) was pumped into an H-Cube® apparatus at a temperature of 60° C., flow rate of 1 ml/min and pressure of 60 bar. The catalyst use was Pd/C 10% packed into a CatCart® The product was isolated from a 25 ml product solution and analysed by HPLC. The solvent was evaporated and the crude was purified by flash-chromatography (DCM/MeOH 85/15) affording the title compound (white solid, 48 mg, Y=97%).

¹H NMR (500 MHz, DMSO-d₆) d ppm −0.23 (s, 9H) 0.29-0.40 (m, 2H) 1.12-1.22 (m, 6H) 2.36 (s, 3H) 2.75 (br. s., 2H) 2.84 (quin, J=7.02 Hz, 1H) 4.64-5.02 (m, 2H) 6.19 (br. s., 1H) 6.54-6.86 (m, 1H) 6.92 (br. s., 1H) 6.99 (d, J=4.88 Hz, 1H) 7.07-7.16 (m, 2H) 7.39 (br. s., 1H) 7.51-7.57 (m, 1H) 8.29 (d, J=4.73 Hz, 1H) 11.81 (br. s., 1H). LCMS: m/z 507[M+H]⁺. HRMS (ESI) calcd for C₂₈H₃₅FN₄O₂Si [M+H]⁺ 507.2586 found 507.2566;

Operating in an analogous way, but employing suitable substituted starting material the following compound was obtained:

### 4-ethyl-2-(2-fluoro-4-methylphenyl)-5-(1H-pyrrolo[2,3-b]pyridin-4-yl)-1-{[2-(trimethylsilyl)ethoxy]methyl}-1H-pyrrole-3-carboxamide (XI)

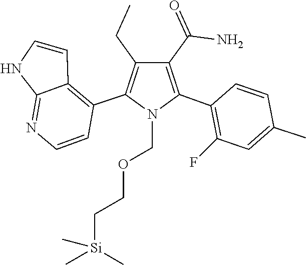 [SMILES: CCc1c(C(N)=O)c(-c2ccc(C)cc2F)n(COCC[Si](C)(C)C)c1-c1ccnc2[nH]ccc12]

¹H NMR (500 MHz, DMSO-d₆) d ppm −0.24 (s, 9H) 0.37 (br. s., 2H) 0.92 (t, J=7.40 Hz, 3H) 2.37 (s, 4H) 2.70-2.91 (m, 2H) 4.87 (br. s., 2H) 6.25 (br. s., 1H) 6.64 (br. s., 1H) 6.92 (br. s., 1H) 7.03 (d, J=4.12 Hz, 1H) 7.08-7.19 (m, 2H) 7.43 (t, J=7.85 Hz, 1H) 7.53 (t, J=2.90 Hz, 1H) 8.29 (d, J=4.88 Hz, 1H) 11.80 (br. s., 1H). LCMS: m/z 493 [M+H]⁺. HRMS (ESI) calcd for C₂₇H₃₃FN₄O₂Si [M+H]⁺ 493.243 found 493.2415;

### Step 7

### 2-(3-chloro-2-fluorophenyl)-5-(1H-pyrrolo[2,3-b]pyridin-4-yl)-1H-pyrrole-3-carboxamide [R1=1H-pyrrolo[2,3-b]pyridin-4-yl, R2=3-chloro-2-fluorophenyl, R3=R4=R5=H] comp 1

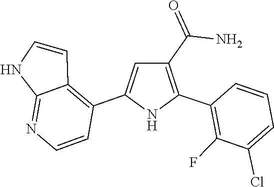 [SMILES: NC(=O)c1cc(-c2ccnc3[nH]ccc23)[nH]c1-c1cccc(Cl)c1F]

To a solution of 2-(3-chloro-2-fluorophenyl)-5-(1H-pyrrolo[2,3-b]pyridin-4-yl)-1-{[2-(trimethylsilyl)ethoxy]methyl}-1H-pyrrole-3-carboxamide (1eq., 60 mg, 0.12 mmol) in DCM (2.6 ml), TFA (75 eq., 9.3 mmol, 711 μl) was added. The reaction mixture was stirred at room temperature for 5 hours. The solvent was removed under reduced pressure and the crude was treated three times with toluene. In the same reactor 2-(3-chloro-2-fluorophenyl)-1-(hydroxymethyl)-5-(1H-pyrrolo[2,3-b]pyridin-4-yl)-1H-pyrrole-3-carboxamide was dissolved in EtOH 95% (0.6 ml) and ammonium hydroxide solution 30-32% (0.3 ml) was added. The reaction mixture was stirred at room temperature for 2 hours, the formation of a precipitate was observed. The solid was filtered and was washed three times with distilled water (to remove CF₃COO⁻ NH₄⁺) and three times with DCM/Et₂O 1/1 to achieve the title compound (pale yellow solid, 36 mg, Y=82%).

¹H NMR (500 MHz, DMSO-d₆) d ppm 1H NMR (500 MHz, DMSO-d6) δ ppm 6.84 (br. s., 1H) 7.01 (dd, J=3.5, 2.0 Hz, 1H) 7.28 (t, J=7.9 Hz, 1H) 7.39 (d, J=5.2 Hz, 1H) 7.43 (d, J=2.7 Hz, 1H) 7.50-7.53 (m, 1H) 7.55-7.57 (m, 1H) 7.58-7.62 (m, 1H) 7.63 (br. s., 1H) 8.20 (d, J=5.0 Hz, 1H) 11.71 (br. s., 1H) 12.03 (d, J=1.8 Hz, 1H). LCMS: m/z 355 [M+H]⁺. HRMS (ESI) calcd for C₁₈H₁₂ClFN₄O [M+H]⁺ 355.0757 found 355.0758;

Operating in an analogous way, but employing suitable substituted starting material the following compounds were obtained:

### 2-(4-chloro-2-fluorophenyl)-5-(1H-pyrrolo[2,3-b]pyridin-4-yl)-1H-pyrrole-3-carboxamide [R1=1H-pyrrolo[2,3-b]pyridin-4-yl, R2=4-chloro-2-fluorophenyl, R3=R4=R5=H] comp 2

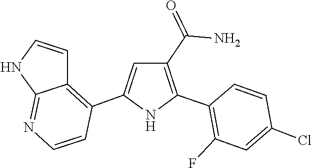 [SMILES: NC(=O)c1cc(-c2ccnc3[nH]ccc23)[nH]c1-c1ccc(Cl)cc1F]

¹H NMR (500 MHz, DMSO-d₆) δ ppm 6.83 (br. s., 1H) 7.00 (dd, J=3.5, 1.8 Hz, 1H) 7.35 (dd, J=8.2, 2.0 Hz, 1H) 7.38 (d, J=5.2 Hz, 1H) 7.42 (d, J=2.6 Hz, 1H) 7.46-7.51 (m, 1H) 7.52-7.56 (m, 1H) 7.57-7.59 (m, 1H) 7.60 (br. s., 1H) 8.20 (d, J=5.0 Hz, 1H) 11.71 (br. s., 1H) 11.98 (d, J=2.0 Hz, 1H). LCMS: m/z 355 [M+H]⁺. HRMS (ESI) calcd for C₁₈H₁₂ClFN₄O[M+H]⁺ 355.0757 found 355.0757;

### 2-(2-chloro-4-fluorophenyl)-5-(1H-pyrrolo[2,3-b]pyridin-4-yl)-1H-pyrrole-3-carboxamide [R1=1H-pyrrolo[2,3-b]pyridin-4-yl, R2=2-chloro-4-fluorophenyl, R3=R4=R5=H] comp 3

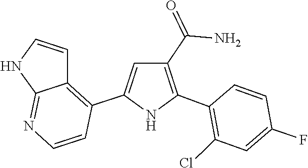 [SMILES: NC(=O)c1cc(-c2ccnc3[nH]ccc23)[nH]c1-c1ccc(F)cc1Cl]

¹H NMR (500 MHz, DMSO-d₆) δ ppm 6.76 (br. s., 1H) 7.02 (dd, J=3.4, 1.9 Hz, 1H) 7.28 (td, J=8.5, 2.7 Hz, 1H) 7.37 (d, J=5.2 Hz, 1H) 7.44 (d, J=2.7 Hz, 1H) 7.49-7.57 (m, 4H) 8.17 (d, J=5.0 Hz, 1H) 11.69 (br. s., 1H) 11.96 (d, J=2.1 Hz, 1H). LCMS: m/z 355 [M+H]⁺. HRMS (ESI) calcd for C₁₈H₁₂ClFN₄O[M+H]⁺ 355.0757 found 355.0756;

### 2-(2,4-difluorophenyl)-5-(1H-pyrrolo[2,3-b]pyridin-4-yl)-1H-pyrrole-3-carboxamide [R1=1H-pyrrolo[2,3-b]pyridin-4-yl, R2=2,4-difluorophenyl, R3=R4=R5=H] comp 4

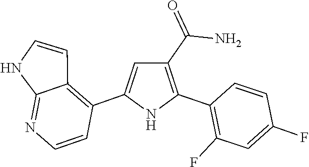 [SMILES: NC(=O)c1cc(-c2ccnc3[nH]ccc23)[nH]c1-c1ccc(F)cc1F]

¹H NMR (500 MHz, DMSO-d₆) δ ppm 6.80 (br. s., 1H) 7.01 (dd, J=3.6, 1.9 Hz, 1H) 7.15 (td, J=8.5, 2.3 Hz, 1H) 7.30 (td, J=9.8, 2.6 Hz, 1H) 7.39 (d, J=5.2 Hz, 1H) 7.42 (d, J=2.7 Hz, 1H) 7.53-7.56 (m, 1H) 7.56-7.62 (m, 2H) 8.19 (d, J=5.0 Hz, 1H) 11.70 (br. s., 1H) 11.96 (d, J=1.8 Hz, 1H). LCMS: m/z 339 [M+H]⁺. HRMS (ESI) calcd for C₁₈H₁₂F₂N₄O[M+H]⁺ 339.1052 found 339.1048;

### 2-[2-chloro-4-(trifluoromethyl)phenyl]-5-(1H-pyrrolo[2,3-b]pyridin-4-yl)-1H-pyrrole-3-carboxamide [R1=1H-pyrrolo[2,3-b]pyridin-4-yl, R2=2-chloro-4-(trifluoromethyl)phenyl, R3=R4=R5=H] comp 5

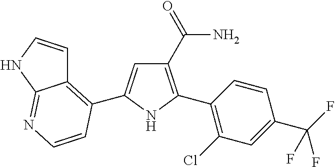 [SMILES: NC(=O)c1cc(-c2ccnc3[nH]ccc23)[nH]c1-c1ccc(C(F)(F)F)cc1Cl]

¹H NMR (500 MHz, DMSO-d₆) δ ppm 6.80 (br. s., 1H) 7.03 (dd, J=3.4, 1.8 Hz, 1H) 7.36 (d, J=5.1 Hz, 1H) 7.48 (d, J=2.4 Hz, 1H) 7.54-7.57 (m, 1H) 7.61 (br. s., 1H) 7.70-7.79 (m, 2H) 7.94 (s, 1H) 8.19 (d, J=5.0 Hz, 1H) 11.69 (br. s., 1H) 12.04 (br. s., 1H). LCMS: m/z 405 [M+H]⁺. HRMS (ESI) calcd for C₁₉H₁₂ClF₃N₄O [M+H]⁺ 405.0725 found 405.0724;

### 2-(2,3-difluorophenyl)-5-(1H-pyrrolo[2,3-b]pyridin-4-yl)-1H-pyrrole-3-carboxamide [R1=1H-pyrrolo[2,3-b]pyridin-4-yl, R2=2,3-difluorophenyl, R3=R4=R5=H] comp 6

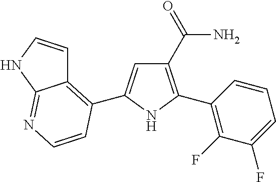 [SMILES: NC(=O)c1cc(-c2ccnc3[nH]ccc23)[nH]c1-c1cccc(F)c1F]

¹H NMR (500 MHz, DMSO-d₆) δ ppm 6.81 (br. s., 1H) 7.01 (dd, J=3.5, 1.8 Hz, 1H) 7.21-7.30 (m, 1H) 7.33-7.49 (m, 4H) 7.55 (t, J=3.1 Hz, 1H) 7.60 (br. s., 1H) 8.20 (d, J=5.0 Hz, 1H) 11.69 (br. s., 1H) 12.01 (br. s., 1H). LCMS: m/z 339 [M+H]⁺. HRMS (ESI) calcd for C₁₈H₁₂F₂N₄O[M+H]⁺ 339.1052 found 339.1046;

### 2-(2,3-dichlorophenyl)-5-(1H-pyrrolo[2,3-b]pyridin-4-yl)-1H-pyrrole-3-carboxamide[R1=1H-pyrrolo[2,3-b]pyridin-4-yl, R2=2,3-dichlorophenyl, R3=R4=R5=H] comp 7

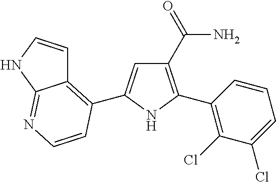 [SMILES: NC(=O)c1cc(-c2ccnc3[nH]ccc23)[nH]c1-c1cccc(Cl)c1Cl]

¹H NMR (500 MHz, DMSO-d₆) δ ppm 6.78 (br. s., 1H) 7.02 (dd, J=3.5, 2.0 Hz, 1H) 7.36 (d, J=5.2 Hz, 1H) 7.39-7.43 (m, 1H) 7.44-7.47 (m, 2H) 7.54-7.56 (m, 1H) 7.57 (br. s., 1H) 7.68 (dd, J=7.9, 1.8 Hz, 1H) 8.18 (d, J=5.2 Hz, 1H) 11.70 (br. s., 1H) 12.01 (d, J=1.8 Hz, 1H). LCMS: m/z 371 [M+H]⁺. HRMS (ESI) calcd for C₁₈H₁₂Cl₂N₄O [M+H]⁺ 371.0461 found 371.0463;

### 2-[4-methyl-2-(trifluoromethyl)phenyl]-5-(1H-pyrrolo[2,3-b]pyridin-4-yl)-1H-pyrrole-3-carboxamide [R1=1H-pyrrolo[2,3-b]pyridin-4-yl, R2=4-methyl-2-(trifluoromethyl)phenyl, R3=R4=R5=H] comp 8

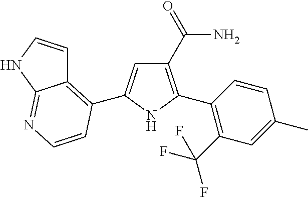 [SMILES: Cc1ccc(-c2[nH]c(-c3ccnc4[nH]ccc34)cc2C(N)=O)c(C(F)(F)F)c1]

¹H NMR (500 MHz, DMSO-d₆) δ ppm 2.46 (s, 3H) 6.66 (br. s., 1H) 7.01 (dd, J=3.51, 1.83 Hz, 1H) 7.33 (d, J=5.19 Hz, 1H) 7.39 (d, J=7.78 Hz, 2H) 7.42 (d, J=2.75 Hz, 1H) 7.48-7.51 (m, 1H) 7.53-7.54 (m, 1H) 7.62 (s, 1H) 8.15 (d, J=5.19 Hz, 1H) 11.67 (br. s., 1H) 11.91 (d, J=1.98 Hz, 1H). LCMS: m/z 385 [M+H]⁺. HRMS (ESI) calcd for C₂₀H₁₅F₃N₄O[M+H]⁺ 385.1271 found 385.1270;

### 2-(2-chloro-4-methylphenyl)-5-(1H-pyrrolo[2,3-b]pyridin-4-yl)-1H-pyrrole-3-carboxamide[R1=1H-pyrrolo[2,3-b]pyridin-4-yl, R2=2-chloro-4-methylphenyl, R3=R4=R5=H] comp 9

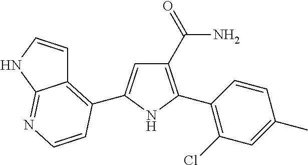 [SMILES: Cc1ccc(-c2[nH]c(-c3ccnc4[nH]ccc34)cc2C(N)=O)c(Cl)c1]

¹H NMR (500 MHz, DMSO-d6) δ ppm 2.35-2.40 (m, 3H) 6.68-6.78 (m, 1H) 7.01 (dd, J=3.5, 1.8 Hz, 1H) 7.20 (dd, J=7.9, 0.8 Hz, 1H) 7.33-7.38 (m, 3H) 7.39-7.43 (m, 2H) 7.52-7.55 (m, 1H) 8.16 (d, J=5.2 Hz, 1H) 11.67 (br. s., 1H) 11.88 (d, J=1.8 Hz, 1H). LCMS: m/z 351 [M+H]⁺. HRMS (ESI) calcd for C₁₁H₁₅ClN₄O[M+H]⁺ 351.1007 found 351.1008;

### 2-(2,3-difluoro-4-methylphenyl)-5-(1H-pyrrolo[2,3-b]pyridin-4-yl)-1H-pyrrole-3-carboxamide[R1=1H-pyrrolo [2,3-b]pyridin-4-yl, R2=2,3-difluoro-4-methylphenyl, R3=R4=R5=H] comp 10

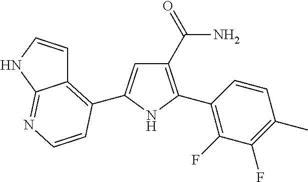 [SMILES: Cc1ccc(-c2[nH]c(-c3ccnc4[nH]ccc34)cc2C(N)=O)c(F)c1F]

¹H NMR (500 MHz, DMSO-d6) δ ppm 2.32-2.36 (m, 3H) 6.82 (br. s., 1H) 7.00 (dd, J=3.4, 1.9 Hz, 1H) 7.12-7.16 (m, 1H) 7.22-7.26 (m, 1H) 7.39 (d, J=5.2 Hz, 1H) 7.41 (d, J=2.6 Hz, 1H) 7.53-7.56 (m, 1H) 7.59 (br. s., 1H) 8.20 (d, J=5.2 Hz, 1H) 11.70 (br. s., 1H) 11.97 (br. s., 1H). LCMS: m/z 353 [M+H]⁺. HRMS (ESI) calcd for C₁₉H₁₄F₂N₄O [M+H]⁺ 353.1209 found 353.121.

### 2-[2-methyl-4-(trifluoromethyl)phenyl]-5-(1H-pyrrolo[2,3-b]pyridin-4-yl)-1H-pyrrole-3-carboxamide [R1=1H-pyrrolo[2,3-b]pyridin-4-yl, R2=2-methyl-4-(trifluoromethyl)phenyl, R3=R4=R5=H] comp 11

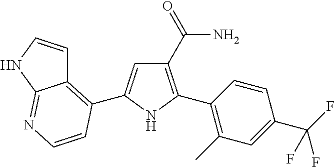 [SMILES: Cc1cc(C(F)(F)F)ccc1-c1[nH]c(-c2ccnc3[nH]ccc23)cc1C(N)=O]

¹H NMR (500 MHz, DMSO-d6) δ ppm 2.28 (s, 3H) 6.80 (br. s., 1H) 7.03 (dd, J=3.5, 2.0 Hz, 1H) 7.38 (d, J=5.2 Hz, 1H) 7.47-7.49 (m, 1H) 7.50-7.53 (m, 1H) 7.53-7.60 (m, 3H) 7.66 (s, 1H) 8.17 (d, J=5.2 Hz, 1H) 11.68 (br. s., 1H) 11.90 (d, J=2.1 Hz, 1H). LCMS: m/z 385 [M+H]⁺. HRMS (ESI) calcd for C₂₀H₁₅F₃N₄O[M+H]⁺ 385.1271 found 385.127;

### 2-(2-fluoro-3-methoxyphenyl)-5-(1H-pyrrolo[2,3-b]pyridin-4-yl)-1H-pyrrole-3-carboxamide [R1=1H-pyrrolo[2,3-b]pyridin-4-yl, R2=2-fluoro-3-methoxyphenyl, R3=R4=R5=H] comp 12

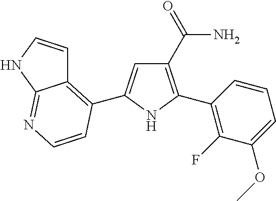 [SMILES: COc1cccc(-c2[nH]c(-c3ccnc4[nH]ccc34)cc2C(N)=O)c1F]

¹H NMR (500 MHz, DMSO-d6) δ ppm 3.87 (s, 3H) 6.77 (br. s., 1H) 7.00 (dd, J=3.5, 1.8 Hz, 1H) 7.03-7.08 (m, 1H) 7.13-7.21 (m, 2H) 7.37-7.41 (m, 2H) 7.51 (br. s., 1H) 7.53-7.55 (m, 1H) 8.18 (d, J=5.0 Hz, 1H) 11.68 (br. s., 1H) 11.93 (d, J=2.0 Hz, 1H). LCMS: m/z 351 [M+H]⁺. HRMS (ESI) calcd for C₁₉H₁₅FN₄O₂[M+H]⁺ 351.1252 found 351.1252;

### 2-(2-chloro-3-fluorophenyl)-5-(1H-pyrrolo[2,3-b]pyridin-4-yl)-1H-pyrrole-3-carboxamide[R1=1H-pyrrolo[2,3-b]pyridin-4-yl, R2=2-chloro-3-fluorophenyl, R3=R4=R5=H] comp 13

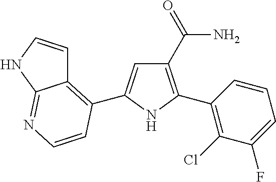 [SMILES: NC(=O)c1cc(-c2ccnc3[nH]ccc23)[nH]c1-c1cccc(F)c1Cl]

¹H NMR (500 MHz, DMSO-d6) δ ppm 6.79 (br. s., 1H) 7.03 (dd, J=3.4, 1.9 Hz, 1H) 7.32-7.36 (m, 1H) 7.38 (d, J=5.2 Hz, 1H) 7.41-7.48 (m, 3H) 7.53-7.62 (m, 2H) 8.18 (d, J=5.2 Hz, 1H) 11.72 (br. s., 1H) 12.02 (br. s., 1H). LCMS: m/z 355 [M+H]⁺. HRMS (ESI) calcd for C₁₈H₁₂ClFN₄O[M+H]⁺ 355.0757 found 355.0755;

### 2-(2-fluoro-3-methylphenyl)-5-(1H-pyrrolo[2,3-b]pyridin-4-yl)-1H-pyrrole-3-carboxamide [R1=1H-pyrrolo[2,3-b]pyridin-4-yl, R2=2-fluoro-3-methylphenyl, R3=R4=R5=H] comp 14

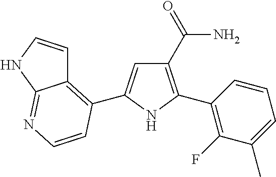 [SMILES: Cc1cccc(-c2[nH]c(-c3ccnc4[nH]ccc34)cc2C(N)=O)c1F]

¹H NMR (500 MHz, DMSO-d6) δ ppm 2.28 (d, J=0.9 Hz, 3H) 6.76 (br. s., 1H) 7.00 (dd, J=3.4, 1.9 Hz, 1H) 7.11-7.16 (m, 1H) 7.32 (dt, J=19.9, 7.2 Hz, 2H) 7.38-7.42 (m, 2H) 7.50 (br. s., 0H) 7.53-7.56 (m, 1H) 8.18 (d, J=5.0 Hz, 1H) 11.68 (br. s., 1H) 11.90 (br. s., 1H). LCMS: m/z 335 [M+H]⁺. HRMS (ESI) calcd for C₁₉H₁₅FN₄O[M+H]⁺ 335.1303 found 335.1299.

### 2-[2-methyl-3-(trifluoromethyl)phenyl]-5-(1H-pyrrolo[2,3-b]pyridin-4-yl)-1H-pyrrole-3-carboxamide[R1=1H-pyrrolo[2,3-b]pyridin-4-yl, R2=2-methyl-3-(trifluoromethyl)phenyl, R3=R4=R5=H] comp 15

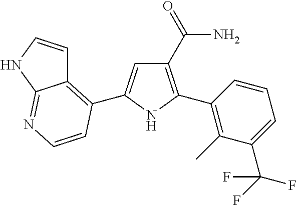 [SMILES: Cc1c(-c2[nH]c(-c3ccnc4[nH]ccc34)cc2C(N)=O)cccc1C(F)(F)F]

¹H NMR (500 MHz, DMSO-d6) ppm 2.27 (s, 3H) 6.90 (br. s., 1H) 7.33 (br. s., 1H) 7.44-7.50 (m, 1H) 7.61 (d, J=7.47 Hz, 1H) 7.69 (d, J=5.80 Hz, 2H) 7.74-7.86 (m, 3H) 8.37 (d, J=6.10 Hz, 1H) 12.34 (br. s., 1H) 12.56 (br. s., 1H). LCMS: m/z 385 [M+H]⁺. HRMS (ESI) calcd for C₂₀H₁₅F₃N₄O[M+H]⁺ 385.1271 found 385.1276;

### 2-[4-methoxy-2-(trifluoromethyl)phenyl]-5-(1H-pyrrolo[2,3-b]pyridin-4-yl)-1H-pyrrole-3-carboxamide [R1=1H-pyrrolo[2,3-b]pyridin-4-yl, R2=4-methoxy-2-(trifluoromethyl)phenyl, R3=R4=R5=H] comp 16

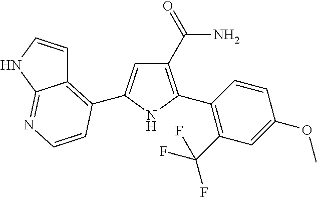 [SMILES: COc1ccc(-c2[nH]c(-c3ccnc4[nH]ccc34)cc2C(N)=O)c(C(F)(F)F)c1]

¹H NMR (500 MHz, DMSO-d6) δ ppm 3.89 (s, 3H) 6.65 (br. s., 1H) 7.01 (dd, J=3.43, 1.91 Hz, 1H) 7.24-7.30 (m, 2H) 7.33 (d, J=5.03 Hz, 2H) 7.40-7.45 (m, 2H) 7.52-7.55 (m, 1H) 8.15 (d, J=5.03 Hz, 1H) 11.66 (br. s., 1H) 11.89 (d, J=2.29 Hz, 1H). LCMS: m/z 401 [M+H]⁺. HRMS (ESI) calcd for C₂₀H₁₅F₃N₄O₂[M+H]⁺ 401.122 found 401.1208;

### 2-[2-chloro-4-(difluoromethoxy)phenyl]-5-(1H-pyrrolo[2,3-b]pyridin-4-yl)-1H-pyrrole-3-carboxamide [R1=1H-pyrrolo[2,3-b]pyridin-4-yl, R2=2-chloro-4-(difluoromethoxy)phenyl, R3=R4=R5=H] comp 17

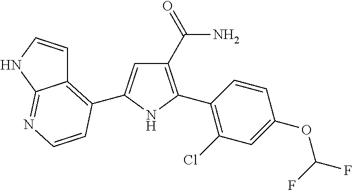 [SMILES: NC(=O)c1cc(-c2ccnc3[nH]ccc23)[nH]c1-c1ccc(OC(F)F)cc1Cl]

¹H NMR (500 MHz, DMSO-d6) δ ppm 3.89 (s, 3H) 6.65 (br. s., 1H) 7.01 (dd, J=3.43, 1.91 Hz, 1H) 7.24-7.30 (m, 2H) 7.33 (d, J=5.03 Hz, 2H) 7.40-7.45 (m, 2H) 7.52-7.55 (m, 3H) 8.15 (d, J=5.03 Hz, 1H) 11.66 (br. s., 1H) 11.89 (d, J=2.29 Hz, 1H). LCMS: m/z 403 [M+H]⁺. HRMS (ESI) calcd for C₁H₁₃ClF₂N₄O₂ [M+H]⁺ 403.07679 found 403.0769;

### 2-(3,4-dichlorophenyl)-5-(1H-pyrrolo[2,3-b]pyridin-4-yl)-1H-pyrrole-3-carboxamide[R1=1H-pyrrolo[2,3-b]pyridin-4-yl, R2=3,4-dichlorophenyl, R3=R4=R5=H] comp 18

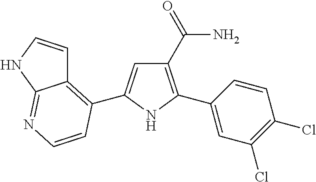 [SMILES: NC(=O)c1cc(-c2ccnc3[nH]ccc23)[nH]c1-c1ccc(Cl)c(Cl)c1]

¹H NMR (500 MHz, DMSO-d6) δ ppm 6.92-7.04 (m, 2H) 7.36 (d, J=2.59 Hz, 1H) 7.47 (d, J=5.03 Hz, 1H) 7.52-7.57 (m, 1H) 7.64-7.75 (m, 3H) 8.00 (d, J=1.98 Hz, 1H) 8.22 (d, J=5.03 Hz, 1H) 11.71 (br. s., 1H) 11.88 (d, J=1.83 Hz, 1H). LCMS: m/z 371 [M+H]⁺. HRMS (ESI) calcd for C₁₈H₁₂Cl₂N₄O [M+H]⁺ 371.0461 found 371.0458;

### 2-(3,4-difluorophenyl)-5-(1H-pyrrolo[2,3-b]pyridin-4-yl)-1H-pyrrole-3-carboxamide [R1=1H-pyrrolo[2,3-b]pyridin-4-yl, R2=3,4-difluorophenyl, R3=R4=R5=H] comp 19

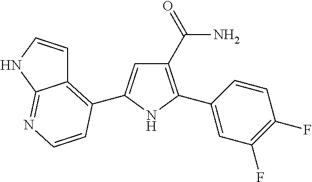 [SMILES: NC(=O)c1cc(-c2ccnc3[nH]ccc23)[nH]c1-c1ccc(F)c(F)c1]

¹H NMR (500 MHz, DMSO-d6) δ ppm 6.88-7.03 (m, 2H) 7.35 (d, J=2.59 Hz, 1H) 7.43-7.61 (m, 4H) 7.66 (br. s., 1H) 7.83 (ddd, J=12.32, 8.12, 1.98 Hz, 1H) 8.21 (d, J=5.03 Hz, 1H) 11.46-11.96 (m, 2H). LCMS: m/z 339 [M+H]⁺. HRMS (ESI) calcd for C₁₈H₁₂F₂N₄O[M+H]⁺ 339.1052 found 339.1049;

### 2-(3-ethoxy-2-fluorophenyl)-5-(1H-pyrrolo[2,3-b]pyridin-4-yl)-1H-pyrrole-3-carboxamide [R1=1H-pyrrolo[2,3-b]pyridin-4-yl, R2=3-ethoxy-2-fluorophenyl, R3=R4=R5=H] comp 20

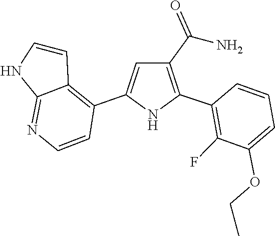 [SMILES: CCOc1cccc(-c2[nH]c(-c3ccnc4[nH]ccc34)cc2C(N)=O)c1F]

¹H NMR (500 MHz, DMSO-d6) δ ppm 1.38 (t, J=7.02 Hz, 3H) 4.13 (q, J=7.02 Hz, 2H) 6.77 (br. s., 1H) 7.00 (dd, J=3.51, 1.98 Hz, 1H) 7.03-7.09 (m, 1H) 7.11-7.20 (m, 2H) 7.36-7.44 (m, 2H) 7.46-7.58 (m, 2H) 8.18 (d, J=5.03 Hz, 1H) 11.36-12.12 (m, 2H). LCMS: m/z 365 [M+H]⁺. HRMS (ESI) calcd for C₂₀H₁₇FN₄O₂[M+H]⁺ 365.1409 found 365.141;

### 2-(4-methyl-3-nitrophenyl)-5-(1H-pyrrolo[2,3-b]pyridin-4-yl)-1H-pyrrole-3-carboxamide [R1=1H-pyrrolo[2,3-b]pyridin-4-yl, R2=4-methyl-3-nitrophenyl, R3=R4=R5=H] comp 21

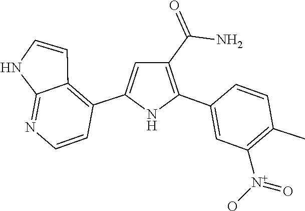

¹H NMR (500 MHz, DMSO-d6) δ ppm 2.57 (s, 3H) 6.96 (br. s., 1H) 7.00 (br. s., 1H) 7.39 (d, J=2.59 Hz, 1H) 7.47 (d, J=5.19 Hz, 1H) 7.50-7.57 (m, 2H) 7.72 (br. s., 1H) 7.97 (dd, J=7.85, 1.75 Hz, 1H) 8.22 (d, J=5.19 Hz, 1H) 8.37 (d, J=1.83 Hz, 1H) 11.43-12.13 (m, 2H). LCMS: m/z 362 [M+H]⁺. HRMS (ESI) calcd for C₁₉H₁₅N50₃ [M+H]⁺ 362.1248 found 362.1241;

### 2-(3-carbamoyl-4-fluorophenyl)-5-(1H-pyrrolo[2,3-b]pyridin-4-yl)-1H-pyrrole-3-carboxamide[R1=1H-pyrrolo [2,3-b]pyridin-4-yl, R2=3-carbamoyl-4-fluorophenyl, R3=R4=R5=H] comp 22

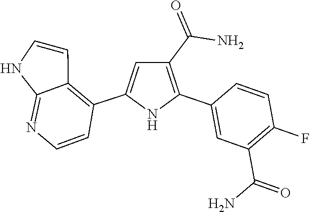 [SMILES: NC(=O)c1cc(-c2[nH]c(-c3ccnc4[nH]ccc34)cc2C(N)=O)ccc1F]

¹H NMR (500 MHz, DMSO-d6) δ ppm 6.87 (br. s., 1H) 7.01 (dd, J=3.36, 1.83 Hz, 1H) 7.31 (dd, J=10.37, 8.69 Hz, 1H) 7.37 (d, J=2.44 Hz, 1H) 7.41-7.51 (m, 1H) 7.52-7.57 (m, 1H) 7.59-7.78 (m, 1H) 7.85 (ddd, J=8.46, 4.88, 2.36 Hz, 1H) 7.96 (dd, J=7.02, 2.29 Hz, 1H) 8.21 (d, J=5.19 Hz, 1H) 11.71 (br. s., 1H) 11.84 (br. s., 1H). LCMS: m/z 364 [M+H]⁺. HRMS (ESI) calcd for C₁₉H₁₄FN₅O₂[M+H]⁺ 364.1205 found 364.1204.

### 2-(2-fluoro-4-methylphenyl)-N-[2-(pyrrolidin-1-yl)ethyl]-5-(1H-pyrrolo[2,3-b]pyridin-4-yl)-1H-pyrrole-3-carboxamide [R1=1H-pyrrolo[2,3-b]pyridin-4-yl, R2=2-fluoro-4-methylphenyl, R3=N-2-(pyrrolidin-1-yl)ethyl, R4=R5=H] comp 23

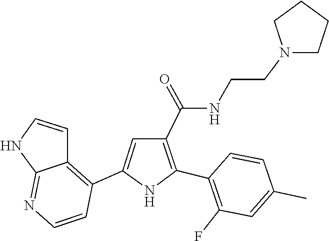 [SMILES: Cc1ccc(-c2[nH]c(-c3ccnc4[nH]ccc34)cc2C(=O)NCCN2CCCC2)c(F)c1]

The compound was isolated as TFA salt.

¹H NMR (500 MHz, DMSO-d6) δ ppm 1.78-1.91 (m, 2H) 1.93-2.06 (m, 2H) 2.37-2.40 (m, 3H) 2.97-3.10 (m, 2H) 3.23-3.31 (m, 2H) 3.47-3.55 (m, 1H) 3.56-3.65 (m, 2H) 6.94-6.98 (m, 1H) 7.04-7.11 (m, 2H) 7.38-7.47 (m, 3H) 7.57-7.60 (m, 1H) 8.17-8.23 (m, 1H) 8.24-8.31 (m, 1H) 9.25-9.63 (m, 1H) 11.67-11.83 (m, 1H) 11.99-12.09 (m, 1H). LCMS: m/z 432 [M+H]⁺. HRMS (ESI) calcd for C₂₅H₂₆FN₅O [M+H]⁺ 432.2194 found 432.2197;

### N-[2-(dimethylamino)ethyl]-2-(2-fluoro-4-methylphenyl)-5-(1H-pyrrolo[2,3-b]pyridin-4-yl)-1H-pyrrole-3-carboxamide [R1=1H-pyrrolo[2,3-b]pyridin-4-yl, R2=2-fluoro-4-methylphenyl, R3=N-2-(dimethylamino) ethyl, R4=R5=H] comp 24

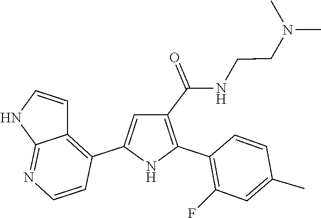 [SMILES: Cc1ccc(-c2[nH]c(-c3ccnc4[nH]ccc34)cc2C(=O)NCCN(C)C)c(F)c1]

The compound was isolated as TFA salt.

¹H NMR (500 MHz, DMSO-d6) δ ppm 2.37-2.41 (m, 3H) 2.78-2.88 (m, 6H) 3.17-3.24 (m, 2H) 3.46-3.56 (m, 2H) 6.92-6.98 (m, 1H) 7.05-7.13 (m, 2H) 7.38-7.46 (m, 3H) 7.55-7.61 (m, 1H) 8.13-8.24 (m, 1H) 8.25-8.32 (m, 1H) 11.59-11.85 (m, 1H) 11.95-12.12 (m, 1H). LCMS: m/z 406 [M+H]⁺. HRMS (ESI) calcd for C₂₃H₂₄FN₅O [M+H]⁺ 406.2038 found 406.2036;

### 2-(2-fluoro-4-methylphenyl)-N-[2-(morpholin-4-yl)ethyl]-5-(1H-pyrrolo[2,3-b]pyridin-4-yl)-1H-pyrrole-3-carboxamide[R1=1H-pyrrolo[2,3-b]pyridin-4-yl, R2=2-fluoro-4-methylphenyl, R3=N-2-(morpholin-4-yl)ethyl, R4=R5=H] comp 25

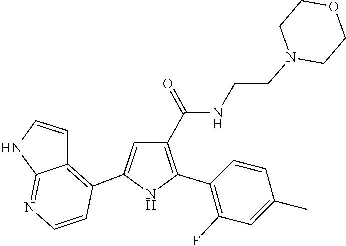 [SMILES: Cc1ccc(-c2[nH]c(-c3ccnc4[nH]ccc34)cc2C(=O)NCCN2CCOCC2)c(F)c1]

¹H NMR (500 MHz, DMSO-d6) δ ppm 2.37-2.39 (m, 3H) 2.9-4.02 (m, 12H) 6.95 (dd, J=3.43, 1.91 Hz, 1H) 7.04-7.12 (m, 2H) 7.32-7.47 (m, 2H) 7.57 (t, J=2.82 Hz, 1H) 8.20 (d, J=5.03 Hz, 1H) 11.47-12.21 (m, 2H). LCMS: m/z 448 [M+H]⁺. HRMS (ESI) calcd for C₂₅H₂₆FN₅O₂[M+H]⁺ 448.2144 found 448.2132.

### N-[(1S,2R)-2-aminocyclohexyl]-2-(2-fluoro-4-methylphenyl)-5-(1H-pyrrolo[2,3-b]pyridin-4-yl)-1H-pyrrole-3-carboxamide [R1=1H-pyrrolo[2,3-b]pyridin-4-yl, R2=2-fluoro-4-methylphenyl, R3=N-(1S,2R)-2-aminocyclohexyl, R4=R5=H] comp 26

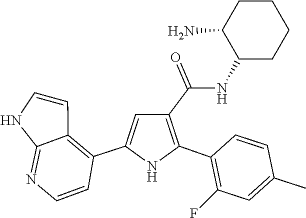

The compound was isolated as TFA salt.

¹H NMR (500 MHz, DMSO-d6) δ ppm 1.30-1.45 (m, 2H) 1.48-1.78 (m, 6H) 2.38 (s, 3H) 4.23 (br. s., 1H) 6.98 (dd, J=3.43, 1.91 Hz, 1H) 7.06-7.15 (m, 2H) 7.40 (d, J=5.19 Hz, 1H) 7.42-7.49 (m, 3H) 7.55-7.62 (m, 1H) 7.71 (br. s., 2H) 8.21 (d, J=5.19 Hz, 1H) 11.74 (br. s., 1H) 11.98 (d, J=2.14 Hz, 1H). LCMS: m/z 432 [M+H]⁺. HRMS (ESI) calcd for C₂₅H₂₅FN₅O [M+H]⁺ 432.2194 found 432.2189;

### 2-(2-fluoro-4-methylphenyl)-N-(furan-2-ylmethyl)-5-(1H-pyrrolo[2,3-b]pyridin-4-yl)-1H-pyrrole-3-carboxamide [R1=1H-pyrrolo[2,3-b]pyridin-4-yl, R2=2-fluoro-4-methylphenyl, R3=N-(furan-2-ylmethyl), R4=R5=H] comp 27

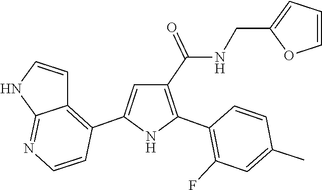 [SMILES: Cc1ccc(-c2[nH]c(-c3ccnc4[nH]ccc34)cc2C(=O)NCc2ccco2)c(F)c1]

¹H NMR (500 MHz, DMSO-d6) δ ppm 2.38 (s, 1H) 4.38 (d, J=5.80 Hz, 1H) 6.20-6.25 (m, 1H) 6.39 (dd, J=3.13, 1.91 Hz, 1H) 7.03-7.11 (m, 1H) 7.37-7.47 (m, 1H) 7.55-7.60 (m, 1H) 8.22 (d, J=5.34 Hz, 1H) 8.50 (t, J=5.87 Hz, 1H) 11.64-12.14 (m, 1H). LCMS: m/z 415 [M+H]⁺. HRMS (ESI) calcd for C₂₄H₁, FN₄O₂[M+H]⁺ 415.1565 found 415.1569;

### N-(fluoroethyl)-2-(2-fluoro-4-methylphenyl)-5-(1H-pyrrolo[2,3-b]pyridin-4-yl)-1H-pyrrole-3-carboxamide[R1=1H-pyrrolo[2,3-b]pyridin-4-yl, R2=2-fluoro-4-methylphenyl, R3=N-(fluoroethyl), R4=R5=H] comp 28

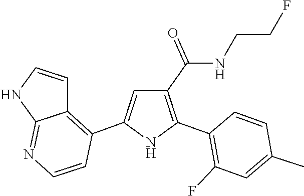 [SMILES: Cc1ccc(-c2[nH]c(-c3ccnc4[nH]ccc34)cc2C(=O)NCCF)c(F)c1]

¹H NMR (500 MHz, DMSO-d6) δ ppm 2.36-2.40 (m, 3H) 3.41-3.54 (m, 2H) 4.38-4.58 (m, 2H) 7.00 (dd, J=3.51, 1.83 Hz, 1H) 7.04-7.10 (m, 2H) 7.38-7.44 (m, 3H) 7.54-7.56 (m, 1H) 8.19 (d, J=5.19 Hz, 1H) 8.24 (t, J=5.64 Hz, 1H) 11.53-12.04 (m, 2H). LCMS: m/z 381 [M+H]⁺. HRMS (ESI) calcd for C₂₁H₁₈F₂N₄O [M+H]⁺ 381.1522 found 381.1518;

### 2-(2-fluoro-4-methylphenyl)-N-[2-(methylamino)ethyl]-5-(1H-pyrrolo[2,3-b]pyridin-4-yl)-1H-pyrrole-3-carboxamide [R1=1H-pyrrolo[2,3-b]pyridin-4-yl, R2=2-fluoro-4-methylphenyl, R3=N-(2-(methylamino)ethyl, R4=R5=H] comp 29

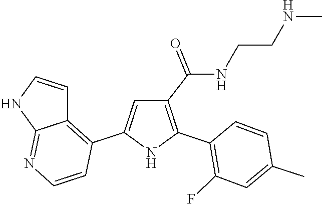 [SMILES: CNCCNC(=O)c1cc(-c2ccnc3[nH]ccc23)[nH]c1-c1ccc(C)cc1F]

¹H NMR (500 MHz, DMSO-d6) δ ppm 2.37-2.40 (m, 3H) 2.58 (s, 3H) 3.04 (t, J=5.95 Hz, 2H) 3.45 (q, J=6.00 Hz, 2H) 6.96 (dd, J=3.43, 1.91 Hz, 1H) 7.04-7.11 (m, 2H) 7.37-7.46 (m, 3H) 7.55-7.60 (m, 1H) 8.20 (d, J=5.03 Hz, 1H) 8.26 (t, J=5.64 Hz, 1H) 8.33 (br. s., 1H) 11.51-12.14 (m, 2H). LCMS: m/z 392 [M+H]⁺. HRMS (ESI) calcd for C₂₂H₂₂FN₅O [M+H]⁺ 392.1881 found 392.1878;

### 2-(2-fluoro-4-methylphenyl)-N-(1-methylpiperidin-4-yl)-5-(1H-pyrrolo[2,3-b]pyridin-4-yl)-1H-pyrrole-3-carboxamide [R1=1H-pyrrolo[2,3-b]pyridin-4-yl, R2=2-fluoro-4-methylphenyl, R3=N-(1-methylpiperidin-4-yl), R4=R5=H] comp 30

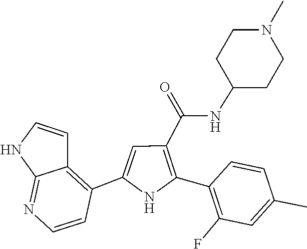 [SMILES: Cc1ccc(-c2[nH]c(-c3ccnc4[nH]ccc34)cc2C(=O)NC2CCN(C)CC2)c(F)c1]

¹H NMR (500 MHz, DMSO-d6) δ ppm 1.45-1.59 (m, 2H) 1.72 (d, J=10.37 Hz, 2H) 1.90 (t, J=11.21 Hz, 2H) 2.15 (s, 3H) 2.35-2.39 (m, 3H) 2.74 (d, J=11.13 Hz, 2H) 3.56-3.68 (m, 1H) 7.00 (dd, J=3.51, 1.98 Hz, 1H) 7.04-7.10 (m, 2H) 7.31-7.43 (m, 3H) 7.52-7.58 (m, 1H) 7.70 (d, J=7.93 Hz, 1H) 8.18 (d, J=5.03 Hz, 1H) 11.53-12.01 (m, 2H).

LCMS: m/z 432 [M+H]⁺. HRMS (ESI) calcd for C₂₅H₂₅FN₅O [M+H]⁺ 432.2194 found 432.2186;

### 2-(dibenzo[b,d]thiophen-4-yl)-5-(1H-pyrrolo[2,3-b]pyridin-4-yl)-1H-pyrrole-3-carboxamide [R1=1H-pyrrolo [2,3-b]pyridin-4-yl, R2=dibenzo[b,d]thiophen-4-yl, R3=R4=R5=H] comp 31

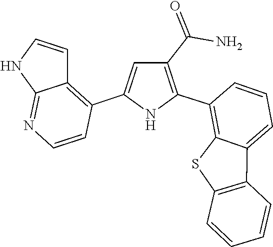 [SMILES: NC(=O)c1cc(-c2ccnc3[nH]ccc23)[nH]c1-c1cccc2c1sc1ccccc12]

¹H NMR (500 MHz, DMSO-d6) δ ppm 6.81 (br. s., 1H) 7.07 (dd, J=3.51, 1.83 Hz, 1H) 7.43 (d, J=5.19 Hz, 1H) 7.46-7.55 (m, 3H) 7.56-7.58 (m, 1H) 7.59-7.64 (m, 1H) 7.96-8.02 (m, 1H) 8.19 (d, J=5.19 Hz, 1H) 8.36-8.46 (m, 2H) 11.71 (br. s., 1H) 12.13 (d, J=1.83 Hz, 1H). LCMS: m/z 409 [M+H]⁺. HRMS (ESI) calcd for C₂₄H₁₅N₄OS [M+H]⁺ 409.1118 found 409.1119;

### 2-(4-methylnaphthalen-1-yl)-5-(1H-pyrrolo[2,3-b]pyridin-4-yl)-1H-pyrrole-3-carboxamide [R1=1H-pyrrolo[2,3-b]pyridin-4-yl, R2=4-methylnaphthalen-1-yl, R3=R4=R5=H] comp 32

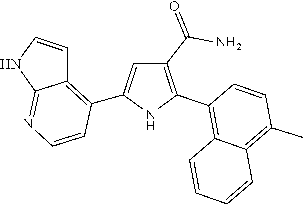 [SMILES: Cc1ccc(-c2[nH]c(-c3ccnc4[nH]ccc34)cc2C(N)=O)c2ccccc12]

¹H NMR (500 MHz, DMSO-d6) δ ppm 2.73 (s, 3H) 6.68 (br. s., 1H) 7.05 (dd, J=3.43, 1.91 Hz, 1H) 7.23 (br. s., 1H) 7.40 (d, J=5.19 Hz, 1H) 7.43-7.48 (m, 3H) 7.51 (d, J=2.75 Hz, 1H) 7.54-7.61 (m, 2H) 7.66 (d, J=8.24 Hz, 1H) 8.09 (d, J=8.39 Hz, 1H) 8.14 (d, J=5.03 Hz, 1H) 11.68 (br. s., 1H) 11.98 (br. s., 1H). LCMS: m/z 367 [M+H]⁺. HRMS (ESI) calcd for C₂₃H₁₈N₄O[M+H]⁺ 367.1554 found 367.1545;

### 2-(3-fluorophenyl)-5-(1H-pyrrolo[2,3-b]pyridin-4-yl)-1H-pyrrole-3-carboxamide [R1=1H-pyrrolo[2,3-b]pyridin-4-yl, R2=3-fluorophenyl, R3=R4=R5=H] comp 33

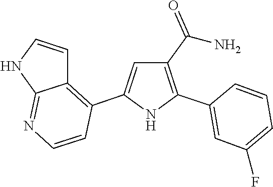 [SMILES: NC(=O)c1cc(-c2ccnc3[nH]ccc23)[nH]c1-c1cccc(F)c1]

¹H NMR (500 MHz, DMSO-d6) δ ppm 6.92 (br. s., 1H) 7.00 (dd, J=3.51, 1.83 Hz, 1H) 7.14-7.23 (m, 1H) 7.34 (d, J=2.59 Hz, 1H) 7.41-7.50 (m, 1H) 7.52-7.61 (m, 3H) 7.62-7.66 (m, 1H) 8.21 (d, J=5.19 Hz, 1H) 11.70 (br. s., 1H) 11.81 (br. s., 1H). LCMS: m/z 321 [M+H]⁺. HRMS (ESI) calcd for C₁₈H₁₃FN₄O[M+H]⁺ 321.1146 found 321.1143;

### 5-(1H-pyrrolo[2,3-b]pyridin-4-yl)-2-[4-(trifluoromethoxy)phenyl]-1H-pyrrole-3-carboxamide [R1=1H-pyrrolo [2,3-b]pyridin-4-yl, R2=4-(trifluoromethoxy)phenyl, R3=R4=R5=H] comp 34

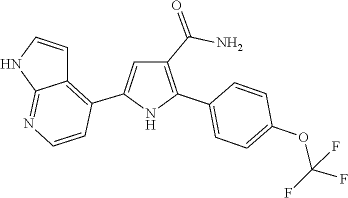 [SMILES: NC(=O)c1cc(-c2ccnc3[nH]ccc23)[nH]c1-c1ccc(OC(F)(F)F)cc1]

¹H NMR (500 MHz, DMSO-d6) δ ppm 6.89 (br. s., 1H) 7.01 (dd, J=3.51, 1.83 Hz, 1H) 7.37 (d, J=2.59 Hz, 1H) 7.41 (d, J=8.24 Hz, 1H) 7.46 (d, J=5.19 Hz, 1H) 7.51-7.57 (m, 1H) 7.64 (br. s., 1H) 7.77-7.85 (m, 1H) 8.20 (d, J=5.03 Hz, 1H) 11.69 (br. s., 1H) 11.83 (d, J=1.98 Hz, 1H). LCMS: m/z 387 [M+H]⁺. HRMS (ESI) calcd for C₁₉H₁₃F₃N₄O₂[M+H]⁺387.1064 found 387.106;

### 2-(1-benzothiophen-3-yl)-5-(1H-pyrrolo[2,3-b]pyridin-4-yl)-1H-pyrrole-3-carboxamide [R1=1H-pyrrolo[2,3-b]pyridin-4-yl, R2=1-benzothiophen-3-yl, R3=R4=R5=H] comp 35

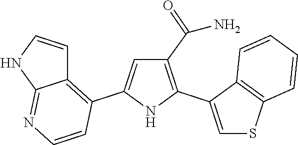 [SMILES: NC(=O)c1cc(-c2ccnc3[nH]ccc23)[nH]c1-c1csc2ccccc12]

¹H NMR (500 MHz, DMSO-d6) δ ppm 6.79-6.82 (m, 1H) 7.03-7.05 (m, 1H) 7.37-7.41 (m, 1H) 7.43-7.45 (m, 1H) 7.47-7.49 (m, 1H) 7.54-7.57 (m, 1H) 7.58-7.61 (m, 1H) 7.90-7.92 (m, 1H) 8.03-8.06 (m, 1H) 8.16-8.20 (m, 1H) 11.66-11.71 (m, 1H) 11.97-12.02 (m, 1H). LCMS: m/z 359 [M+H]⁺. HRMS (ESI) calcd for C₂₀H₁₄N₄OS [M+H]⁺359.0961 found 359.0962;

### 2-(2,3-dihydro-1,4-benzodioxin-6-yl)-5-(1H-pyrrolo[2,3-b]pyridin-4-yl)-1H-pyrrole-3-carboxamide [R1=1H-pyrrolo[2,3-b]pyridin-4-yl, R2=2,3-dihydro-1,4-benzodioxin-6-yl, R3=R4=R5=H] comp 36

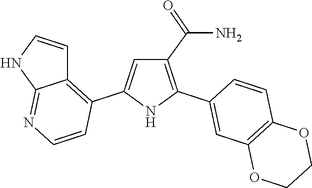 [SMILES: NC(=O)c1cc(-c2ccnc3[nH]ccc23)[nH]c1-c1ccc2c(c1)OCCO2]

¹H NMR (500 MHz, DMSO-d6) δ ppm 4.28 (s, 4H) 6.81 (br. s., 1H) 6.88 (d, J=8.39 Hz, 1H) 6.97 (dd, J=3.51, 1.83 Hz, 1H) 7.18 (dd, J=8.39, 2.14 Hz, 1H) 7.24 (d, J=2.14 Hz, 1H) 7.27 (d, J=2.74 Hz, 1H) 7.46 (d, J=5.19 Hz, 1H) 7.51-7.52 (m, 1H) 8.18 (d, J=5.03 Hz, 1H) 11.58-11.67 (m, 2H). LCMS: m/z 361 [M+H]⁺. HRMS (ESI) calcd for C₂₀H₁₅N₄O₃ [M+H]⁺ 361.1295 found 361.1295;

### 2-(4-fluoro-2-methylphenyl)-5-(1H-pyrrolo[2,3-b]pyridin-4-yl)-1H-pyrrole-3-carboxamide[R1=1H-pyrrolo[2,3-b]pyridin-4-yl, R2=4-fluoro-2-methylphenyl, R3=R4=R5=H] comp 37

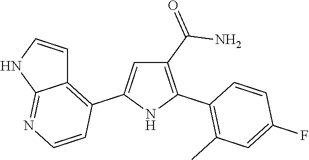 [SMILES: Cc1cc(F)ccc1-c1[nH]c(-c2ccnc3[nH]ccc23)cc1C(N)=O]

¹H NMR (500 MHz, DMSO-d6) δ ppm 2.20 (s, 3H) 6.73 (br. s., 1H) 7.01 (dd, J=3.51, 1.98 Hz, 1H) 7.06 (d, J=2.75 Hz, 1H) 7.15 (dd, J=10.22, 2.75 Hz, 1H) 7.33 (dd, J=8.46, 6.18 Hz, 1H) 7.36-7.40 (m, 1H) 7.43 (d, J=2.59 Hz, 1H) 7.52-7.54 (m, 1H) 8.15 (d, J=5.19 Hz, 1H) 11.66 (br. s., 1H) 11.81 (d, J=1.98 Hz, 1H). LCMS: m/z 335 [M+H]⁺. HRMS (ESI) calcd for C₁H₁₅FN₄O[M+H]⁺ 335.1303 found 335.13;

### 2-(2-fluoro-4-methylphenyl)-4-iodo-5-(1H-pyrrolo[2,3-b]pyridin-4-yl)-1H-pyrrole-3-carboxamide (1) [R1=1H-pyrrolo[2,3-b]pyridin-4-yl, R2=2-fluoro-4-methylphenyl, R3=R4=H, R5=iodo] comp 38

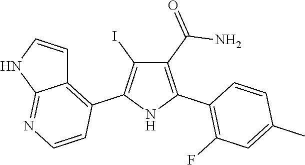 [SMILES: Cc1ccc(-c2[nH]c(-c3ccnc4[nH]ccc34)c(I)c2C(N)=O)c(F)c1]

¹H NMR (500 MHz, DMSO-d6) δ ppm 2.35 (s, 3H) 6.54 (dd, J=3.43, 1.91 Hz, 1H) 7.07 (d, J=7.78 Hz, 1H) 7.11 (d, J=11.44 Hz, 1H) 7.17 (br. s., 1H) 7.23 (br. s., 1H) 7.26 (d, J=5.03 Hz, 1H) 7.42 (t, J=7.85 Hz, 1H) 7.51-7.60 (m, 1H) 8.29 (d, J=5.03 Hz, 1H) 11.78 (br. s., 1H) 11.99 (s, 1H). LCMS: m/z 461 [M+H]⁺. HRMS (ESI) calcd for C₁₉H₁₄FlN₄O [M+H]⁺ 461.0269 found 461.0263;

### 2-(2-fluoro-4-methylphenyl)-4-bromo-5-(1H-pyrrolo[2,3-b]pyridin-4-yl)-1H-pyrrole-3-carboxamide[R1=1H-pyrrolo[2,3-b]pyridin-4-yl, R2=2-fluoro-4-methylphenyl, R3=R4=H, R5=bromo] comp 39

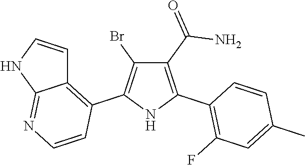 [SMILES: Cc1ccc(-c2[nH]c(-c3ccnc4[nH]ccc34)c(Br)c2C(N)=O)c(F)c1]

¹H NMR (500 MHz, DMSO-d6) δ ppm 2.36 (s, 3H) 6.58 (dd, J=3.43, 1.91 Hz, 1H) 7.08 (d, J=7.78 Hz, 1H) 7.12 (d, J=11.44 Hz, 1H) 7.23 (br. s., 1H) 7.25 (d, J=4.88 Hz, 1H) 7.28 (br. s., 1H) 7.44 (t, J=7.85 Hz, 1H) 7.53-7.58 (m, 1H) 8.28 (d, J=5.03 Hz, 1H) 11.79 (br. s., 1H) 12.00 (s, 1H). LCMS: m/z 413 [M+H]⁺. HRMS (ESI) calcd for C₁₉H₁₄FBrN₄O[M+H]⁺ 413.0408 found 413.0407;

### 4-ethyl-2-(2-fluoro-4-methylphenyl)-5-(1H-pyrrolo[2,3-b]pyridin-4-yl)-1H-pyrrole-3-carboxamide [R1=1H-pyrrolo[2,3-b]pyridin-4-yl, R2=4-fluoro-2-methylphenyl, R3=R4=R5=ethyl] comp 40

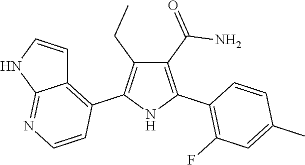 [SMILES: CCc1c(-c2ccnc3[nH]ccc23)[nH]c(-c2ccc(C)cc2F)c1C(N)=O]

¹H NMR (500 MHz, DMSO-d₆) δ ppm 1.05 (t, J=7.40 Hz, 3H) 2.35 (s, 3H) 2.71 (q, J=7.37 Hz, 2H) 6.51 (dd, J=3.43, 1.91 Hz, 1H) 6.95 (s, 2H) 7.02-7.11 (m, 3H) 7.44 (t, J=7.93 Hz, 1H) 7.50 (t, J=1.00 Hz, 1H) 8.24 (d, J=4.88 Hz, 1 H) 11.28 (s, 1H) 11.70 (br. s., 1H). LCMS: m/z 363 [M+H]⁺. HRMS (ESI) calcd for C₂₁H₁₉FN₄O[M+H]⁺ 363.1616 found 363.1601;

### 2-(2-fluoro-4-methylphenyl)-4-(propan-2-yl)-5-(1H-pyrrolo[2,3-b]pyridin-4-yl)-1H-pyrrole-3-carboxamide [R1=1H-pyrrolo[2,3-b]pyridin-4-yl, R2=4-fluoro-2-methylphenyl, R3=R4=H, R5=propan-2-yl] comp 41

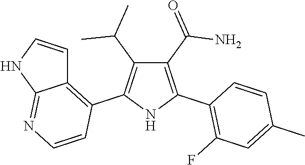 [SMILES: Cc1ccc(-c2[nH]c(-c3ccnc4[nH]ccc34)c(C(C)C)c2C(N)=O)c(F)c1]

¹H NMR (500 MHz, DMSO-d₆) d ppm 1.31 (d, J=7.02 Hz, 6H) 2.34 (s, 3H) 3.10 (spt, J=7.00 Hz, 1H) 6.51 (dd, J=3.36, 1.98 Hz, 1H) 6.89-7.14 (m, 5H) 7.39 (t, J=7.93 Hz, 1H) 7.51 (t, J=2.90 Hz, 1H) 8.24 (d, J=4.88 Hz, 1H) 11.18 (s, 1H) 11.71 (br. s., 1H). LCMS: m/z 377 [M+H]⁺. HRMS (ESI) calcd for C₂₂H₂₁FN₄O[M+H]⁺ 377.1772 found 377.1767;

### Example B

### Step 8

### 2-(2,4-dichlorophenyl)-1-{[2-(trimethylsilyl)ethoxy]methyl}-1H-pyrrole-3-carbonitrile (XIII)

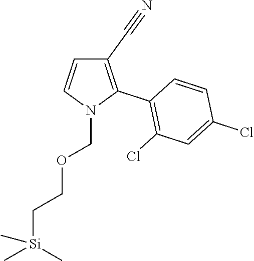 [SMILES: C[Si](C)(C)CCOCn1ccc(C#N)c1-c1ccc(Cl)cc1Cl]

To a solution of 2-(2,4-dichlorophenyl)-1H-pyrrole-3-carbonitrile (1 eq., 1.0 g, 4.22 mmol) in THE dry (20 ml) at T=0° C. NaH 60% in mineral oil (1.7 eq., 287 mg, 7.17 mmol) was added. The reaction mixture was stirred at T=0° C. for 20 minutes and SEMCl (1.8 eq., 1.35 ml, 7.59 mmol) was added. After 10 minutes at T=0° C. the reaction was warmed at room temperature and was stirred for 3 hours. Distilled water was added, and the product was extracted with DCM. The organic layer was washed with distilled water and brine, dried over anhydrous Na₂SO₄ and evaporated to dryness. The crude was purified by flash-chromatography (Hex/AcOEt 9/1) affording the title compound (light yellow oil, 1.45 g, Y=94%).

¹H NMR (500 MHz, DMSO-d₆) d ppm −0.13-−0.06 (m, 9H) 0.65-0.75 (m, 2H) 3.19-3.31 (m, 2H) 5.01-5.07 (m, 1H) 5.19-5.22 (m, 1H) 6.64-6.69 (m, 1H) 7.24-7.29 (m, 1H) 7.54-7.57 (m, 1H) 7.59-7.62 (m, 1H) 7.87-7.90 (m, 1H). LCMS: m/z 367 [M+H]⁺. HRMS (ESI) calcd for C₁₇H₂₁Cl₂N₂OSi [M+H]⁺ 367.0795 found 367.08;

Operating in an analogous way, but employing 2-(2-fluoro-4-methylphenyl)-1H-pyrrole-3-carbonitrile as starting material the following compound was obtained:

### 2-(2-fluoro-4-methylphenyl)-1-{[2-(trimethylsilyl)ethoxy]methyl}-1H-pyrrole-3-carbonitrile (XIII)

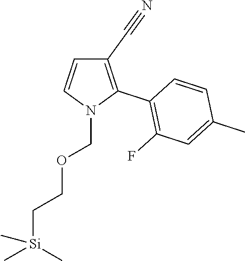

1H NMR (500 MHz, DMSO-d6) d ppm −0.15-−0.07 (m, 9H) 0.66-0.72 (m, 2H) 2.40 (s, 3H) 3.18-3.32 (m, 2H) 5.17 (s, 2H) 6.65 (d, J=3.20 Hz, 1H) 7.17-7.21 (m, 1H) 7.23-7.27 (m, 2H) 7.40 (t, J=7.78 Hz, 1H). LCMS: m/z 331 [M+H]⁺. HRMS (ESI) calcd for C₁₈H₂₄FN₂OSi [M+H]⁺ 331.1637 found 331.1628;

### 2-(2,4-dichlorophenyl)-1-(phenylsulfonyl)-1H-pyrrole-3-carbonitrile (XIII)

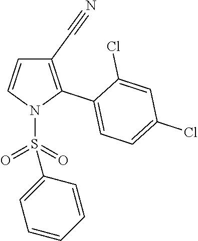 [SMILES: N#Cc1ccn(S(=O)(=O)c2ccccc2)c1-c1ccc(Cl)cc1Cl]

To a solution of 2-(2,4-dichlorophenyl)-1H-pyrrole-3-carbonitrile (1 eq., 1.0 g, 4.22 mmol) in DMF dry (10 ml) at T=0° C., NaH 60% in mineral oil (1.3 eq., 219 mg, 5.49 mmol) was added. The reaction mixture was stirred at T=0° C. for 20 minutes and Benzenesulfonyl chloridel (1.2 eq., 0.64 ml, 5.06 mmol) was added. The reaction was stirred at T=0° C. for 2.5 hours. Distilled water was added at T=0° C. and the product was extracted with AcOEt. The organic layer was washed with distilled water and brine, dried over anhydrous Na₂SO₄ and evaporated to dryness. The crude was purified by flash-chromatography (Hex/AcOEt 9/1-Hex/AcOEt 8/2) affording the title compound (white solid, 1.32 g, Y=83%). 1H NMR (500 MHz, DMSO-d6) δ ppm 6.96 (d, J=3.51 Hz, 1H) 7.37 (d, J=8.24 Hz, 1H) 7.58 (dd, J=8.24, 2.14 Hz, 1H) 7.60-7.66 (m, 4H) 7.78 (d, J=2.13 Hz, 1H) 7.81 (tt, J=5.85, 2.76 Hz, 1H) 7.85 (d, J=3.51 Hz, 1H). LCMS: m/z 378 [M+H]⁺. HRMS (ESI) calcd for C₁₇H₁₁Cl₂N₂O₂S [M+H]⁺ 378.9732 found 378.9744;

### Step 9

### 5-bromo-2-(2,4-dichlorophenyl)-1-{[2-(trimethylsilyl)ethoxy]methyl}-1H-pyrrole-3-carbonitrile (XIV)

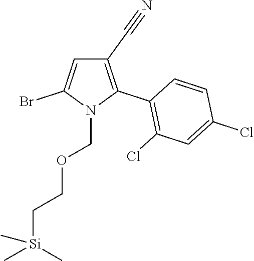 [SMILES: C[Si](C)(C)CCOCn1c(Br)cc(C#N)c1-c1ccc(Cl)cc1Cl]

To a solution of 2-(2,4-dichlorophenyl)-1-{[2-(trimethylsilyl)ethoxy]methyl}-1H-pyrrole-3-carbonitrile (1 eq., 1.44 g, 3.93 mmol) in MeOH (20 ml) and THE (10 ml) at T=0° C., 0.5 eq. of N-bromosuccinimide (349.5 mg, 1.96 mmol) was added. The reaction mixture was stirred at T=0° C. for 30 minutes and then 0.5 eq. of N-bromosuccinimide (349.5 mg, 1.96 mmol) was added. The reaction was stirred for 1 hour and 30 minutes at T=0° C. Distilled water was added and the product was extracted 3 times with AcOEt. The organic layer was washed with brine, dried over anhydrous Na₂SO₄ and evaporated to dryness. The crude was purified by flash-chromatography (Hex/AcOEt 95/5) affording the title compound (transparent—pale yellow oil, 1.44 g, Y=82%).

1H NMR (500 MHz, DMSO-d6) d ppm −0.15-−0.06 (m, 9H) 0.69 (t, J=8.16 Hz, 2H) 3.17-3.32 (m, 2H) 5.06 (d, J=11.44 Hz, 1H) 5.21 (d, J=11.44 Hz, 1H) 6.96 (s, 1H) 7.59-7.67 (m, 2H) 7.92 (d, J=1.98 Hz, 1H). LCMS: m/z 444 [M+H]⁺. HRMS (ESI) calcd for C₁₇H₂₀BrCl₂N₂OSi [M+H]⁺ 444.99 found 444.9915;

Operating in an analogous way, but employing 2-(2-fluoro-4-methylphenyl)-1-{[2-(trimethylsilyl)ethoxy]methyl}-1H-pyrrole-3-carbonitrile as starting material the following compound was obtained:

### 5-bromo-2-(2-fluoro-4-methylphenyl)-1-{[2-(trimethylsilyl)ethoxy]methyl}-1H-pyrrole-3-carbonitrile (XIV)

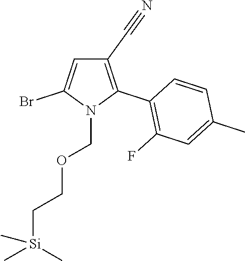

1H NMR (500 MHz, DMSO-d6) d ppm −0.13-−0.09 (m, 9H) 0.63-0.71 (m, 2H) 2.41 (s, 3H) 3.17-3.27 (m, 2H) 5.18 (br. s., 2H) 6.89-6.97 (m, 1H) 7.16-7.23 (m, 1H) 7.25-7.32 (m, 1H) 7.39-7.45 (m, 1H). LCMS: m/z 409 [M+H]⁺.

HRMS (ESI) calcd for C₁₈H₂₃BrFN₂OSi [M+H]⁺ 409.0742 found 409.0743;

### 5-bromo-2-(2,4-dichlorophenyl)-1-(phenylsulfonyl)-1H-pyrrole-3-carbonitrile (XIV)

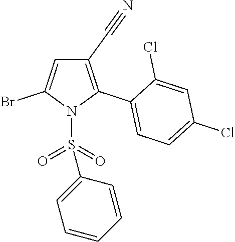 [SMILES: N#Cc1cc(Br)n(S(=O)(=O)c2ccccc2)c1-c1ccc(Cl)cc1Cl]

LCMS: m/z 454 [M+H]⁺. HRMS (ESI) calcd for C₁₇H₁₀BrCl₂N₂O₂S [M+H]⁺ 454.9018 found 454.9125;

### Step 10

### 2-(2,4-dichlorophenyl)-5-(1H-pyrazol-4-yl)-1-{[2-(trimethylsilyl)ethoxy]methyl}-1H-pyrrole-3-carbonitrile (XV)

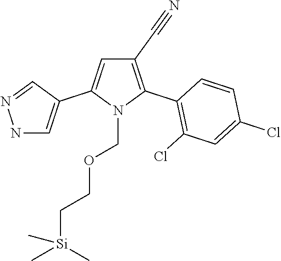 [SMILES: C[Si](C)(C)CCOCn1c(-c2cn[nH]c2)cc(C#N)c1-c1ccc(Cl)cc1Cl]

In a reactor, under argon atmosphere, 5-bromo-2-(2,4-dichlorophenyl)-1-{[2-(trimethylsilyl)ethoxy]methyl}-1H-pyrrole-3-carbonitrile (1 eq., 100 mg, 0.22 mmol), 4-(4,4,5,5-Tetramethyl-[1,3,2]dioxaborolan-2-yl)-1H-pyrazole (1.5 eq., 99 mg, 0.34 mmol), Na₂CO₃ (3 eq., 71 mg, 0.67 mmol), 1,4-dioxane degassed (4 ml) and distilled water degassed (1 ml) were added. After three cycles of vacuum/argon, the catalyst [1,1′-Bis (diphenylphosphino)ferrocene]dichloropalladium(II) (1:1) (0.1 eq., 18 mg, 0.022 mmol) was added. After three cycles of vacuum/argon the reaction mixture was heated at T=100° C. for 2 hour. Distilled water was added and the product was extracted with AcOEt (3 times). The organic layer was washed with distilled water and brine, dried over anhydrous Na₂SO₄ and evaporated to dryness. The crude was purified by flash-chromatography (DCM/MeOH 98/2) affording the title compound (yellow solid, 43 mg, Y=45%).

1H NMR (401 MHz, DMSO-d6) d ppm −0.12 (s, 9H) 0.65 (t, J=8.30 Hz, 2H) 1.28-1.28 (m, 1H) 3.10-3.21 (m, 2H) 4.99 (d, J=11.35 Hz, 1H) 5.21 (d, J=11.35 Hz, 1H) 6.78 (s, 1H) 7.59-7.63 (m, 1H) 7.63-7.67 (m, 1H) 7.78 (s, 1H) 7.91 (d, J=1.71 Hz, 1H) 8.02 (s, 1H). LCMS: m/z 433 [M+H]⁺. HRMS (ESI) calcd for C₂₀H₂₃Cl₂N₄OSi [M+H]⁺ 433.1013 found 433.1014;

Operating in an analogous way, but employing suitable substituted starting material the following compounds were obtained:

### 2-(2,4-dichlorophenyl)-5-(3-methyl-1H-pyrazol-4-yl)-1-{[2-(trimethylsilyl)ethoxy]methyl}-1H-pyrrole-3-carbonitrile (XV)

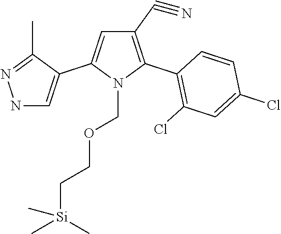 [SMILES: Cc1n[nH]cc1-c1cc(C#N)c(-c2ccc(Cl)cc2Cl)n1COCC[Si](C)(C)C]

1H NMR (500 MHz, DMSO-d6) d ppm −0.13 (s, 9H) 0.61 (t, J=8.31 Hz, 2H) 2.15-2.29 (m, 3H) 3.07 (q, J=7.83 Hz, 2H) 4.91 (d, J=11.29 Hz, 1H) 5.09 (d, J=10.98 Hz, 1H) 6.61-6.72 (m, 1H) 7.57-7.68 (m, 3H) 7.84-7.96 (m, 2H) 12.72-12.97 (m, 1H). LCMS: m/z 447 [M+H]⁺. HRMS (ESI) calcd for C₂₁H₂₅Cl₂N₄OSi [M+H]⁺ 447.1169 found 447.1173;

### 5-(2-amino-1,3-thiazol-4-yl)-2-(2,4-dichlorophenyl)-1-{[2-(trimethylsilyl)ethoxy]methyl}-1H-pyrrole-3-carbonitrile (XV)

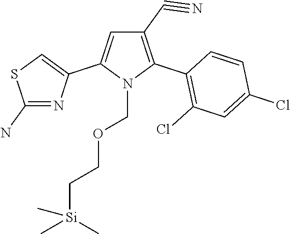 [SMILES: C[Si](C)(C)CCOCn1c(-c2csc(N)n2)cc(C#N)c1-c1ccc(Cl)cc1Cl]

LCMS: m/z 465 [M+H]⁺. HRMS (ESI) calcd for C₂₀H₂₃Cl₂N₄OSSi [M+H]⁺ 465.0733 found 465.0764;

### 2-(2,4-dichlorophenyl)-5-(1H-pyrrolo[2,3-b]pyridin-4-yl)-1-{[2-(trimethylsilyl)ethoxy]methyl}-1H-pyrrole-3-carbonitrile (XV)

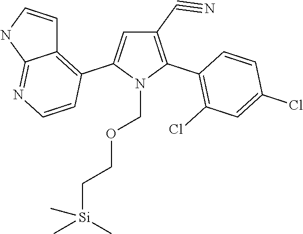 [SMILES: C[Si](C)(C)CCOCn1c(-c2ccnc3[nH]ccc23)cc(C#N)c1-c1ccc(Cl)cc1Cl]

LCMS: m/z 483 [M+H]⁺. HRMS (ESI) calcd for C₂₄H₂₅Cl₂N₄OSi [M+H]⁺ 483.1169 found 483.1185;

### 2-(2,4-dichlorophenyl)-5-(1H-pyrazolo[3,4-b]pyridin-4-yl)-1-{[2-(trimethylsilyl)ethoxy]methyl}-1H-pyrrole-3-carbonitrile (XV)

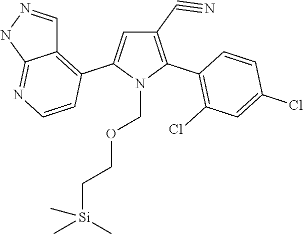 [SMILES: C[Si](C)(C)CCOCn1c(-c2ccnc3[nH]ncc23)cc(C#N)c1-c1ccc(Cl)cc1Cl]

LCMS: m/z 484 [M+H]⁺. HRMS (ESI) calcd for C₂₃H₂₄Cl₂N₅OSi [M+H]⁺ 484.1122 found 484.1157;

### 2-(2,4-dichlorophenyl)-5-[3-(trifluoromethyl)-1H-pyrazol-4-yl]-1-{[2-(trimethylsilyl)ethoxy]methyl}-1H-pyrrole-3-carbonitrile (XV)

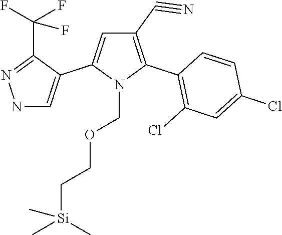 [SMILES: C[Si](C)(C)CCOCn1c(-c2c[nH]nc2C(F)(F)F)cc(C#N)c1-c1ccc(Cl)cc1Cl]

LCMS: m/z 501 [M+H]⁺. HRMS (ESI) calcd for C₂₁H₂₂Cl₂F₃N₄OSi [M+H]⁺ 501.0887 found 501.0894;

Operating in an analogous way, but employing 5-bromo-2-(2,4-dichlorophenyl)-1-(phenylsulfonyl)-1H-pyrrole-3-carbonitrile as starting material, the following compound was obtained:

### 5-(6-aminopyrimidin-4-yl)-2-(2,4-dichlorophenyl)-1-(phenylsulfonyl)-1H-pyrrole-3-carbonitrile (XV)

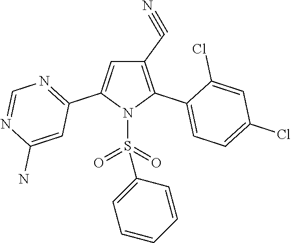 [SMILES: N#Cc1cc(-c2cc(N)ncn2)n(S(=O)(=O)c2ccccc2)c1-c1ccc(Cl)cc1Cl]

¹H NMR (500 MHz, DMSO-d6) δ ppm 6.60 (d, J=0.91 Hz, 1H) 7.12 (s, 1H) 7.14 (br. s., 1H) 7.41 (d, J=8.24 Hz, 1H) 7.56 (dd, J=8.31, 2.06 Hz, 1H) 7.58-7.63 (m, 2H) 7.76 (dd, J=8.46, 0.99 Hz, 2H) 7.78 (d, J=1.98 Hz, 1H) 7.80 (t, J=7.50 Hz, 0H) 8.40 (d, J=0.92 Hz, 1H). LCMS: m/z 470 [M+H]⁺. HRMS (ESI) calcd for C₂₁H₁₄Cl₂N₅O₂S [M+H]⁺ 470.0240 found 470.0256;

Operating in an analogous way, but employing 5-bromo-2-(2-fluoro-4-methylphenyl)-1-{[2-(trimethylsilyl)ethoxy]methyl}-1H-pyrrole-3-carbonitrile as starting material, the following compounds were obtained:

### 2-(2-fluoro-4-methylphenyl)-5-(1H-pyrazol-4-yl)-1-{[2-(trimethylsilyl)ethoxy]methyl}-1H-pyrrole-3-carbonitrile (XV)

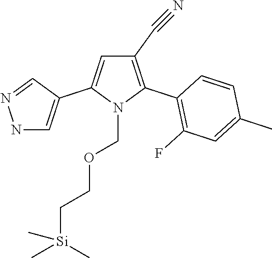 [SMILES: Cc1ccc(-c2c(C#N)cc(-c3cn[nH]c3)n2COCC[Si](C)(C)C)c(F)c1]

¹H NMR (500 MHz, DMSO-d6) d ppm −0.15-−0.12 (m, 9H) 0.59-0.69 (m, 2H) 2.41 (s, 3H) 3.09-3.19 (m, 2H) 5.14 (br. s., 2H) 6.76 (s, 1H) 7.22 (d, J=8.24 Hz, 1H) 7.29 (d, J=10.83 Hz, 1H) 7.43 (t, J=7.85 Hz, 1H) 7.77 (s, 1H) 8.01 (s, 1H) 13.14 (br. s., 1H). LCMS: m/z 397 [M+H]⁺. HRMS (ESI) calcd for C₂₁H₂FN₄OSi [M+H]⁺ 397.1855 found 397.1857;

### 5-(3,5-dimethyl-1H-pyrazol-4-yl)-2-(2-fluoro-4-methylphenyl)-1-{[2-(trimethylsilyl)ethoxy]methyl}-1H-pyrrole-3-carbonitrile (XV)

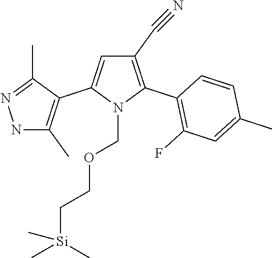 [SMILES: Cc1ccc(-c2c(C#N)cc(-c3c(C)n[nH]c3C)n2COCC[Si](C)(C)C)c(F)c1]

¹H NMR (500 MHz, DMSO-d6) d ppm −0.16 (s, 9H) 0.46-0.58 (m, 2H) 2.02 (s, 3H) 2.10 (s, 3H) 2.41 (s, 3H) 2.86-3.00 (m, 2H) 4.93 (s, 2H) 6.56 (s, 1H) 7.20 (d, J=7.63 Hz, 1H) 7.26 (d, J=10.98 Hz, 1H) 7.46 (t, J=7.78 Hz, 1H) 12.50 (s, 1H). LCMS: m/z 425 [M+H]⁺. HRMS (ESI) calcd for C₂₃H₃₀FN₄OSi [M+H]⁺ 425.2168 found 425.2172;

### 2-(2-fluoro-4-methylphenyl)-5-(1-methyl-1H-pyrazol-4-yl)-1-{[2-(trimethylsilyl)ethoxy]methyl}-1H-pyrrole-3-carbonitrile (XV)

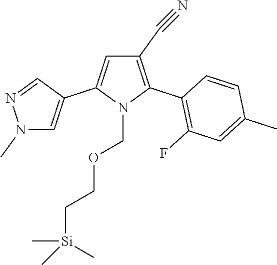

¹H NMR (500 MHz, DMSO-d6) d ppm −0.13 (s, 9H) 0.65 (dd, J=9.00, 7.63 Hz, 2H) 2.41 (s, 3H) 3.08-3.19 (m, 2H) 3.88 (s, 3H) 5.14 (br. s., 2H) 6.74 (s, 1H) 7.22 (dsxt, J=7.78, 0.80, 0.80, 0.80, 0.80, 0.80 Hz, 1H) 7.29 (d, J=10.98 Hz, 1H) 7.43 (t, J=7.85 Hz, 1H) 7.71 (d, J=0.61 Hz, 1H) 7.96 (s, 1H). LCMS: m/z 411 [M+H]⁺. HRMS (ESI) calcd for C₂₂H₂₅FN₄OSi [M+H]⁺ 411.2011 found 411.2011;

### 2-(2-fluoro-4-methylphenyl)-5-(3-methyl-1H-pyrazol-4-yl)-1-{[2-(trimethylsilyl)ethoxy]methyl}-1H-pyrrole-3-carbonitrile (XV)

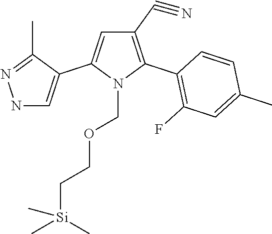 [SMILES: Cc1ccc(-c2c(C#N)cc(-c3c[nH]nc3C)n2COCC[Si](C)(C)C)c(F)c1]

LCMS: m/z 411 [M+H]⁺. HRMS (ESI) calcd for C₂₂H₂₅FN₄OSi [M+H]⁺ 411.2011 found 411.2035;

### 2-(2-fluoro-4-methylphenyl)-5-(1H-pyrrolo[2,3-b]pyridin-4-yl)-1-{[2-(trimethylsilyl)ethoxy]methyl}-1H-pyrrole-3-carbonitrile (XV)

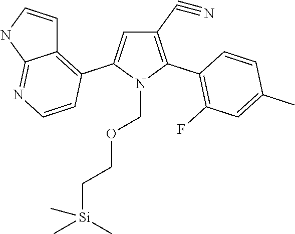 [SMILES: Cc1ccc(-c2c(C#N)cc(-c3ccnc4[nH]ccc34)n2COCC[Si](C)(C)C)c(F)c1]

¹H NMR (500 MHz, DMSO-d6) d ppm −0.27-−0.19 (m, 9H) 0.40-0.55 (m, 2H) 2.43 (s, 3H) 2.91-3.01 (m, 2H) 5.23 (br. s., 2H) 6.53 (dd, J=3.43, 1.91 Hz, 1H) 7.01 (s, 1H) 7.21-7.28 (m, 2H) 7.32 (d, J=10.83 Hz, 1H) 7.54-7.62 (m, 2H) 8.30 (d, J=4.88 Hz, 1H) 11.90 (br. s., 1H). LCMS: m/z 447 [M+H]⁺. HRMS (ESI) calcd for C₂₅H₂₈FN₄OSi [M+H]⁺ 447.2011 found 447.2020;

### Step 11

### 2-(2,4-dichlorophenyl)-5-(1H-pyrazol-4-yl)-1-{[2-(trimethylsilyl)ethoxy]methyl}-1H-pyrrole-3-carboxamide (XVI)

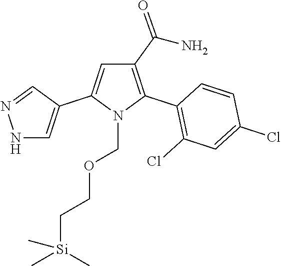 [SMILES: C[Si](C)(C)CCOCn1c(-c2cn[nH]c2)cc(C(N)=O)c1-c1ccc(Cl)cc1Cl]

To a solution of 2-(2,4-dichlorophenyl)-5-(1H-pyrazol-4-yl)-1-{[2-(trimethylsilyl)ethoxy]methyl}-1H-pyrrole-3-carbonitrile (1 eq., 220 mg, 0.51 mmol) in toluene (7 ml) acetaldoxime (20 eq., 0.62 ml, 10.16 mmol) and InCl₃ (0.1 eq., 11 mg, 0.051 mmol) were added. The reaction mixture was heated at T=100° C. for 4 hours. Distilled water was added and the product was extracted with AcOEt (3 times). The organic layer was washed with distilled water and brine, dried over anhydrous Na₂SO₄ and evaporated to dryness. The crude was purified by flash-chromatography (DCM/MeOH 95/5) affording the title compound (white solid, 106 g, Y=47%).

¹H NMR (500 MHz, DMSO-d6) d ppm −0.14-−0.10 (m, 8H) 0.59-0.69 (m, 2H) 3.05-3.17 (m, 2H) 4.81 (d, J=11.13 Hz, 1H) 5.06 (d, J=11.29 Hz, 1H) 6.71-6.76 (m, 2H) 7.18 (br. s., 1H) 7.38-7.42 (m, 1H) 7.47-7.50 (m, 2H) 7.70 (d, J=2.14 Hz, 1H) 7.92 (d, J=4.27 Hz, 1H) 13.05 (br. s., 1H). LCMS: m/z 451 [M+H]⁺. HRMS (ESI) calcd for C₂₀H₂₅Cl₂N₄O₂Si [M+H]⁺ 451.1119 found 451.1119;

Operating in an analogous way, but employing suitable substituted starting material the following compounds were obtained:

### 2-(2,4-dichlorophenyl)-5-(3-methyl-1H-pyrazol-4-yl)-1-{[2-(trimethylsilyl)ethoxy]methyl}-1H-pyrrole-3-carboxamide (XVI)

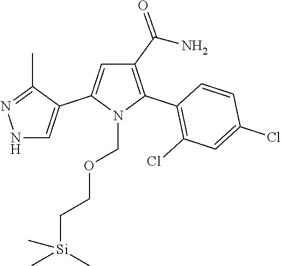 [SMILES: Cc1n[nH]cc1-c1cc(C(N)=O)c(-c2ccc(Cl)cc2Cl)n1COCC[Si](C)(C)C]

¹H NMR (500 MHz, DMSO-d6) d ppm −0.22-−0.04 (m, 9H) 0.61 (t, J=8.39 Hz, 2H) 2.26 (br. s., 3H) 2.89-3.13 (m, 2H) 4.73 (d, J=10.98 Hz, 1H) 4.95 (d, J=10.98 Hz, 1H) 6.48-6.69 (m, 1H) 6.73 (br. s., 1H) 7.22 (br. s., 1H) 7.40 (d, J=8.20 Hz, 1H) 7.47 (dd, J=8.20, 2.10 Hz, 1H) 7.68 (d, J=2.14 Hz, 1H) 12.46-13.03 (m, 1H). LCMS: m/z 465 [M+H]⁺. HRMS (ESI) calcd for C₂₁H₂₇Cl₂N₄O₂Si [M+H]⁺ 465.1275 found 465.1289;

### 5-(2-amino-1,3-thiazol-4-yl)-2-(2,4-dichlorophenyl)-1-{[2-(trimethylsilyl)ethoxy]methyl}-1H-pyrrole-3-carbonitrile (XVI)

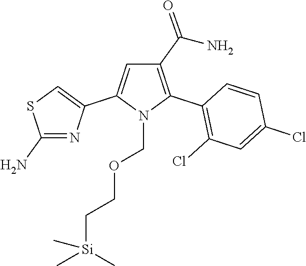 [SMILES: C[Si](C)(C)CCOCn1c(-c2csc(N)n2)cc(C(N)=O)c1-c1ccc(Cl)cc1Cl]

LCMS: m/z 483 [M+H]⁺. HRMS (ESI) calcd for C₂₀H₂₄Cl₂N₄O₂SSi [M+H]⁺ 483.0839 found 483.0852;

### 2-(2,4-dichlorophenyl)-5-(1H-pyrrolo[2,3-b]pyridin-4-yl)-1-{[2-(trimethylsilyl)ethoxy]methyl}-1H-pyrrole-3-carboxamide (XVI)

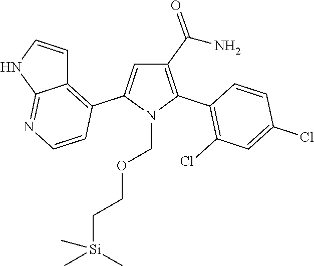 [SMILES: C[Si](C)(C)CCOCn1c(-c2ccnc3[nH]ccc23)cc(C(N)=O)c1-c1ccc(Cl)cc1Cl]

LCMS: m/z 501 [M+H]⁺. HRMS (ESI) calcd for C₂₄H₂₇Cl₂N₄O₂Si [M+H]⁺ 501.1275 found 501.1268.

### 2-(2,4-dichlorophenyl)-5-(1H-pyrazolo[3,4-b]pyridin-4-yl)-1-{[2-(trimethylsilyl)ethoxy]methyl}-1H-pyrrole-3-carboxamide (XVI)

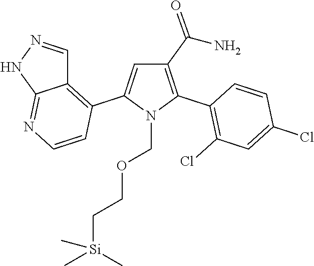 [SMILES: C[Si](C)(C)CCOCn1c(-c2ccnc3[nH]ncc23)cc(C(N)=O)c1-c1ccc(Cl)cc1Cl]

LCMS: m/z 502 [M+H]⁺. HRMS (ESI) calcd for C₂₃H₂₆Cl₂N₅O₂Si [M+H]⁺ 502.1227 found 502.1236.

### 2-(2,4-dichlorophenyl)-5-[3-(trifluoromethyl)-1H-pyrazol-4-yl]-1-{[2-(trimethylsilyl)ethoxy]methyl}-1H-pyrrole-3-carboxamide (XVI)

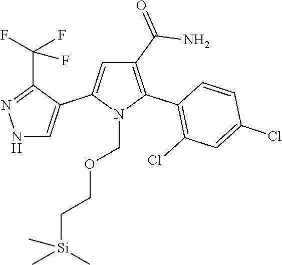 [SMILES: C[Si](C)(C)CCOCn1c(-c2c[nH]nc2C(F)(F)F)cc(C(N)=O)c1-c1ccc(Cl)cc1Cl]

LCMS: m/z 519 [M+H]⁺. HRMS (ESI) calcd for C₂₁H₂₄Cl₂F₃N₄O₂Si [M+H]⁺ 519.0992 found 519.0996.

### 5-(6-aminopyrimidin-4-yl)-2-(2,4-dichlorophenyl)-1-(phenylsulfonyl)-1H-pyrrole-3-carboxamide (XVI)

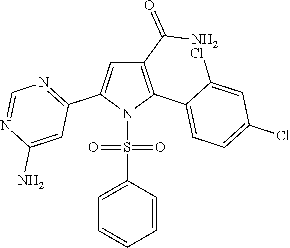 [SMILES: NC(=O)c1cc(-c2cc(N)ncn2)n(S(=O)(=O)c2ccccc2)c1-c1ccc(Cl)cc1Cl]

LCMS: m/z 488 [M+H]⁺. HRMS (ESI) calcd for C₂₁H₁₆Cl₂N₅O₃S [M+H]⁺ 488.0345 found 488.0352;

### 2-(2-fluoro-4-methylphenyl)-5-(1H-pyrazol-4-yl)-1-{[2-(trimethylsilyl)ethoxy]methyl}-1H-pyrrole-3-carboxamide (XVI)

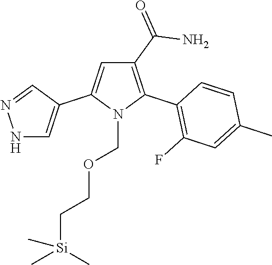 [SMILES: Cc1ccc(-c2c(C(N)=O)cc(-c3cn[nH]c3)n2COCC[Si](C)(C)C)c(F)c1]

LCMS: m/z 415 [M+H]⁺. HRMS (ESI) calcd for C₂₁H₂₅FN₄O₂Si [M+H]⁺ 415.1960 found 415.1975.

### 5-(3,5-dimethyl-1H-pyrazol-4-yl)-2-(2-fluoro-4-methylphenyl)-1-{[2-(trimethylsilyl)ethoxy]methyl}-1H-pyrrole-3-carboxamide (XVI)

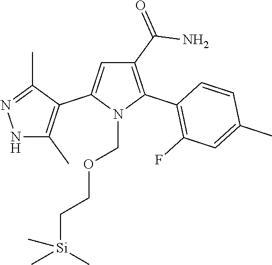 [SMILES: Cc1ccc(-c2c(C(N)=O)cc(-c3c(C)n[nH]c3C)n2COCC[Si](C)(C)C)c(F)c1]

¹H NMR (500 MHz, DMSO-d6) d ppm −0.15 (s, 9H) 0.42-0.60 (m, 2H) 2.05 (br. s., 6H) 2.36 (s, 3H) 2.89 (q, J=8.80 Hz, 2H) 4.56-4.95 (m, 2H) 6.48 (s, 1H) 6.69 (br. s., 1H) 7.02 (br. s., 1H) 7.03-7.08 (m, 2H) 7.26 (t, J=7.85 Hz, 1H) 12.38 (br. s., 1H). LCMS: m/z 443 [M+H]⁺. HRMS (ESI) calcd for C₂₃H₃₂FN₄O₂Si [M+H]⁺ 443.2273 found 443.2272;

### 2-(2-fluoro-4-methylphenyl)-5-(1-methyl-1H-pyrazol-4-yl)-1-{[2-(trimethylsilyl)ethoxy]methyl}-1H-pyrrole-3-carboxamide (XVI)

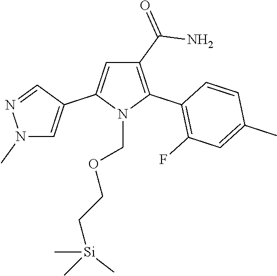

¹H NMR (500 MHz, DMSO-d6) d ppm −0.12 (s, 9H) 0.64 (t, J=8.31 Hz, 2H) 2.37 (s, 3H) 2.97-3.09 (m, 1H) 3.12 (t, J=8.24 Hz, 1H) 3.88 (s, 3H) 4.80-4.98 (m, 1H) 4.98-5.18 (m, 1H) 6.68 (s, 1H) 6.70 (br. s., 1H) 7.04 (br. s., 1H) 7.05-7.11 (m, 2H) 7.23 (t, J=7.85 Hz, 1H) 7.61 (d, J=0.61 Hz, 1H) 7.86 (s, 1H). LCMS: m/z 429 [M+H]⁺. HRMS (ESI) calcd for C₂₂H₃₀FN₄O₂Si [M+H]⁺ 429.2117 found 429.2116;

### 2-(2-fluoro-4-methylphenyl)-5-(3-methyl-1H-pyrazol-4-yl)-1-{[2-(trimethylsilyl)ethoxy]methyl}-1H-pyrrole-3-carboxamide (XVI)

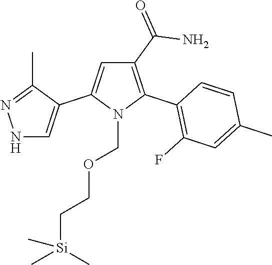 [SMILES: Cc1ccc(-c2c(C(N)=O)cc(-c3c[nH]nc3C)n2COCC[Si](C)(C)C)c(F)c1]

LCMS: m/z 429 [M+H]⁺. HRMS (ESI) calcd for C₂₂H₃₀FN₄O₂Si [M+H]⁺ 429.2117 found 429.2116;

### 2-(2-fluoro-4-methylphenyl)-5-(1H-pyrrolo[2,3-b]pyridin-4-yl)-1-{[2-(trimethylsilyl)ethoxy]methyl}-1H-pyrrole-3-carboxamide (XVI)

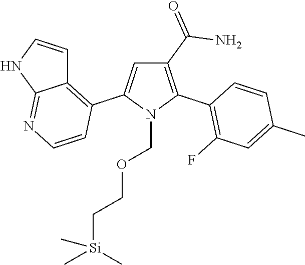 [SMILES: Cc1ccc(-c2c(C(N)=O)cc(-c3ccnc4[nH]ccc34)n2COCC[Si](C)(C)C)c(F)c1]

¹H NMR (500 MHz, DMSO-d6) d ppm −0.21-−0.17 (m, 9H) 0.55 (t, J=8.39 Hz, 2H) 2.38 (s, 3H) 2.87-3.10 (m, 2H) 4.95-5.22 (m, 2H) 6.66 (br. s., 0H) 6.80 (br. s., 1H) 7.09 (d, J=9.15 Hz, 2H) 7.24 (d, J=5.03 Hz, 1H) 7.31-7.41 (m, 2H) 7.54-7.58 (m, 1H) 8.26 (d, J=4.88 Hz, 1H) 11.80 (br. s., 1H). LCMS: m/z 465 [M+H]⁺. HRMS (ESI) calcd for C₂₅H₃₀FN₄O₂Si [M+H]⁺ 465.2117 found 465.2132;

### Step 12

### 5-(6-aminopyrimidin-4-yl)-2-(2,4-dichlorophenyl)-1H-pyrrole-3-carboxamide [R1=6-aminopyrimidin-4-yl, R2=2,4-dichlorophenyl, R3=R4=R5=H] comp 42

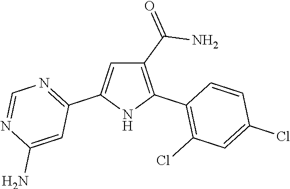 [SMILES: NC(=O)c1cc(-c2cc(N)ncn2)[nH]c1-c1ccc(Cl)cc1Cl]

To a solution of 5-(6-aminopyrimidin-4-yl)-2-(2,4-dichlorophenyl)-1-(phenylsulfonyl)-1H-pyrrole-3-carboxamide (1eq., 100 mg, 0.20 mmol) in THE (2.3 ml) and H₂O (1.8 ml), LiOH (4 eq., 0.8 mmol, 33.5 mg) was added. The reaction mixture was stirred at reflux for 4 hours. The solvent was partially removed under reduced pressure and HCl 2N was added to the mixture. The reaction was stirred at room temperature for 30 minutes and the title compound (white solid, 67 mg, Y=95%) was collected by filtration.

¹H NMR (500 MHz, DMSO-d6) δ ppm 6.65 (s, 1H) 6.76 (br. s., 1H) 6.91 (br. s., 2H) 7.29 (s, 1H) 7.35 (br. s., 1H) 7.40-7.47 (m, 2H) 7.65 (d, J=1.68 Hz, 1H) 8.34 (s, 1H) 12.09 (br. s., 1H). LCMS: m/z 348 [M+H]⁺. HRMS (ESI) calcd for C₁₅H₁₂Cl₂N₅O [M+H]⁺ 348.0414 found 348.0424;

### 2-(2,4-dichlorophenyl)-5-(1H-pyrazol-4-yl)-1H-pyrrole-3-carboxamide [R1=1H-pyrazol-4-yl, R2=2,4-dichlorophenyl, R3=R4=R5=H] comp 43

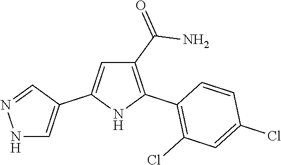 [SMILES: NC(=O)c1cc(-c2cn[nH]c2)[nH]c1-c1ccc(Cl)cc1Cl]

To a solution of 2-(2,4-dichlorophenyl)-5-(1H-pyrazol-4-yl)-1-{[2-(trimethylsilyl)ethoxy]methyl}-1H-pyrrole-3-carboxamide (1eq., 100 mg, 0.22 mmol) in DCM (4.9 ml), TFA (75 eq., 16.5 mmol, 1.26 ml) was added. The reaction mixture was stirred at room temperature for 5 hours. The solvent was removed under reduced pressure and the crude was treated three times with toluene. In the same reactor 2-(2,4-dichlorophenyl)-1-(hydroxymethyl)-5-(1H-pyrazol-4-yl)-1H-pyrrole-3-carboxamide was dissolved in EtOH 95% (8.5 ml) and ammonium hydroxide solution 30-32% (1.2 ml) was added. The reaction mixture was stirred at room temperature for 2 hours. The solvent was removed under reduced pressure and the solid was washed five times with distilled water (to remove CF3000—NH₄+) and three times with DCM/Et₂O 1/1 to achieve the title compound (pale-yellow solid, 31 mg, Y=44%).

¹H NMR (500 MHz, DMSO-d6) δ ppm 6.65 (br. s., 1H) 6.67 (d, J=2.6 Hz, 1H) 7.09 (br. s., 1H) 7.40-7.48 (m, 2H) 7.66 (dd, J=1.4, 0.8 Hz, 1H) 7.73 (br. s., 1H) 7.93 (br. s., 1H) 11.46 (s, 1H) 12.84 (br. s., 1H). LCMS: m/z 321 [M+H]⁺. HRMS (ESI) calcd for C₄H₁₁Cl₂N₄O [M+H]⁺ 321.0305 found 321.0305;

Operating in an analogous way, but employing suitable substituted starting material the following compounds were obtained:

### 2-(2,4-dichlorophenyl)-5-(3-methyl-1H-pyrazol-4-yl)-1H-pyrrole-3-carboxamide [R1=3-methyl-1H-pyrazol-4-yl, R2=2,4-dichlorophenyl, R3=R4=R5=H] comp 44

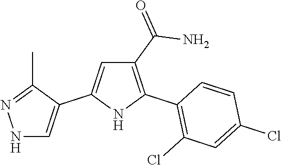 [SMILES: Cc1n[nH]cc1-c1cc(C(N)=O)c(-c2ccc(Cl)cc2Cl)[nH]1]

¹H NMR (500 MHz, DMSO-d6) δ ppm 6.61 (br. s., 1H) 6.64 (br. s., 2H) 7.20 (br. s., 1H) 7.44 (d, J=1.2 Hz, 2H) 7.65 (s, 1H) 11.15-11.47 (m, 1H) 12.30-12.76 (m, 1H). LCMS: m/z 335 [M+H]⁺. HRMS (ESI) calcd for C₁₅H₁₃Cl₂N₄O [M+H]⁺335.0461 found 335.0464;

### 5-(2-amino-1,3-thiazol-4-yl)-2-(2,4-dichlorophenyl)-1H-pyrrole-3-carboxamide [R1=2-amino-1,3-thiazol-4-yl, R2=2,4-dichlorophenyl, R3=R4=R5=H] comp 45

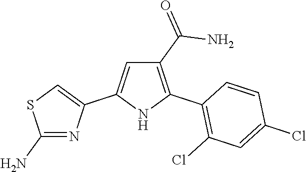 [SMILES: NC(=O)c1cc(-c2csc(N)n2)[nH]c1-c1ccc(Cl)cc1Cl]

¹H NMR (500 MHz, DMSO-d6) δ ppm 6.70 (br. s., 1H) 6.75 (s, 1H) 6.83 (d, J=2.59 Hz, 1H) 7.19 (br. s., 1H) 7.42-7.45 (m, 1H) 7.45-7.47 (m, 1H) 7.67 (d, J=1.98 Hz, 1H) 11.70 (br. s., 1H). LCMS: m/z 353 [M+H]⁺. HRMS (ESI) calcd for C₁₄H₁₁Cl₂N₄OS [M+H]⁺ 353.0025 found 353.0023;

### 2-(2,4-dichlorophenyl)-5-(1H-pyrrolo[2,3-b]pyridin-4-yl)-1H-pyrrole-3-carboxamide [R1=1H-pyrrolo[2,3-b]pyridin-4-yl, R2=2,4-dichlorophenyl, R3=R4=R5=H] comp 46

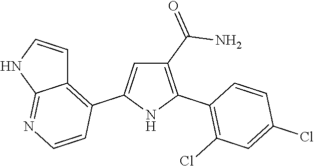 [SMILES: NC(=O)c1cc(-c2ccnc3[nH]ccc23)[nH]c1-c1ccc(Cl)cc1Cl]

¹H NMR (500 MHz, DMSO-d6) δ ppm 6.78 (br. s., 1H) 7.02 (dd, J=3.5, 1.8 Hz, 1H) 7.36 (d, J=5.2 Hz, 1H) 7.43-7.52 (m, 3H) 7.53-7.59 (m, 2H) 7.70 (d, J=2.0 Hz, 1H) 8.18 (d, J=5.0 Hz, 1H) 9.50-9.51 (m, 1H) 11.69 (br. s., 1H) 11.98 (d, J=1.7 Hz, 1H). LCMS: m/z 371 [M+H]⁺. HRMS (ESI) calcd for C₁₈H₁₂Cl₂N₄O [M+H]⁺ 371.0461 found 371.0462;

### 2-(2,4-dichlorophenyl)-5-(1H-pyrazolo[3,4-b]pyridin-4-yl)-1H-pyrrole-3-carboxamide [R1=1H-pyrazolo[3,4-b]pyridin-4-yl, R2=2,4-dichlorophenyl, R3=R4=R5=H] comp 47

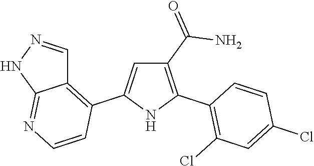 [SMILES: NC(=O)c1cc(-c2ccnc3[nH]ncc23)[nH]c1-c1ccc(Cl)cc1Cl]

¹H NMR (500 MHz, DMSO-d6) δ ppm 6.88 (br. s., 1H) 7.46 (d, J=4.9 Hz, 1H) 7.48-7.51 (m, 1H) 7.51-7.54 (m, 1H) 7.59 (br. s., 1H) 7.67 (d, J=2.6 Hz, 1H) 7.72 (d, J=2.0 Hz, 1H) 8.46 (d, J=4.9 Hz, 1H) 8.67 (d, J=1.1 Hz, 1H) 12.23 (br. s., 1H) 13.68 (s, 1H). LCMS: m/z 372 [M+H]⁺. HRMS (ESI) calcd for C₁₇H₁₂Cl₂N₅O [M+H]⁺ 372.0414 found 372.0417;

### 2-(2,4-dichlorophenyl)-5-[3-(trifluoromethyl)-1H-pyrazol-4-yl]-1H-pyrrole-3-carboxamide [R1=3-(trifluoromethyl)-1H-pyrazol-4-yl, R2=2,4-dichlorophenyl, R3=R4=R5=H] comp 48

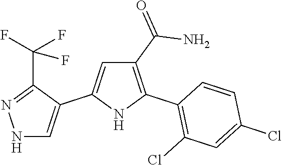 [SMILES: NC(=O)c1cc(-c2c[nH]nc2C(F)(F)F)[nH]c1-c1ccc(Cl)cc1Cl]

¹H NMR (500 MHz, DMSO-d6) δ ppm 6.68 (d, J=1.7 Hz, 2H) 7.22 (br. s., 1H) 7.41-7.50 (m, 2H) 7.67 (dd, J=1.7, 0.6 Hz, 1H) 8.15 (s, 1H) 11.53 (br. s., 1H) 13.61 (br. s., 1H). LCMS: m/z 389 [M+H]⁺. HRMS (ESI) calcd for C₁₅H₁₀Cl₂F₃N₄O[M+H]⁺ 389.0178 found 389.0184;

### 2-(2-fluoro-4-methylphenyl)-5-(1H-pyrazol-4-yl)-1H-pyrrole-3-carboxamide [R1=1H-pyrazol-4-yl, R2=2-fluoro-4-methylphenyl, R3=R4=R5=H] comp 49

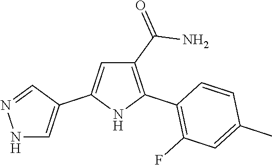 [SMILES: Cc1ccc(-c2[nH]c(-c3cn[nH]c3)cc2C(N)=O)c(F)c1]

¹H NMR (500 MHz, DMSO-d6) δ ppm 2.39 (s, 3H) 6.65 (br. s, 1H) 6.66 (d, J=2.6 Hz, 1H) 7.04 (br. s., 1H) 7.05-7.12 (m, 2H) 7.37 (t, J=7.9 Hz, 1H) 7.78 (s, 1H) 7.98 (s, 1H) 11.38 (br. s., 1H) 12.86 (br. s., 1H). LCMS: m/z 285 [M+H]⁺. HRMS (ESI) calcd for C₁₅H₁₄FN₄O[M+H]⁺ 285.1146 found 285.1147;

### 5-(3,5-dimethyl-1H-pyrazol-4-yl)-2-(2-fluoro-4-methylphenyl)-1H-pyrrole-3-carboxamide [R1=3,5-dimethyl-1H-pyrazol-4-yl, R2=2-fluoro-4-methylphenyl, R3=R4=R5=H] comp 50

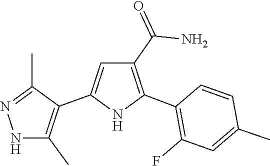 [SMILES: Cc1ccc(-c2[nH]c(-c3c(C)n[nH]c3C)cc2C(N)=O)c(F)c1]

¹H NMR (500 MHz, DMSO-d6) δ ppm 2.20 (br. s., 3H) 2.25 (br. s., 3H) 2.34 (s, 3H) 6.43 (d, J=2.7 Hz, 1H) 6.62 (br. s., 1H) 6.98-7.04 (m, 2H) 7.07 (br. s., 1H) 7.35 (t, J=8.0 Hz, 1H) 10.97 (d, J=2.0 Hz, 1H) 12.24 (s, 1H). LCMS: m/z 313 [M+H]⁺. HRMS (ESI) calcd for C₁₇H₁₈FN₄O[M+H]⁺ 313.1459 found 313.1456;

### 2-(2-fluoro-4-methylphenyl)-5-(1-methyl-1H-pyrazol-4-yl)-1H-pyrrole-3-carboxamide [R1=1-methyl-1H-pyrazol-4-yl, R2=2-fluoro-4-methylphenyl, R3=R4=R5=H] comp 51

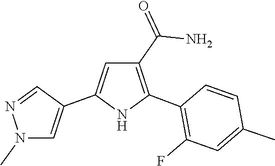

¹H NMR (500 MHz, DMSO-d6) δ ppm 2.35 (s, 3H) 3.84 (s, 3H) 6.60 (d, J=2.7 Hz, 1H) 6.63 (br. s., 1H) 7.01 (br. s., 1H) 7.03 (d, J=7.0 Hz, 1H) 7.05 (d, J=10.4 Hz, 1H) 7.33 (t, J=7.9 Hz, 1H) 7.68 (d, J=0.6 Hz, 1H) 7.88 (s, 1H) 11.37 (br. s., 1H). LCMS: m/z 299 [M+H]⁺. HRMS (ESI) calcd for C₁₆H₁₆FN₄O[M+H]⁺ 299.1303 found 299.1299;

### 2-(2-fluoro-4-methylphenyl)-5-(3-methyl-1H-pyrazol-4-yl)-1H-pyrrole-3-carboxamide [R1=3-methyl-1H-pyrazol-4-yl, R2=2-fluoro-4-methylphenyl, R3=R4=R5=H] comp 52

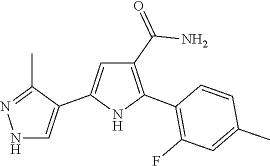 [SMILES: Cc1ccc(-c2[nH]c(-c3c[nH]nc3C)cc2C(N)=O)c(F)c1]

¹H NMR (500 MHz, DMSO-d6) δ ppm 2.35 (s, 6H) 6.51-6.68 (m, 2H) 7.00-7.07 (m, 2H) 7.10 (br. s., 1H) 7.33 (t, J=7.9 Hz, 1H) 7.75 (br. s., 1H) 11.23 (br. s., 1H) 12.18-12.82 (m, 1H). LCMS: m/z 299 [M+H]⁺. HRMS (ESI) calcd for C₁₅H₁₅FN₄O[M+H]⁺ 299.1303 found 299.1299;

### 2-(2-fluoro-4-methylphenyl)-5-(1H-pyrrolo[2,3-b]pyridin-4-yl)-1H-pyrrole-3-carboxamide [R1=1H-pyrrolo[2,3-b]pyridin-4-yl), R2=2-fluoro-4-methylphenyl, R3=R4=R5=H] comp 53

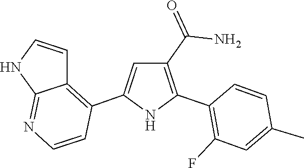 [SMILES: Cc1ccc(-c2[nH]c(-c3ccnc4[nH]ccc34)cc2C(N)=O)c(F)c1]

¹H NMR (500 MHz, DMSO-d₆) δ ppm 2.38 (s, 3H) 6.81 (br. s., 1H) 7.02-7.15 (m, 3H) 7.38-7.44 (m, 1H) 7.47-7.57 (m, 3H) 7.63 (t, J=2.90 Hz, 1H) 8.26 (d, J=5.49 Hz, 1H) 11.89-12.15 (m, 2H). LCMS: m/z 335 [M+H]⁺. HRMS (ESI) calcd for C₁₉H₁₅FN₄O[M+H]⁺ 335.1303 found 335.1302;

### Example C

### Step 13

### 1-(2-{[tert-butyl(dimethyl)silyl]oxy}ethyl)-2-(2,4-dichlorophenyl)-1H-pyrrole-3-carbonitrile (XVIII)

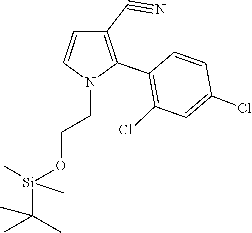 [SMILES: CC(C)(C)[Si](C)(C)OCCn1ccc(C#N)c1-c1ccc(Cl)cc1Cl]

To a solution of 2-(2,4-dichlorophenyl)-1H-pyrrole-3-carbonitrile (1 eq., 300 mg, 1.27 mmol) in THE dry (6 ml) at T=0° C. NaH 60% in mineral oil (1.8 eq., 92 mg, 2.29 mmol) was added. The reaction mixture was stirred at T=0° C. for 20 minutes and (2-Bromo-ethoxy)-tert-butyl-dimethyl-silane (XVII) (1.8 eq., 0.49 ml, 2.29 mmol) was added. After 10 minutes at T=0° C. the reaction was warmed at room temperature and was stirred for 6 hours. Distilled water was added and the product was extracted with DCM. The organic layer was washed with distilled water and brine, dried over anhydrous Na₂SO₄ and evaporated to dryness. The crude was purified by flash-chromatography (Hex/AcOEt 9/1) affording the title compound (light yellow oil, 336 mg, Y=67%).

¹H NMR (500 MHz, DMSO-d6) d ppm −0.14-−0.08 (m, 6H) 0.73-0.80 (m, 9H) 3.54-3.78 (m, 3H) 3.89-3.97 (m, 1H) 6.63 (d, J=3.05 Hz, 1H) 7.14 (d, J=3.05 Hz, 1H) 7.52 (d, J=8.39 Hz, 1H) 7.63 (dd, J=8.24, 2.14 Hz, 1H) 7.89 (d, J=2.14 Hz, 1H). LCMS: m/z 395 [M+H]⁺. HRMS (ESI) calcd for C₁₉H₂₅Cl₂N₂OSi [M+H]⁺ 395.1108 found 395.1110;

Operating in an analogous way, but employing suitable substituted starting material as intermediate (XVII) the following compounds were obtained:

### 2-(2,4-dichlorophenyl)-1-(3,3,3-trifluoropropyl)-1H-pyrrole-3-carbonitrile (XVIII)

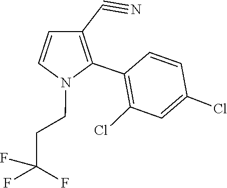 [SMILES: N#Cc1ccn(CCC(F)(F)F)c1-c1ccc(Cl)cc1Cl]

LCMS: m/z 333 [M+H]⁺. HRMS (ESI) calcd for C₁₄H₉Cl₂F₃N₂ [M+H]⁺ 333.0168 found 333.0172;

### 2-(2,4-dichlorophenyl)-1-methyl-1H-pyrrole-3-carbonitrile (XVIII)

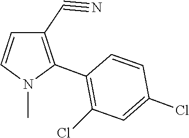 [SMILES: Cn1ccc(C#N)c1-c1ccc(Cl)cc1Cl]

LCMS: m/z 251 [M+H]⁺. HRMS (ESI) calcd for C₁₂H₈Cl₂N₂[M+H]⁺ 251.0137 found 251.0139;

### 2-(2,4-dichlorophenyl)-1-ethyl-1H-pyrrole-3-carbonitrile (XVIII)

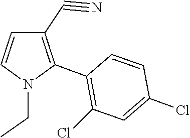 [SMILES: CCn1ccc(C#N)c1-c1ccc(Cl)cc1Cl]

LCMS: m/z 265 [M+H]⁺. HRMS (ESI) calcd for C₁₃H₁₀Cl₂N₂[M+H]⁺ 265.0294 found 265.0297;

### Step 14

### 5-bromo-2-(2,4-dichlorophenyl)-1-(2-hydroxyethyl)-1H-pyrrole-3-carbonitrile (XIX)

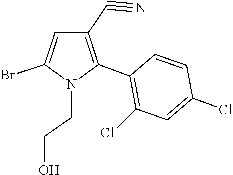 [SMILES: N#Cc1cc(Br)n(CCO)c1-c1ccc(Cl)cc1Cl]

To a solution of 1-(2-{[tert-butyl(dimethyl)silyl]oxy}ethyl)-2-(2,4-dichlorophenyl)-1H-pyrrole-3-carbonitrile (1 eq., 330 mg, 0.84 mmol) in MeOH (4 ml) and THE (2 ml) at T=0° C., 0.5 eq. of N-bromosuccinimide (74.5 mg, 0.42 mmol) was added. The reaction mixture was stirred at T=0° C. for 30 minutes and then 0.5 eq. of N-bromosuccinimide (74.5 mg, 0.42 mmol) was added. The reaction was stirred for 1 hour and 30 minutes at T=0° C. Distilled water was added and the product was extracted 3 times with AcOEt. The organic layer was washed with brine, dried over anhydrous Na₂SO₄ and evaporated to dryness. The crude was purified by flash-chromatography (Hex/AcOEt 95/5) affording the title compound (transparent—pale yellow oil, 360 mg, Y=48%).

LCMS: m/z 358 [M+H]⁺. HRMS (ESI) calcd for C₁₃H₁₀BrCl₂N₂O [M+H]⁺ 358.9348 found 358.9352;

Operating in an analogous way, but employing suitable substituted starting material the following compounds were obtained:

### 5-bromo-2-(2,4-dichlorophenyl)-1-(3,3,3-trifluoropropyl)-1H-pyrrole-3-carbonitrile (XIX)

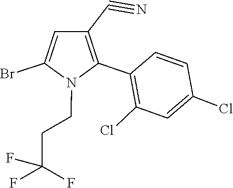 [SMILES: N#Cc1cc(Br)n(CCC(F)(F)F)c1-c1ccc(Cl)cc1Cl]

LCMS: m/z 410 [M+H]⁺. HRMS (ESI) calcd for C₁₄H₉BrCl₂F₃N₂ [M+H]⁺ 410.9273 found 410.9274;

### 5-bromo-2-(2,4-dichlorophenyl)-1-methyl-1H-pyrrole-3-carbonitrile (XIX)

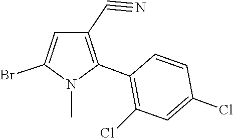 [SMILES: Cn1c(Br)cc(C#N)c1-c1ccc(Cl)cc1Cl]

LCMS: m/z 328 [M+H]⁺. HRMS (ESI) calcd for C₁₂H₇BrCl₂N₂[M+H]⁺ 328.9242 found 328.9240;

### 5-bromo-2-(2,4-dichlorophenyl)-1-ethyl-1H-pyrrole-3-carbonitrile (XIX)

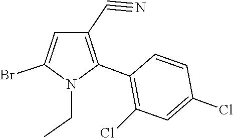 [SMILES: CCn1c(Br)cc(C#N)c1-c1ccc(Cl)cc1Cl]

LCMS: m/z 342 [M+H]⁺. HRMS (ESI) calcd for C₁₃H₉BrCl₂N₂[M+H]⁺ 342.9399 found 342.9398;

### Step 15

### 2-(2,4-dichlorophenyl)-1-(2-hydroxyethyl)-5-(1H-pyrrolo[2,3-b]pyridin-4-yl)-1H-pyrrole-3-carbonitrile (XX)

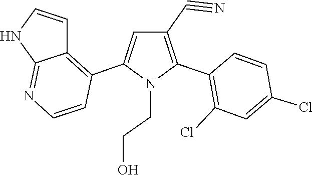 [SMILES: N#Cc1cc(-c2ccnc3[nH]ccc23)n(CCO)c1-c1ccc(Cl)cc1Cl]

In a reactor, under argon atmosphere, 5-bromo-2-(2,4-dichlorophenyl)-1-(2-hydroxyethyl)-1H-pyrrole-3-carbonitrile (1 eq., 140 mg, 0.39 mmol), 4-(4,4,5,5-Tetramethyl-[1,3,2]dioxaborolan-2-yl)-1H-pyrrolo[2,3-b]pyridine (1.5 eq., 142 mg, 0.58 mmol), Na₂CO₃ (3 eq., 124 mg, 1.17 mmol), 1,4-dioxane degassed (4 ml) and distilled water degassed (1 ml) were added. After three cycles of vacuum/argon, the catalyst [1,1′-Bis(diphenylphosphino)ferrocene]dichloropalladium(II) (1:1) (0.1 eq., 32 mg, 0.039 mmol) was added. After three cycles of vacuum/argon the reaction mixture was heated at T=100° C. for 3 hours. Distilled water was added, and the product was extracted with AcOEt (3 times). The organic layer was washed with distilled water and brine, dried over anhydrous Na₂SO₄ and evaporated to dryness. The crude was purified by flash-chromatography (AcOEt) affording the title compound (yellow solid, 110 mg, Y=71%).

¹H NMR (500 MHz, DMSO-d6) δ ppm 3.01 (quind, J=11.74, 11.74, 11.74, 11.74, 6.10 Hz, 2H) 3.77 (dt, J=14.03, 6.86 Hz, 1H) 3.99-4.07 (m, 1H) 4.63 (t, J=5.57 Hz, 1H) 6.42-6.44 (m, 1H) 6.89 (s, 1H) 7.18 (d, J=5.0 Hz, 1H) 7.59-7.61 (m, 1H) 7.67-7.69 (m, 1H) 7.74-7.72 (m, 1H) 7.94 (d, J=2.0 Hz, 1H) 8.30 (d, J=5.0 Hz, 1H) 11.91 (br. s., 1H). LCMS: m/z 397 [M+H]⁺. HRMS (ESI) calcd for C₂₀H₁₅Cl₂N₄O [M+H]⁺ 397.0618 found 397.0623;

Operating in an analogous way, but employing suitable substituted starting material the following compounds were obtained:

### 2-(2,4-dichlorophenyl)-5-(1H-pyrrolo[2,3-b]pyridin-4-yl)-1-(3,3,3-trifluoropropyl)-1H-pyrrole-3-carbonitrile (XX)

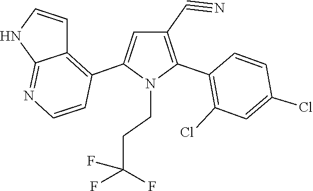 [SMILES: N#Cc1cc(-c2ccnc3[nH]ccc23)n(CCC(F)(F)F)c1-c1ccc(Cl)cc1Cl]

LCMS: m/z 449 [M+H]⁺. HRMS (ESI) calcd for C₂₁H₁₄Cl₂F₃N₄ [M+H]⁺ 449.0542 found 449.0541;

### 2-(2,4-dichlorophenyl)-1-methyl-5-(1H-pyrrolo[2,3-b]pyridin-4-yl)-1H-pyrrole-3-carbonitrile (XX)

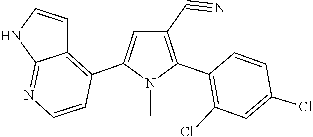 [SMILES: Cn1c(-c2ccnc3[nH]ccc23)cc(C#N)c1-c1ccc(Cl)cc1Cl]

LCMS: m/z 367 [M+H]⁺. HRMS (ESI) calcd for C₁₉H₁₂Cl₂N₄[M+H]⁺ 367.0512 found 367.0513;

### 2-(2,4-dichlorophenyl)-1-ethyl-5-(1H-pyrrolo[2,3-b]pyridin-4-yl)-1H-pyrrole-3-carbonitrile (XX)

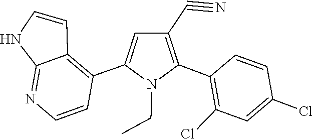 [SMILES: CCn1c(-c2ccnc3[nH]ccc23)cc(C#N)c1-c1ccc(Cl)cc1Cl]

LCMS: m/z 381 [M+H]⁺. HRMS (ESI) calcd for C₂₀H₁₄Cl₂N₄[M+H]⁺ 381.0668 found 381.0670;

### Step 15

### 2-(2,4-dichlorophenyl)-1-(2-hydroxyethyl)-5-(1H-pyrrolo[2,3-b]pyridin-4-yl)-1H-pyrrole-3-carboxamide [R1=1H-pyrrolo[2,3-b]pyridin-4-yl), R2=2,4-dichlorophenyl, R3=R5=H, R4=2-hydroxyethyl,] comp 54

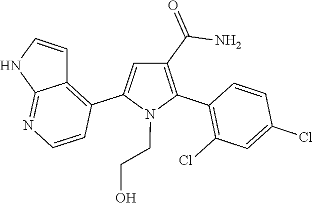 [SMILES: NC(=O)c1cc(-c2ccnc3[nH]ccc23)n(CCO)c1-c1ccc(Cl)cc1Cl]

To a solution of 2 2-(2,4-dichlorophenyl)-1-(2-hydroxyethyl)-5-(1H-pyrrolo[2,3-b]pyridin-4-yl)-1H-pyrrole-3-carbonitrile (1 eq., 100 mg, 0.25 mmol) in toluene (3.6 ml) acetaldoxime (20 eq., 0.307 ml, 5.05 mmol) and InCl₃ (0.1 eq., 5.6 mg, 0.025 mmol) were added. The reaction mixture was heated at T=100° C. for 1 hours. Distilled water was added and the product was extracted with AcOEt (3 times). The organic layer was washed with distilled water and brine, dried over anhydrous Na₂SO₄ and evaporated to dryness. The crude was purified by flash-chromatography (DCM/EtOH 95/5) affording the title compound (white solid, 21 mg, Y=20%).

¹H NMR (500 MHz, DMSO-d6) δ ppm 2.94-3.07 (m, 2H) 3.68 (dt, J=14.2, 7.1 Hz, 1H) 3.88-3.97 (m, 1H) 6.53 (dd, J=3.4, 1.8 Hz, 1H) 6.74 (br. s., 1H) 6.93 (s, 1H) 7.18 (d, J=5.0 Hz, 1H) 7.33 (br. s., 1H) 7.50-7.52 (m, 1H) 7.53-7.55 (m, 1H) 7.57-7.60 (m, 1H) 7.75 (d, J=2.0 Hz, 1H) 8.30 (d, J=5.0 Hz, 1H) 11.91 (br. s., 1H). LCMS: m/z 415 [M+H]⁺. HRMS (ESI) calcd for C₂₀H₁₇Cl₂N₄O₂ [M+H]⁺ 415.0723 found 415.0722;

Operating in an analogous way, but employing suitable substituted starting material the following compounds were obtained:

### 2-(2,4-dichlorophenyl)-5-(1H-pyrrolo[2,3-b]pyridin-4-yl)-1-(3,3,3-trifluoropropyl)-1H-pyrrole-3-carboxamide [R1=1H-pyrrolo[2,3-b]pyridin-4-yl), R2=2,4-dichlorophenyl, R3=R5=H, R4=3,3,3-trifluoropropyl] comp 55

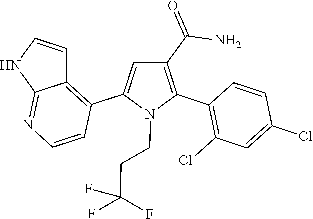 [SMILES: NC(=O)c1cc(-c2ccnc3[nH]ccc23)n(CCC(F)(F)F)c1-c1ccc(Cl)cc1Cl]

¹H NMR (500 MHz, DMSO-d6) δ ppm 1.99-2.17 (m, 2H) 3.88-3.97 (m, 1H) 4.11-4.20 (m, 1H) 6.51 (dd, J=3.4, 1.9 Hz, 1H) 6.80 (br. s., 1H) 6.97 (s, 1H) 7.14 (d, J=4.9 Hz, 1H) 7.39 (br. s., 1H) 7.53-7.56 (m, 1H) 7.57-7.61 (m, 2H) 7.79 (d, J=2.0 Hz, 1H) 8.30 (d, J=4.9 Hz, 1H) 11.86 (br. s., 1H). LCMS: m/z 467 [M+H]⁺. HRMS (ESI) calcd for C₂₁H₁₆Cl₂F₃N₄O[M+H]⁺ 467.0648 found 467.0656;

### 2-(2,4-dichlorophenyl)-1-methyl-5-(1H-pyrrolo[2,3-b]pyridin-4-yl)-1H-pyrrole-3-carboxamide [R1=1H-pyrrolo[2,3-b]pyridin-4-yl), R2=2,4-dichlorophenyl, R3=R5=H, R4=methyl] comp 56

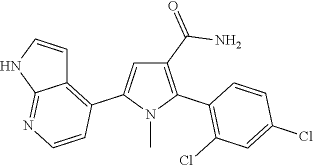 [SMILES: Cn1c(-c2ccnc3[nH]ccc23)cc(C(N)=O)c1-c1ccc(Cl)cc1Cl]

¹H NMR (500 MHz, DMSO-d₆) δ ppm 3.30 (s, 1H) 6.60 (dd, J=3.43, 1.91 Hz, 1H) 6.74 (br. s., 1H) 7.02 (s, 1H) 7.11 (d, J=4.88 Hz, 1H) 7.38 (br. s., 1H) 7.50-7.52 (m, 2H) 7.56 (t, J=1.00 Hz, 1H) 7.76 (s, 1H) 8.27 (d, J=4.88 Hz, 1H) 11.81 (br. s., 1H). LCMS: m/z 385 [M+H]⁺. HRMS (ESI) calcd for C₁₉H₁₄Cl₂N₄O [M+H]⁺ 385.0618 found 385.0616;

### 2-(2,4-dichlorophenyl)-1-ethyl-5-(1H-pyrrolo[2,3-b]pyridin-4-yl)-1H-pyrrole-3-carboxamide [R1=1H-pyrrolo[2,3-b]pyridin-4-yl), R2=2,4-dichlorophenyl, R3=R5=H, R4=ethyl] comp 57

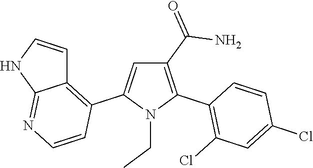 [SMILES: CCn1c(-c2ccnc3[nH]ccc23)cc(C(N)=O)c1-c1ccc(Cl)cc1Cl]

¹H NMR (500 MHz, DMSO-d₆) δ ppm 0.73 (t, J=7.17 Hz, 3H) 3.61-3.90 (m, 2H) 6.51 (dd, J=3.36, 1.83 Hz, 1H) 6.72 (br. s., 1H) 6.93 (s, 1H) 7.10 (d, J=4.88 Hz, 1H) 7.32 (br. s., 1H) 7.48-7.59 (m, 3H) 7.75 (d, J=1.83 Hz, 1H) 8.28 (d, J=5.03 Hz, 1H) 11.83 (br. s., 1H). LCMS: m/z 399 [M+H]⁺. HRMS (ESI) calcd for C₂₀H₁₆Cl₂N₄O [M+H]⁺ 399.0774 found 399.0767.

1. A compound of formula (I): 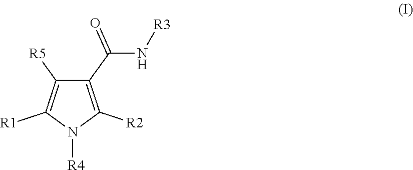
wherein:
R1 is a heteroaryl group selected from the group consisting of: 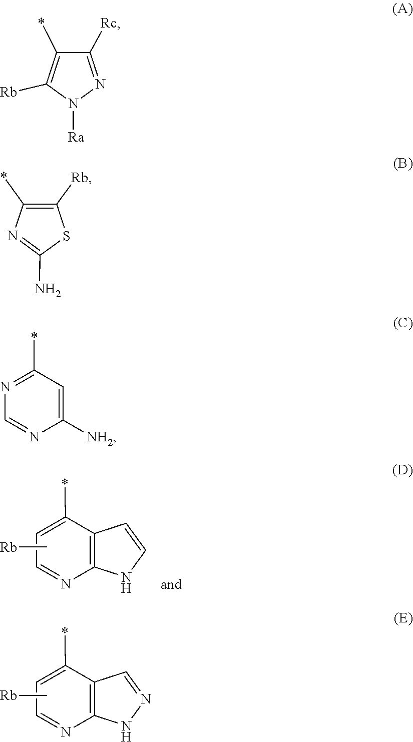
wherein:
Ra, Rb and Rc are independently hydrogen, an optionally substituted straight or branched (C₁-C₆) alkyl or an optionally substituted straight or branched (C₂-C₆) alkenyl;
R2 is a substituted aryl or a substituted heteroaryl ring bearing from one up to three substituents selected from halogen, nitro, amino, (C₁-C₆) alkyl amino, aminocarbonyl, an optionally substituted straight or branched (C₁-C₆) alkyl, an optionally substituted straight or branched (C₁-C₆) alkoxy, an optionally substituted straight or branched polyfluorinated (C₁-C₆) alkyl and optionally substituted straight or branched polyfluorinated (C₁-C₆) alkoxy;
provided that, 2,5-disubstituted phenyl group is excluded;
R3 is hydrogen, an optionally substituted straight or branched (C₁-C₄) alkyl, an optionally substituted (C₃-C₆) cycloalkyl group or an optionally substituted (C₅-C₆) heterocyclyl group;
R4 is hydrogen, an optionally substituted straight or branched (C₁-C₆) alkyl or an optionally substituted straight or branched (C₂-C₆) alkenyl; and
R5 is hydrogen, halogen or an optionally substituted straight or branched (C₁-C₃) alkyl;
or a pharmaceutically acceptable salt thereof.

2. A compound of the formula (I) according to claim 1 or a pharmaceutically acceptable salt thereof wherein:
Ra, Rb and Rc are independently hydrogen or an optionally substituted straight or branched (C1-C6) alkyl; and
R2 is a 2,4-disubstituted phenyl, 4,6-disubstituted pyridin-3-yl, 2,6-disubstituted pyridin-3-yl or 3,5-disubstituted pyridine-2-yl.

3. A compound of the formula (I) according to claim 2 or a pharmaceutically acceptable salt thereof wherein:
R2 is a 2,4-disubstituted phenyl;
R3 is hydrogen or an optionally substituted straight or branched (C₁-C₄) alkyl chain; and
R5 is hydrogen.

4. A compound of the formula (I) according to claim 3 or a pharmaceutically acceptable salt thereof wherein:
R4 is hydrogen.

5. A compound (cpd) according to claim 1 or a pharmaceutically acceptable salt thereof, selected from the group consisting of:
2-(3-chloro-2-fluorophenyl)-5-(1H-pyrrolo[2,3-b]pyridin-4-yl)-1H-pyrrole-3-carboxamide (comp 1);
2-(4-chloro-2-fluorophenyl)-5-(1H-pyrrolo[2,3-b]pyridin-4-yl)-1H-pyrrole-3-carboxamide (comp 2);
2-(2-chloro-4-fluorophenyl)-5-(1H-pyrrolo[2,3-b]pyridin-4-yl)-1H-pyrrole-3-carboxamide (comp 3);
2-(2,4-difluorophenyl)-5-(1H-pyrrolo[2,3-b]pyridin-4-yl)-1H-pyrrole-3-carboxamide (comp 4);
2-[2-chloro-4-(trifluoromethyl)phenyl]-5-(1H-pyrrolo [2,3-b]pyridin-4-yl)-1H-pyrrole-3-carboxamide (comp 5);
2-(2,3-difluorophenyl)-5-(1H-pyrrolo[2,3-b]pyridin-4-yl)-1H-pyrrole-3-carboxamide (comp 6);
2-(2,3-dichlorophenyl)-5-(1H-pyrrolo[2,3-b]pyridin-4-yl)-1H-pyrrole-3-carboxamide (comp 7);
2-[4-methyl-2-(trifluoromethyl)phenyl]-5-(1H-pyrrolo[2,3-b]pyridin-4-yl)-1H-pyrrole-3-carboxamide (comp 8);
2-(2-chloro-4-methylphenyl)-5-(1H-pyrrolo [2,3-b]pyridin-4-yl)-1H-pyrrole-3-carboxamide (comp 9);
2-(2,3-difluoro-4-methylphenyl)-5-(1H-pyrrolo[2,3-b]pyridin-4-yl)-1H-pyrrole-3-carboxamide (comp 10);
2-[2-methyl-4-(trifluoromethyl)phenyl]-5-(1H-pyrrolo[2,3-b]pyridin-4-yl)-1H-pyrrole-3-carboxamide (comp 11);
2-(2-fluoro-3-methoxyphenyl)-5-(1H-pyrrolo [2,3-b]pyridin-4-yl)-1H-pyrrole-3-carboxamide (comp 12);
2-(2-chloro-3-fluorophenyl)-5-(1H-pyrrolo[2,3-b]pyridin-4-yl)-1H-pyrrole-3-carboxamide (comp 13);
2-(2-fluoro-3-methylphenyl)-5-(1H-pyrrolo[2,3-b]pyridin-4-yl)-1H-pyrrole-3-carboxamide (comp 14);
2-[2-methyl-3-(trifluoromethyl)phenyl]-5-(1H-pyrrolo[2,3-b]pyridin-4-yl)-1H-pyrrole-3-carboxamide (comp 15);
2-[4-methoxy-2-(trifluoromethyl)phenyl]-5-(1H-pyrrolo[2,3-b]pyridin-4-yl)-1H-pyrrole-3-carboxamide (comp 16);
2-[2-chloro-4-(difluoromethoxy)phenyl]-5-(1H-pyrrolo [2,3-b]pyridin-4-yl)-1H-pyrrole-3-carboxamide (comp 17);
2-(3,4-dichlorophenyl)-5-(1H-pyrrolo[2,3-b]pyridin-4-yl)-1H-pyrrole-3-carboxamide (comp 18);
2-(3,4-difluorophenyl)-5-(1H-pyrrolo[2,3-b]pyridin-4-yl)-1H-pyrrole-3-carboxamide (comp 19);
2-(3-ethoxy-2-fluorophenyl)-5-(1H-pyrrolo[2,3-b]pyridin-4-yl)-1H-pyrrole-3-carboxamide (comp 20);
2-(4-methyl-3-nitrophenyl)-5-(1H-pyrrolo[2,3-b]pyridin-4-yl)-1H-pyrrole-3-carboxamide (comp 21);
2-(3-carbamoyl-4-fluorophenyl)-5-(1H-pyrrolo[2,3-b]pyridin-4-yl)-1H-pyrrole-3-carboxamide (comp 22);
2-(2-fluoro-4-methylphenyl)-N-[2-(pyrrolidin-1-yl)ethyl]-5-(1H-pyrrolo[2,3-b]pyridin-4-yl)-1H-pyrrole-3-carboxamide (comp 23);
N-[2-(dimethylamino)ethyl]-2-(2-fluoro-4-methylphenyl)-5-(1H-pyrrolo[2,3-b]pyridin-4-yl)-1H-pyrrole-3-carboxamide (comp 24);
2-(2-fluoro-4-methylphenyl)-N-[2-(morpholin-4-yl)ethyl]-5-(1H-pyrrolo[2,3-b]pyridin-4-yl)-1H-pyrrole-3-carboxamide (comp 25);
N-[(1S,2R)-2-aminocyclohexyl]-2-(2-fluoro-4-methylphenyl)-5-(1H-pyrrolo[2,3-b]pyridin-4-yl)-1H-pyrrole-3-carboxamide (comp 26);
2-(2-fluoro-4-methylphenyl)-N-(furan-2-ylmethyl)-5-(1H-pyrrolo[2,3-b]pyridin-4-yl)-1H-pyrrole-3-carboxamide (comp 27);
N-(fluoroethyl)-2-(2-fluoro-4-methylphenyl)-5-(1H-pyrrolo [2,3-b]pyridin-4-yl)-1H-pyrrole-3-carboxamide (comp 28);
2-(2-fluoro-4-methylphenyl)-N-[2-(methylamino)ethyl]-5-(1H-pyrrolo[2,3-b]pyridin-4-yl)-1H-pyrrole-3-carboxamide (comp 29);
comp2-(2-fluoro-4-methylphenyl)-N-(1-methylpiperidin-4-yl)-5-(1H-pyrrolo[2,3-b]pyridin-4-yl)-1H-pyrrole-3-carboxamide (comp 30);
2-(dibenzo[b,d]thiophen-4-yl)-5-(1H-pyrrolo[2,3-b]pyridin-4-yl)-1H-pyrrole-3-carboxamide (comp 31);
2-(4-methylnaphthalen-1-yl)-5-(1H-pyrrolo [2,3-b]pyridin-4-yl)-1H-pyrrole-3-carboxamide (comp 32);
2-(3-fluorophenyl)-5-(1H-pyrrolo[2,3-b]pyridin-4-yl)-1H-pyrrole-3-carboxamide (comp 33);
5-(1H-pyrrolo[2,3-b]pyridin-4-yl)-2-[4-(trifluoromethoxy)phenyl]-1H-pyrrole-3-carboxamide (comp 34);
2-(2,3-dihydro-1,4-benzodioxin-6-yl)-5-(1H-pyrrolo[2,3-b]pyridin-4-yl)-1H-pyrrole-3-carboxamide (comp 36);
2-(4-fluoro-2-methylphenyl)-5-(1H-pyrrolo[2,3-b]pyridin-4-yl)-1H-pyrrole-3-carboxamide (comp 37);
2-(2-fluoro-4-methylphenyl)-4-iodo-5-(1H-pyrrolo [2,3-b]pyridin-4-yl)-1H-pyrrole-3-carboxamide (comp 38);
4-bromo-2-(2-fluoro-4-methylphenyl)-5-(1H-pyrrolo[2,3-b]pyridin-4-yl)-1H-pyrrole-3-carboxamide (comp 39);
4-ethyl-2-(2-fluoro-4-methylphenyl)-5-(1H-pyrrolo [2,3-b]pyridin-4-yl)-1H-pyrrole-3-carboxamide (comp 40);
2-(2-fluoro-4-methylphenyl)-4-(propan-2-yl)-5-(1H-pyrrolo[2,3-b]pyridin-4-yl)-1H-pyrrole-3-carboxamide (comp 41);
5-(6-aminopyrimidin-4-yl)-2-(2,4-dichlorophenyl)-1H-pyrrole-3-carboxamide (comp 42) 2-(2,4-dichlorophenyl)-5-(1H-pyrazol-4-yl)-1H-pyrrole-3-carboxamide (comp 43);
2-(2,4-dichlorophenyl)-5-(3-methyl-1H-pyrazol-4-yl)-1H-pyrrole-3-carboxamide (comp 44);
5-(2-amino-1,3-thiazol-4-yl)-2-(2,4-dichlorophenyl)-1H-pyrrole-3-carboxamide (comp 45);
2-(2,4-dichlorophenyl)-5-(1H-pyrrolo[2,3-b]pyridin-4-yl)-1H-pyrrole-3-carboxamide (comp 46);
2-(2,4-dichlorophenyl)-5-(1H-pyrazolo[3,4-b]pyridin-4-yl)-1H-pyrrole-3-carboxamide (comp 47);
2-(2,4-dichlorophenyl)-5-[3-(trifluoromethyl)-1H-pyrazol-4-yl]-1H-pyrrole-3-carboxamide (comp 48);
2-(2-fluoro-4-methylphenyl)-5-(1H-pyrazol-4-yl)-1H-pyrrole-3-carboxamide (comp 49);
5-(3,5-dimethyl-1H-pyrazol-4-yl)-2-(2-fluoro-4-methylphenyl)-1H-pyrrole-3-carboxamide (comp 50);
2-(2-fluoro-4-methylphenyl)-5-(1-methyl-1H-pyrazol-4-yl)-1H-pyrrole-3-carboxamide (comp 51);
2-(2-fluoro-4-methylphenyl)-5-(3-methyl-1H-pyrazol-4-yl)-1H-pyrrole-3-carboxamide (comp 52);
2-(2-fluoro-4-methylphenyl)-5-(1H-pyrrolo[2,3-b]pyridin-4-yl)-1H-pyrrole-3-carboxamide (comp 53);
2-(2,4-dichlorophenyl)-1-(2-hydroxyethyl)-5-(1H-pyrrolo[2,3-b]pyridin-4-yl)-1H-pyrrole-3-carboxamide (comp 54);
2-(2,4-dichlorophenyl)-5-(1H-pyrrolo[2,3-b]pyridin-4-yl)-1-(3,3,3-trifluoropropyl)-1H-pyrrole-3-carboxamide (comp 55);
2-(2,4-dichlorophenyl)-1-methyl-5-(1H-pyrrolo[2,3-b]pyridin-4-yl)-1H-pyrrole-3-carboxamide (comp 56); and
2-(2,4-dichlorophenyl)-1-ethyl-5-(1H-pyrrolo [2,3-b]pyridin-4-yl)-1H-pyrrole-3-carboxamide (comp 57).

6. A process for the preparation of a compound of formula (I) or a pharmaceutical acceptable salt thereof, as defined in claim 1, said process comprises:
Step 1) metal-catalyzed coupling reaction of a compound of formula (II): 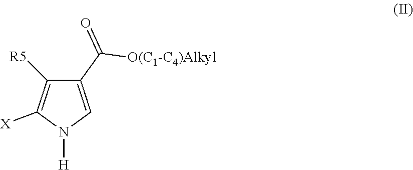
wherein R5 is hydrogen or an optionally substituted straight or branched (C₁-C₃) alkyl and X is halogen, with a suitable organoboronic acid derivative of formula (III): 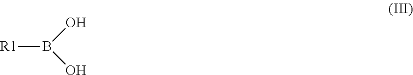
wherein R1 is as defined in claim 1;
Step 2) halogenation of the so obtained compound of formula (IV): 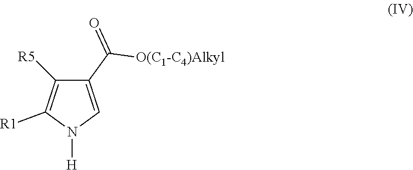
wherein R1 and R5 are as defined above in Step 1, thus to obtain a compound of formula (V): 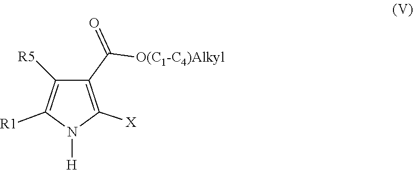
wherein R1 and R5 are as defined above in Step 1 and X is halogen;
Step 3) metal-catalyzed coupling reaction of a compound of formula (V) with a suitable organoboronic acid derivative of formula (VI): 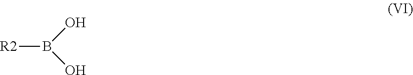
wherein R2 is as defined in claim 1, so to obtain a compound of formula (VII): 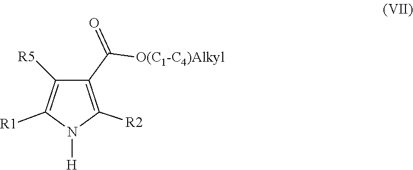
wherein R1 and R5 are as defined above in Step 1 and R2 is as defined in Step 3;
a compound of formula (VII) obtained from Step 3, wherein R5 is hydrogen, can be converted in another compound of formula (VII), wherein R5 is halogen (X), according to conv.1 below:
conv. 1) 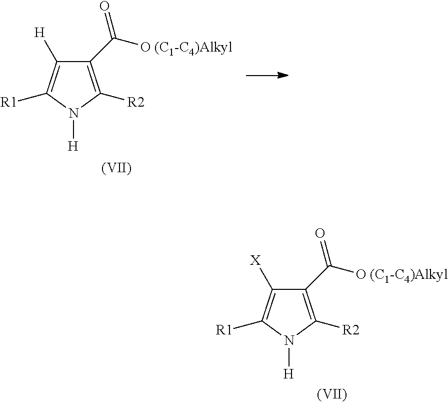
following the conditions already reported in Step 2 above;
Step 4) protection of the compound of formula (VII) obtained from Step 3 or conv.1:
wherein R1 and R2 are as defined above in Step 1 and Step 3, respectively and R5 is hydrogen, halogen or an optionally substituted straight or branched (C₁-C₃) alkyl, by reaction with the suitable protecting group, so to obtain the carboxylic ester of formula (VIII): 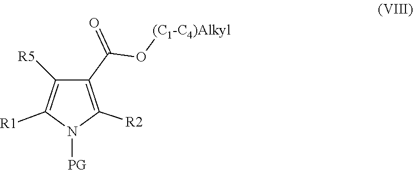
wherein R1, R2 and R5 are as defined above and PG is a protecting group such as trimethylsilylethoxymethyl (SEM), tert-Butyloxycarbonyl (BOC) or benzenesulfonyl;
Step 5) hydrolysis under basic condition of the carboxylic ester of formula (VIII), so to yield the carboxylic acid of formula (IX): 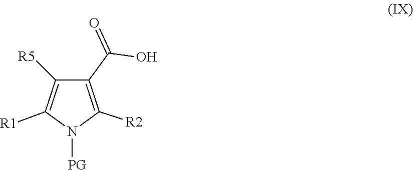
wherein R1, R2, R5 and PG are as defined above in Step 4;
Step 6) amidation of the intermediate of formula (IX) by reaction with an amine derivative of formula (X):
H₂N—R3 (X)
wherein R3 is as defined in claim 1;
Step 7) deprotection of the resultant compound of formula (XI): 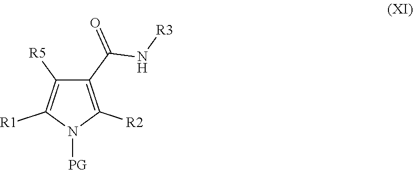
wherein R1, R2, R5 and PG are as defined above under Step 5 and R3 is as defined under Step 6, to give a compound of formula (I): 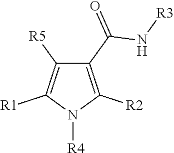
wherein R1, R2, R3 are as defined in claim 1 and R4 is hydrogen; or
an intermediate compound of formula (VIII) wherein R5 is halogen, can be converted into an intermediate of formula (XI), according to a process comprising the following conversions:
conv. 2) converting a compound of formula (VIII): 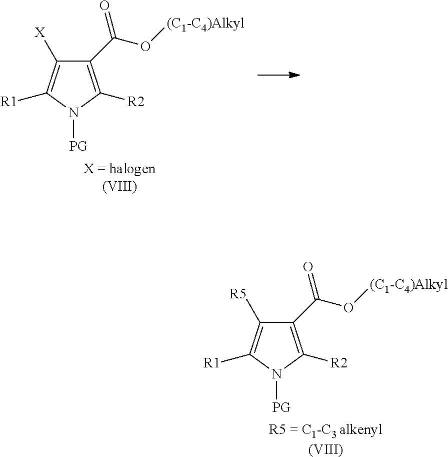
wherein R1 and R2 are as defined in claim 1, into a compound of formula (VIII) wherein R5 is an optionally substituted straight or branched (C₁-C₃) alkenyl chain, following the condition known in the art for palladium-catalyzed reaction, already reported in Step 3;
reacting the so obtained compound (VIII): 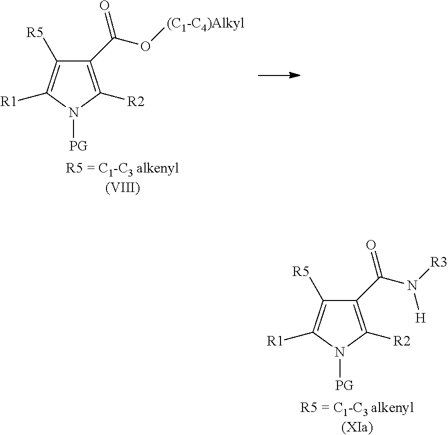
under conditions reported in step 5 and 6, thus to obtain a compound (XIa) wherein R1, R2 and R5 are as defined above;
conv. 3) converting the so obtained compound of formula (XIa): 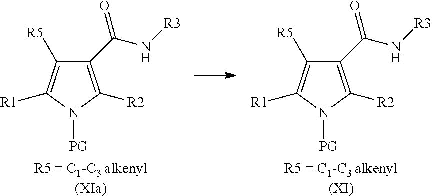
into a compound of formula (XI) wherein R1, R2 and R3 are as defined in claim 1 and R5 is an optionally substituted straight or branched (C₁-C₃) alkyl, following the condition known in the art for reduction of double bond/hydrogenation; or
alternatively, the compound of formula (I) wherein R1 and R2, are as defined in claim 1, R3 and R4 are hydrogen and R5 is hydrogen or an optionally substituted straight or branched (C₁-C₃) alkyl, can be prepared accordingly to a process comprising the following steps:
Step 8) protection of a compound of formula (XII): 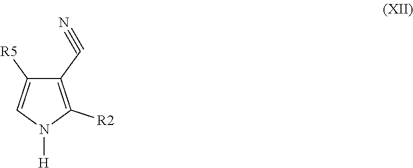
wherein R2 is as defined in claim 1 and R5 is hydrogen or an optionally substituted straight or branched (C₁-C₃) alkyl;
Step 9) halogenation of the so obtained compound of formula (XIII): 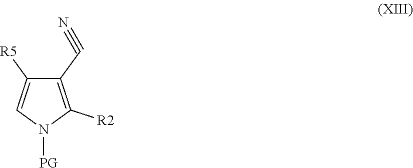
wherein R2 and R5 are as defined above under Step 8 and PG is a protecting group such as SEM, BOC or benzenesulfonyl;
Step 10) metal-catalyzed coupling reaction of the resultant compound of formula (XIV): 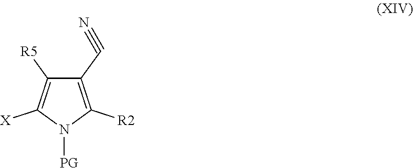
wherein R2, R5 and PG are as defined above in Step 9 and X is halogen, with a suitable organoboronic acid derivative of formula (III): 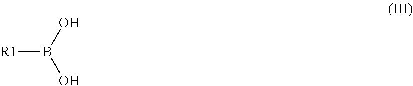
wherein R1 is as defined in claim 1;
Step 11) hydrolysis of the so obtained compound of formula (XV): 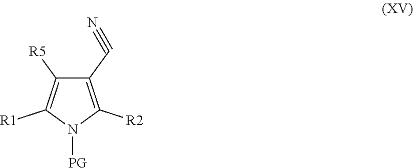
wherein R1, R2, R5 and PG are as defined above in Step 10, thus to yield the corresponding amide intermediate of formula (XVI); 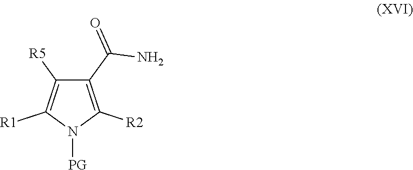
Step 12) deprotection of the compound of formula (XVI), to give a compound of formula (I): 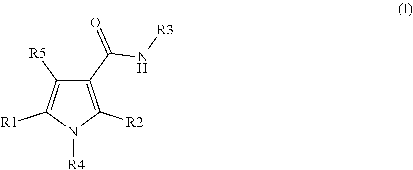
wherein R1 and R2 are as defined in claim 1, R3 and R4 are hydrogen and R5 is as defined above under Step 8; or
alternatively, the compound of formula (I) wherein R1, R2 and R4 are as defined in claim 1, R3 is hydrogen and R5 is hydrogen or an optionally substituted straight or branched (C₁-C₄) alkyl chain, can be prepared accordingly to a process comprising the following steps:
Step 13) reaction of a derivative of formula (XII): 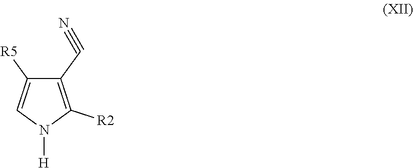
wherein R2 is as defined in claim 1 and R5 is hydrogen or an optionally substituted straight or branched (C₁-C₄) alkyl with a halo derivative of formula (XVII):
R4-X (XVII)
wherein R4 is an optionally substituted straight or branched C₁-C₆ alkyl, or an optionally substituted straight or branched C₂-C₆ alkenyl and X is halogen, in the presence of a base or by addition of a metal catalyst;
Step 14) halogenation of the so obtained compound of formula (XVIII): 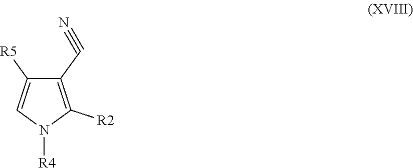
wherein R2, R4 and R5 are as defined above in Step 13;
Step 15) metal-catalyzed coupling reaction of the resultant compound of formula (XIX): 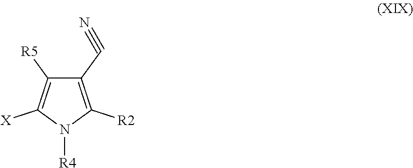
wherein X is halogen and R2, R3, and R4 are as defined above in Step 13, with a suitable organoboronic acid derivative of formula (III): 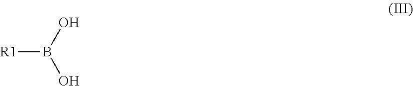
wherein R1 is as defined in claim 1;
Step 16) hydrolysis of the so obtained intermediate of formula (XX): 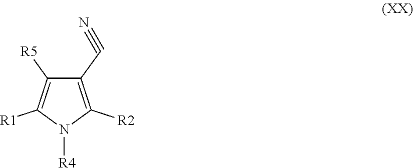
to give a compound of formula (I): 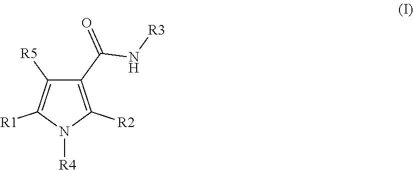
wherein R1 and R2 are as defined in claim 1, R3 is hydrogen, R4 is as defined in Step 13 and R5 is hydrogen or an optionally substituted straight or branched (C₁-C₄) alkyl chain.

7.-10. (canceled)

11. A pharmaceutical composition comprising a compound of formula (I) or a pharmaceutically acceptable salt thereof, as defined in claim 1, and at least one pharmaceutically acceptable excipient, carrier or diluent.

12. A pharmaceutical composition according to claim 11 further comprising one or more chemotherapeutic agents.

13. An in vitro method for inhibiting Cdc7 kinase activity which comprises contacting the said protein with an effective amount of a compound of formula (I) as defined in claim 1 or a pharmaceutically acceptable salt thereof.

14. A product or kit comprising a compound of formula (I) or a pharmaceutically acceptable salt thereof, as defined in claim 1, and one or more chemotherapeutic agents, as a combined preparation for simultaneous, separate or sequential use in anticancer therapy.

15. (canceled)

16. A method of treating a disease caused by and/or associated with dysregulated Cdc7 kinase activity, which comprises administering to a mammal, in need thereof, an effective amount of a compound of formula (I) as defined in claim 1 or a pharmaceutically salt thereof.

17. The method, according to claim 16-, wherein the mammal in need thereof is a human.

18. The method according to claim 16 or 17, wherein the disease is selected from a group consisting of cancer and cell proliferative disorders.

19. The method according to claim 18 wherein said cancer is selected from the group consisting of carcinomas, such as
bladder, breast, kidney, liver, colon, lung, including small cell lung cancer, esophagus, gall-bladder, ovary, pancreas, stomach, cervix, prostate, head and neck and skin, including squamous cell carcinoma; hematopoietic tumors of lymphoid lineage including leukemia, acute lymphocitic leukemia, acute lymphoblastic leukemia, B-cell lymphoma, angioimmunoblastic T-cell lymphoma, Hodgkin's lymphoma, non-Hodgkin's lymphoma, hairy cell lymphoma mantle cell lymphoma and Burkitt's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including glioma, glioblastoma, glioblastoma multiforme, astrocytoma, oligodendroglioma, paraglioma, neuroblastoma, and schwannomas; and other tumors, including melanoma, seminoma, teratocarcinoma, osteosarcoma, xerodermapigmentosum, keratoxanthoma, thyroid cancers, such as papillary thyroid carcinoma and medullary thyroid carcinoma, Kaposi's sarcoma, chondrosarcoma, cholangiocarcinoma, head and neck tumors.

20. The method according to claim 16 in combination with radiation therapy, target therapy, immunotherapy or a chemotherapy regimen.

21.-24. (canceled)
